(19) United States
(12) Patent Application Publication
(10) Pub. No.: US 2023/0183687 A1
(43) Pub. Date: Jun. 15, 2023

(54) MODIFIED GUIDE RNAS FOR NEISSERIA MENINGITIDIS CAS9

(71) Applicant: UNIVERSITY OF MASSACHUSETTS, Boston, MA (US)

(72) Inventors: Erik Joseph Sontheimer, Auburndale, MA (US); Jonathan Kenneth Watts, Worcester, MA (US); Anastasia Khvorova, Westborough, MA (US); Nathan Bamidele, Shrewsbury, MA (US)

(21) Appl. No.: 17/964,611

(22) Filed: Oct. 12, 2022

Related U.S. Application Data
(60) Provisional application No. 63/255,315, filed on Oct. 13, 2021.

(51) Int. Cl. C12N 15/11; C12N 9/22; C12N 15/90

(52) U.S. Cl. CPC C12N 15/11; C12N 15/111; C12N 9/22; C12N 15/907; C12N 2310/20; C12N 2800/80; C12N 2310/314

(57) ABSTRACT

Chemically modified Neisseria meningitidis (Nme) crRNAs, tracrRNAs, and sgRNAs are provided. Methods of using the Nme crRNAs, tracrRNAs, and sgRNAs for genome editing with a Nme CRISPR nuclease and kits for performing the same are also provided.

### RELATED APPLICATIONS

This application claims the benefit of U.S. Provisional Application No. 63/255,315, filed Oct. 13, 2021, the content of which is incorporated by reference in its entirety for all purposes.

### STATEMENT REGARDING FEDERALLY SPONSORED RESEARCH OR DEVELOPMENT

This invention was made with government support under Grant Nos. TR002668, GM113686, and GM143879 awarded by the National Institutes of Health. The Government has certain rights in the invention.

### SEQUENCE LISTING

The content of the electronically submitted Sequence Listing in XML format (Name: 734838_UM9-272_ST26.xml; Size: 9,253,628 bytes; and Date of Creation: Feb. 27, 2023) is incorporated herein by reference in its entirety.

### FIELD OF THE INVENTION

This disclosure relates to compositions and methods of modified guide RNAs for CRISPR genome editing.

### BACKGROUND

The versatility of Cas9 for genome editing derives from its RNA-guided nature. Existing guide RNAs suffer from several limitations, which limit their utility in therapeutic applications. For example, existing guide RNAs may be subject to rapid degradation in circulation and within cells. Moreover, chemical modifications of guide RNAs may reduce stability and editing efficiency. Accordingly, there exists a need in the art for optimized guide RNAs that retain efficient genome editing activity in vivo and ex vivo when paired with a CRISPR nuclease, such as Cas9.

### SUMMARY

The present disclosure provides chemically modified guide RNAs for Neisseria meningitidis (Nme) Cas9-mediated CRISPR genome editing. In certain embodiments, the guide RNAs of the disclosure are heavily or fully chemically modified. The guide RNA of the disclosure may confer several advantages in vivo or ex vivo, including stability, improved potency, and/or reduced off-target effects. Furthermore, in certain embodiments, the modified RNAs of the disclosure have reduced immunogenicity, e.g., a reduced ability to induce innate immune responses.

In certain aspects, the disclosure provides a chemically modified Neisseria meningitidis (Nme) guide RNA (gRNA) comprising: (a) a crRNA portion comprising (i) a guide sequence capable of hybridizing to a target polynucleotide sequence, and (ii) a repeat sequence; and (b) a tracrRNA portion comprising an anti-repeat nucleotide sequence that is complementary to the repeat sequence, wherein the crRNA portion comprises at least 40% modified nucleotides, and wherein the gRNA is capable of binding to an Nme Cas9 nuclease or a variant thereof.

In certain embodiments, the Nme Cas9 nuclease is Nme1Cas9, Nme2Cas9, or Nme3Cas9. In certain embodiments, the gRNA is capable of binding to an Nme Cas9 nuclease or variant thereof comprising an amino acid sequence with at least 80% identity to an amino acid sequence set forth in SEQ ID NO: 1, 2, or 3. In certain embodiments, the crRNA portion binds to an Nme Cas9 nuclease or variant thereof through one or more nucleobases at positions 2, 11, 12, 14, 17, 20, 34, 35, and 36 from the 5′ end of the crRNA portion. In certain embodiments, the repeat sequence of the crRNA portion binds to an Nme Cas9 nuclease or variant thereof through one or more nucleobases at positions 10, 11, and 12 from the 5′ end of the repeat sequence of the crRNA portion. In certain embodiments, the guide sequence of the crRNA portion binds to an Nme Cas9 nuclease or variant thereof through one or more nucleobases at positions 5, 8, 11, 13, 14, and 23 from the 3′ end of the guide sequence of the crRNA portion.

In certain embodiments, the tracrRNA portion comprises at least one modified nucleotide.

In certain embodiments, the modified nucleotides each independently comprise a modification of a ribose group, a phosphate group, a nucleobase, or a combination thereof.

In certain embodiments, each modification of the ribose group is independently selected from the group consisting of 2′-O-methyl, 2′-fluoro, 2′-deoxy, 2′-O-(2-methoxyethyl) (MOE), 2′-NH₂ (2′-amino), 4′-thio, a bicyclic nucleotide, a locked nucleic acid (LNA), a 2′-(S)-constrained ethyl (S-cEt), a constrained MOE, and a 2′-O,4′-C-aminomethylene bridged nucleic acid (2′,4′-BNA^{NC}).

In certain embodiments, at least 50% of the ribose groups are chemically modified. In certain embodiments, at least 40% of the ribose groups in the guide sequence of the crRNA portion are chemically modified. In certain embodiments, at least 40% of the ribose groups in the repeat sequence of the crRNA portion are chemically modified. In certain embodiments, at least 80% of the ribose groups are chemically modified. In certain embodiments, at least 80% of the ribose groups in the guide sequence of the crRNA portion are chemically modified. In certain embodiments, at least 90% of the ribose groups are chemically modified. In certain embodiments, at least 90% of the ribose groups in the guide sequence of the crRNA portion are chemically modified. In certain embodiments, 100% of the ribose groups are chemically modified. In certain embodiments, 100% of the ribose groups in the guide sequence of the crRNA portion are chemically modified.

In certain embodiments, each modification of the phosphate group is independently selected from the group consisting of a phosphorothioate, phosphonoacetate (PACE), thiophosphonoacetate (thioPACE), amide, triazole, phosphonate, and phosphotriester modification.

In certain embodiments, each modification of the nucleobase group is independently selected from the group consisting of 2-thiouridine, 4-thiouridine, N⁶-methyladenosine, pseudouridine, 2,6-diaminopurine, inosine, thymidine, 5-methylcytosine, 5-substituted pyrimidine, isoguanine, isocytosine, and halogenated aromatic groups.

In certain embodiments, the gRNA comprises at least 90% modified nucleotides. In certain embodiments, the gRNA 100% modified nucleotides.

In certain embodiments, at least one nucleotide of the crRNA portion comprises a 2′-deoxy chemical modification. In certain embodiments, one or more of the nucleotides at positions 7, 26, 33, 34, 35, 36, and 37 from the 5′ end of the crRNA portion comprises a 2′-deoxy chemical modification. In certain embodiments, one or more of the nucleotides at positions 12, 15, 16, 23, and 42 from the 3′ end of the crRNA portion comprises a 2′-deoxy chemical modification. In certain embodiments, the nucleotide at position 7 from the 5′ end of the guide sequence of the crRNA portion comprises a 2′-deoxy chemical modification. In certain embodiments, the nucleotide at position 18 from the 3′ end of the guide sequence of the crRNA portion comprises a 2′-deoxy chemical modification. In certain embodiments, the nucleotides at positions 2, 9, 10, 11, 12. and 13 from the 5′ end of the repeat sequence of the crRNA portion comprises a 2′-deoxy chemical modification.

In certain embodiments, at least one nucleotide of the crRNA portion comprises a 2′-fluoro chemical modification. In certain embodiments, one or more of the nucleotides at positions 12, 14, 16, 19, 20, and 21 from the 5′ end of the crRNA portion comprises a 2′-fluoro chemical modification. In certain embodiments, one or more of the nucleotides at positions 28, 29, 30, 33, 35, and 37 from the 3′ end of the crRNA portion comprises a 2′-fluoro chemical modification. In certain embodiments, one or more of the nucleotides at positions 4, 6, 9, 11, and 13 from the 3′ end of the guide sequence of the crRNA portion comprises a 2′-fluoro chemical modification. In certain embodiments, one or more of the nucleotides at positions 1, 5, 6, 7, and 8 from the 5′ end of the repeat sequence of the crRNA portion comprise a 2′-fluoro chemical modification. In certain embodiments, the nucleotides at positions 33 and 35 from the 3′ end of the crRNA portion comprises a 2′-fluoro chemical modification and position 42 from the 3′ end of the crRNA portion comprises a 2′-deoxy chemical modification. In certain embodiments, the nucleotides at positions 9 and 11 from the 3′ end of the guide sequence of the crRNA portion comprises a 2′-fluoro chemical modification and position 18 from the 3′ end of the guide sequence of the crRNA portion comprises a 2′-deoxy chemical modification.

In certain embodiments, the guide sequence of the crRNA portion is between 18 and 26 nucleotides in length. In certain embodiments, the repeat sequence of the crRNA portion is between 12 and 24 nucleotides in length. In certain embodiments, the repeat sequence of the crRNA portion is 12, 18, or 24 nucleotides in length. In certain embodiments, the crRNA portion is between 32 and 48 nucleotides in length.

In certain embodiments, the tracrRNA portion is between 60 and 100 nucleotides in length.

In certain embodiments, the chemically modified Nme gRNA comprises at least one chemical modification in a crRNA portion nucleotide sequence of: (N)ₓ GUUGUAGCUCCCUUUCUC (SEQ ID NO: 8) (CR42); or (N)ₓ GUUGUAGCUCCCUUUCUCAUUUCG (SEQ ID NO: 9) (CR48), wherein N corresponds to any nucleotide and x corresponds to any integer between 18 and 26.

In certain embodiments, the chemically modified Nme gRNA comprises a crRNA portion modification pattern selected from the group consisting of:

| | |
|---|---|
| CR48-2 | (mN)#(mN)#(mN)#(mN)(mN)(rN)(rN)(rN)(rN)(rN)(rN)(rN)(rN)(rN)(rN)(rN)(rN)(r |
| | N)(rN)(rN)(rN)(rN)(rN)(rN)(rG)(rU)(rU)(rG)(rU)(rA)(rG)(rC)(rU)(rC)(rC)(rC)(rU) |
| | (mU)(mU)(mC)(mU)(mC)(mA)(mU)(mU)#(mU)#(mC)#(mG) (SEQ ID NO: 13); |
| CR48-154 | (mN)#(mN)#(mN)#(mN)(mN)(mN)(dN)#(mN)(mN)(mN)(mN)(fN)(mN)(fN)(mN)( |
| | fN)(mN)(mN)(rN)(rN)(rN)(mN)(rN)(rN)(mG)(rU)(rU)(rG)(mU)(mA)(mG)(mC)(r |
| | U)(rC)(rC)(rC)(rU)(mU)(mU)(mC)(mU)(mC)(mA)(mU)(mU)#(mU)#(mC)#(mG) |
| | (SEQ ID NO: 14); |
| CR48-155 | (mN)#(mN)#(mN)#(mN)(mN)(mN)(dN)#(mN)(mN)(mN)(mN)(mN)(mN)(fN)(mN) |
| | (fN)(mN)(mN)(rN)(rN)(rN)(mN)(rN)(rN)(mG)(rU)(rU)(rG)(mU)(mA)(mG)(mC)(r |
| | U)(rC)(rC)(rC)(rU)(mU)(mU)(mC)(mU)(mC)(mA)(mU)(mU)#(mU)#(mC)#(mG) |
| | (SEQ ID NO: 15); |
| CR48-190 | (mN)#(mN)#(mN)#(mN)(mN)(mN)(dN)#(mN)(mN)(mN)(mN)(fN)(mN)(fN)(mN)( |
| | fN)(mN)(mN)(rN)(fN)(mN)(mN)(mN)(mN)(mG)(rU)(rU)(rG)(mU)(mA)(mG)(mC) |
| | (rU)(rC)(rC)(rC)(rU)(mU)(mU)(mC)(mU)(mC)(mA)(mU)(mU)#(mU)#(mC)#(mG) |
| | (SEQ ID NO: 16); |
| CR48-191 | (mN)#(mN)#(mN)#(mN)(mN)(mN)(dN)#(mN)(mN)(mN)(mN)(fN)(mN)(fN)(mN)( |
| | fN)(mN)(mN)(rN)(mN)(fN)(mN)(mN)(mN)(mG)(rU)(rU)(rG)(mU)(mA)(mG)(mC) |
| | (rU)(rC)(rC)(rC)(rU)(mU)(mU)(mC)(mU)(mC)(mA)(mU)(mU)#(mU)#(mC)#(mG) |
| | (SEQ ID NO: 17); |
| CR48-192 | (mN)#(mN)#(mN)#(mN)(mN)(mN)(dN)#(mN)(mN)(mN)(mN)(mN)(mN)(fN)(mN) |
| | (fN)(mN)(mN)(rN)(fN)(mN)(mN)(mN)(mN)(mG)(rU)(rU)(rG)(mU)(mA)(mG)(nC |
| | )(rU)(rC)(rC)(rC)(rU)(mU)(mU)(mC)(mU)(mC)(mA)(mU)(mU)#(mU)#(mC)#(mG |
| | ) (SEQ ID NO: 18); |
| CR48-193 | (mN)#(mN)#(mN)#(mN)(mN)(mN)(dN)#(mN)(mN)(mN)(mN)(mN)(mN)(fN)(mN) |
| | (fN)(mN)(mN)(rN)(mN)(fN)(mN)(mN)(mN)(mG)(rU)(rU)(rG)(mU)(mA)(mG)(mC |
| | )(rU)(rC)(rC)(rC)(rU)(mU)(mU)(mC)(mU)(mC)(mA)(mU)(mU)#(mU)#(mC)#(mG |
| | ) (SEQ ID NO: 19); |
| CR48-194 | (mN)#(mN)#(mN)#(mN)(mN)(mN)(dN)#(mN)(mN)(mN)(mN)(fN)(mN)(fN)(mN)( |
| | fN)(mN)(mN)(rN)#(fN)(mN)(mN)(mN)(mN)(mG)(rU)(rU)(rG)(mU)(mA)(mG)(m |
| | C)(rU)(rC)(rC)(rC)(rU)(mU)(mU)(mC)(mU)(mC)(mA)(mU)(mU)#(mU)#(mC)#(m |
| | G) (SEQ ID NO: 20); |
| CR48-195 | (mN)#(mN)#(mN)#(mN)(mN)(mN)(dN)#(mN)(mN)(mN)(mN)(fN)(mN)(fN)(mN)( |
| | fN)(mN)(mN)(rN)#(mN)(fN)(mN)(mN)(mN)(mG)(rU)(rU)(rG)(mU)(mA)(mG)(m |
| | C)(rU)(rC)(rC)(rC)(rU)(mU)(mU)(mC)(mU)(mC)(mA)(mU)(mU)#(mU)#(mC)#(m |
| | G) (SEQ ID NO: 21); |
| CR48-196 | (mN)#(mN)#(mN)#(mN)(mN)(mN)(dN)#(mN)(mN)(mN)(mN)(mN)(mN)(fN)(mN) |
| | (fN)(mN)(mN)(rN)#(fN)(mN)(mN)(mN)(mN)(mG)(rU)(rU)(rG)(mU)(mA)(mG)(m |
| | C)(rU)(rC)(rC)(rC)(rU)(mU)(mU)(mC)(mU)(mC)(mA)(mU)(mU)#(mU)#(mC)#(m |
| | G) (SEQ ID NO: 22); |
| CR48-197 | (mN)#(mN)#(mN)#(mN)(mN)(mN)(dN)#(mN)(mN)(mN)(mN)(mN)(mN)(fN)(mN) |
| | (fN)(mN)(mN)(rN)#(mN)(fN)(mN)(mN)(mN)(mG)(rU)(rU)(rG)(mU)(mA)(mG)(m |
| | C)(rU)(rC)(rC)(rC)(rU)(mU)(mU)(mC)(mU)(mC)(mA)(mU)(mU)#(mU)#(mC)#(m |
| | G) (SEQ ID NO: 23); |
| CR48-198 | (mN)#(mN)#(mN)#(mN)(mN)(mN)(dN)#(mN)(mN)(mN)(mN)(fN)(mN)(fN)(mN)( |
| | fN)(mN)(mN)(mN)(fN)(mN)(mN)(mN)(mN)(mG)(rU)(rU)(rG)(mU)(mA)(mG)(m |
| | C)(rU)(rC)(rC)(rC)(rU)(mU)(mU)(mC)(mU)(mC)(mA)(mU)(mU)#(mU)#(mC)#(m |
| | G) (SEQ ID NO: 24); |
| CR48-199 | (mN)#(mN)#(mN)#(mN)(mN)(mN)(dN)#(mN)(mN)(mN)(mN)(fN)(mN)(fN)(mN)( |
| | fN)(mN)(mN)(mN)(mN)(fN)(mN)(mN)(mN)(mG)(rU)(rU)(rG)(mU)(mA)(mG)(m |
| | C)(rU)(rC)(rC)(rC)(rU)(mU)(mU)(mC)(mU)(mC)(mA)(mU)(mU)#(mU)#(mC)#(m |
| | G) (SEQ ID NO: 25); |
| CR48-200 | (mN)#(mN)#(mN)#(mN)(mN)(mN)(dN)#(mN)(mN)(mN)(mN)(mN)(mN)(fN)(mN) |
| | (fN)(mN)(mN)(mN)(fN)(mN)(mN)(mN)(mN)(mG)(rU)(rU)(rG)(mU)(mA)(mG)(m |
| | C)(rU)(rC)(rC)(rC)(rU)(mU)(mU)(mC)(mU)(mC)(mA)(mU)(mU)#(mU)#(mC)#(m |
| | G) (SEQ ID NO: 26); |
| CR48-201 | (mN)#(mN)#(mN)#(mN)(mN)(mN)(dN)#(mN)(mN)(mN)(mN)(mN)(mN)(fN)(mN) |
| | (fN)(mN)(mN)(mN)(mN)(fN)(mN)(mN)(mN)(mG)(rU)(rU)(rG)(mU)(mA)(mG)(m |
| | C)(rU)(rC)(rC)(rC)(rU)(mU)(mU)(mC)(mU)(mC)(mA)(mU)(mU)#(mU)#(mC)#(m |
| | G) (SEQ ID NO: 27); |
| CR48-202 | (mN)#(mN)#(mN)#(mN)(mN)(mN)(dN)#(mN)(mN)(mN)(mN)(fN)(mN)(fN)(mN)( |
| | fN)(mN)(mN)(fN)(fN)(mN)(mN)(mN)(mN)(mG)(rU)(rU)(rG)(mU)(mA)(mG)(mC) |
| | (rU)(rC)(rC)(rC)(rU)(mU)(mU)(mC)(mU)(mC)(mA)(mU)(mU)#(mU)#(mC)#(mG) |
| | (SEQ ID NO: 28); |
| CR48-203 | (mN)#(mN)#(mN)#(mN)(mN)(mN)(dN)#(mN)(mN)(mN)(mN)(fN)(mN)(fN)(mN)( |
| | fN)(mN)(mN)(fN)(mN)(fN)(mN)(mN)(mN)(mG)(rU)(rU)(rG)(mU)(mA)(mG)(mC) |
| | (rU)(rC)(rC)(rC)(rU)(mU)(mU)(mC)(mU)(mC)(mA)(mU)(mU)#(mU)#(mC)#(mG) |
| | (SEQ ID NO: 29); |
| CR48-204 | (mN)#(mN)#(mN)#(mN)(mN)(mN)(dN)#(mN)(mN)(mN)(mN)(mN)(mN)(fN)(mN) |
| | (fN)(mN)(mN)(fN)(fN)(mN)(mN)(mN)(mN)(mG)(rU)(rU)(rG)(mU)(mA)(mG)(mC |
| | )(rU)(rC)(rC)(rC)(rU)(mU)(mU)(mC)(mU)(mC)(mA)(mU)(mU)#(mU)#(mC)#(mG |
| | ) (SEQ ID NO: 30); |
| CR48-205 | (mN)#(mN)#(mN)#(mN)(mN)(mN)(dN)#(mN)(mN)(mN)(mN)(mN)(mN)(fN)(mN) |
| | (fN)(mN)(mN)(fN)(mN)(fN)(mN)(mN)(mN)(mG)(rU)(rU)(rG)(mU)(mA)(mG)(mC |
| | )(rU)(rC)(rC)(rC)(rU)(mU)(mU)(mC)(mU)(mC)(mA)(mU)(mU)#(mU)#(mC)#(mG |
| | ) (SEQ ID NO: 31); |
| CR48-213 | (mN)#(mN)#(mN)#(mN)(mN)(mN)(dN)#(mN)(mN)(mN)(mN)(mN)(mN)(fN)(mN) |
| | (fN)(mN)(mN)(rN)(rN)(rN)(mN)(rN)(rN)(mG)(dT)#(rU)(mG)(mU)(mA)(mG)(mC) |
| | (rU)(rC)(rC)(rC)(rU)(mU)(mU)(mC)(mU)(mC)(mA)(mU)(mU)#(mU)#(mC)#(mG) |
| | (SEQ ID NO: 32); |
| CR48-214 | (mN)#(mN)#(mN)#(mN)(mN)(mN)(dN)#(mN)(mN)(mN)(mN)(mN)(mN)(fN)(mN) |
| | (fN)(mN)(mN)(rN)(rN)(rN)(mN)(rN)(rN)(mG)(dT)#(rU)(mG)(mU)(mA)(mG)(mC) |
| | (rU)(rC)(mC)(rC)(rU)(mU)(mU)(mC)(mU)(mC)(mA)(mU)(mU)#(mU)#(mC)#(mG |
| | ) (SEQ ID NO: 33); |
| CR48-215 | (mN)#(mN)#(mN)#(mN)(mN)(mN)(dN)#(mN)(mN)(mN)(mN)(mN)(mN)(fN)(mN) |
| | (fN)(mN)(mN)(rN)(rN)(rN)(mN)(rN)(rN)(mG)(dT)#(rU)(mG)(mU)(mA)(mG)(mC) |
| | (rU)(rC)(rC)(mC)(rU)(mU)(mU)(mC)(mU)(mC)(mA)(mU)(mU)#(mU)#(mC)#(mG |
| | ) (SEQ ID NO: 34); |
| CR48-216 | (mN)#(mN)#(mN)#(mN)(mN)(mN)(dN)#(mN)(mN)(mN)(mN)(mN)(mN)(fN)(mN) |
| | (fN)(mN)(mN)(rN)(rN)(rN)(mN)(rN)(rN)(mG)(dT)#(rU)(mG)(mU)(mA)(mG)(mC) |
| | (rU)(rC)(mC)(mC)(rU)(mU)(mU)(mC)(mU)(mC)(mA)(mU)(mU)#(mU)#(mC)#(m |
| | G) (SEQ ID NO: 35); |
| CR48-217 | (mN)#(mN)#(mN)#(mN)(mN)(mN)(dN)#(mN)(mN)(mN)(mN)(mN)(mN)(fN)(mN) |
| | (fN)(mN)(mN)(rN)(rN)(rN)(mN)(rN)(rN)(mG)(rU)(rU)(rG)(mU)(mA)(mG)(mC)(d |
| | T)#(rC)(rC)(rC)(rU)(mU)(mU)(mC)(mU)(mC)(mA)(mU)(mU)#(mU)#(mC)#(mG) |
| | (SEQ ID NO: 36); |
| CR48-218 | (mN)#(mN)#(mN)#(mN)(mN)(mN)(dN)#(mN)(mN)(mN)(mN)(mN)(mN)(fN)(mN) |
| | (fN)(mN)(mN)(rN)(rN)(rN)(mN)(rN)(rN)(mG)(rU)(rU)(rG)(mU)(mA)(mG)(mC)(r |
| | U)(dC)#(rC)(rC)(rU)(mU)(mU)(mC)(mU)(mC)(mA)(mU)(mU)#(mU)#(mC)#(mG) |
| | (SEQ ID NO: 37); |
| CR48-219 | (mN)#(mN)#(mN)#(mN)(mN)(mN)(dN)#(mN)(mN)(mN)(mN)(mN)(mN)(fN)(mN) |
| | (fN)(mN)(mN)(rN)(rN)(rN)(mN)(rN)(rN)(mG)(rU)(rU)(rG)(mU)(mA)(mG)(mC)(r |
| | U)(rC)(dC)#(rC)(rU)(mU)(mU)(mC)(mU)(mC)(mA)(mU)(mU)#(mU)#(mC)#(mG) |
| | (SEQ ID NO: 38); |
| CR48-220 | (mN)#(mN)#(mN)#(mN)(mN)(mN)(dN)#(mN)(mN)(mN)(mN)(mN)(mN)(fN)(mN) |
| | (fN)(mN)(mN)(rN)(rN)(rN)(mN)(rN)(rN)(mG)(rU)(rU)(rG)(mU)(mA)(mG)(mC)(r |
| | U)(rC)(rC)(dC)#(rU)(mU)(mU)(mC)(mU)(mC)(mA)(mU)(mU)#(mU)#(mC)#(mG) |
| | (SEQ ID NO: 39); |
| CR48-221 | (mN)#(mN)#(mN)#(mN)(mN)(mN)(dN)#(mN)(mN)(mN)(mN)(mN)(mN)(fN)(mN) |
| | (fN)(mN)(mN)(rN)(rN)(rN)(mN)(rN)(rN)(mG)(rU)(rU)(rG)(mU)(mA)(mG)(mC)(r |
| | U)(rC)(rC)(rC)(dT)#(mU)(mU)(mC)(mU)(mC)(mA)(mU)(mU)#(mU)#(mC)#(mG) |
| | (SEQ ID NO: 40); |
| CR48-222 | (mN)#(mN)#(mN)#(mN)(mN)(mN)(dN)#(mN)(mN)(mN)(mN)(mN)(mN)(fN)(mN) |
| | (fN)(mN)(mN)(rN)(rN)(rN)(mN)(rN)(rN)(mG)(rU)(rU)(rG)(mU)(mA)(mG)(mC)(d |
| | T)#(rC)(dC)#(rC)(dT)#(mU)(mU)(mC)(mU)(mC)(mA)(mU)(mU)#(mU)#(mC)#(m |
| | G) (SEQ ID NO: 41); |
| CR48-223 | (mN)#(mN)#(mN)#(mN)(mN)(mN)(dN)#(mN)(mN)(mN)(mN)(mN)(mN)(fN)(mN) |
| | (fN)(mN)(mN)(rN)(rN)(rN)(mN)(rN)(rN)(mG)(dT)#(rU)(rG)(mU)(mA)(mG)(mC)( |
| | dT)#(dC)#(dC)#(rC)(dT)#(mU)(mU)(mC)(mU)(mC)(mA)(mU)(mU)#(mU)#(mC)# |
| | (mG) (SEQ ID NO: 42); |
| CR48-224 | (mN)#(mN)#(mN)#(mN)(mN)(mN)(dN)#(mN)(mN)(mN)(mN)(mN)(mN)(fN)(mN) |
| | (fN)(mN)(mN)(rN)(rN)(rN)(mN)(rN)(rN)(mG)(dT)#(rU)(mG)(mU)(mA)(mG)(mC) |
| | (dT)#(rC)(mC)(rC)(dT)#(mU)(mU)(mC)(mU)(mC)(mA)(mU)(mU)#(mU)#(mC)#( |
| | mG) (SEQ ID NO: 43); |
| CR48-225 | (mN)#(mN)#(mN)#(mN)(mN)(mN)(dN)#(mN)(mN)(mN)(mN)(mN)(mN)(fN)(mN) |
| | (fN)(mN)(mN)(rN)(rN)(rN)(mN)(rN)(rN)(mG)(dT)#(rU)(mG)(mU)(mA)(mG)(mC) |
| | (dT)#(rC)(dC)#(mC)(dT)#(mU)(mU)(mC)(mU)(mC)(mA)(mU)(mU)#(mU)#(mC)# |
| | (mG) (SEQ ID NO: 44); |
| CR48-226 | (mN)#(mN)#(mN)#(mN)(mN)(mN)(dN)#(mN)(mN)(mN)(mN)(mN)(mN)(fN)(mN) |
| | (fN)(mN)(mN)(rN)(rN)(rN)(mN)(rN)(rN)(mG)(dT)#(rU)(mG)(mU)(mA)(mG)(mC) |
| | (dT)#(rC)(mC)(mC)(dT)#(mU)(mU)(mC)(mU)(mC)(mA)(mU)(mU)#(mU)#(mC)#( |
| | mG) (SEQ ID NO: 45); |
| CR48-227 | (mN)#(mN)#(mN)#(mN)(mN)(mN)(dN)#(mN)(mN)(mN)(mN)(mN)(mN)(fN)(mN) |
| | (fN)(mN)(mN)(rN)(rN)(rN)(mN)(rN)(rN)(mG)(dT)#(rU)(mG)(mU)(mA)(mG)(mC) |
| | (dT)#(dC)#(mC)(mC)(dT)#(mU)(mU)(mC)(mU)(mC)(mA)(mU)(mU)#(mU)#(mC) |
| | #(mG) (SEQ ID NO: 46); |
| CR48-199- | (mN)#(mN)#(mN)#(mN)(mN)(mN)(dN)#(mN)(mN)(mN)(mN)(fN)(mN)(fN)(mN)( |
| V1.1 | fN)(mN)(mN)(mN)(mN)(fN)(mN)(mN)(mN)(mG)(rU)(rU)(mG)(mU)(mA)(mG)(m |
| | C)(rU)(rC)(rC)(dC)#(rU)(mU)(mU)(mC)(mU)(mC)(mA)(mU)(mU)#(mU)#(mC)#( |
| | mG) (SEQ ID NO: 47); |
| CR48-199- | (mN)#(mN)#(mN)#(mN)(mN)(mN)(dN)#(mN)(mN)(mN)(mN)(fN)(mN)(fN)(mN)( |
| V1.2 | fN)(mN)(mN)(mN)(mN)(fN)(mN)(mN)(mN)(mG)(rU)(rU)(mG)(mU)(mA)(mG)(m |
| | C)(rU)(rC)(dC)#(rC)(rU)(mU)(mU)(mC)(mU)(mC)(mA)(mU)(mU)#(mU)#(mC)#( |
| | mG) (SEQ ID NO: 48); |
| CR48-199- | (mN)#(mN)#(mN)#(mN)(mN)(mN)(dN)#(mN)(mN)(mN)(mN)(fN)(mN)(fN)(mN)( |
| V1.3 | fN)(mN)(mN)(mN)(mN)(fN)(mN)(mN)(mN)(mG)(rU)(rU)(mG)(mU)(mA)(mG)(m |
| | C)(rU)(rC)(rC)(rC)(dT)#(mU)(mU)(mC)(mU)(mC)(mA)(mU)(mU)#(mU)#(mC)#( |
| | mG) (SEQ ID NO: 49); |
| CR48-199- | (mN)#(mN)#(mN)#(mN)(mN)(mN)(dN)#(mN)(mN)(mN)(mN)(fN)(mN)(fN)(mN)( |
| V1.4 | fN)(mN)(mN)(mN)(mN)(fN)(mN)(mN)(mN)(mG)(rU)(rU)(mG)(mU)(mA)(mG)(m |
| | C)(rU)(rC)(mC)(rC)(rU)(mU)(mU)(mC)(mU)(mC)(mA)(mU)(mU)#(mU)#(mC)#( |
| | mG) (SEQ ID NO: 50); |
| CR48-199- | (mN)#(mN)#(mN)#(mN)(mN)(mN)(dN)#(mN)(mN)(mN)(mN)(fN)(mN)(fN)(mN)( |
| V1.5 | fN)(mN)(mN)(mN)(mN)(fN)(mN)(mN)(mN)(mG)(rU)(rU)(mG)(mU)(mA)(mG)(m |
| | C)(rU)(rC)(dC)#(dC)#(rU)(mU)(mU)(mC)(mU)(mC)(mA)(mU)(mU)#(mU)#(mC)# |
| | (mG) (SEQ ID NO: 51); |
| CR48-199- | (mN)#(mN)#(mN)#(mN)(mN)(mN)(dN)#(mN)(mN)(mN)(mN)(fN)(mN)(fN)(mN)( |
| V1.6 | fN)(mN)(mN)(mN)(mN)(fN)(mN)(mN)(mN)(mG)(rU)(rU)(mG)(mU)(mA)(mG)(m |
| | C)(rU)(rC)(dC)#(rC)(dT)#(mU)(mU)(mC)(mU)(mC)(mA)(mU)(mU)#(mU)#(mC)# |
| | (mG) (SEQ ID NO: 52); |
| CR48-199- | (mN)#(mN)#(mN)#(mN)(mN)(mN)(dN)#(mN)(mN)(mN)(mN)(fN)(mN)(fN)(mN)( |
| V1.7 | fN)(mN)(mN)(mN)(mN)(fN)(mN)(mN)(mN)(mG)(rU)(rU)(mG)(mU)(mA)(mG)(m |
| | C)(rU)(rC)(dC)#(dC)#(dT)#(mU)(mU)(mC)(mU)(mC)(mA)(mU)(mU)#(mU)#(mC |
| | )#(mG) (SEQ ID NO: 53); |
| | (mN)#(mN)#(mN)#(mN)(mN)(mN)(dN)#(mN)(mN)(mN)(mN)(fN)(mN)(fN)(mN)( |
| CR48-199- | fN)(mN)(mN)(mN)(mN)(fN)(mN)(mN)(mN)(mG)(rU)(rU)(mG)(mU)(mA)(mG)(m |
| V1.8 | C)(rU)(rC)(mC)(dC)#(rU)(mU)(mU)(mC)(mU)(mC)(mA)(mU)(mU)#(mU)#(mC)# |
| | (mG) (SEQ ID NO: 54); |
| CR48-199- | (mN)#(mN)#(mN)#(mN)(mN)(mN)(dN)#(mN)(mN)(mN)(mN)(fN)(mN)(fN)(mN)( |
| V1.9 | fN)(mN)(mN)(mN)(mN)(fN)(mN)(mN)(mN)(mG)(rU)(rU)(mG)(mU)(mA)(mG)(m |
| | C)(rU)(rC)(mC)(rC)(dT)#(mU)(mU)(mC)(mU)(mC)(mA)(mU)(mU)#(mU)#(mC)#( |
| | mG) (SEQ ID NO: 55); |
| CR48-199- | (mN)#(mN)#(mN)#(mN)(mN)(mN)(dN)#(mN)(mN)(mN)(mN)(fN)(mN)(fN)(mN)( |
| V1.10 | fN)(mN)(mN)(mN)(mN)(fN)(mN)(mN)(mN)(mG)(rU)(rU)(mG)(mU)(mA)(mG)(m |
| | C)(rU)(rC)(mC)(dC)#(dT)#(mU)(mU)(mC)(mU)(mC)(mA)(mU)(mU)#(mU)#(mC) |
| | #(mG) (SEQ ID NO: 56); |
| CR48-199- | (mN)#(mN)#(mN)#(mN)(mN)(mN)(dN)#(mN)(mN)(mN)(mN)(fN)(mN)(fN)(mN)( |
| V1.11 | fN)(mN)(mN)(mN)(mN)(fN)(mN)(mN)(mN)(mG)(rU)(rU)(mG)(mU)(mA)(mG)(m |
| | C)(rU)(rC)(mC)(mC)(rU)(mU)(mU)(mC)(mU)(mC)(mA)(mU)(mU)#(mU)#(mC)#( |
| | mG) (SEQ ID NO: 57); |
| CR48-199- | (mN)#(mN)#(mN)#(mN)(mN)(mN)(dN)#(mN)(mN)(mN)(mN)(fN)(mN)(fN)(mN)( |
| V1.12 | fN)(mN)(mN)(mN)(mN)(fN)(mN)(mN)(mN)(mG)(rU)(rU)(mG)(mU)(mA)(mG)(m |
| | C)(rU)(rC)(mC)(mC)(dT)#(mU)(mU)(mC)(mU)(mC)(mA)(mU)(mU)#(mU)#(mC) |
| | #(mG) (SEQ ID NO: 58); |
| CR48-199- | (mN)#(mN)#(mN)#(mN)(mN)(mN)(dN)#(mN)(mN)(mN)(mN)(fN)(mN)(fN)(mN)( |
| V1.13 | fN)(mN)(mN)(mN)(mN)(fN)(mN)(mN)(mN)(mG)(rU)(rU)(mG)(mU)(mA)(mG)(m |
| | C)(dT)#(rC)(rC)(dC)#(rU)(mU)(mU)(mC)(mU)(mC)(mA)(mU)(mU)#(mU)#(mC)# |
| | (mG) (SEQ ID NO: 59); |
| CR48-199- | (mN)#(mN)#(mN)#(mN)(mN)(mN)(dN)#(mN)(mN)(mN)(mN)(fN)(mN)(fN)(mN)( |
| V1.14 | fN)(mN)(mN)(mN)(mN)(fN)(mN)(mN)(mN)(mG)(rU)(rU)(mG)(mU)(mA)(mG)(m |
| | C)(dT)#(rC)(dC)#(rC)(rU)(mU)(mU)(mC)(mU)(mC)(mA)(mU)(mU)#(mU)#(mC)# |
| | (mG) (SEQ ID NO: 60); |
| CR48-199- | (mN)#(mN)#(mN)#(mN)(mN)(mN)(dN)#(mN)(mN)(mN)(mN)(fN)(mN)(fN)(mN)( |
| V1.15 | fN)(mN)(mN)(mN)(mN)(fN)(mN)(mN)(mN)(mG)(rU)(rU)(mG)(mU)(mA)(mG)(m |
| | C)(dT)#(rC)(rC)(rC)(dT)#(mU)(mU)(mC)(mU)(mC)(mA)(mU)(mU)#(mU)#(mC)# |
| | (mG) (SEQ ID NO: 61); |
| CR48-199- | (mN)#(mN)#(mN)#(mN)(mN)(mN)(dN)#(mN)(mN)(mN)(mN)(fN)(mN)(fN)(mN)( |
| V1.16 | fN)(mN)(mN)(mN)(mN)(fN)(mN)(mN)(mN)(mG)(rU)(rU)(mG)(mU)(mA)(mG)(m |
| | C)(dT)#(rC)(mC)(rC)(rU)(mU)(mU)(mC)(mU)(mC)(mA)(mU)(mU)#(mU)#(mC)#( |
| | mG) (SEQ ID NO: 62); |
| CR48-199- | (mN)#(mN)#(mN)#(mN)(mN)(mN)(dN)#(mN)(mN)(mN)(mN)(fN)(mN)(fN)(mN)( |
| V1.17 | fN)(mN)(mN)(mN)(mN)(fN)(mN)(mN)(mN)(mG)(rU)(rU)(mG)(mU)(mA)(mG)(m |
| | C)(dT)#(rC)(dC)#(dC)#(rU)(mU)(mU)(mC)(mU)(mC)(mA)(mU)(mU)#(mU)#(mC |
| | )#(mG) (SEQ ID NO: 63); |
| CR48-199- | (mN)#(mN)#(mN)#(mN)(mN)(mN)(dN)#(mN)(mN)(mN)(mN)(fN)(mN)(fN)(mN)( |
| V1.18 | fN)(mN)(mN)(mN)(mN)(fN)(mN)(mN)(mN)(mG)(rU)(rU)(mG)(mU)(mA)(mG)(m |
| | C)(dT)#(rC)(dC)#(rC)(dT)#(mU)(mU)(mC)(mU)(mC)(mA)(mU)(mU)#(mU)#(mC) |
| | #(mG) (SEQ ID NO: 64); |
| CR48-199- | (mN)#(mN)#(mN)#(mN)(mN)(mN)(dN)#(mN)(mN)(mN)(mN)(fN)(mN)(fN)(mN)( |
| V1.19 | fN)(mN)(mN)(mN)(mN)(fN)(mN)(mN)(mN)(mG)(rU)(rU)(mG)(mU)(mA)(mG)(m |
| | C)(dT)#(rC)(dC)#(dC)#(dT)#(mU)(mU)(mC)(mU)(mC)(mA)(mU)(mU)#(mU)#(m |
| | C)#(mG) (SEQ ID NO: 65); |
| CR48-199- | (mN)#(mN)#(mN)#(mN)(mN)(mN)(dN)#(mN)(mN)(mN)(mN)(fN)(mN)(fN)(mN)( |
| V1.20 | fN)(mN)(mN)(mN)(mN)(fN)(mN)(mN)(mN)(mG)(rU)(rU)(mG)(mU)(mA)(mG)(m |
| | C)(dT)#(rC)(mC)(dC)#(rU)(mU)(mU)(mC)(mU)(mC)(mA)(mU)(mU)#(mU)#(mC) |
| | #(mG) (SEQ ID NO: 66); |
| CR48-199- | (mN)#(mN)#(mN)#(mN)(mN)(mN)(dN)#(mN)(mN)(mN)(mN)(fN)(mN)(fN)(mN)( |
| V1.21 | fN)(mN)(mN)(mN)(mN)(fN)(mN)(mN)(mN)(mG)(rU)(rU)(mG)(mU)(mA)(mG)(m |
| | C)(dT)#(rC)(mC)(rC)(dT)#(mU)(mU)(mC)(mU)(mC)(mA)(mU)(mU)#(mU)#(mC) |
| | #(mG) (SEQ ID NO: 67); |
| CR48-199- | (mN)#(mN)#(mN)#(mN)(mN)(mN)(dN)#(mN)(mN)(mN)(mN)(fN)(mN)(fN)(mN)( |
| V1.22 | fN)(mN)(mN)(mN)(mN)(fN)(mN)(mN)(mN)(mG)(rU)(rU)(mG)(mU)(mA)(mG)(m |
| | C)(dT)#(rC)(mC)(dC)#(dT)#(mU)(mU)(mC)(mU)(mC)(mA)(mU)(mU)#(mU)#(m |
| | C)#(mG) (SEQ ID NO: 68); |
| CR48-199- | (mN)#(mN)#(mN)#(mN)(mN)(mN)(dN)#(mN)(mN)(mN)(mN)(fN)(mN)(fN)(mN)( |
| V1.23 | fN)(mN)(mN)(mN)(mN)(fN)(mN)(mN)(mN)(mG)(rU)(rU)(mG)(mU)(mA)(mG)(m |
| | C)(dT)#(rC)(mC)(mC)(rU)(mU)(mU)(mC)(mU)(mC)(mA)(mU)(mU)#(mU)#(mC) |
| | #(mG) (SEQ ID NO: 69); |
| CR48-199- | (mN)#(mN)#(mN)#(mN)(mN)(mN)(dN)#(mN)(mN)(mN)(mN)(fN)(mN)(fN)(mN)( |
| V1.24 | fN)(mN)(mN)(mN)(mN)(fN)(mN)(mN)(mN)(mG)(rU)(rU)(mG)(mU)(mA)(mG)(m |
| | C)(dT)#(rC)(mC)(mC)(dT)#(mU)(mU)(mC)(mU)(mC)(mA)(mU)(mU)#(mU)#(mC |
| | )#(mG) (SEQ ID NO: 70); |
| CR48-199- | (mN)#(mN)#(mN)#(mN)(mN)(mN)(dN)#(mN)(mN)(mN)(mN)(fN)(mN)(fN)(mN)( |
| V1.25 | fN)(mN)(mN)(mN)(mN)(fN)(mN)(mN)(mN)(mG)(dT)#(rU)(mG)(mU)(mA)(mG)( |
| | mC)(dT)#(rC)(rC)(dC)#(rU)(mU)(mU)(mC)(mU)(mC)(mA)(mU)(mU)#(mU)#(mC |
| | )#(mG) (SEQ ID NO: 71); |
| CR48-199- | (mN)#(mN)#(mN)#(mN)(mN)(mN)(dN)#(mN)(mN)(mN)(mN)(fN)(mN)(fN)(mN)( |
| V1.26 | fN)(mN)(mN)(mN)(mN)(fN)(mN)(mN)(mN)(mG)(dT)#(rU)(mG)(mU)(mA)(mG)( |
| | mC)(dT)#(rC)(dC)#(rC)(rU)(mU)(mU)(mC)(mU)(mC)(mA)(mU)(mU)#(mU)#(mC |
| | )#(mG) (SEQ ID NO: 72); |
| CR48-199- | (mN)#(mN)#(mN)#(mN)(mN)(mN)(dN)#(mN)(mN)(mN)(mN)(fN)(mN)(fN)(mN)( |
| V1.27 | fN)(mN)(mN)(mN)(mN)(fN)(mN)(mN)(mN)(mG)(dT)#(rU)(mG)(mU)(mA)(mG)( |
| | mC)(dT)#(rC)(rC)(rC)(dT)#(mU)(mU)(mC)(mU)(mC)(mA)(mU)(mU)#(mU)#(mC |
| | )#(mG) (SEQ ID NO: 73); |
| CR48-199- | (mN)#(mN)#(mN)#(mN)(mN)(mN)(dN)#(mN)(mN)(mN)(mN)(fN)(mN)(fN)(mN)( |
| V1.28 | fN)(mN)(mN)(mN)(mN)(fN)(mN)(mN)(mN)(mG)(dT)#(rU)(mG)(mU)(mA)(mG)( |
| | mC)(dT)#(rC)(mC)(rC)(rU)(mU)(mU)(mC)(mU)(mC)(mA)(mU)(mU)#(mU)#(mC) |
| | #(mG) (SEQ ID NO: 74); |
| CR48-199- | (mN)#(mN)#(mN)#(mN)(mN)(mN)(dN)#(mN)(mN)(mN)(mN)(fN)(mN)(fN)(mN)( |
| V1.29 | fN)(mN)(mN)(mN)(mN)(fN)(mN)(mN)(mN)(mG)(dT)#(rU)(mG)(mU)(mA)(mG)( |
| | mC)(dT)#(rC)(dC)#(dC)#(rU)(mU)(mU)(mC)(mU)(mC)(mA)(mU)(mU)#(mU)#(m |
| | C)#(mG) (SEQ ID NO: 75); |
| CR48-199- | (mN)#(mN)#(mN)#(mN)(mN)(mN)(dN)#(mN)(mN)(mN)(mN)(fN)(mN)(fN)(mN)( |
| V1.30 | fN)(mN)(mN)(mN)(mN)(fN)(mN)(mN)(mN)(mG)(dT)#(rU)(mG)(mU)(mA)(mG)( |
| | mC)(dT)#(rC)(dC)#(rC)(dT)#(mU)(mU)(mC)(mU)(mC)(mA)(mU)(mU)#(mU)#(m |
| | C)#(mG) (SEQ ID NO: 76); |
| CR48-199- | (mN)#(mN)#(mN)#(mN)(mN)(mN)(dN)#(mN)(mN)(mN)(mN)(fN)(mN)(fN)(mN)( |
| V1.31 | fN)(mN)(mN)(mN)(mN)(fN)(mN)(mN)(mN)(mG)(dT)#(rU)(mG)(mU)(mA)(mG)( |
| | mC)(dT)#(rC)(dC)#(dC)#(dT)#(mU)(mU)(mC)(mU)(mC)(mA)(mU)(mU)#(mU)#( |
| | mC)#(mG) (SEQ ID NO: 77); |
| CR48-199- | (mN)#(mN)#(mN)#(mN)(mN)(mN)(dN)#(mN)(mN)(mN)(mN)(fN)(mN)(fN)(mN)( |
| V1.32 | fN)(mN)(mN)(mN)(mN)(fN)(mN)(mN)(mN)(mG)(dT)#(rU)(mG)(mU)(mA)(mG)( |
| | mC)(dT)#(rC)(mC)(dC)#(rU)(mU)(mU)(mC)(mU)(mC)(mA)(mU)(mU)#(mU)#(m |
| | C)#(mG) (SEQ ID NO: 78); |
| CR48-199- | (mN)#(mN)#(mN)#(mN)(mN)(mN)(dN)#(mN)(mN)(mN)(mN)(fN)(mN)(fN)(mN)( |
| V1.33 | fN)(mN)(mN)(mN)(mN)(fN)(mN)(mN)(mN)(mG)(dT)#(rU)(mG)(mU)(mA)(mG)( |
| | mC)(dT)#(rC)(mC)(rC)(dT)#(mU)(mU)(mC)(mU)(mC)(mA)(mU)(mU)#(mU)#(m |
| | C)#(mG) (SEQ ID NO: 79); |
| CR48-199- | (mN)#(mN)#(mN)#(mN)(mN)(mN)(dN)#(mN)(mN)(mN)(mN)(fN)(mN)(fN)(mN)( |
| V1.34 | fN)(mN)(mN)(mN)(mN)(fN)(mN)(mN)(mN)(mG)(dT)#(rU)(mG)(mU)(mA)(mG)( |
| | mC)(dT)#(rC)(mC)(dC)#(dT)#(mU)(mU)(mC)(mU)(mC)(mA)(mU)(mU)#(mU)#( |
| | mC)#(mG) (SEQ ID NO: 80); |
| CR48-199- | (mN)#(mN)#(mN)#(mN)(mN)(mN)(dN)#(mN)(mN)(mN)(mN)(fN)(mN)(fN)(mN)( |
| V1.35 | fN)(mN)(mN)(mN)(mN)(fN)(mN)(mN)(mN)(mG)(dT)#(rU)(mG)(mU)(mA)(mG)( |
| | mC)(dT)#(rC)(mC)(mC)(rU)(mU)(mU)(mC)(mU)(mC)(mA)(mU)(mU)#(mU)#(m |
| | C)#(mG) (SEQ ID NO: 81); |
| CR48-199- | (mN)#(mN)#(mN)#(mN)(mN)(mN)(dN)#(mN)(mN)(mN)(mN)(fN)(mN)(fN)(mN)( |
| V1.36 | fN)(mN)(mN)(mN)(mN)(fN)(mN)(mN)(mN)(mG)(dT)#(rU)(mG)(mU)(mA)(mG)( |
| | mC)(dT)#(rC)(mC)(mC)(dT)#(mU)(mU)(mC)(mU)(mC)(mA)(mU)(mU)#(mU)#(m |
| | C)#(mG) (SEQ ID NO: 82); |
| CR48-203- | (mN)#(mN)#(mN)#(mN)(mN)(mN)(dN)#(mN)(mN)(mN)(mN)(fN)(mN)(fN)(mN)( |
| V1.7 | fN)(mN)(mN)(fN)(mN)(fN)(mN)(mN)(mN)(mG)(rU)(rU)(mG)(mU)(mA)(mG)(m |
| | C)(rU)(rC)(dC)#(dC)#(dT)#(mU)(mU)(mC)(mU)(mC)(mA)(mU)(mU)#(mU)#(mC |
| | )#(mG) (SEQ ID NO: 83); |
| CR48-203- | (mN)#(mN)#(mN)#(mN)(mN)(mN)(dN)#(mN)(mN)(mN)(mN)(fN)(mN)(fN)(mN)( |
| V1.10 | fN)(mN)(mN)(fN)(mN)(fN)(mN)(mN)(mN)(mG)(rU)(rU)(mG)(mU)(mA)(mG)(m |
| | C)(rU)(rC)(mC)(dC)#(dT)#(mU)(mU)(mC)(mU)(mC)(mA)(mU)(mU)#(mU)#(mC) |
| | #(mG) (SEQ ID NO: 84); |
| CR48-203- | (mN)#(mN)#(mN)#(mN)(mN)(mN)(dN)#(mN)(mN)(mN)(mN)(fN)(mN)(fN)(mN)( |
| V1.12 | fN)(mN)(mN)(fN)(mN)(fN)(mN)(mN)(mN)(mG)(rU)(rU)(mG)(mU)(mA)(mG)(m |
| | C)(rU)(rC)(mC)(mC)(dT)#(mU)(mU)(mC)(mU)(mC)(mA)(mU)(mU)#(mU)#(mC) |
| | #(mG) (SEQ ID NO: 85); |
| CR48-203- | (mN)#(mN)#(mN)#(mN)(mN)(mN)(dN)#(mN)(mN)(mN)(mN)(fN)(mN)(fN)(mN)( |
| V1.18 | fN)(mN)(mN)(fN)(mN)(fN)(mN)(mN)(mN)(mG)(rU)(rU)(mG)(mU)(mA)(mG)(m |
| | C)(dT)#(rC)(dC)#(rC)(dT)#(mU)(mU)(mC)(mU)(mC)(mA)(mU)(mU)#(mU)#(mC) |
| | #(mG) (SEQ ID NO: 86); |
| CR48-203- | (mN)#(mN)#(mN)#(mN)(mN)(mN)(dN)#(mN)(mN)(mN)(mN)(fN)(mN)(fN)(mN)( |
| V1.19 | fN)(mN)(mN)(fN)(mN)(fN)(mN)(mN)(mN)(mG)(rU)(rU)(mG)(mU)(mA)(mG)(m |
| | C)(dT)#(rC)(dC)#(dC)#(dT)#(mU)(mU)(mC)(mU)(mC)(mA)(mU)(mU)#(mU)#(m |
| | C)#(mG) (SEQ ID NO: 87); |
| CR48-203- | (mN)#(mN)#(mN)#(mN)(mN)(mN)(dN)#(mN)(mN)(mN)(mN)(fN)(mN)(fN)(mN)( |
| V1.22 | fN)(mN)(mN)(fN)(mN)(fN)(mN)(mN)(mN)(mG)(rU)(rU)(mG)(mU)(mA)(mG)(m |
| | C)(dT)#(rC)(mC)(dC)#(dT)#(mU)(mU)(mC)(mU)(mC)(mA)(mU)(mU)#(mU)#(m |
| | C)#(mG) (SEQ ID NO: 88); |
| CR48-203- | (mN)#(mN)#(mN)#(mN)(mN)(mN)(dN)#(mN)(mN)(mN)(mN)(fN)(mN)(fN)(mN)( |
| V1.24 | fN)(mN)(mN)(fN)(mN)(fN)(mN)(mN)(mN)(mG)(rU)(rU)(mG)(mU)(mA)(mG)(m |
| | C)(dT)#(rC)(mC)(mC)(dT)#(mU)(mU)(mC)(mU)(mC)(mA)(mU)(mU)#(mU)#(mC |
| | )#(mG) (SEQ ID NO: 89); |
| CR48-203- | (mN)#(mN)#(mN)#(mN)(mN)(mN)(dN)#(mN)(mN)(mN)(mN)(fN)(mN)(fN)(mN)( |
| V1.30 | fN)(mN)(mN)(fN)(mN)(fN)(mN)(mN)(mN)(mG)(dT)#(rU)(mG)(mU)(mA)(mG)(m |
| | C)(dT)#(rC)(dC)#(rC)(dT)#(mU)(mU)(mC)(mU)(mC)(mA)(mU)(mU)#(mU)#(mC) |
| | #(mG) (SEQ ID NO: 90); |
| CR48-203- | (mN)#(mN)#(mN)#(mN)(mN)(mN)(dN)#(mN)(mN)(mN)(mN)(fN)(mN)(fN)(mN)( |
| V1.31 | fN)(mN)(mN)(fN)(mN)(fN)(mN)(mN)(mN)(mG)(dT)#(rU)(mG)(mU)(mA)(mG)(m |
| | C)(dT)#(rC)(dC)#(dC)#(dT)#(mU)(mU)(mC)(mU)(mC)(mA)(mU)(mU)#(mU)#(m |
| | C)#(mG) (SEQ ID NO: 91); |
| CR48-203- | (mN)#(mN)#(mN)#(mN)(mN)(mN)(dN)#(mN)(mN)(mN)(mN)(fN)(mN)(fN)(mN)( |
| V1.34 | fN)(mN)(mN)(fN)(mN)(fN)(mN)(mN)(mN)(mG)(dT)#(rU)(mG)(mU)(mA)(mG)(m |
| | C)(dT)#(rC)(mC)(dC)#(dT)#(mU)(mU)(mC)(mU)(mC)(mA)(mU)(mU)#(mU)#(m |
| | C)#(mG) (SEQ ID NO: 92); or |
| CR48-203- | (mN)#(mN)#(mN)#(mN)(mN)(mN)(dN)#(mN)(mN)(mN)(mN)(fN)(mN)(fN)(mN)( |
| V1.36 | fN)(mN)(mN)(fN)(mN)(fN)(mN)(mN)(mN)(mG)(dT)#(rU)(mG)(mU)(mA)(mG)(m |
| | C)(dT)#(rC)(mC)(mC)(dT)#(mU)(mU)(mC)(mU)(mC)(mA)(mU)(mU)#(mU)#(mC |
| | )#(mG) (SEQ ID NO: 93) |

wherein rN=RNA, mN=2′-O-methyl RNA, fN=2′-fluoro RNA, dN=2′-deoxy RNA, N #N=phosphorothioate linkage, and N=any nucleotide.

In certain embodiments, the chemically modified Nme gRNA comprises a tracrRNA portion nucleotide sequence of:

(SEQ ID NO: 10)
CGAAAUGAGAACCGUUGCUACAAUAAGGCCGUCUGAAAAGAUGUGCCG
CAACGCUCUGCCCCUUAAAGCUUCUGCUUUAAGGGGCAUCGUUUA
(TR93);
or
(SEQ ID NO: 11)
AGAACCGUUGCUACAAUAAGGCCGUCUGAAAAGAUGUGCCGCAACGCU
CUGCCCCUUAAAGCUCCUGCUUUAAGGGGCAUCGUUUA
(TR87).

In certain embodiments, the chemically modified Nme gRNA comprises a tracrRNA portion modification pattern selected from any one of:

(SEQ ID NO: 94)
(mC)#(mG)#(mA)#(rA)(rA)(rU)(rG)(rA)(rG
)(rA)(rA)(rC)(rC)(rG)(rU)(rU)(rG)(rC)(
rU)(rA)(rC)(rA)(rA)(rU)(rA)(rA)(rG)(rG
)(rC)(rC)(rG)(rU)(rC)(rU)(rG)(rA)(rA)(
rA)(rA)(rG)(rA)(rU)(rG)(rU)(rG)(rC)(rC
)(rG)(rC)(rA)(rA)(rC)(rG)(rC)(rU)(rC)(
rU)(rG)(rC)(rC)(rC)(rC)(rU)(rU)(rA)(rA
)(rA)(rG)(rC)(rU)(rC)(rC)(rU)(rG)(rC)(
rU)(rU)(rU)(rA)(rA)(rG)(rG)(rG)(rG)(rC
)(rA)(rU)(rC)(rG)(rU)#(mU)#(mU)#(mA)
(TR93-0);
(SEQ ID NO: 95)
(mC)#(mG)#(mA)#(rA)(rA)(rU)(rG)(rA)(rG
)(rA)(rA)(rC)(rC)(rG)(rU)(rU)(rG)(rC)(
rU)(rA)(rC)(rA)(rA)(rU)(rA)(rA)(rG)(rG
)(rC)(rC)(rG)(rU)(rC)(rU)(mG)(mA)(mA)(
mA)(rA)(rG)(rA)(rU)(rG)(rU)(rG)(rC)(rC
)(rG)(rC)(rA)(rA)(rC)(rG)(rC)(rU)(rC)(
rU)(rG)(rC)(rC)(rC)(rC)(rU)(rU)(rA)(rA
)(rA)(rG)(rC)(mU)(mC)(mC)(mU)(rG)(rC)(
rU)(rU)(rU)(rA)(rA)(rG)(rG)(rG)(rG)(rC
)(rA)(rU)(rC)(rG)(rU)#(mU)#(mU)#(mA)
(TR93-1);
(SEQ ID NO: 96)
(mC)#(mG)#(mA)#(mA)(mA)(mU)(mG)(mA)(mG
)(mA)(mA)(mC)(rC)(rG)(rU)(rU)(rG)(rC)(
rU)(rA)(rC)(rA)(rA)(rU)(rA)(rA)(rG)(rG
)(rC)(rC)(rG)(mU)(mC)(mU)(mG)(mA)(mA)(
mA)(mA)(mG)(rA)(rU)(rG)(rU)(rG)(rC)(rC
)(rG)(rC)(rA)(rA)(rC)(rG)(rC)(rU)(rC)(
rU)(rG)(rC)(rC)(mC)(mC)(mU)(mU)(mA)(mA
)(mA)(mG)(mC)(mU)(mC)(mC)(mU)(mG)(mC)(
mU)(mU)(mU)(mA)(mA)(mG)(mG)(rG)(rG)(rC
)(rA)(rU)(mC)(mG)(mU)#(mU)#(mU)#(mA)
(TR93-2), wherein rN = RNA, mN =
2′-O-methyl RNA, IN = 2′-fluoro RNA,
N#N = phosphorothioate linkage, and N =
any nucleotide.

In one aspect, the disclosure provides a chemically modified Neisseria meningitidis (Nme) guide RNA (gRNA) comprising: (a) a crRNA portion comprising (i) a guide sequence capable of hybridizing to a target polynucleotide sequence, and (ii) a repeat sequence; and (b) a tracrRNA portion comprising an anti-repeat nucleotide sequence that is complementary to the repeat sequence, wherein the crRNA portion comprises a modification pattern selected from the group consisting of:

| | |
|---|---|
| CR48-2 | (mN)#(mN)#(mN)#(mN)(mN)(rN)(rN)(rN)(rN)(rN)(rN)(rN)(rN)(rN)(rN)(rN)(rN)(r |
| | N)(rN)(rN)(rN)(rN)(rN)(rN)(rG)(rU)(rU)(rG)(rU)(rA)(rG)(rC)(rU)(rC)(rC)(rC) |
| | (rU)(mU)(mU)(mC)(mU)(mC)(mA)(mU)(mU)#(mU)#(mC)#(mG)(SEQ ID NO: 13); |
| CR48-154 | (mN)#(mN)#(mN)#(mN)(mN)(mN)(dN)#(mN)(mN)(mN)(mN)(fN)(mN)(fN)(mN)( |
| | fN)(mN)(mN)(rN)(rN)(rN)(mN)(rN)(rN)(mG)(rU)(rU)(rG)(mU)(mA)(mG)(mC)(r |
| | U)(rC)(rC)(rC)(rU)(mU)(mU)(mC)(mU)(mC)(mA)(mU)(mU)#(mU)#(mC)#(mG) |
| | (SEQ ID NO: 14); |
| CR48-155 | (mN)#(mN)#(mN)#(mN)(mN)(mN)(dN)#(mN)(mN)(mN)(mN)(mN)(mN)(fN)(mN) |
| | (fN)(mN)(mN)(rN)(rN)(rN)(mN)(rN)(rN)(mG)(rU)(rU)(rG)(mU)(mA)(mG)(mC)(r |
| | U)(rC)(rC)(rC)(rU)(mU)(mU)(mC)(mU)(mC)(mA)(mU)(mU)#(mU)#(mC)#(mG) |
| | (SEQ ID NO: 15); |
| CR48-190 | (mN)#(mN)#(mN)#(mN)(mN)(mN)(dN)#(mN)(mN)(mN)(mN)(fN)(mN)(fN)(mN)( |
| | fN)(mN)(mN)(rN)(fN)(mN)(mN)(mN)(mN)(mG)(rU)(rU)(rG)(mU)(mA)(mG)(mC) |
| | (rU)(rC)(rC)(rC)(rU)(mU)(mU)(mC)(mU)(mC)(mA)(mU)(mU)#(mU)#(mC)#(mG) |
| | (SEQ ID NO: 16); |
| CR48-191 | (mN)#(mN)#(mN)#(mN)(mN)(mN)(dN)#(mN)(mN)(mN)(mN)(fN)(mN)(fN)(mN)( |
| | fN)(mN)(mN)(rN)(mN)(fN)(mN)(mN)(mN)(mG)(rU)(rU)(rG)(mU)(mA)(mG)(mC) |
| | (rU)(rC)(rC)(rC)(rU)(mU)(mU)(mC)(mU)(mC)(mA)(mU)(mU)#(mU)#(mC)#(mG) |
| | (SEQ ID NO: 17); |
| CR48-192 | (mN)#(mN)#(mN)#(mN)(mN)(mN)(dN)#(mN)(mN)(mN)(mN)(mN)(mN)(fN)(mN) |
| | (fN)(mN)(mN)(rN)(fN)(mN)(mN)(mN)(mN)(mG)(rU)(rU)(rG)(mU)(mA)(mG)(mC |
| | )(rU)(rC)(rC)(rC)(rU)(mU)(mU)(mC)(mU)(mC)(mA)(mU)(mU)#(mU)#(mC)#(mG |
| | )(SEQ ID NO: 18); |
| CR48-193 | (mN)#(mN)#(mN)#(mN)(mN)(mN)(dN)#(mN)(mN)(mN)(mN)(mN)(mN)(fN)(mN) |
| | (fN)(mN)(mN)(rN)(mN)(fN)(mN)(mN)(mN)(mG)(rU)(rU)(rG)(mU)(mA)(mG)(mC |
| | )(rU)(rC)(rC)(rC)(rU)(mU)(mU)(mC)(mU)(mC)(mA)(mU)(mU)#(mU)#(mC)#(mG |
| | )(SEQ ID NO: 19); |
| CR48-194 | (mN)#(mN)#(mN)#(mN)(mN)(mN)(dN)#(mN)(mN)(mN)(mN)(fN)(mN)(fN)(mN)( |
| | IN)(mN)(mN)(rN)#(fN)(mN)(mN)(mN)(mN)(mG)(rU)(rU)(rG)(mU)(mA)(mG)(m |
| | C)(rU)(rC)(rC)(rC)(rU)(mU)(mU)(mC)(mU)(mC)(mA)(mU)(mU)#(mU)#(mC)#(m |
| | G)(SEQ ID NO: 20); |
| CR48-195 | (mN)#(mN)#(mN)#(mN)(mN)(mN)(dN)#(mN)(mN)(mN)(mN)(fN)(mN)(fN)(mN)( |
| | IN)(mN)(mN)(rN)#(mN)(IN)(mN)(mN)(mN)(mG)(rU)(rU)(rG)(mU)(mA)(mG)(m |
| | C)(rU)(rC)(rC)(rC)(rU)(mU)(mU)(mC)(mU)(mC)(mA)(mU)(mU)#(mU)#(mC)#(m |
| | G)(SEQ ID NO: 21); |
| CR48-196 | (mN)#(mN)#(mN)#(mN)(mN)(mN)(dN)#(mN)(mN)(mN)(mN)(mN)(mN)(fN)(mN) |
| | (fN)(mN)(mN)(rN)#(fN)(mN)(mN)(mN)(mN)(mG)(rU)(rU)(rG)(mU)(mA)(mG)(m |
| | C)(rU)(rC)(rC)(rC)(rU)(mU)(mU)(mC)(mU)(mC)(mA)(mU)(mU)#(mU)#(mC)#(m |
| | G)(SEQ ID NO: 22); |
| CR48-197 | (mN)#(mN)#(mN)#(mN)(mN)(mN)(dN)#(mN)(mN)(mN)(mN)(mN)(mN)(fN)(mN) |
| | (fN)(mN)(mN)(rN)#(mN)(fN)(mN)(mN)(mN)(mG)(rU)(rU)(rG)(mU)(mA)(mG)(m |
| | C)(rU)(rC)(rC)(rC)(rU)(mU)(mU)(mC)(mU)(mC)(mA)(mU)(mU)#(mU)#(mC)#(m |
| | G)(SEQ ID NO: 23); |
| CR48-198 | (mN)#(mN)#(mN)#(mN)(mN)(mN)(dN)#(mN)(mN)(mN)(mN)(fN)(mN)(fN)(mN)( |
| | fN)(mN)(mN)(mN)(fN)(mN)(mN)(mN)(mN)(mG)(rU)(rU)(rG)(mU)(mA)(mG)(m |
| | C)(rU)(rC)(rC)(rC)(rU)(mU)(mU)(mC)(mU)(mC)(mA)(mU)(mU)#(mU)#(mC)#(m |
| | G)(SEQ ID NO: 24); |
| CR48-199 | (mN)#(mN)#(mN)#(mN)(mN)(mN)(dN)#(mN)(mN)(mN)(mN)(fN)(mN)(fN)(mN)( |
| | fN)(mN)(mN)(mN)(mN)(fN)(mN)(mN)(mN)(mG)(rU)(rU)(rG)(mU)(mA)(mG)(m |
| | C)(rU)(rC)(rC)(rC)(rU)(mU)(mU)(mC)(mU)(mC)(mA)(mU)(mU)#(mU)#(mC)#(m |
| | G)(SEQ ID NO: 25); |
| CR48-200 | (mN)#(mN)#(mN)#(mN)(mN)(mN)(dN)#(mN)(mN)(mN)(mN)(mN)(mN)(fN)(mN) |
| | (fN)(mN)(mN)(mN)(fN)(mN)(mN)(mN)(mN)(mG)(rU)(rU)(rG)(mU)(mA)(mG)(m |
| | C)(rU)(rC)(rC)(rC)(rU)(mU)(mU)(mC)(mU)(mC)(mA)(mU)(mU)#(mU)#(mC)#(m |
| | G)(SEQ ID NO: 26); |
| CR48-201 | (mN)#(mN)#(mN)#(mN)(mN)(mN)(dN)#(mN)(mN)(mN)(mN)(mN)(mN)(fN)(mN) |
| | (fN)(mN)(mN)(mN)(mN)(fN)(mN)(mN)(mN)(mG)(rU)(rU)(rG)(mU)(mA)(mG)(m |
| | C)(rU)(rC)(rC)(rC)(rU)(mU)(mU)(mC)(mU)(mC)(mA)(mU)(mU)#(mU)#(mC)#(m |
| | G)(SEQ ID NO: 27); |
| CR48-202 | (mN)#(mN)#(mN)#(mN)(mN)(mN)(dN)#(mN)(mN)(mN)(mN)(fN)(mN)(fN)(mN)( |
| | fN)(mN)(mN)(fN)(fN)(mN)(mN)(mN)(mN)(mG)(rU)(rU)(rG)(mU)(mA)(mG)(mC) |
| | (rU)(rC)(rC)(rC)(rU)(mU)(mU)(mC)(mU)(mC)(mA)(mU)(mU)#(mU)#(mC)#(mG) |
| | (SEQ ID NO: 28); |
| CR48-203 | (mN)#(mN)#(mN)#(mN)(mN)(mN)(dN)#(mN)(mN)(mN)(mN)(fN)(mN)(fN)(mN)( |
| | fN)(mN)(mN)(fN)(mN)(fN)(mN)(mN)(mN)(mG)(rU)(rU)(rG)(mU)(mA)(mG)(mC) |
| | (rU)(rC)(rC)(rC)(rU)(mU)(mU)(mC)(mU)(mC)(mA)(mU)(mU)#(mU)#(mC)#(mG) |
| | (SEQ ID NO: 29); |
| CR48-204 | (mN)#(mN)#(mN)#(mN)(mN)(mN)(dN)#(mN)(mN)(mN)(mN)(mN)(mN)(fN)(mN) |
| | (fN)(mN)(mN)(fN)(fN)(mN)(mN)(mN)(mN)(mG)(rU)(rU)(rG)(mU)(mA)(mG)(mC |
| | )(rU)(rC)(rC)(rC)(rU)(mU)(mU)(mC)(mU)(mC)(mA)(mU)(mU)#(mU)#(mC)#(mG |
| | )(SEQ ID NO: 30); |
| CR48-205 | (mN)#(mN)#(mN)#(mN)(mN)(mN)(dN)#(mN)(mN)(mN)(mN)(mN)(mN)(fN)(mN) |
| | (fN)(mN)(mN)(fN)(mN)(fN)(mN)(mN)(mN)(mG)(rU)(rU)(rG)(mU)(mA)(mG)(mC |
| | )(rU)(rC)(rC)(rC)(rU)(mU)(mU)(mC)(mU)(mC)(mA)(mU)(mU)#(mU)#(mC)#(mG |
| | )(SEQ ID NO: 31); |
| CR48-213 | (mN)#(mN)#(mN)#(mN)(mN)(mN)(dN)#(mN)(mN)(mN)(mN)(mN)(mN)(fN)(mN) |
| | (IN)(mN)(mN)(rN)(rN)(rN)(mN)(rN)(rN)(mG)(dT)#(rU)(mG)(mU)(mA)(mG)(mC) |
| | (rU)(rC)(rC)(rC)(rU)(mU)(mU)(mC)(mU)(mC)(mA)(mU)(mU)#(mU)#(mC)#(mG) |
| | (SEQ ID NO: 32); |
| CR48-214 | (mN)#(mN)#(mN)#(mN)(mN)(mN)(dN)#(mN)(mN)(mN)(mN)(mN)(mN)(fN)(mN) |
| | (IN)(mN)(mN)(rN)(rN)(rN)(mN)(rN)(rN)(mG)(dT)#(rU)(mG)(mU)(mA)(mG)(mC) |
| | (rU)(rC)(mC)(rC)(rU)(mU)(mU)(mC)(mU)(mC)(mA)(mU)(mU)#(mU)#(mC)#(mG |
| | )(SEQ ID NO: 33); |
| CR48-215 | (mN)#(mN)#(mN)#(mN)(mN)(mN)(dN)#(mN)(mN)(mN)(mN)(mN)(mN)(fN)(mN) |
| | (IN)(mN)(mN)(rN)(rN)(rN)(mN)(rN)(rN)(mG)(dT)#(rU)(mG)(mU)(mA)(mG)(mC) |
| | (rU)(rC)(rC)(mC)(rU)(mU)(mU)(mC)(mU)(mC)(mA)(mU)(mU)#(mU)#(mC)#(mG |
| | )(SEQ ID NO: 34); |
| CR48-216 | (mN)#(mN)#(mN)#(mN)(mN)(mN)(dN)#(mN)(mN)(mN)(mN)(mN)(mN)(fN)(mN) |
| | (IN)(mN)(mN)(rN)(rN)(rN)(mN)(rN)(rN)(mG)(dT)#(rU)(mG)(mU)(mA)(mG)(mC) |
| | (rU)(rC)(mC)(mC)(rU)(mU)(mU)(mC)(mU)(mC)(mA)(mU)(mU)#(mU)#(mC)#(m |
| | G)(SEQ ID NO: 35); |
| CR48-217 | (mN)#(mN)#(mN)#(mN)(mN)(mN)(dN)#(mN)(mN)(mN)(mN)(mN)(mN)(fN)(mN) |
| | (fN)(mN)(mN)(rN)(rN)(rN)(mN)(rN)(rN)(mG)(rU)(rU)(rG)(mU)(mA)(mG)(mC)(d |
| | T)#(rC)(rC)(rC)(rU)(mU)(mU)(mC)(mU)(mC)(mA)(mU)(mU)#(mU)#(mC)#(mG) |
| | (SEQ ID NO: 36); |
| CR48-218 | (mN)#(mN)#(mN)#(mN)(mN)(mN)(dN)#(mN)(mN)(mN)(mN)(mN)(mN)(fN)(mN) |
| | (fN)(mN)(mN)(rN)(rN)(rN)(mN)(rN)(rN)(mG)(rU)(rU)(rG)(mU)(mA)(mG)(mC)(r |
| | U)(dC)#(rC)(rC)(rU)(mU)(mU)(mC)(mU)(mC)(mA)(mU)(mU)#(mU)#(mC)#(mG) |
| | (SEQ ID NO: 37); |
| CR48-219 | (mN)#(mN)#(mN)#(mN)(mN)(mN)(dN)#(mN)(mN)(mN)(mN)(mN)(mN)(fN)(mN) |
| | (fN)(mN)(mN)(rN)(rN)(rN)(mN)(rN)(rN)(mG)(rU)(rU)(rG)(mU)(mA)(mG)(mC)(r |
| | U)(rC)(dC)#(rC)(rU)(mU)(mU)(mC)(mU)(mC)(mA)(mU)(mU)#(mU)#(mC)#(mG) |
| | (SEQ ID NO: 38); |
| CR48-220 | (mN)#(mN)#(mN)#(mN)(mN)(mN)(dN)#(mN)(mN)(mN)(mN)(mN)(mN)(fN)(mN) |
| | (fN)(mN)(mN)(rN)(rN)(rN)(mN)(rN)(rN)(mG)(rU)(rU)(rG)(mU)(mA)(mG)(mC)(r |
| | U)(rC)(rC)(dC)#(rU)(mU)(mU)(mC)(mU)(mC)(mA)(mU)(mU)#(mU)#(mC)#(mG) |
| | (SEQ ID NO: 39); |
| CR48-221 | (mN)#(mN)#(mN)#(mN)(mN)(mN)(dN)#(mN)(mN)(mN)(mN)(mN)(mN)(fN)(mN) |
| | (fN)(mN)(mN)(rN)(rN)(rN)(mN)(rN)(rN)(mG)(rU)(rU)(rG)(mU)(mA)(mG)(mC)(r |
| | U)(rC)(rC)(rC)(dT)#(mU)(mU)(mC)(mU)(mC)(mA)(mU)(mU)#(mU)#(mC)#(mG) |
| | (SEQ ID NO: 40); |
| CR48-222 | (mN)#(mN)#(mN)#(mN)(mN)(mN)(dN)#(mN)(mN)(mN)(mN)(mN)(mN)(fN)(mN) |
| | (fN)(mN)(mN)(rN)(rN)(rN)(mN)(rN)(rN)(mG)(rU)(rU)(rG)(mU)(mA)(mG)(mC)(d |
| | T)#(rC)(dC)#(rC)(dT)#(mU)(mU)(mC)(mU)(mC)(mA)(mU)(mU)#(mU)#(mC)#(m |
| | G)(SEQ ID NO: 41); |
| CR48-223 | (mN)#(mN)#(mN)#(mN)(mN)(mN)(dN)#(mN)(mN)(mN)(mN)(mN)(mN)(fN)(mN) |
| | (fN)(mN)(mN)(rN)(rN)(rN)(mN)(rN)(rN)(mG)(dT)#(rU)(rG)(mU)(mA)(mG)(mC)( |
| | dT)#(dC)#(dC)#(rC)(dT)#(mU)(mU)(mC)(mU)(mC)(mA)(mU)(mU)#(mU)#(mC)# |
| | (mG)(SEQ ID NO: 42); |
| CR48-224 | (mN)#(mN)#(mN)#(mN)(mN)(mN)(dN)#(mN)(mN)(mN)(mN)(mN)(mN)(fN)(mN) |
| | (IN)(mN)(mN)(rN)(rN)(rN)(mN)(rN)(rN)(mG)(dT)#(rU)(mG)(mU)(mA)(mG)(mC) |
| | (dT)#(rC)(mC)(rC)(dT)#(mU)(mU)(mC)(mU)(mC)(mA)(mU)(mU)#(mU)#(mC)#( |
| | mG)(SEQ ID NO: 43); |
| CR48-225 | (mN)#(mN)#(mN)#(mN)(mN)(mN)(dN)#(mN)(mN)(mN)(mN)(mN)(mN)(fN)(mN) |
| | (IN)(mN)(mN)(rN)(rN)(rN)(mN)(rN)(rN)(mG)(dT)#(rU)(mG)(mU)(mA)(mG)(mC) |
| | (dT)#(rC)(dC)#(mC)(dT)#(mU)(mU)(mC)(mU)(mC)(mA)(mU)(mU)#(mU)#(mC)# |
| | (mG)(SEQ ID NO: 44); |
| CR48-226 | |
| | (fN)(mN)(mN)(rN)(rN)(rN)(mN)(rN)(rN)(mG)(dT)#(rU)(mG)(mU)(mA)(mG)(mC) |
| | (dT)#(rC)(mC)(mC)(dT)#(mU)(mU)(mC)(mU)(mC)(mA)(mU)(mU)#(mU)#(mC)#( |
| | mG)(SEQ ID NO: 45); |
| CR48-227 | (mN)#(mN)#(mN)#(mN)(mN)(mN)(dN)#(mN)(mN)(mN)(mN)(mN)(mN)(fN)(mN) |
| | (IN)(mN)(mN)(rN)(rN)(rN)(mN)(rN)(rN)(mG)(dT)#(rU)(mG)(mU)(mA)(mG)(mC) |
| | (dT)#(dC)#(mC)(mC)(dT)#(mU)(mU)(mC)(mU)(mC)(mA)(mU)(mU)#(mU)#(mC) |
| | #(mG)(SEQ ID NO: 46); |
| | (mN)#(mN)#(mN)#(mN)(mN)(mN)(dN)#(mN)(mN)(mN)(mN)(fN)(mN)(fN)(mN)( |
| CR48-199- | IN)(mN)(mN)(mN)(mN)(IN)(mN)(mN)(mN)(mG)(rU)(rU)(mG)(mU)(mA)(mG)(m |
| V1.1 | C)(rU)(rC)(rC)(dC)#(rU)(mU)(mU)(mC)(mU)(mC)(mA)(mU)(mU)#(mU)#(mC)#( |
| | mG)(SEQ ID NO: 47); |
| | (mN)#(mN)#(mN)#(mN)(mN)(mN)(dN)#(mN)(mN)(mN)(mN)(fN)(mN)(fN)(mN)( |
| CR48-199- | IN)(mN)(mN)(mN)(mN)(IN)(mN)(mN)(mN)(mG)(rU)(rU)(mG)(mU)(mA)(mG)(m |
| V1.2 | C)(rU)(rC)(dC)#(rC)(rU)(mU)(mU)(mC)(mU)(mC)(mA)(mU)(mU)#(mU)#(mC)#( |
| | mG)(SEQ ID NO: 48); |
| | (mN)#(mN)#(mN)#(mN)(mN)(mN)(dN)#(mN)(mN)(mN)(mN)(fN)(mN)(fN)(mN)( |
| CR48-199- | IN)(mN)(mN)(mN)(mN)(IN)(mN)(mN)(mN)(mG)(rU)(rU)(mG)(mU)(mA)(mG)(m |
| V1.3 | C)(rU)(rC)(rC)(rC)(dT)#(mU)(mU)(mC)(mU)(mC)(mA)(mU)(mU)#(mU)#(mC)#( |
| | mG)(SEQ ID NO: 49); |
| | (mN)#(mN)#(mN)#(mN)(mN)(mN)(dN)#(mN)(mN)(mN)(mN)(fN)(mN)(fN)(mN)( |
| CR48-199- | IN)(mN)(mN)(mN)(mN)(IN)(mN)(mN)(mN)(mG)(rU)(rU)(mG)(mU)(mA)(mG)(m |
| V1.4 | C)(rU)(rC)(mC)(rC)(rU)(mU)(mU)(mC)(mU)(mC)(mA)(mU)(mU)#(mU)#(mC)#( |
| | mG)(SEQ ID NO: 50); |
| | (mN)#(mN)#(mN)#(mN)(mN)(mN)(dN)#(mN)(mN)(mN)(mN)(fN)(mN)(fN)(mN)( |
| CR48-199- | IN)(mN)(mN)(mN)(mN)(IN)(mN)(mN)(mN)(mG)(rU)(rU)(mG)(mU)(mA)(mG)(m |
| V1.5 | C)(rU)(rC)(dC)#(dC)#(rU)(mU)(mU)(mC)(mU)(mC)(mA)(mU)(mU)#(mU)#(mC)# |
| | (mG)(SEQ ID NO: 51); |
| | (mN)#(mN)#(mN)#(mN)(mN)(mN)(dN)#(mN)(mN)(mN)(mN)(fN)(mN)(fN)(mN)( |
| CR48-199- | IN)(mN)(mN)(mN)(mN)(IN)(mN)(mN)(mN)(mG)(rU)(rU)(mG)(mU)(mA)(mG)(m |
| V1.6 | C)(rU)(rC)(dC)#(rC)(dT)#(mU)(mU)(mC)(mU)(mC)(mA)(mU)(mU)#(mU)#(mC)# |
| | (mG)(SEQ ID NO: 52); |
| | (mN)#(mN)#(mN)#(mN)(mN)(mN)(dN)#(mN)(mN)(mN)(mN)(fN)(mN)(fN)(mN)( |
| CR48-199- | IN)(mN)(mN)(mN)(mN)(IN)(mN)(mN)(mN)(mG)(rU)(rU)(mG)(mU)(mA)(mG)(m |
| V1.7 | C)(rU)(rC)(dC)#(dC)#(dT)#(mU)(mU)(mC)(mU)(mC)(mA)(mU)(mU)#(mU)#(mC |
| | )#(mG)(SEQ ID NO: 53); |
| | (mN)#(mN)#(mN)#(mN)(mN)(mN)(dN)#(mN)(mN)(mN)(mN)(fN)(mN)(fN)(mN)( |
| CR48-199- | IN)(mN)(mN)(mN)(mN)(IN)(mN)(mN)(mN)(mG)(rU)(rU)(mG)(mU)(mA)(mG)(m |
| V1.8 | C)(rU)(rC)(mC)(dC)#(rU)(mU)(mU)(mC)(mU)(mC)(mA)(mU)(mU)#(mU)#(mC)# |
| | (mG)(SEQ ID NO: 54); |
| | (mN)#(mN)#(mN)#(mN)(mN)(mN)(dN)#(mN)(mN)(mN)(mN)(fN)(mN)(fN)(mN)( |
| CR48-199- | IN)(mN)(mN)(mN)(mN)(IN)(mN)(mN)(mN)(mG)(rU)(rU)(mG)(mU)(mA)(mG)(m |
| V1.9 | C)(rU)(rC)(mC)(rC)(dT)#(mU)(mU)(mC)(mU)(mC)(mA)(mU)(mU)#(mU)#(mC)#( |
| | mG)(SEQ ID NO: 55); |
| | (mN)#(mN)#(mN)#(mN)(mN)(mN)(dN)#(mN)(mN)(mN)(mN)(fN)(mN)(fN)(mN)( |
| CR48-199- | IN)(mN)(mN)(mN)(mN)(IN)(mN)(mN)(mN)(mG)(rU)(rU)(mG)(mU)(mA)(mG)(m |
| Vl.10 | C)(rU)(rC)(mC)(dC)#(dT)#(mU)(mU)(mC)(mU)(mC)(mA)(mU)(mU)#(mU)#(mC) |
| | #(mG)(SEQ ID NO: 56); |
| | (mN)#(mN)#(mN)#(mN)(mN)(mN)(dN)#(mN)(mN)(mN)(mN)(fN)(mN)(fN)(mN)( |
| CR48-199- | IN)(mN)(mN)(mN)(mN)(IN)(mN)(mN)(mN)(mG)(rU)(rU)(mG)(mU)(mA)(mG)(m |
| V1.11 | C)(rU)(rC)(mC)(mC)(rU)(mU)(mU)(mC)(mU)(mC)(mA)(mU)(mU)#(mU)#(mC)#( |
| | mG)(SEQ ID NO: 57); |
| | (mN)#(mN)#(mN)#(mN)(mN)(mN)(dN)#(mN)(mN)(mN)(mN)(fN)(mN)(fN)(mN)( |
| CR48-199- | IN)(mN)(mN)(mN)(mN)(IN)(mN)(mN)(mN)(mG)(rU)(rU)(mG)(mU)(mA)(mG)(m |
| V1.12 | C)(rU)(rC)(mC)(mC)(dT)#(mU)(mU)(mC)(mU)(mC)(mA)(mU)(mU)#(mU)#(mC) |
| | #(mG)(SEQ ID NO: 58); |
| | (mN)#(mN)#(mN)#(mN)(mN)(mN)(dN)#(mN)(mN)(mN)(mN)(fN)(mN)(fN)(mN)( |
| CR48-199- | fN)(mN)(mN)(mN)(mN)(fN)(mN)(mN)(mN)(mG)(rU)(rU)(mG)(mU)(mA)(mG)(m |
| V1.13 | C)(dT)#(rC)(rC)(dC)#(rU)(mU)(mU)(mC)(mU)(mC)(mA)(mU)(mU)#(mU)#(mC)# |
| | (mG)(SEQ ID NO: 59); |
| | (mN)#(mN)#(mN)#(mN)(mN)(mN)(dN)#(mN)(mN)(mN)(mN)(fN)(mN)(fN)(mN)( |
| CR48-199- | fN)(mN)(mN)(mN)(mN)(fN)(mN)(mN)(mN)(mG)(rU)(rU)(mG)(mU)(mA)(mG)(m |
| Vl.14 | C)(dT)#(rC)(dC)#(rC)(rU)(mU)(mU)(mC)(mU)(mC)(mA)(mU)(mU)#(mU)#(mC)# |
| | (mG)(SEQ ID NO: 60); |
| | (mN)#(mN)#(mN)#(mN)(mN)(mN)(dN)#(mN)(mN)(mN)(mN)(fN)(mN)(fN)(mN)( |
| CR48-199- | fN)(mN)(mN)(mN)(mN)(fN)(mN)(mN)(mN)(mG)(rU)(rU)(mG)(mU)(mA)(mG)(m |
| V1.15 | C)(dT)#(rC)(rC)(rC)(dT)#(mU)(mU)(mC)(mU)(mC)(mA)(mU)(mU)#(mU)#(mC)# |
| | (mG)(SEQ ID NO: 61); |
| | (mN)#(mN)#(mN)#(mN)(mN)(mN)(dN)#(mN)(mN)(mN)(mN)(fN)(mN)(fN)(mN)( |
| CR48-199- | fN)(mN)(mN)(mN)(mN)(fN)(mN)(mN)(mN)(mG)(rU)(rU)(mG)(mU)(mA)(mG)(m |
| V1.16 | C)(dT)#(rC)(mC)(rC)(rU)(mU)(mU)(mC)(mU)(mC)(mA)(mU)(mU)#(mU)#(mC)#( |
| | mG)(SEQ ID NO: 62); |
| | (mN)#(mN)#(mN)#(mN)(mN)(mN)(dN)#(mN)(mN)(mN)(mN)(fN)(mN)(fN)(mN)( |
| CR48-199- | fN)(mN)(mN)(mN)(mN)(fN)(mN)(mN)(mN)(mG)(rU)(rU)(mG)(mU)(mA)(mG)(m |
| V1.17 | C)(dT)#(rC)(dC)#(dC)#(rU)(mU)(mU)(mC)(mU)(mC)(mA)(mU)(mU)#(mU)#(mC |
| | )#(mG)(SEQ ID NO: 63); |
| | (mN)#(mN)#(mN)#(mN)(mN)(mN)(dN)#(mN)(mN)(mN)(mN)(fN)(mN)(fN)(mN)( |
| CR48-199- | fN)(mN)(mN)(mN)(mN)(fN)(mN)(mN)(mN)(mG)(rU)(rU)(mG)(mU)(mA)(mG)(m |
| V1.18 | C)(dT)#(rC)(dC)#(rC)(dT)#(mU)(mU)(mC)(mU)(mC)(mA)(mU)(mU)#(mU)#(mC) |
| | #(mG)(SEQ ID NO: 64); |
| | (mN)#(mN)#(mN)#(mN)(mN)(mN)(dN)#(mN)(mN)(mN)(mN)(fN)(mN)(fN)(mN)( |
| CR48-199- | fN)(mN)(mN)(mN)(mN)(fN)(mN)(mN)(mN)(mG)(rU)(rU)(mG)(mU)(mA)(mG)(m |
| V1.19 | C)(dT)#(rC)(dC)#(dC)#(dT)#(mU)(mU)(mC)(mU)(mC)(mA)(mU)(mU)#(mU)#(m |
| | C)#(mG)(SEQ ID NO: 65); |
| | (mN)#(mN)#(mN)#(mN)(mN)(mN)(dN)#(mN)(mN)(mN)(mN)(fN)(mN)(fN)(mN)( |
| CR48-199- | fN)(mN)(mN)(mN)(mN)(fN)(mN)(mN)(mN)(mG)(rU)(rU)(mG)(mU)(mA)(mG)(m |
| V1.20 | C)(dT)#(rC)(mC)(dC)#(rU)(mU)(mU)(mC)(mU)(mC)(mA)(mU)(mU)#(mU)#(mC) |
| | #(mG)(SEQ ID NO: 66); |
| | (mN)#(mN)#(mN)#(mN)(mN)(mN)(dN)#(mN)(mN)(mN)(mN)(fN)(mN)(fN)(mN)( |
| CR48-199- | fN)(mN)(mN)(mN)(mN)(fN)(mN)(mN)(mN)(mG)(rU)(rU)(mG)(mU)(mA)(mG)(m |
| V1.21 | C)(dT)#(rC)(mC)(rC)(dT)#(mU)(mU)(mC)(mU)(mC)(mA)(mU)(mU)#(mU)#(mC) |
| | #(mG)(SEQ ID NO: 67); |
| | (mN)#(mN)#(mN)#(mN)(mN)(mN)(dN)#(mN)(mN)(mN)(mN)(fN)(mN)(fN)(mN)( |
| CR48-199- | fN)(mN)(mN)(mN)(mN)(fN)(mN)(mN)(mN)(mG)(rU)(rU)(mG)(mU)(mA)(mG)(m |
| V1.22 | C)(dT)#(rC)(mC)(dC)#(dT)#(mU)(mU)(mC)(mU)(mC)(mA)(mU)(mU)#(mU)#(m |
| | C)#(mG)(SEQ ID NO: 68); |
| | (mN)#(mN)#(mN)#(mN)(mN)(mN)(dN)#(mN)(mN)(mN)(mN)(fN)(mN)(fN)(mN)( |
| CR48-199- | fN)(mN)(mN)(mN)(mN)(fN)(mN)(mN)(mN)(mG)(rU)(rU)(mG)(mU)(mA)(mG)(m |
| V1.23 | C)(dT)#(rC)(mC)(mC)(rU)(mU)(mU)(mC)(mU)(mC)(mA)(mU)(mU)#(mU)#(mC) |
| | #(mG)(SEQ ID NO: 69); |
| | (mN)#(mN)#(mN)#(mN)(mN)(mN)(dN)#(mN)(mN)(mN)(mN)(fN)(mN)(fN)(mN)( |
| CR48-199- | fN)(mN)(mN)(mN)(mN)(fN)(mN)(mN)(mN)(mG)(rU)(rU)(mG)(mU)(mA)(mG)(m |
| V1.24 | C)(dT)#(rC)(mC)(mC)(dT)#(mU)(mU)(mC)(mU)(mC)(mA)(mU)(mU)#(mU)#(mC |
| | )#(mG)(SEQ ID NO: 70); |
| | (mN)#(mN)#(mN)#(mN)(mN)(mN)(dN)#(mN)(mN)(mN)(mN)(fN)(mN)(fN)(mN)( |
| CR48-199- | fN)(mN)(mN)(mN)(mN)(fN)(mN)(mN)(mN)(mG)(dT)#(rU)(mG)(mU)(mA)(mG)( |
| V1.25 | mC)(dT)#(rC)(rC)(dC)#(rU)(mU)(mU)(mC)(mU)(mC)(mA)(mU)(mU)#(mU)#(mC |
| | )#(mG)(SEQ ID NO: 71); |
| | (mN)#(mN)#(mN)#(mN)(mN)(mN)(dN)#(mN)(mN)(mN)(mN)(fN)(mN)(fN)(mN)( |
| CR48-199- | fN)(mN)(mN)(mN)(mN)(fN)(mN)(mN)(mN)(mG)(dT)#(rU)(mG)(mU)(mA)(mG)( |
| V1.26 | mC)(dT)#(rC)(dC)#(rC)(rU)(mU)(mU)(mC)(mU)(mC)(mA)(mU)(mU)#(mU)#(mC |
| | )#(mG)(SEQ ID NO: 72); |
| | (mN)#(mN)#(mN)#(mN)(mN)(mN)(dN)#(mN)(mN)(mN)(mN)(fN)(mN)(fN)(mN)( |
| CR48-199- | fN)(mN)(mN)(mN)(mN)(fN)(mN)(mN)(mN)(mG)(dT)#(rU)(mG)(mU)(mA)(mG)( |
| V1.27 | mC)(dT)#(rC)(rC)(rC)(dT)#(mU)(mU)(mC)(mU)(mC)(mA)(mU)(mU)#(mU)#(mC |
| | )#(mG)(SEQ ID NO: 73); |
| | (mN)#(mN)#(mN)#(mN)(mN)(mN)(dN)#(mN)(mN)(mN)(mN)(fN)(mN)(fN)(mN)( |
| CR48-199- | fN)(mN)(mN)(mN)(mN)(fN)(mN)(mN)(mN)(mG)(dT)#(rU)(mG)(mU)(mA)(mG)( |
| V1.28 | mC)(dT)#(rC)(mC)(rC)(rU)(mU)(mU)(mC)(mU)(mC)(mA)(mU)(mU)#(mU)#(mC) |
| | #(mG)(SEQ ID NO: 74); |
| | (mN)#(mN)#(mN)#(mN)(mN)(mN)(dN)#(mN)(mN)(mN)(mN)(fN)(mN)(IN)(mN)( |
| CR48-199- | IN)(mN)(mN)(mN)(mN)(fN)(mN)(mN)(mN)(mG)(dT)#(rU)(mG)(mU)(mA)(mG)( |
| V1.29 | mC)(dT)#(rC)(dC)#(dC)#(rU)(mU)(mU)(mC)(mU)(mC)(mA)(mU)(mU)#(mU)#(m |
| | C)#(mG)(SEQ ID NO: 75); |
| | (mN)#(mN)#(mN)#(mN)(mN)(mN)(dN)#(mN)(mN)(mN)(mN)(fN)(mN)(IN)(mN)( |
| CR48-199- | IN)(mN)(mN)(mN)(mN)(fN)(mN)(mN)(mN)(mG)(dT)#(rU)(mG)(mU)(mA)(mG)( |
| V1.30 | mC)(dT)#(rC)(dC)#(rC)(dT)#(mU)(mU)(mC)(mU)(mC)(mA)(mU)(mU)#(mU)#(m |
| | C)#(mG)(SEQ ID NO: 76); |
| | (mN)#(mN)#(mN)#(mN)(mN)(mN)(dN)#(mN)(mN)(mN)(mN)(fN)(mN)(IN)(mN)( |
| CR48-199- | IN)(mN)(mN)(mN)(mN)(fN)(mN)(mN)(mN)(mG)(dT)#(rU)(mG)(mU)(mA)(mG)( |
| V1.31 | mC)(dT)#(rC)(dC)#(dC)#(dT)#(mU)(mU)(mC)(mU)(mC)(mA)(mU)(mU)#(mU)#( |
| | mC)#(mG)(SEQ ID NO: 77); |
| | (mN)#(mN)#(mN)#(mN)(mN)(mN)(dN)#(mN)(mN)(mN)(mN)(fN)(mN)(IN)(mN)( |
| CR48-199- | IN)(mN)(mN)(mN)(mN)(fN)(mN)(mN)(mN)(mG)(dT)#(rU)(mG)(mU)(mA)(mG)( |
| V1.32 | mC)(dT)#(rC)(mC)(dC)#(rU)(mU)(mU)(mC)(mU)(mC)(mA)(mU)(mU)#(mU)#(m |
| | C)#(mG)(SEQ ID NO: 78); |
| | (mN)#(mN)#(mN)#(mN)(mN)(mN)(dN)#(mN)(mN)(mN)(mN)(fN)(mN)(IN)(mN)( |
| CR48-199- | IN)(mN)(mN)(mN)(mN)(fN)(mN)(mN)(mN)(mG)(dT)#(rU)(mG)(mU)(mA)(mG)( |
| V1.33 | mC)(dT)#(rC)(mC)(rC)(dT)#(mU)(mU)(mC)(mU)(mC)(mA)(mU)(mU)#(mU)#(m |
| | C)#(mG)(SEQ ID NO: 79); |
| | (mN)#(mN)#(mN)#(mN)(mN)(mN)(dN)#(mN)(mN)(mN)(mN)(fN)(mN)(IN)(mN)( |
| CR48-199- | IN)(mN)(mN)(mN)(mN)(fN)(mN)(mN)(mN)(mG)(dT)#(rU)(mG)(mU)(mA)(mG)( |
| V1.34 | mC)(dT)#(rC)(mC)(dC)#(dT)#(mU)(mU)(mC)(mU)(mC)(mA)(mU)(mU)#(mU)#( |
| | mC)#(mG)(SEQ ID NO: 80); |
| | (mN)#(mN)#(mN)#(mN)(mN)(mN)(dN)#(mN)(mN)(mN)(mN)(fN)(mN)(IN)(mN)( |
| CR48-199- | IN)(mN)(mN)(mN)(mN)(fN)(mN)(mN)(mN)(mG)(dT)#(rU)(mG)(mU)(mA)(mG)( |
| V1.35 | mC)(dT)#(rC)(mC)(mC)(rU)(mU)(mU)(mC)(mU)(mC)(mA)(mU)(mU)#(mU)#(m |
| | C)#(mG)(SEQ ID NO: 81); |
| | (mN)#(mN)#(mN)#(mN)(mN)(mN)(dN)#(mN)(mN)(mN)(mN)(fN)(mN)(IN)(mN)( |
| CR48-199- | IN)(mN)(mN)(mN)(mN)(fN)(mN)(mN)(mN)(mG)(dT)#(rU)(mG)(mU)(mA)(mG)( |
| V1.36 | mC)(dT)#(rC)(mC)(mC)(dT)#(mU)(mU)(mC)(mU)(mC)(mA)(mU)(mU)#(mU)#(m |
| | C)#(mG)(SEQ ID NO: 82); |
| | (mN)#(mN)#(mN)#(mN)(mN)(mN)(dN)#(mN)(mN)(mN)(mN)(fN)(mN)(IN)(mN)( |
| CR48-203- | fN)(mN)(mN)(IN)(mN)(fN)(mN)(mN)(mN)(mG)(rU)(rU)(mG)(mU)(mA)(mG)(m |
| V1.7 | C)(rU)(rC)(dC)#(dC)#(dT)#(mU)(mU)(mC)(mU)(mC)(mA)(mU)(mU)#(mU)#(mC |
| | )#(mG)(SEQ ID NO: 83); |
| | (mN)#(mN)#(mN)#(mN)(mN)(mN)(dN)#(mN)(mN)(mN)(mN)(fN)(mN)(IN)(mN)( |
| CR48-203- | fN)(mN)(mN)(IN)(mN)(fN)(mN)(mN)(mN)(mG)(rU)(rU)(mG)(mU)(mA)(mG)(m |
| Vl.10 | C)(rU)(rC)(mC)(dC)#(dT)#(mU)(mU)(mC)(mU)(mC)(mA)(mU)(mU)#(mU)#(mC) |
| | #(mG)(SEQ ID NO: 84); |
| | (mN)#(mN)#(mN)#(mN)(mN)(mN)(dN)#(mN)(mN)(mN)(mN)(fN)(mN)(IN)(mN)( |
| CR48-203- | fN)(mN)(mN)(IN)(mN)(fN)(mN)(mN)(mN)(mG)(rU)(rU)(mG)(mU)(mA)(mG)(m |
| V1.12 | C)(rU)(rC)(mC)(mC)(dT)#(mU)(mU)(mC)(mU)(mC)(mA)(mU)(mU)#(mU)#(mC) |
| | #(mG)(SEQ ID NO: 85); |
| | (mN)#(mN)#(mN)#(mN)(mN)(mN)(dN)#(mN)(mN)(mN)(mN)(fN)(mN)(IN)(mN)( |
| CR48-203- | fN)(mN)(mN)(IN)(mN)(fN)(mN)(mN)(mN)(mG)(rU)(rU)(mG)(mU)(mA)(mG)(m |
| V1.18 | C)(dT)#(rC)(dC)#(rC)(dT)#(mU)(mU)(mC)(mU)(mC)(mA)(mU)(mU)#(mU)#(mC) |
| | #(mG)(SEQ ID NO: 86); |
| | (mN)#(mN)#(mN)#(mN)(mN)(mN)(dN)#(mN)(mN)(mN)(mN)(fN)(mN)(fN)(mN)( |
| CR48-203- | fN)(mN)(mN)(IN)(mN)(IN)(mN)(mN)(mN)(mG)(rU)(rU)(mG)(mU)(mA)(mG)(m |
| V1.19 | C)(dT)#(rC)(dC)#(dC)#(dT)#(mU)(mU)(mC)(mU)(mC)(mA)(mU)(mU)#(mU)#(m |
| | C)#(mG)(SEQ ID NO: 87); |
| | (mN)#(mN)#(mN)#(mN)(mN)(mN)(dN)#(mN)(mN)(mN)(mN)(fN)(mN)(IN)(mN)( |
| CR48-203- | fN)(mN)(mN)(IN)(mN)(fN)(mN)(mN)(mN)(mG)(rU)(rU)(mG)(mU)(mA)(mG)(m |
| V1.22 | C)(dT)#(rC)(mC)(dC)#(dT)#(mU)(mU)(mC)(mU)(mC)(mA)(mU)(mU)#(mU)#(m |
| | C)#(mG)(SEQ ID NO: 88); |
| | (mN)#(mN)#(mN)#(mN)(mN)(mN)(dN)#(mN)(mN)(mN)(mN)(fN)(mN)(IN)(mN)( |
| CR48-203- | fN)(mN)(mN)(IN)(mN)(fN)(mN)(mN)(mN)(mG)(rU)(rU)(mG)(mU)(mA)(mG)(m |
| V1.24 | C)(dT)#(rC)(mC)(mC)(dT)#(mU)(mU)(mC)(mU)(mC)(mA)(mU)(mU)#(mU)#(mC |
| | )#(mG)(SEQ ID NO: 89); |
| | (mN)#(mN)#(mN)#(mN)(mN)(mN)(dN)#(mN)(mN)(mN)(mN)(fN)(mN)(IN)(mN)( |
| CR48-203- | IN)(mN)(mN)(IN)(mN)(IN)(mN)(mN)(mN)(mG)(dT)#(rU)(mG)(mU)(mA)(mG)(m |
| V1.30 | C)(dT)#(rC)(dC)#(rC)(dT)#(mU)(mU)(mC)(mU)(mC)(mA)(mU)(mU)#(mU)#(mC) |
| | #(mG)(SEQ ID NO: 90); |
| | (mN)#(mN)#(mN)#(mN)(mN)(mN)(dN)#(mN)(mN)(mN)(mN)(fN)(mN)(IN)(mN)( |
| CR48-203- | IN)(mN)(mN)(IN)(mN)(IN)(mN)(mN)(mN)(mG)(dT)#(rU)(mG)(mU)(mA)(mG)(m |
| V1.31 | C)(dT)#(rC)(dC)#(dC)#(dT)#(mU)(mU)(mC)(mU)(mC)(mA)(mU)(mU)#(mU)#(m |
| | C)#(mG)(SEQ ID NO: 91); |
| | (mN)#(mN)#(mN)#(mN)(mN)(mN)(dN)#(mN)(mN)(mN)(mN)(fN)(mN)(IN)(mN)( |
| CR48-203- | IN)(mN)(mN)(fN)(mN)(fN)(mN)(mN)(mN)(mG)(dT)#(rU)(mG)(mU)(mA)(mG)(m |
| V1.34 | C)(dT)#(rC)(mC)(dC)#(dT)#(mU)(mU)(mC)(mU)(mC)(mA)(mU)(mU)#(mU)#(m |
| | C)#(mG)(SEQ ID NO: 92); or |
| | (mN)#(mN)#(mN)#(mN)(mN)(mN)(dN)#(mN)(mN)(mN)(mN)(fN)(mN)(IN)(mN)( |
| CR48-203- | IN)(mN)(mN)(fN)(mN)(fN)(mN)(mN)(mN)(mG)(dT)#(rU)(mG)(mU)(mA)(mG)(m |
| V1.36 | C)(dT)#(rC)(mC)(mC)(dT)#(mU)(mU)(mC)(mU)(mC)(mA)(mU)(mU)#(mU)#(mC |
| | )#(mG)(SEQ ID NO: 93), |

wherein rN=RNA, mN=2′-O-methyl RNA, fN=2′-fluoro RNA, dN=2′-deoxy RNA, N #N=phosphorothioate linkage, and N=any nucleotide.

In certain embodiments, the tracrRNA portion comprises one or more modified nucleotides each independently selected from a modification of a ribose group, a phosphate group, a nucleobase, or a combination thereof.

In certain embodiments, each modification of the ribose group is independently selected from the group consisting of 2′-O-methyl, 2′-fluoro, 2′-deoxy, 2′-O-(2-methoxyethyl) (MOE), 2′-NH₂ (2′-amino), 4′-thio, a bicyclic nucleotide, a locked nucleic acid (LNA), a 2′-(S)-constrained ethyl (S-cEt), a constrained MOE, and a 2′-O,4′-C-aminomethylene bridged nucleic acid (2′,4′-BNA^{N} c).

In certain embodiments, at least 40% of the ribose groups are chemically modified. In certain embodiments, at least 80% of the ribose groups are chemically modified. In certain embodiments, 100% of the ribose groups are chemically modified.

In certain embodiments, each modification of the phosphate group is independently selected from the group consisting of a phosphorothioate, phosphonoacetate (PACE), thiophosphonoacetate (thioPACE), amide, triazole, phosphonate, and phosphotriester modification.

In certain embodiments, each modification of the nucleobase group is independently selected from the group consisting of 2-thiouridine, 4-thiouridine, N⁶-methyladenosine, pseudouridine, 2,6-diaminopurine, inosine, thymidine, 5-methylcytosine, 5-substituted pyrimidine, isoguanine, isocytosine, and halogenated aromatic groups.

In certain embodiments, the tracrRNA portion comprises at least 40% modified nucleotides. In certain embodiments, the tracrRNA portion comprises at least 80% modified nucleotides. In certain embodiments, the tracrRNA portion comprises at least 90% modified nucleotides. In certain embodiments, the tracrRNA portion comprises 100% chemically modified nucleotides.

In certain embodiments, the chemically modified Nme gRNA comprises a tracrRNA portion modification pattern selected from any one of:

(SEQ ID NO: 94)
(mC)#(mG)#(mA)#(rA)(rA)(rU)(rG)(rA)(rG)
(rA)(rA)(rC)(rC)(rG)(rU)(rU)(rG)(rC)(rU
)(rA)(rC)(rA)(rA)(rU)(rA)(rA)(rG)(rG)(r
C)(rC)(rG)(rU)(rC)(rU)(rG)(rA)(rA)(rA)(
rA)(rG)(rA)(rU)(rG)(rU)(rG)(rC)(rC)(rG)
(rC)(rA)(rA)(rC)(rG)(rC)(rU)(rC)(rU)(rG
)(rC)(rC)(rC)(rC)(rU)(rU)(rA)(rA)(rA)(r
G)(rC)(rU)(rC)(rC)(rU)(rG)(rC)(rU)(rU)(
rU)(rA)(rA)(rG)(rG)(rG)(rG)(rC)(rA)(rU)
(rC)(rG)(rU)#(mU)#(mU)#(mA)
(TR93-0);
(SEQ ID NO: 95)
(mC)#(mG)#(mA)#(rA)(rA)(rU)(rG)(rA)(rG)
(rA)(rA)(rC)(rC)(rG)(rU)(rU)(rG)(rC)(rU
)(rA)(rC)(rA)(rA)(rU)(rA)(rA)(rG)(rG)(r
C)(rC)(rG)(rU)(rC)(rU)(mG)(mA)(mA)(mA)(
rA)(rG)(rA)(rU)(rG)(rU)(rG)(rC)(rC)(rG)
(rC)(rA)(rA)(rC)(rG)(rC)(rU)(rC)(rU)(rG
)(rC)(rC)(rC)(rC)(rU)(rU)(rA)(rA)(rA)(r
G)(rC)(mU)(mC)(mC)(mU)(rG)(rC)(rU)(rU)(
rU)(rA)(rA)(rG)(rG)(rG)(rG)(rC)(rA)(rU)
(rC)(rG)(rU)#(mU)#(mU)#(mA)
(TR93-1);
(SEQ ID NO: 96)
(mC)#(mG)#(mA)#(mA)(mA)(mU)(mG)(mA)(mG)
(mA)(mA)(mC)(rC)(rG)(rU)(rU)(rG)(rC)(rU
)(rA)(rC)(rA)(rA)(rU)(rA)(rA)(rG)(rG)(r
C)(rC)(rG)(mU)(mC)(mU)(mG)(mA)(mA)(mA)(
mA)(mG)(rA)(rU)(rG)(rU)(rG)(rC)(rC)(rG)
(rC)(rA)(rA)(rC)(rG)(rC)(rU)(rC)(rU)(rG
)(rC)(rC)(mC)(mC)(mU)(mU)(mA)(mA)(mA)(m
G)(mC)(mU)(mC)(mC)(mU)(mG)(mC)(mU)(mU)(
mU)(mA)(mA)(mG)(mG)(rG)(rG)(rC)(rA)(rU)
(mC)(mG)(mU)#(mU)#(mU)#(mA)
(TR93-2), wherein rN = RNA, mN =
2′-O-methyl RNA, fN = 2′-fluoro RNA,
N#N = phosphorothioate linkage,
and N = any nucleotide.

In one aspect, the disclosure provides a chemically modified Neisseria meningitidis (Nme) guide RNA (gRNA) comprising: (a) a crRNA portion comprising (i) a guide sequence capable of hybridizing to a target polynucleotide sequence, and (ii) a repeat sequence; and (b) a tracrRNA portion comprising an anti-repeat nucleotide sequence that is complementary to the repeat sequence, wherein the tracrRNA portion comprises a modification pattern selected from anyone of:

(SEQ ID NO: 94)
(mC)#(mG)#(mA)#(rA)(rA)(rU)(rG)(rA)(rG)
(rA)(rA)(rC)(rC)(rG)(rU)(rU)(rG)(rC)(rU
)(rA)(rC)(rA)(rA)(rU)(rA)(rA)(rG)(rG)(r
C)(rC)(rG)(rU)(rC)(rU)(rG)(rA)(rA)(rA)(
rA)(rG)(rA)(rU)(rG)(rU)(rG)(rC)(rC)(rG)
(rC)(rA)(rA)(rC)(rG)(rC)(rU)(rC)(rU)(rG
)(rC)(rC)(rC)(rC)(rU)(rU)(rA)(rA)(rA)(r
G)(rC)(rU)(rC)(rC)(rU)(rG)(rC)(rU)(rU)(
rU)(rA)(rA)(rG)(rG)(rG)(rG)(rC)(rA)(rU)
(rC)(rG)(rU)#(mU)#(mU)#(mA)
(TR93-0);
(SEQ ID NO: 95)
(mC)#(mG)#(mA)#(rA)(rA)(rU)(rG)(rA)(rG)
(rA)(rA)(rC)(rC)(rG)(rU)(rU)(rG)(rC)(rU
)(rA)(rC)(rA)(rA)(rU)(rA)(rA)(rG)(rG)(r
C)(rC)(rG)(rU)(rC)(rU)(mG)(mA)(mA)(mA)(
rA)(rG)(rA)(rU)(rG)(rU)(rG)(rC)(rC)(rG)
(rC)(rA)(rA)(rC)(rG)(rC)(rU)(rC)(rU)(rG
)(rC)(rC)(rC)(rC)(rU)(rU)(rA)(rA)(rA)(r
G)(rC)(mU)(mC)(mC)(mU)(rG)(rC)(rU)(rU)(
rU)(rA)(rA)(rG)(rG)(rG)(rG)(rC)(rA)(rU)
(rC)(rG)(rU)#(mU)#(mU)#(mA)(TR93-1);
(SEQ ID NO: 96)
(mC)#(mG)#(mA)#(mA)(mA)(mU)(mG)(mA)(mG)
(mA)(mA)(mC)(rC)(rG)(rU)(rU)(rG)(rC)(rU
)(rA)(rC)(rA)(rA)(rU)(rA)(rA)(rG)(rG)(r
C)(rC)(rG)(mU)(mC)(mU)(mG)(mA)(mA)(mA)(
mA)(mG)(rA)(rU)(rG)(rU)(rG)(rC)(rC)(rG)
(rC)(rA)(rA)(rC)(rG)(rC)(rU)(rC)(rU)(rG
)(rC)(rC)(mC)(mC)(mU)(mU)(mA)(mA)(mA)(m
G)(mC)(mU)(mC)(mC)(mU)(mG)(mC)(mU)(mU)(
mU)(mA)(mA)(mG)(mG)(rG)(rG)(rC)(rA)(rU)
(mC)(mG)(mU)#(mU)#(mU)#(mA)
(TR93-2), wherein rN = RNA, mN =
2′-O-methyl RNA, IN = 2′-fluoro
RNA, N#N = phosphorothioate
linkage, and N = any
nucleotide.

In certain embodiments, the crRNA portion comprises one or more modified nucleotides each independently selected from a modification of a ribose group, a phosphate group, a nucleobase, or a combination thereof.

In certain embodiments, each modification of the ribose group is independently selected from the group consisting of 2′-O-methyl, 2′-fluoro, 2′-deoxy, 2′-O-(2-methoxyethyl) (MOE), 2′-NH₂ (2′-amino), 4′-thio, a bicyclic nucleotide, a locked nucleic acid (LNA), a 2′-(S)-constrained ethyl (S-cEt), a constrained MOE, and a 2′-O,4′-C-aminomethylene bridged nucleic acid (2′,4′-BNA^{NC}).

In certain embodiments, at least 50% of the ribose groups are chemically modified. In certain embodiments, at least 80% of the ribose groups are chemically modified. In certain embodiments, 100% of the ribose groups are chemically modified.

In certain embodiments, each modification of the phosphate group is independently selected from the group consisting of a phosphorothioate, phosphonoacetate (PACE), thiophosphonoacetate (thioPACE), amide, triazole, phosphonate, or phosphotriester modification.

In certain embodiments, each modification of the nucleobase group is independently selected from the group consisting of 2-thiouridine, 4-thiouridine, N⁶-methyladenosine, pseudouridine, 2,6-diaminopurine, inosine, thymidine, 5-methylcytosine, 5-substituted pyrimidine, isoguanine, isocytosine, and halogenated aromatic groups.

In certain embodiments, the crRNA portion comprises at least 50% modified nucleotides. In certain embodiments, the crRNA portion comprises at least 80% modified nucleotides. In certain embodiments, the crRNA portion comprises at least 90% modified nucleotides. In certain embodiments, the crRNA portion comprises 100% chemically modified nucleotides.

In certain embodiments, the chemically modified Nme gRNA comprises a crRNA portion modification pattern selected from the group consisting of:

| | |
|---|---|
| CR48-2 | (mN)#(mN)#(mN)#(mN)(mN)(rN)(rN)(rN)(rN)(rN)(rN)(rN)(rN)(rN)(rN)(rN)(rN) |
| | (rN)(rN)(rN)(rN)(rN)(rN)(rN)(rG)(rU)(rU)(rG)(rU)(rA)(rG)(rC)(rU)(rC)(rC) |
| | (rC)(rU)(mU)(mU)(mC)(mU)(mC)(mA)(mU)(mU)#(mU)#(mC)#(mG)(SEQ ID NO: 13); |
| CR48-154 | (mN)#(mN)#(mN)#(mN)(mN)(mN)(dN)#(mN)(mN)(mN)(mN)(fN)(mN)(fN)(mN)( |
| | fN)(mN)(mN)(rN)(rN)(rN)(mN)(rN)(rN)(mG)(rU)(rU)(rG)(mU)(mA)(mG)(mC)(r |
| | U)(rC)(rC)(rC)(rU)(mU)(mU)(mC)(mU)(mC)(mA)(mU)(mU)#(mU)#(mC)#(mG) |
| | (SEQ ID NO: 14); |
| CR48-155 | (mN)#(mN)#(mN)#(mN)(mN)(mN)(dN)#(mN)(mN)(mN)(mN)(mN)(mN)(fN)(mN) |
| | (fN)(mN)(mN)(rN)(rN)(rN)(mN)(rN)(rN)(mG)(rU)(rU)(rG)(mU)(mA)(mG)(mC)(r |
| | U)(rC)(rC)(rC)(rU)(mU)(mU)(mC)(mU)(mC)(mA)(mU)(mU)#(mU)#(mC)#(mG) |
| | (SEQ ID NO: 15); |
| CR48-190 | (mN)#(mN)#(mN)#(mN)(mN)(mN)(dN)#(mN)(mN)(mN)(mN)(fN)(mN)(fN)(mN)( |
| | fN)(mN)(mN)(rN)(fN)(mN)(mN)(mN)(mN)(mG)(rU)(rU)(rG)(mU)(mA)(mG)(mC) |
| | (rU)(rC)(rC)(rC)(rU)(mU)(mU)(mC)(mU)(mC)(mA)(mU)(mU)#(mU)#(mC)#(mG) |
| | (SEQ ID NO: 16); |
| CR48-191 | (mN)#(mN)#(mN)#(mN)(mN)(mN)(dN)#(mN)(mN)(mN)(mN)(fN)(mN)(fN)(mN)( |
| | fN)(mN)(mN)(rN)(mN)(fN)(mN)(mN)(mN)(mG)(rU)(rU)(rG)(mU)(mA)(mG)(mC) |
| | (rU)(rC)(rC)(rC)(rU)(mU)(mU)(mC)(mU)(mC)(mA)(mU)(mU)#(mU)#(mC)#(mG) |
| | (SEQ ID NO: 17); |
| CR48-192 | (mN)#(mN)#(mN)#(mN)(mN)(mN)(dN)#(mN)(mN)(mN)(mN)(mN)(mN)(fN)(mN) |
| | (fN)(mN)(mN)(rN)(fN)(mN)(mN)(mN)(mN)(mG)(rU)(rU)(rG)(mU)(mA)(mG)(mC |
| | )(rU)(rC)(rC)(rC)(rU)(mU)(mU)(mC)(mU)(mC)(mA)(mU)(mU)#(mU)#(mC)#(mG |
| | )(SEQ ID NO: 18); |
| CR48-193 | (mN)#(mN)#(mN)#(mN)(mN)(mN)(dN)#(mN)(mN)(mN)(mN)(mN)(mN)(fN)(mN) |
| | (fN)(mN)(mN)(rN)(mN)(fN)(mN)(mN)(mN)(mG)(rU)(rU)(rG)(mU)(mA)(mG)(mC |
| | )(rU)(rC)(rC)(rC)(rU)(mU)(mU)(mC)(mU)(mC)(mA)(mU)(mU)#(mU)#(mC)#(mG |
| | )(SEQ ID NO: 19); |
| CR48-194 | (mN)#(mN)#(mN)#(mN)(mN)(mN)(dN)#(mN)(mN)(mN)(mN)(fN)(mN)(fN)(mN)( |
| | IN)(mN)(mN)(rN)#(fN)(mN)(mN)(mN)(mN)(mG)(rU)(rU)(rG)(mU)(mA)(mG)(m |
| | C)(rU)(rC)(rC)(rC)(rU)(mU)(mU)(mC)(mU)(mC)(mA)(mU)(mU)#(mU)#(mC)#(m |
| | G)(SEQ ID NO: 20); |
| CR48-195 | (mN)#(mN)#(mN)#(mN)(mN)(mN)(dN)#(mN)(mN)(mN)(mN)(fN)(mN)(fN)(mN)( |
| | IN)(mN)(mN)(rN)#(mN)(IN)(mN)(mN)(mN)(mG)(rU)(rU)(rG)(mU)(mA)(mG)(m |
| | C)(rU)(rC)(rC)(rC)(rU)(mU)(mU)(mC)(mU)(mC)(mA)(mU)(mU)#(mU)#(mC)#(m |
| | G)(SEQ ID NO: 21); |
| CR48-196 | (mN)#(mN)#(mN)#(mN)(mN)(mN)(dN)#(mN)(mN)(mN)(mN)(mN)(mN)(fN)(mN) |
| | (fN)(mN)(mN)(rN)#(fN)(mN)(mN)(mN)(mN)(mG)(rU)(rU)(rG)(mU)(mA)(mG)(m |
| | C)(rU)(rC)(rC)(rC)(rU)(mU)(mU)(mC)(mU)(mC)(mA)(mU)(mU)#(mU)#(mC)#(m |
| | G)(SEQ ID NO: 22); |
| CR48-197 | (mN)#(mN)#(mN)#(mN)(mN)(mN)(dN)#(mN)(mN)(mN)(mN)(mN)(mN)(fN)(mN) |
| | (fN)(mN)(mN)(rN)#(mN)(fN)(mN)(mN)(mN)(mG)(rU)(rU)(rG)(mU)(mA)(mG)(m |
| | C)(rU)(rC)(rC)(rC)(rU)(mU)(mU)(mC)(mU)(mC)(mA)(mU)(mU)#(mU)#(mC)#(m |
| | G)(SEQ ID NO: 23); |
| CR48-198 | (mN)#(mN)#(mN)#(mN)(mN)(mN)(dN)#(mN)(mN)(mN)(mN)(fN)(mN)(fN)(mN)( |
| | fN)(mN)(mN)(mN)(fN)(mN)(mN)(mN)(mN)(mG)(rU)(rU)(rG)(mU)(mA)(mG)(m |
| | C)(rU)(rC)(rC)(rC)(rU)(mU)(mU)(mC)(mU)(mC)(mA)(mU)(mU)#(mU)#(mC)#(m |
| | G)(SEQ ID NO: 24); |
| CR48-199 | (mN)#(mN)#(mN)#(mN)(mN)(mN)(dN)#(mN)(mN)(mN)(mN)(fN)(mN)(fN)(mN)( |
| | fN)(mN)(mN)(mN)(mN)(fN)(mN)(mN)(mN)(mG)(rU)(rU)(rG)(mU)(mA)(mG)(m |
| | C)(rU)(rC)(rC)(rC)(rU)(mU)(mU)(mC)(mU)(mC)(mA)(mU)(mU)#(mU)#(mC)#(m |
| | G)(SEQ ID NO: 25); |
| CR48-200 | (mN)#(mN)#(mN)#(mN)(mN)(mN)(dN)#(mN)(mN)(mN)(mN)(mN)(mN)(fN)(mN) |
| | (fN)(mN)(mN)(mN)(fN)(mN)(mN)(mN)(mN)(mG)(rU)(rU)(rG)(mU)(mA)(mG)(m |
| | C)(rU)(rC)(rC)(rC)(rU)(mU)(mU)(mC)(mU)(mC)(mA)(mU)(mU)#(mU)#(mC)#(m |
| | G)(SEQ ID NO: 26); |
| CR48-201 | (mN)#(mN)#(mN)#(mN)(mN)(mN)(dN)#(mN)(mN)(mN)(mN)(mN)(mN)(fN)(mN) |
| | (fN)(mN)(mN)(mN)(mN)(fN)(mN)(mN)(mN)(mG)(rU)(rU)(rG)(mU)(mA)(mG)(m |
| | C)(rU)(rC)(rC)(rC)(rU)(mU)(mU)(mC)(mU)(mC)(mA)(mU)(mU)#(mU)#(mC)#(m |
| | G)(SEQ ID NO: 27); |
| CR48-202 | (mN)#(mN)#(mN)#(mN)(mN)(mN)(dN)#(mN)(mN)(mN)(mN)(fN)(mN)(fN)(mN)( |
| | fN)(mN)(mN)(fN)(fN)(mN)(mN)(mN)(mN)(mG)(rU)(rU)(rG)(mU)(mA)(mG)(mC) |
| | (rU)(rC)(rC)(rC)(rU)(mU)(mU)(mC)(mU)(mC)(mA)(mU)(mU)#(mU)#(mC)#(mG) |
| | (SEQ ID NO: 28); |
| CR48-203 | (mN)#(mN)#(mN)#(mN)(mN)(mN)(dN)#(mN)(mN)(mN)(mN)(fN)(mN)(fN)(mN)( |
| | fN)(mN)(mN)(fN)(mN)(fN)(mN)(mN)(mN)(mG)(rU)(rU)(rG)(mU)(mA)(mG)(mC) |
| | (rU)(rC)(rC)(rC)(rU)(mU)(mU)(mC)(mU)(mC)(mA)(mU)(mU)#(mU)#(mC)#(mG) |
| | (SEQ ID NO: 29); |
| CR48-204 | (mN)#(mN)#(mN)#(mN)(mN)(mN)(dN)#(mN)(mN)(mN)(mN)(mN)(mN)(fN)(mN) |
| | (fN)(mN)(mN)(fN)(fN)(mN)(mN)(mN)(mN)(mG)(rU)(rU)(rG)(mU)(mA)(mG)(mC |
| | )(rU)(rC)(rC)(rC)(rU)(mU)(mU)(mC)(mU)(mC)(mA)(mU)(mU)#(mU)#(mC)#(mG |
| | )(SEQ ID NO: 30); |
| CR48-205 | (mN)#(mN)#(mN)#(mN)(mN)(mN)(dN)#(mN)(mN)(mN)(mN)(mN)(mN)(fN)(mN) |
| | (fN)(mN)(mN)(fN)(mN)(fN)(mN)(mN)(mN)(mG)(rU)(rU)(rG)(mU)(mA)(mG)(mC |
| | )(rU)(rC)(rC)(rC)(rU)(mU)(mU)(mC)(mU)(mC)(mA)(mU)(mU)#(mU)#(mC)#(mG |
| | )(SEQ ID NO: 31); |
| CR48-213 | (mN)#(mN)#(mN)#(mN)(mN)(mN)(dN)#(mN)(mN)(mN)(mN)(mN)(mN)(fN)(mN) |
| | (IN)(mN)(mN)(rN)(rN)(rN)(mN)(rN)(rN)(mG)(dT)#(rU)(mG)(mU)(mA)(mG)(mC) |
| | (rU)(rC)(rC)(rC)(rU)(mU)(mU)(mC)(mU)(mC)(mA)(mU)(mU)#(mU)#(mC)#(mG) |
| | (SEQ ID NO: 32); |
| CR48-214 | (mN)#(mN)#(mN)#(mN)(mN)(mN)(dN)#(mN)(mN)(mN)(mN)(mN)(mN)(fN)(mN) |
| | (IN)(mN)(mN)(rN)(rN)(rN)(mN)(rN)(rN)(mG)(dT)#(rU)(mG)(mU)(mA)(mG)(mC) |
| | (rU)(rC)(mC)(rC)(rU)(mU)(mU)(mC)(mU)(mC)(mA)(mU)(mU)#(mU)#(mC)#(mG |
| | )(SEQ ID NO: 33); |
| CR48-215 | (mN)#(mN)#(mN)#(mN)(mN)(mN)(dN)#(mN)(mN)(mN)(mN)(mN)(mN)(fN)(mN) |
| | (IN)(mN)(mN)(rN)(rN)(rN)(mN)(rN)(rN)(mG)(dT)#(rU)(mG)(mU)(mA)(mG)(mC) |
| | (rU)(rC)(rC)(mC)(rU)(mU)(mU)(mC)(mU)(mC)(mA)(mU)(mU)#(mU)#(mC)#(mG |
| | )(SEQ ID NO: 34); |
| CR48-216 | (mN)#(mN)#(mN)#(mN)(mN)(mN)(dN)#(mN)(mN)(mN)(mN)(mN)(mN)(fN)(mN) |
| | (IN)(mN)(mN)(rN)(rN)(rN)(mN)(rN)(rN)(mG)(dT)#(rU)(mG)(mU)(mA)(mG)(mC) |
| | (rU)(rC)(mC)(mC)(rU)(mU)(mU)(mC)(mU)(mC)(mA)(mU)(mU)#(mU)#(mC)#(m |
| | G)(SEQ ID NO: 35); |
| CR48-217 | (mN)#(mN)#(mN)#(mN)(mN)(mN)(dN)#(mN)(mN)(mN)(mN)(mN)(mN)(fN)(mN) |
| | (fN)(mN)(mN)(rN)(rN)(rN)(mN)(rN)(rN)(mG)(rU)(rU)(rG)(mU)(mA)(mG)(mC)(d |
| | T)#(rC)(rC)(rC)(rU)(mU)(mU)(mC)(mU)(mC)(mA)(mU)(mU)#(mU)#(mC)#(mG) |
| | (SEQ ID NO: 36); |
| CR48-218 | (mN)#(mN)#(mN)#(mN)(mN)(mN)(dN)#(mN)(mN)(mN)(mN)(mN)(mN)(fN)(mN) |
| | (fN)(mN)(mN)(rN)(rN)(rN)(mN)(rN)(rN)(mG)(rU)(rU)(rG)(mU)(mA)(mG)(mC)(r |
| | U)(dC)#(rC)(rC)(rU)(mU)(mU)(mC)(mU)(mC)(mA)(mU)(mU)#(mU)#(mC)#(mG) |
| | (SEQ ID NO: 37); |
| CR48-219 | (mN)#(mN)#(mN)#(mN)(mN)(mN)(dN)#(mN)(mN)(mN)(mN)(mN)(mN)(fN)(mN) |
| | (fN)(mN)(mN)(rN)(rN)(rN)(mN)(rN)(rN)(mG)(rU)(rU)(rG)(mU)(mA)(mG)(mC)(r |
| | U)(rC)(dC)#(rC)(rU)(mU)(mU)(mC)(mU)(mC)(mA)(mU)(mU)#(mU)#(mC)#(mG) |
| | (SEQ ID NO: 38); |
| CR48-220 | (mN)#(mN)#(mN)#(mN)(mN)(mN)(dN)#(mN)(mN)(mN)(mN)(mN)(mN)(fN)(mN) |
| | (fN)(mN)(mN)(rN)(rN)(rN)(mN)(rN)(rN)(mG)(rU)(rU)(rG)(mU)(mA)(mG)(mC)(r |
| | U)(rC)(rC)(dC)#(rU)(mU)(mU)(mC)(mU)(mC)(mA)(mU)(mU)#(mU)#(mC)#(mG) |
| | (SEQ ID NO: 39); |
| CR48-221 | (mN)#(mN)#(mN)#(mN)(mN)(mN)(dN)#(mN)(mN)(mN)(mN)(mN)(mN)(fN)(mN) |
| | (fN)(mN)(mN)(rN)(rN)(rN)(mN)(rN)(rN)(mG)(rU)(rU)(rG)(mU)(mA)(mG)(mC)(r |
| | U)(rC)(rC)(rC)(dT)#(mU)(mU)(mC)(mU)(mC)(mA)(mU)(mU)#(mU)#(mC)#(mG) |
| | (SEQ ID NO: 40); |
| CR48-222 | (mN)#(mN)#(mN)#(mN)(mN)(mN)(dN)#(mN)(mN)(mN)(mN)(mN)(mN)(fN)(mN) |
| | (fN)(mN)(mN)(rN)(rN)(rN)(mN)(rN)(rN)(mG)(rU)(rU)(rG)(mU)(mA)(mG)(mC)(d |
| | T)#(rC)(dC)#(rC)(dT)#(mU)(mU)(mC)(mU)(mC)(mA)(mU)(mU)#(mU)#(mC)#(m |
| | G)(SEQ ID NO: 41); |
| CR48-223 | (mN)#(mN)#(mN)#(mN)(mN)(mN)(dN)#(mN)(mN)(mN)(mN)(mN)(mN)(fN)(mN) |
| | (fN)(mN)(mN)(rN)(rN)(rN)(mN)(rN)(rN)(mG)(dT)#(rU)(rG)(mU)(mA)(mG)(mC)( |
| | dT)#(dC)#(dC)#(rC)(dT)#(mU)(mU)(mC)(mU)(mC)(mA)(mU)(mU)#(mU)#(mC)# |
| | (mG)(SEQ ID NO: 42); |
| CR48-224 | (mN)#(mN)#(mN)#(mN)(mN)(mN)(dN)#(mN)(mN)(mN)(mN)(mN)(mN)(fN)(mN) |
| | (fN)(mN)(mN)(rN)(rN)(rN)(mN)(rN)(rN)(mG)(dT)#(rU)(mG)(mU)(mA)(mG)(mC) |
| | (dT)#(rC)(mC)(rC)(dT)#(mU)(mU)(mC)(mU)(mC)(mA)(mU)(mU)#(mU)#(mC)#( |
| | mG)(SEQ ID NO: 43); |
| CR48-225 | (mN)#(mN)#(mN)#(mN)(mN)(mN)(dN)#(mN)(mN)(mN)(mN)(mN)(mN)(IN)(mN) |
| | (IN)(mN)(mN)(rN)(rN)(rN)(mN)(rN)(rN)(mG)(dT)#(rU)(mG)(mU)(mA)(mG)(mC) |
| | (dT)#(rC)(dC)#(mC)(dT)#(mU)(mU)(mC)(mU)(mC)(mA)(mU)(mU)#(mU)#(mC)# |
| | (mG)(SEQ ID NO: 44); |
| CR48-226 | (mN)#(mN)#(mN)#(mN)(mN)(mN)(dN)#(mN)(mN)(mN)(mN)(mN)(mN)(fN)(mN) |
| | (IN)(mN)(mN)(rN)(rN)(rN)(mN)(rN)(rN)(mG)(dT)#(rU)(mG)(mU)(mA)(mG)(mC) |
| | (dT)#(rC)(mC)(mC)(dT)#(mU)(mU)(mC)(mU)(mC)(mA)(mU)(mU)#(mU)#(mC)#( |
| | mG)(SEQ ID NO: 45); |
| CR48-227 | (mN)#(mN)#(mN)#(mN)(mN)(mN)(dN)#(mN)(mN)(mN)(mN)(mN)(mN)(fN)(mN) |
| | (IN)(mN)(mN)(rN)(rN)(rN)(mN)(rN)(rN)(mG)(dT)#(rU)(mG)(mU)(mA)(mG)(mC) |
| | (dT)#(dC)#(mC)(mC)(dT)#(mU)(mU)(mC)(mU)(mC)(mA)(mU)(mU)#(mU)#(mC) |
| | #(mG)(SEQ ID NO: 46); |
| | (mN)#(mN)#(mN)#(mN)(mN)(mN)(dN)#(mN)(mN)(mN)(mN)(fN)(mN)(fN)(mN)( |
| CR48-199- | IN)(mN)(mN)(mN)(mN)(IN)(mN)(mN)(mN)(mG)(rU)(rU)(mG)(mU)(mA)(mG)(m |
| V1.1 | C)(rU)(rC)(rC)(dC)#(rU)(mU)(mU)(mC)(mU)(mC)(mA)(mU)(mU)#(mU)#(mC)#( |
| | mG)(SEQ ID NO: 47); |
| | (mN)#(mN)#(mN)#(mN)(mN)(mN)(dN)#(mN)(mN)(mN)(mN)(fN)(mN)(fN)(mN)( |
| CR48-199- | IN)(mN)(mN)(mN)(mN)(IN)(mN)(mN)(mN)(mG)(rU)(rU)(mG)(mU)(mA)(mG)(m |
| V1.2 | C)(rU)(rC)(dC)#(rC)(rU)(mU)(mU)(mC)(mU)(mC)(mA)(mU)(mU)#(mU)#(mC)#( |
| | mG)(SEQ ID NO: 48); |
| | (mN)#(mN)#(mN)#(mN)(mN)(mN)(dN)#(mN)(mN)(mN)(mN)(fN)(mN)(fN)(mN)( |
| CR48-199- | IN)(mN)(mN)(mN)(mN)(IN)(mN)(mN)(mN)(mG)(rU)(rU)(mG)(mU)(mA)(mG)(m |
| V1.3 | C)(rU)(rC)(rC)(rC)(dT)#(mU)(mU)(mC)(mU)(mC)(mA)(mU)(mU)#(mU)#(mC)#( |
| | mG)(SEQ ID NO: 49); |
| | (mN)#(mN)#(mN)#(mN)(mN)(mN)(dN)#(mN)(mN)(mN)(mN)(fN)(mN)(fN)(mN)( |
| CR48-199- | IN)(mN)(mN)(mN)(mN)(IN)(mN)(mN)(mN)(mG)(rU)(rU)(mG)(mU)(mA)(mG)(m |
| V1.4 | C)(rU)(rC)(mC)(rC)(rU)(mU)(mU)(mC)(mU)(mC)(mA)(mU)(mU)#(mU)#(mC)#( |
| | mG)(SEQ ID NO: 50); |
| | (mN)#(mN)#(mN)#(mN)(mN)(mN)(dN)#(mN)(mN)(mN)(mN)(fN)(mN)(fN)(mN)( |
| CR48-199- | IN)(mN)(mN)(mN)(mN)(IN)(mN)(mN)(mN)(mG)(rU)(rU)(mG)(mU)(mA)(mG)(m |
| V1.5 | C)(rU)(rC)(dC)#(dC)#(rU)(mU)(mU)(mC)(mU)(mC)(mA)(mU)(mU)#(mU)#(mC)# |
| | (mG)(SEQ ID NO: 51); |
| | (mN)#(mN)#(mN)#(mN)(mN)(mN)(dN)#(mN)(mN)(mN)(mN)(fN)(mN)(fN)(mN)( |
| CR48-199- | IN)(mN)(mN)(mN)(mN)(IN)(mN)(mN)(mN)(mG)(rU)(rU)(mG)(mU)(mA)(mG)(m |
| V1.6 | C)(rU)(rC)(dC)#(rC)(dT)#(mU)(mU)(mC)(mU)(mC)(mA)(mU)(mU)#(mU)#(mC)# |
| | (mG)(SEQ ID NO: 52); |
| | (mN)#(mN)#(mN)#(mN)(mN)(mN)(dN)#(mN)(mN)(mN)(mN)(fN)(mN)(fN)(mN)( |
| CR48-199- | IN)(mN)(mN)(mN)(mN)(IN)(mN)(mN)(mN)(mG)(rU)(rU)(mG)(mU)(mA)(mG)(m |
| V1.7 | C)(rU)(rC)(dC)#(dC)#(dT)#(mU)(mU)(mC)(mU)(mC)(mA)(mU)(mU)#(mU)#(mC |
| | )#(mG)(SEQ ID NO: 53); |
| | (mN)#(mN)#(mN)#(mN)(mN)(mN)(dN)#(mN)(mN)(mN)(mN)(fN)(mN)(fN)(mN)( |
| CR48-199- | IN)(mN)(mN)(mN)(mN)(IN)(mN)(mN)(mN)(mG)(rU)(rU)(mG)(mU)(mA)(mG)(m |
| V1.8 | C)(rU)(rC)(mC)(dC)#(rU)(mU)(mU)(mC)(mU)(mC)(mA)(mU)(mU)#(mU)#(mC)# |
| | (mG)(SEQ ID NO: 54); |
| | (mN)#(mN)#(mN)#(mN)(mN)(mN)(dN)#(mN)(mN)(mN)(mN)(fN)(mN)(fN)(mN)( |
| CR48-199- | IN)(mN)(mN)(mN)(mN)(IN)(mN)(mN)(mN)(mG)(rU)(rU)(mG)(mU)(mA)(mG)(m |
| V1.9 | C)(rU)(rC)(mC)(rC)(dT)#(mU)(mU)(mC)(mU)(mC)(mA)(mU)(mU)#(mU)#(mC)#( |
| | mG)(SEQ ID NO: 55); |
| | (mN)#(mN)#(mN)#(mN)(mN)(mN)(dN)#(mN)(mN)(mN)(mN)(fN)(mN)(fN)(mN)( |
| CR48-199- | fN)(mN)(mN)(mN)(mN)(fN)(mN)(mN)(mN)(mG)(rU)(rU)(mG)(mU)(mA)(mG)(m |
| Vl.10 | C)(rU)(rC)(mC)(dC)#(dT)#(mU)(mU)(mC)(mU)(mC)(mA)(mU)(mU)#(mU)#(mC) |
| | #(mG)(SEQ ID NO: 56); |
| | (mN)#(mN)#(mN)#(mN)(mN)(mN)(dN)#(mN)(mN)(mN)(mN)(fN)(mN)(fN)(mN)( |
| CR48-199- | fN)(mN)(mN)(mN)(mN)(fN)(mN)(mN)(mN)(mG)(rU)(rU)(mG)(mU)(mA)(mG)(m |
| V1.11 | C)(rU)(rC)(mC)(mC)(rU)(mU)(mU)(mC)(mU)(mC)(mA)(mU)(mU)#(mU)#(mC)#( |
| | mG)(SEQ ID NO: 57); |
| | (mN)#(mN)#(mN)#(mN)(mN)(mN)(dN)#(mN)(mN)(mN)(mN)(fN)(mN)(fN)(mN)( |
| CR48-199- | fN)(mN)(mN)(mN)(mN)(fN)(mN)(mN)(mN)(mG)(rU)(rU)(mG)(mU)(mA)(mG)(m |
| V1.12 | C)(rU)(rC)(mC)(mC)(dT)#(mU)(mU)(mC)(mU)(mC)(mA)(mU)(mU)#(mU)#(mC) |
| | #(mG)(SEQ ID NO: 58); |
| | (mN)#(mN)#(mN)#(mN)(mN)(mN)(dN)#(mN)(mN)(mN)(mN)(fN)(mN)(fN)(mN)( |
| CR48-199- | fN)(mN)(mN)(mN)(mN)(fN)(mN)(mN)(mN)(mG)(rU)(rU)(mG)(mU)(mA)(mG)(m |
| Vl.13 | C)(dT)#(rC)(rC)(dC)#(rU)(mU)(mU)(mC)(mU)(mC)(mA)(mU)(mU)#(mU)#(mC)# |
| | (mG)(SEQ ID NO: 59); |
| | (mN)#(mN)#(mN)#(mN)(mN)(mN)(dN)#(mN)(mN)(mN)(mN)(fN)(mN)(fN)(mN)( |
| CR48-199- | fN)(mN)(mN)(mN)(mN)(fN)(mN)(mN)(mN)(mG)(rU)(rU)(mG)(mU)(mA)(mG)(m |
| V1.14 | C)(dT)#(rC)(dC)#(rC)(rU)(mU)(mU)(mC)(mU)(mC)(mA)(mU)(mU)#(mU)#(mC)# |
| | (mG)(SEQ ID NO: 60); |
| | (mN)#(mN)#(mN)#(mN)(mN)(mN)(dN)#(mN)(mN)(mN)(mN)(fN)(mN)(fN)(mN)( |
| CR48-199- | fN)(mN)(mN)(mN)(mN)(fN)(mN)(mN)(mN)(mG)(rU)(rU)(mG)(mU)(mA)(mG)(m |
| V1.15 | C)(dT)#(rC)(rC)(rC)(dT)#(mU)(mU)(mC)(mU)(mC)(mA)(mU)(mU)#(mU)#(mC)# |
| | (mG)(SEQ ID NO: 61); |
| | (mN)#(mN)#(mN)#(mN)(mN)(mN)(dN)#(mN)(mN)(mN)(mN)(fN)(mN)(fN)(mN)( |
| CR48-199- | fN)(mN)(mN)(mN)(mN)(fN)(mN)(mN)(mN)(mG)(rU)(rU)(mG)(mU)(mA)(mG)(m |
| V1.16 | C)(dT)#(rC)(mC)(rC)(rU)(mU)(mU)(mC)(mU)(mC)(mA)(mU)(mU)#(mU)#(mC)#( |
| | mG)(SEQ ID NO: 62); |
| | (mN)#(mN)#(mN)#(mN)(mN)(mN)(dN)#(mN)(mN)(mN)(mN)(fN)(mN)(fN)(mN)( |
| CR48-199- | fN)(mN)(mN)(mN)(mN)(fN)(mN)(mN)(mN)(mG)(rU)(rU)(mG)(mU)(mA)(mG)(m |
| V1.17 | C)(dT)#(rC)(dC)#(dC)#(rU)(mU)(mU)(mC)(mU)(mC)(mA)(mU)(mU)#(mU)#(mC |
| | )#(mG)(SEQ ID NO: 63); |
| | (mN)#(mN)#(mN)#(mN)(mN)(mN)(dN)#(mN)(mN)(mN)(mN)(fN)(mN)(fN)(mN)( |
| CR48-199- | fN)(mN)(mN)(mN)(mN)(fN)(mN)(mN)(mN)(mG)(rU)(rU)(mG)(mU)(mA)(mG)(m |
| V1.18 | C)(dT)#(rC)(dC)#(rC)(dT)#(mU)(mU)(mC)(mU)(mC)(mA)(mU)(mU)#(mU)#(mC) |
| | #(mG)(SEQ ID NO: 64); |
| | (mN)#(mN)#(mN)#(mN)(mN)(mN)(dN)#(mN)(mN)(mN)(mN)(fN)(mN)(fN)(mN)( |
| CR48-199- | fN)(mN)(mN)(mN)(mN)(fN)(mN)(mN)(mN)(mG)(rU)(rU)(mG)(mU)(mA)(mG)(m |
| V1.19 | C)(dT)#(rC)(dC)#(dC)#(dT)#(mU)(mU)(mC)(mU)(mC)(mA)(mU)(mU)#(mU)#(m |
| | C)#(mG)(SEQ ID NO: 65); |
| | (mN)#(mN)#(mN)#(mN)(mN)(mN)(dN)#(mN)(mN)(mN)(mN)(fN)(mN)(fN)(mN)( |
| CR48-199- | fN)(mN)(mN)(mN)(mN)(fN)(mN)(mN)(mN)(mG)(rU)(rU)(mG)(mU)(mA)(mG)(m |
| V1.20 | C)(dT)#(rC)(mC)(dC)#(rU)(mU)(mU)(mC)(mU)(mC)(mA)(mU)(mU)#(mU)#(mC) |
| | #(mG)(SEQ ID NO: 66); |
| | (mN)#(mN)#(mN)#(mN)(mN)(mN)(dN)#(mN)(mN)(mN)(mN)(fN)(mN)(fN)(mN)( |
| CR48-199- | fN)(mN)(mN)(mN)(mN)(fN)(mN)(mN)(mN)(mG)(rU)(rU)(mG)(mU)(mA)(mG)(m |
| V1.21 | C)(dT)#(rC)(mC)(rC)(dT)#(mU)(mU)(mC)(mU)(mC)(mA)(mU)(mU)#(mU)#(mC) |
| | #(mG)(SEQ ID NO: 67); |
| | (mN)#(mN)#(mN)#(mN)(mN)(mN)(dN)#(mN)(mN)(mN)(mN)(fN)(mN)(fN)(mN)( |
| CR48-199- | fN)(mN)(mN)(mN)(mN)(fN)(mN)(mN)(mN)(mG)(rU)(rU)(mG)(mU)(mA)(mG)(m |
| V1.22 | C)(dT)#(rC)(mC)(dC)#(dT)#(mU)(mU)(mC)(mU)(mC)(mA)(mU)(mU)#(mU)#(m |
| | C)#(mG)(SEQ ID NO: 68); |
| | (mN)#(mN)#(mN)#(mN)(mN)(mN)(dN)#(mN)(mN)(mN)(mN)(fN)(mN)(fN)(mN)( |
| CR48-199- | fN)(mN)(mN)(mN)(mN)(fN)(mN)(mN)(mN)(mG)(rU)(rU)(mG)(mU)(mA)(mG)(m |
| V1.23 | C)(dT)#(rC)(mC)(mC)(rU)(mU)(mU)(mC)(mU)(mC)(mA)(mU)(mU)#(mU)#(mC) |
| | #(mG)(SEQ ID NO: 69); |
| | (mN)#(mN)#(mN)#(mN)(mN)(mN)(dN)#(mN)(mN)(mN)(mN)(fN)(mN)(fN)(mN)( |
| CR48-199- | fN)(mN)(mN)(mN)(mN)(fN)(mN)(mN)(mN)(mG)(rU)(rU)(mG)(mU)(mA)(mG)(m |
| V1.24 | C)(dT)#(rC)(mC)(mC)(dT)#(mU)(mU)(mC)(mU)(mC)(mA)(mU)(mU)#(mU)#(mC |
| | )#(mG)(SEQ ID NO: 70); |
| | (mN)#(mN)#(mN)#(mN)(mN)(mN)(dN)#(mN)(mN)(mN)(mN)(fN)(mN)(fN)(mN)( |
| CR48-199- | fN)(mN)(mN)(mN)(mN)(fN)(mN)(mN)(mN)(mG)(dT)#(rU)(mG)(mU)(mA)(mG)( |
| V1.25 | mC)(dT)#(rC)(rC)(dC)#(rU)(mU)(mU)(mC)(mU)(mC)(mA)(mU)(mU)#(mU)#(mC |
| | )#(mG)(SEQ ID NO: 71); |
| | (mN)#(mN)#(mN)#(mN)(mN)(mN)(dN)#(mN)(mN)(mN)(mN)(fN)(mN)(IN)(mN)( |
| CR48-199- | fN)(mN)(mN)(mN)(mN)(fN)(mN)(mN)(mN)(mG)(dT)#(rU)(mG)(mU)(mA)(mG)( |
| V1.26 | mC)(dT)#(rC)(dC)#(rC)(rU)(mU)(mU)(mC)(mU)(mC)(mA)(mU)(mU)#(mU)#(mC |
| | )#(mG)(SEQ ID NO: 72); |
| | (mN)#(mN)#(mN)#(mN)(mN)(mN)(dN)#(mN)(mN)(mN)(mN)(fN)(mN)(IN)(mN)( |
| CR48-199- | fN)(mN)(mN)(mN)(mN)(fN)(mN)(mN)(mN)(mG)(dT)#(rU)(mG)(mU)(mA)(mG)( |
| V1.27 | mC)(dT)#(rC)(rC)(rC)(dT)#(mU)(mU)(mC)(mU)(mC)(mA)(mU)(mU)#(mU)#(mC |
| | )#(mG)(SEQ ID NO: 73); |
| | (mN)#(mN)#(mN)#(mN)(mN)(mN)(dN)#(mN)(mN)(mN)(mN)(fN)(mN)(IN)(mN)( |
| CR48-199- | fN)(mN)(mN)(mN)(mN)(fN)(mN)(mN)(mN)(mG)(dT)#(rU)(mG)(mU)(mA)(mG)( |
| V1.28 | mC)(dT)#(rC)(mC)(rC)(rU)(mU)(mU)(mC)(mU)(mC)(mA)(mU)(mU)#(mU)#(mC) |
| | #(mG)(SEQ ID NO: 74); |
| | (mN)#(mN)#(mN)#(mN)(mN)(mN)(dN)#(mN)(mN)(mN)(mN)(fN)(mN)(IN)(mN)( |
| CR48-199- | fN)(mN)(mN)(mN)(mN)(fN)(mN)(mN)(mN)(mG)(dT)#(rU)(mG)(mU)(mA)(mG)( |
| V1.29 | mC)(dT)#(rC)(dC)#(dC)#(rU)(mU)(mU)(mC)(mU)(mC)(mA)(mU)(mU)#(mU)#(m |
| | C)#(mG)(SEQ ID NO: 75); |
| | (mN)#(mN)#(mN)#(mN)(mN)(mN)(dN)#(mN)(mN)(mN)(mN)(fN)(mN)(IN)(mN)( |
| CR48-199- | fN)(mN)(mN)(mN)(mN)(fN)(mN)(mN)(mN)(mG)(dT)#(rU)(mG)(mU)(mA)(mG)( |
| V1.30 | mC)(dT)#(rC)(dC)#(rC)(dT)#(mU)(mU)(mC)(mU)(mC)(mA)(mU)(mU)#(mU)#(m |
| | C)#(mG)(SEQ ID NO: 76); |
| | (mN)#(mN)#(mN)#(mN)(mN)(mN)(dN)#(mN)(mN)(mN)(mN)(fN)(mN)(IN)(mN)( |
| CR48-199- | fN)(mN)(mN)(mN)(mN)(fN)(mN)(mN)(mN)(mG)(dT)#(rU)(mG)(mU)(mA)(mG)( |
| V1.31 | mC)(dT)#(rC)(dC)#(dC)#(dT)#(mU)(mU)(mC)(mU)(mC)(mA)(mU)(mU)#(mU)#( |
| | mC)#(mG)(SEQ ID NO: 77); |
| | (mN)#(mN)#(mN)#(mN)(mN)(mN)(dN)#(mN)(mN)(mN)(mN)(fN)(mN)(IN)(mN)( |
| CR48-199- | fN)(mN)(mN)(mN)(mN)(fN)(mN)(mN)(mN)(mG)(dT)#(rU)(mG)(mU)(mA)(mG)( |
| V1.32 | mC)(dT)#(rC)(mC)(dC)#(rU)(mU)(mU)(mC)(mU)(mC)(mA)(mU)(mU)#(mU)#(m |
| | C)#(mG)(SEQ ID NO: 78); |
| | (mN)#(mN)#(mN)#(mN)(mN)(mN)(dN)#(mN)(mN)(mN)(mN)(fN)(mN)(IN)(mN)( |
| CR48-199- | fN)(mN)(mN)(mN)(mN)(fN)(mN)(mN)(mN)(mG)(dT)#(rU)(mG)(mU)(mA)(mG)( |
| V1.33 | mC)(dT)#(rC)(mC)(rC)(dT)#(mU)(mU)(mC)(mU)(mC)(mA)(mU)(mU)#(mU)#(m |
| | C)#(mG)(SEQ ID NO: 79); |
| | (mN)#(mN)#(mN)#(mN)(mN)(mN)(dN)#(mN)(mN)(mN)(mN)(fN)(mN)(IN)(mN)( |
| CR48-199- | fN)(mN)(mN)(mN)(mN)(fN)(mN)(mN)(mN)(mG)(dT)#(rU)(mG)(mU)(mA)(mG)( |
| V1.34 | mC)(dT)#(rC)(mC)(dC)#(dT)#(mU)(mU)(mC)(mU)(mC)(mA)(mU)(mU)#(mU)#( |
| | mC)#(mG)(SEQ ID NO: 80); |
| | (mN)#(mN)#(mN)#(mN)(mN)(mN)(dN)#(mN)(mN)(mN)(mN)(fN)(mN)(IN)(mN)( |
| CR48-199- | fN)(mN)(mN)(mN)(mN)(fN)(mN)(mN)(mN)(mG)(dT)#(rU)(mG)(mU)(mA)(mG)( |
| V1.35 | mC)(dT)#(rC)(mC)(mC)(rU)(mU)(mU)(mC)(mU)(mC)(mA)(mU)(mU)#(mU)#(m |
| | C)#(mG)(SEQ ID NO: 81); |
| | (mN)#(mN)#(mN)#(mN)(mN)(mN)(dN)#(mN)(mN)(mN)(mN)(fN)(mN)(fN)(mN)( |
| CR48-199- | fN)(mN)(mN)(mN)(mN)(fN)(mN)(mN)(mN)(mG)(dT)#(rU)(mG)(mU)(mA)(mG)( |
| V1.36 | mC)(dT)#(rC)(mC)(mC)(dT)#(mU)(mU)(mC)(mU)(mC)(mA)(mU)(mU)#(mU)#(m |
| | C)#(mG)(SEQ ID NO: 82); |
| | (mN)#(mN)#(mN)#(mN)(mN)(mN)(dN)#(mN)(mN)(mN)(mN)(fN)(mN)(fN)(mN)( |
| CR48-203- | fN)(mN)(mN)(fN)(mN)(fN)(mN)(mN)(mN)(mG)(rU)(rU)(mG)(mU)(mA)(mG)(m |
| V1.7 | C)(rU)(rC)(dC)#(dC)#(dT)#(mU)(mU)(mC)(mU)(mC)(mA)(mU)(mU)#(mU)#(mC |
| | )#(mG)(SEQ ID NO: 83); |
| | (mN)#(mN)#(mN)#(mN)(mN)(mN)(dN)#(mN)(mN)(mN)(mN)(fN)(mN)(fN)(mN)( |
| CR48-203- | fN)(mN)(mN)(N)(mN)(fN)(mN)(mN)(mN)(mG)(rU)(rU)(mG)(mU)(mA)(mG)(m |
| Vl.10 | C)(rU)(rC)(mC)(dC)#(dT)#(mU)(mU)(mC)(mU)(mC)(mA)(mU)(mU)#(mU)#(mC) |
| | #(mG)(SEQ ID NO: 84); |
| | (mN)#(mN)#(mN)#(mN)(mN)(mN)(dN)#(mN)(mN)(mN)(mN)(fN)(mN)(fN)(mN)( |
| CR48-203- | fN)(mN)(mN)(fN)(mN)(fN)(mN)(mN)(mN)(mG)(rU)(rU)(mG)(mU)(mA)(mG)(m |
| V1.12 | C)(rU)(rC)(mC)(mC)(dT)#(mU)(mU)(mC)(mU)(mC)(mA)(mU)(mU)#(mU)#(mC) |
| | #(mG)(SEQ ID NO: 85); |
| | (mN)#(mN)#(mN)#(mN)(mN)(mN)(dN)#(mN)(mN)(mN)(mN)(fN)(mN)(IN)(mN)( |
| CR48-203- | fN)(mN)(mN)(IN)(mN)(fN)(mN)(mN)(mN)(mG)(rU)(rU)(mG)(mU)(mA)(mG)(m |
| V1.18 | C)(dT)#(rC)(dC)#(rC)(dT)#(mU)(mU)(mC)(mU)(mC)(mA)(mU)(mU)#(mU)#(mC) |
| | #(mG)(SEQ ID NO: 86); |
| | (mN)#(mN)#(mN)#(mN)(mN)(mN)(dN)#(mN)(mN)(mN)(mN)(fN)(mN)(IN)(mN)( |
| CR48-203- | fN)(mN)(mN)(IN)(mN)(fN)(mN)(mN)(mN)(mG)(rU)(rU)(mG)(mU)(mA)(mG)(m |
| V1.19 | C)(dT)#(rC)(dC)#(dC)#(dT)#(mU)(mU)(mC)(mU)(mC)(mA)(mU)(mU)#(mU)#(m |
| | C)#(mG)(SEQ ID NO: 87); |
| | (mN)#(mN)#(mN)#(mN)(mN)(mN)(dN)#(mN)(mN)(mN)(mN)(fN)(mN)(IN)(mN)( |
| CR48-203- | fN)(mN)(mN)(IN)(mN)(fN)(mN)(mN)(mN)(mG)(rU)(rU)(mG)(mU)(mA)(mG)(m |
| V1.22 | C)(dT)#(rC)(mC)(dC)#(dT)#(mU)(mU)(mC)(mU)(mC)(mA)(mU)(mU)#(mU)#(m |
| | C)#(mG)(SEQ ID NO: 88); |
| | (mN)#(mN)#(mN)#(mN)(mN)(mN)(dN)#(mN)(mN)(mN)(mN)(fN)(mN)(IN)(mN)( |
| CR48-203- | fN)(mN)(mN)(IN)(mN)(fN)(mN)(mN)(mN)(mG)(rU)(rU)(mG)(mU)(mA)(mG)(m |
| V1.24 | C)(dT)#(rC)(mC)(mC)(dT)#(mU)(mU)(mC)(mU)(mC)(mA)(mU)(mU)#(mU)#(mC |
| | )#(mG)(SEQ ID NO: 89); |
| | (mN)#(mN)#(mN)#(mN)(mN)(mN)(dN)#(mN)(mN)(mN)(mN)(fN)(mN)(IN)(mN)( |
| CR48-203- | fN)(mN)(mN)(IN)(mN)(fN)(mN)(mN)(mN)(mG)(dT)#(rU)(mG)(mU)(mA)(mG)(m |
| V1.30 | C)(dT)#(rC)(dC)#(rC)(dT)#(mU)(mU)(mC)(mU)(mC)(mA)(mU)(mU)#(mU)#(mC) |
| | #(mG)(SEQ ID NO: 90); |
| | (mN)#(mN)#(mN)#(mN)(mN)(mN)(dN)#(mN)(mN)(mN)(mN)(fN)(mN)(IN)(mN)( |
| CR48-203- | fN)(mN)(mN)(IN)(mN)(fN)(mN)(mN)(mN)(mG)(dT)#(rU)(mG)(mU)(mA)(mG)(m |
| V1.31 | C)(dT)#(rC)(dC)#(dC)#(dT)#(mU)(mU)(mC)(mU)(mC)(mA)(mU)(mU)#(mU)#(m |
| | C)#(mG)(SEQ ID NO: 91); |
| | (mN)#(mN)#(mN)#(mN)(mN)(mN)(dN)#(mN)(mN)(mN)(mN)(fN)(mN)(IN)(mN)( |
| CR48-203- | fN)(mN)(mN)(IN)(mN)(fN)(mN)(mN)(mN)(mG)(dT)#(rU)(mG)(mU)(mA)(mG)(m |
| V1.34 | C)(dT)#(rC)(mC)(dC)#(dT)#(mU)(mU)(mC)(mU)(mC)(mA)(mU)(mU)#(mU)#(m |
| | C)#(mG)(SEQ ID NO: 92); or |
| | (mN)#(mN)#(mN)#(mN)(mN)(mN)(dN)#(mN)(mN)(mN)(mN)(fN)(mN)(fN)(mN)( |
| CR48-203- | fN)(mN)(mN)(fN)(mN)(fN)(mN)(mN)(mN)(mG)(dT)#(rU)(mG)(mU)(mA)(mG)(m |
| V1.36 | C)(dT)#(rC)(mC)(mC)(dT)#(mU)(mU)(mC)(mU)(mC)(mA)(mU)(mU)#(mU)#(mC |
| | )#(mG)(SEQ ID NO: 93), |

wherein rN=RNA, mN=2′-O-methyl RNA, fN=2′-fluoro RNA, N #N=phosphorothioate linkage, and N=any nucleotide.

In certain embodiments, the chemically modified Nme gRNA further comprises a nucleotide or non-nucleotide loop or linker linking the 3′ end of the crRNA portion to the 5′ end of the tracrRNA portion.

In certain embodiments, the nucleotide loop is chemically modified. In certain embodiments, the nucleotide loop comprises the nucleotide sequence of GAAA. In certain embodiments, the nucleotide loop comprises the nucleotide sequence of (mG)(mA)(mA)(mA), wherein mN corresponds to a 2′-O-methyl RNA and N corresponds to any nucleotide. In certain embodiments, the non-nucleotide linker comprises an azide linker, an ethylene glycol oligomer, a tetrazine linker, an alkyl chain, a peptide, an amide, or a carbamate.

In one aspect, the disclosure provides a chemically modified Neisseria meningitidis (Nme) single guide RNA (sgRNA) comprising one or more chemical modifications.

In certain embodiments, the sgRNA is between 99 and 145 nucleotides in length. In certain embodiments, the sgRNA is 145 nucleotides in length, 121 nucleotides in length, 111 nucleotides in length, 107 nucleotides in length, 105 nucleotides in length, 103 nucleotides in length, 102 nucleotides in length, 101 nucleotides in length, 100 nucleotides in length, or 99 nucleotides in length.

In certain embodiments, the one or more chemical modifications are each independently selected from a modification of a ribose group, a phosphate group, a nucleobase, or a combination thereof.

In certain embodiments, each modification of the ribose group is independently selected from the group consisting of 2′-O-methyl, 2′-fluoro, 2′-deoxy, 2′-O-(2-methoxyethyl) (MOE), 2′-NH₂ (2′-amino), 4′-thio, a bicyclic nucleotide, a locked nucleic acid (LNA), a 2′-(S)-constrained ethyl (S-cEt), a constrained MOE, and a 2′-O,4′-C-aminomethylene bridged nucleic acid (2′,4′-BNA^{NC}).

In certain embodiments, at least 40% of the ribose groups are chemically modified. In certain embodiments, at least 80% of the ribose groups are chemically modified. In certain embodiments, 100% of the ribose groups are chemically modified.

In certain embodiments, each modification of the phosphate group is independently selected from the group consisting of a phosphorothioate, phosphonoacetate (PACE), thiophosphonoacetate (thioPACE), amide, triazole, phosphonate, or phosphotriester modification.

In certain embodiments, each modification of the nucleobase group is independently selected from the group consisting of 2-thiouridine, 4-thiouridine, N⁶-methyladenosine, pseudouridine, 2,6-diaminopurine, inosine, thymidine, 5-methylcytosine, 5-substituted pyrimidine, isoguanine, isocytosine, and halogenated aromatic groups.

In certain embodiments, the chemically modified Nme sgRNA comprises at least 40% modified nucleotides. In certain embodiments, the chemically modified Nme sgRNA comprises at least 80% modified nucleotides. In certain embodiments, the chemically modified Nme sgRNA comprises at least 90% modified nucleotides. In certain embodiments, the chemically modified Nme sgRNA comprises 100% modified nucleotides.

In certain embodiments, the chemically modified Nme sgRNA comprises between 2 and 100 modified nucleotides.

In certain embodiments, the chemically modified Nme sgRNA comprises at least one chemical modification in the nucleotide sequence of any one of:

(SEQ ID NO: 97)
(N)ₓGUUGUAGCUCCCUUUCUCAUUUCGGAAACGA
AAUGAGAACCGUUGCUACAAUAAGGCCGUCUGAAA
AGAUGUGCCGCAACGCUCUGCCCCUUAAAGCUUCU
GCUUUAAGGGGCAUCGUUUA
(SG-145NT);
(SEQ ID NO: 98)
(N)ₓGUUGUAGCUCCCGAAACGUUGCUACAAUAAG
GCCGUCUGAAAAGAUGUGCCGCAACGCUCUGCCCC
UUAAAGCUUCUGCUUUAAGGGGCAUCGUU
(SG-121NTv1);
(SEQ ID NO: 99)
(N)ₓGUUGUAGCUCCCGAAACGUUGCUACAAUAAG
GCCGUCUGAAAAGAUGUGCCGCAACGCUCUGCCCC
UUAAAGCUUCUGCUUUAAGGGGCAUCGUU
UA
(SG-121NTv2);
(SEQ ID NO: 100)
(N)ₓGUUGUAGCUCCCGAAACGUUGCUACAAUAAG
GCCGUCUGAAAAGAUGUGCCGCAACGCUCUGCCCC
UUUUCUAAGGGGCAUCGUUUA
(SG-111NT);
(SEQ ID NO: 101)
(N)ₓGUUGUAGCUCCCGAAACGUUGCUACAAUAAG
GCCGUCUGAAAAGAUGUGCCGCAACGCUCUGCCCC
UUUCUAGGGGCAUCGUU
(SG-107NT);
(SEQ ID NO: 102)
(N)ₓGUUGUAGCUCCCGAAACGUUGCUACAAUAAG
GCCGUCUGAAAAGAUGUGCCGCAACGCUCUGCCCC
UUCUGGGGCAUCGUU
(SG-105NTv1);
(SEQ ID NO: 103)
(N)ₓGUUGUAGCUCCCGAAACGUUGCUACAAUAAG
GCCGUCUGAAAAGAUGUGCCGCAACGCUCUGCCCC
UUUUCUAAGGGGCAU
(SG-105NTv2);
(SEQ ID NO: 104)
(N)ₓGUUGUAGCUCCCGAAACGUUGCUACAAUAAG
GCCGUCUGAAAAGAUGUGCCGCAACGCUCUGCCUU
CUGGCAUCGUUUA
(SG-103NTv1);
(SEQ ID NO: 105)
(N)ₓGUUGUAGCUCCCGAAACGUUGCUACAAUAAG
GCCGUCUGAAAAGAUGUGCCGCAACGCUCUGCCCU
UCUGGGCAUCGUU
(SG-103NTv2);
(SEQ ID NO: 106)
(N)ₓGUUGUAGCUCCCGAAACGUUGCUACAAUAAG
GCCGUCUGAAAAGAUGUGCCGCAACGCUCUGCUUC
UGCAUCGUUUA
(SG-101NTv1);
or
(SEQ ID NO: 107)
(N)ₓGUUGUAGCUCCCGAAACGUUGCUACAAUAAG
GCCGUCUGAAAAGAUGUGCCGCAACGCUCUGCUUC
UGCAUCGUU
(SG-99NT),

wherein N corresponds to any nucleotide and x corresponds to any integer between 18 and 26.

In one aspect, the disclosure provides a chemically modified Neisseria meningitidis (Nme) single guide RNA (sgRNA) comprising a chemical modification pattern selected from anyone of:

(SEQ ID NO: 108)
(mN)#(mN)#(mN)#(rN)(rN)(rN)(rN)(rN)(rN)
(rN)(rN)(rN)(rN)(rN)(rN)(rN)(rN)(rN)(rN
)(rN)(rN)(rN)(rN)(rN)(rG)(rU)(rU)(rG)(r
U)(rA)(rG)(rC)(rU)(rC)(rC)(rC)(mG)(mA)(
mA)(mA)(rC)(rG)(rU)(rU)(rG)(rC)(rU)(rA)
(rC)(rA)(rA)(rU)(rA)(rA)(rG)(rG)(rC)(rC
)(rG)(rU)(mC)(mU)(mG)(mA)(mA)(mA)(mA)(m
G)(rA)(rU)(rG)(rU)(rG)(rC)(rC)(rG)(rC)(
rA)(rA)(rC)(rG)(rC)(rU)(rC)(rU)(rG)(rC)
(rC)(mU)(mU)(mC)(mU)(rG)(rG)(rC)(rA)(rU
)(rC)(rG)(rU)#(mU)#(mU)#(mA)
(SG-1);
(SEQ ID NO: 109)
(mN)#(mN)#(mN)#(rN)(rN)(rN)(rN)(rN)(rN)
(rN)(rN)(rN)(rN)(rN)(rN)(rN)(rN)(rN)(rN
)(rN)(rN)(rN)(rN)(rN)(rG)(rU)(rU)(rG)(r
U)(mA)(mG)(mC)(rU)(rC)(rC)(rC)(mG)(mA)(
mA)(mA)(rC)(rG)(rU)(rU)(rG)(rC)(rU)(rA)
(rC)(rA)(rA)(rU)(rA)(rA)(rG)(rG)(mC)(mC
)(rG)(mU)(mC)(mU)(mG)(mA)(mA)(mA)(mA)(m
G)(rA)(rU)(rG)(rU)(rG)(rC)(rC)(rG)(rC)(
rA)(rA)(rC)(rG)(rC)(mU)(mC)(mU)(mG)(rC)
(rC)(mU)(mU)(mC)(mU)(rG)(rG)(rC)(rA)(rU
)(mC)(mG)(mU)#(mU)#(mU)#(mA)
(SG-2);
(SEQ ID NO: 110)
(mN)#(mN)#(mN)#(mN)(mN)(rN)(rN)(rN)(mN)
(mN)(mN)(rN)(mN)(rN)(rN)(rN)(rN)(mN)(rN
)(rN)(rN)(mN)(rN)(rN)(mG)(rU)(rU)(rG)(m
U)(mA)(mG)(mC)(rU)(rC)(rC)(rC)(mG)(mA)(
mA)(mA)(mC)(rG)(mU)(rU)(mG)(mC)(rU)(mA)
(mC)(rA)(rA)(rU)(rA)(mA)(rG)(mG)(mC)(mC
)(mG)(mU)(mC)(mU)(mG)(mA)(mA)(mA)(mA)(m
G)(mA)(mU)(rG)(mU)(mG)(mC)(mC)(mG)(mC)(
mA)(mA)(mC)(mG)(mC)(mU)(mC)(mU)(mG)(mC)
(mC)(mU)(mU)(mC)(mU)(rG)(mG)(rC)(mA)(mU
)(mC)(mG)(mU)#(mU)#(mU)#(mA)
(SG-3);
(SEQ ID NO: 111)
(mN)#(mN)#(mN)#(mN)(mN)(rN)(rN)(rN)(mN)
(mN)(mN)(rN)(mN)(rN)(rN)(mN)(rN)(mN)(rN
)(rN)(rN)(mN)(rN)(rN)(mG)(rU)(rU)(rG)(m
U)(mA)(mG)(mC)(rU)(rC)(rC)(rC)(mG)(mA)(
mA)(mA)(mC)(rG)(mU)(rU)(mG)(mC)(rU)(mA)
(mC)(rA)(rA)(rU)(rA)(mA)(rG)(mG)(fC)(mC
)(fG)(mU)(fC)(mU)(fG)(mA)(fA)(mA)(fA)(m
G)(fA)(mU)(rG)(mU)(fG)(mC)(fC)(mG)(fC)(
mA)(fA)(mC)(fG)(mC)(fU)(mC)(fU)(mG)(fC)
(mC)(fU)(mU)(fC)(mU)(rG)(mG)(rC)(mA)(fU
)(mC)(fG)(mU)#(mU)#(mU)#(mA)
(SG-4);
(SEQ ID NO: 112)
(mN)#(mN)#(mN)#(mN)(mN)(rN)(rN)(rN)(mN)
(mN)(mN)(rN)(mN)(rN)(rN)(rN)(rN)(mN)(rN
)(rN)(rN)(mN)(rN)(rN)(mG)(rU)(rU)(rG)(m
U)(mA)(mG)(mC)(rU)(rC)(rC)(rC)(mG)(mA)(
mA)(mA)(rC)(rG)(rU)(rU)(rG(rC)(rU)(rA)(
rC)(rA)(rA)(rU)(rA)(rA)(rG)(rG)(mC)(mC)
(rG)(mU)(mC(mU)(mG)(mA)(mA)(mA)(mA)(mG)
(rA)(rU)(rG)(rU)(rG)(rC)(rC)(rG)(rC)(rA
(rA)(rC)(rG)(rC)(mU)(mC)(mU)(mG)(rC)(rC
)(mU)(mU)(mC)(mU)(rG)(rG)(rC)(rA)(rU)(m
C)(mG)(mU)#(mU)#(mU)#(mA)
(SG-5);
(SEQ ID NO: 113)
(mN)#(mN)#(mN)#(rN)(mN)(rN)(rN)(rN)(mN)
(mN)(rN)(rN)(mN)(rN)(rN)(rN)(rN)(mN)(rN
)(rN)(rN)(mN)(rN)(rN)(mG)(rU)(rU)(rG)(r
U)(mA)(mG)(mC)(rU)(rC)(rC)(rC)(mG)(mA)(
mA)(mA)(rC)(rG)(rU)(rU)(rG)(rC)(rU)(rA)
(rC)(rA)(rA)(rU)(rA)(rA)(rG)(rG)(mC)(mC
)(rG)(mU)(mC)(mU)(mG)(mA)(mA)(mA)(mA)(m
G)(rA)(rU)(rG)(rU)(rG)(rC)(rC)(rG)(rC)(
rA)(rA)(rC)(rG)(rC)(mU)(mC)(mU)(mG)(rC)
(rC)(mU)(mU)(mC)(mU)(rG)(rG)(rC)(rA)(rU
)(mC)(mG)(mU)#(mU)#(mU)#(mA)
(SG-6);
(SEQ ID NO: 114)
(mN)#(mN)#(mN)#(rN)(rN)(rN)(rN)(rN)(rN)
(rN)(rN)(rN)(rN)(rN)(rN)(rN)(rN)(mN)(rN
)(rN)(rN)(mN)(rN)(rN)(rG)(rU)(rU)(rG)(r
U)(mA)(mG)(mC)(rU)(rC)(rC)(rC)(mG)(mA)(
mA)(mA)(rC)(rG)(rU)(rU)(rG)(rC)(rU)(rA)
(rC)(rA)(rA)(rU)(rA)(rA)(rG)(rG)(mC)(mC
)(rG)(mU)(mC)(mU)(mG)(mA)(mA)(mA)(mA)(m
G)(rA)(rU)(rG)(rU)(rG)(rC)(rC)(rG)(rC)(
rA)(rA)(rC)(rG)(rC)(mU)(mC)(mU)(mG)(rC)
(rC)(mU)(mU)(mC)(mU)(rG)(rG)(rC)(rA)(rU
)(mC)(mG)(mU)#(mU)#(mU)#(mA)
(SG-7);
(SEQ ID NO: 115)
(mN)#(mN)#(mN)#(mN)(mN)(mN)(mN)(mN)(mN)
(mN)(mN)(mN)(mN)(mN)(rN)(rN)(rN)(rN)(rN)
(rN)(rN)(rN)(rN)(rN)(rG)(rU)(rU)(rG)(rU
)(mA)(mG)(mC)(rU)(rC)(rC)(rC)(mG)(mA)(m
A)(mA)(rC)(rG)(rU)(rU)(rG)(rC)(rU)(rA)(
rC)(rA(rA)(rU)(rA)(rA)(rG)(rG)(mC)(mC)(
rG)(mU)(mC)(mU)(mG)(mA)(mA)(mA)(mA)(mG)
(rA)(rU)(rG)(rU)(rG)(rC)(rC)(rG)(rC)(rA
)(rA)(rC)(rG)(rC)(mU)(mC)(mU)(mG)(rC)(r
C)(mU)(mU)(mC)(mU)(rG)(rG)(rC)(rA)(rU)(
mC)(mG)(mU)#(mU)#(mU)#(mA)
(SG-8);
(SEQ ID NO: 116)
(mN)#(mN)#(mN)#(mN)(mN)(mN)(mN)(mN)(mN)
(mN)(mN)(mN)(mN)(mN)(rN)(rN)(rN)(mN)(rN
)(rN)(rN)(mN)(rN)(rN)(mG)(rU)(rU)(rG)(m
U)(mA)(mG)(mC)(rU)(rC)(rC)(rC)(mG)(mA)(
mA)(mA)(rC)(rG)(rU)(rU)(rG)(rC)(rU)(rA)
(rC)(rA)(rA)(rU)(rA)(rA)(rG)(rG)(mC)(mC
)(rG)(mU)(mC)(mU)(mG)(mA)(mA)(mA)(mA)(m
G)(rA)(rU)(rG)(rU)(rG)(rC)(rC)(rG)(rC)(
rA)(rA)(rC)(rG)(rC)(mU)(mC)(mU)(mG)(rC)
(rC)(mU)(mU)(mC)(mU)(rG)(rG)(rC)(rA)(rU
)(mC)(mG)(mU)#(mU)#(mU)#(mA)
(SG-9);
(SEQ ID NO: 117)
(mN)#(mN)#(mN)#(mN)(mN)(rN)(rN)(rN)(mN)
(mN)(mN)(rN)(mN)(rN)(rN)(rN)(rN)(mN)(rN
)(rN)(rN)(mN)(rN)(rN)(mG)(rU)(rU)(rG)(m
U)(mA)(mG)(mC)(rU)(rC)(rC)(rC)(mG)(mA)(
mA)(mA)(rC)(rG)(rU)(rU)(rG)(rC)(rU)(rA)
(rC)(rA)(rA)(rU)(rA)(mA)(rG)(mG)(mC)(mC
)(mG)(mU)(mC)(mU)(mG)(mA)(mA)(mA)(mA)(m
G)(mA)(mU)(rG)(rU)(mG)(mC)(mC)(rG)(rC)(
rA)(rA)(rC)(rG)(rC)(mU)(mC)(mU)(mG)(rC)
(rC)(mU)(mU)(mC)(mU)(rG)(rG)(rC)(rA)(rU
)(mC)(mG)(mU)#(mU)#(mU)#(mA)
(SG-10);
(SEQ ID NO: 118)
(mN)#(mN)#(mN)#(mN)(mN)(rN)(rN)(rN)(mN)
(mN)(mN)(rN)(mN)(rN)(rN)(rN)(rN)(mN)(rN
)(rN)(rN)(mN)(rN)(rN)(mG)(rU)(rU)(rG)(m
U)(mA)(mG)(mC)(rU)(rC)(rC)(rC)(mG)(mA)(
mA)(mA)(rC)(rG)(rU)(rU)(rG)(rC)(rU)(rA)
(rC)(rA)(rA)(rU)(rA)(rA)(rG)(rG)(mC)(mC
)(rG)(mU)(mC)(mU)(mG)(mA)(mA)(mA)(mA)(m
G)(rA)(rU)(rG)(rU)(rG)(rC)(rC)(mG)(rC)(
mA)(mA)(mC)(mG)(mC)(mU)(mC)(mU)(mG)(mC)
(mC)(mU)(mU)(mC)(mU)(rG)(mG)(rC)(mA)(rU
)(mC)(mG)(mU)#(mU)#(mU)#(mA)
(SG-11);
(SEQ ID NO: 119)
(mN)#(mN)#(mN)#(mN)(mN)(rN)(rN)(rN)(mN)
(mN)(mN)(rN)(mN)(rN)(rN)(rN)(rN)(mN)(rN
)(rN)(rN)(mN)(rN)(rN)(fG)(rU)(rU)(rG)(f
U)(fA)(fG)(fC)(rU)(rC)(rC)(rC)(mG)(mA)(
mA)(mA)(rC)(rG)(rU)(rU)(rG)(rC)(rU)(rA)
(rC)(rA)(rA)(rU)(rA)(rA)(rG)(rG)(fC)(fC
)(rG)(fU)(fC)(fU)(mG)(mA)(mA)(mA)(fA)(f
G)(rA)(rU)(rG)(rU)(rG)(rC)(rC)(rG)(rC)(
rA)(rA)(rC)(rG)(rC)(mU)(mC)(fU)(fG)(rC)
(rC)(mU)(mU)(mC)(mU)(rG)(rG)(rC)(rA)(rU
)(fC)(fG)(fU)#(fU)#(mU)#(mA)
(SG-12);
(SEQ ID NO: 120)
(mN)#(mN)#(mN)#(mN)(mN)(rN)(rN)(rN)(mN)
(mN)(mN)(rN)(mN)(rN)(rN)(rN)(rN)(mN)(rN
)(rN)(rN)(mN)(rN)(rN)(fG)(rU)(rU)(rG)(f
U)(fA)(fG)(fC)(rU)(rC)(rC)(rC)(mG)(mA)(
mA)(mA)(mC)(rG)(mU)(rU)(fG)(fC)(rU)(fA)
(fC)(rA)(rA)(rU)(rA)(rA)(rG)(rG)(mC)(mC
)(rG)(mU)(mC)(mU)(mG)(mA)(mA)(mA)(mA)(m
G)(rA)(rU)(rG)(rU)(rG)(rC)(rC)(rG)(rC)(
rA)(rA)(rC)(rG)(rC)(mU)(mC)(mU)(mG)(rC)
(rC)(mU)(mU)(mC)(mU)(rG)(rG)(rC)(rA)(rU
)(mC)(mG)(mU)#(mU)#(mU)#(mA)
(SG-13);
(SEQ ID NO: 121)
(mN)#(mN)#(mN)#(mN)(mN)(rN)(rN)(rN)(mN)
(mN)(mN)(rN)(mN)(rN)(rN)(rN)(rN)(mN)(rN
)(rN)(rN)(mN)(rN)(rN)(fG)(rU)(rU)(rG)(f
U)(fA)(fG)(fC)(rU)(rC)(rC)(rC)(mG)(mA)(
mA)(mA)(mC)(rG)(mU)(rU)(IG)(fC)(rU)(fA)
(fC)(rA)(rA)(rU)(rA)(TA)(rG)(rG)(fC)(fC
)(rG)(fU)(fC)(fU)(mG)(mA)(mA)(mA)(fA)(f
G)(rA)(rU)(rG)(rU)(rG)(rC)(rC)(rG)(rC)(
rA)(rA)(rC)(rG)(rC)(mU)(mC)(fU)(fG)(rC)
(rC)(mU)(mU)(mC)(mU)(rG)(rG)(rC)(rA)(rU
)(fC)(fG)(fU)#(fU)#(mU)#(mA)
(SG-14);
(SEQ ID NO: 122)
mN#mN#mN#rNrNrNrNrNrNrNrNrNrNrNrNrNrNrN
rNrNrNrNrNrNrGrUrUrGrUrArGrCrUrCrCrCrGr
ArArArCrGrUrUrGrCrUrArCrArArUrArArGrGrC
rCrGrUrCrUrGrArArArArGrArUrGrUrGrCrCrGr
CrArArCrGrCrUrCrUrGrCrCrCrCrUrUrArArArG
rCrUrCrCrUrGrCrUrUrUrArArGrGrGrGrCrArUr
CrGrUmU#mU#mA
(SG121-0);
(SEQ ID NO: 123)
mN#mN#mN#mNmNrNrNrNrNrNrNrNrNrNrNrNrNrN
rNrNrNrNrNrNrGrUrUrGrUrArGrCrUrCrCrCmGm
AmAmArCrGrUrUrGrCrUrArCrArArUrArArGrGrC
rCrGmUmCmUmGmAmAmAmAmGrArUrGrUrGrCrCrGr
CrArArCrGrCrUrCrUrGrCrCmCmCmUmUmAmAmAmG
mCmUmCmCmUmGmCmUmUmUmAmAmGmGrGrGrCrArUm
CmGmU#mU#mU#mA
(SG121-2);
(SEQ ID NO: 124)
mN#mN#mN#mNmNrNrNrNmNmNmNrNmNrNrNrNrNmN
rNrNrNmNrNrNmGrUrUrGmUmAmGmCrUrCrCrCmGm
AmAmArCrGrUrUrGrCrUrArCrArArUrArArGrGrC
rCrGmUmCmUmGmAmAmAmAmGrArUrGrUrGrCrCrGr
CrArArCrGrCrUrCrUrGrCrCmCmCmUmUmAmAmAmG
mCmUmCmCmUmGmCmUmUmUmAmAmGmGrGrGrCrArUm
CmGmU#mU#mU#mA
(SG121-3);
and
(SEQ ID NO: 125)
mN#mN#mN#mNmNmNmNmNmNmNmNmNmNmNrNrNrNmN
rNrNrNmNrNrNmGrUrUrGmUmAmGmCrUrCrCrCmGm
AmAmArCrGrUrUrGrCrUrArCrArArUrArArGrGrC
rCrGmUmCmUmGmAmAmAmAmGrArUrGrUrGrCrCrGr
CrArArCrGrCrUrCrUrGrCrCmCmCmUmUmAmAmAmG
mCmUmCmCmUmGmCmUmUmUmAmAmGmGrGrGrCrArUm
CmGmU#mU#mU#mA
(SG121-4),

wherein rN=RNA, mN=2′-O-methyl RNA, fN=2′-fluoro RNA, N #N=phosphorothioate linkage, and N=any nucleotide.

In certain embodiments, the chemically modified Nme gRNA or sgRNA further comprise at least one moiety conjugated to the gRNA or sgRNA.

In certain embodiments, the at least one moiety is conjugated to at least one of the 5′ end of the crRNA portion, the 3′ end of the crRNA portion, the 5′ end of the tracrRNA portion, the 3′ end of the tracrRNA portion, the 5′ end of the sgRNA, and the 3′ end of the sgRNA.

In certain embodiments, the at least one moiety increases cellular uptake of the gRNA or sgRNA. In certain embodiments, the at least one moiety promotes specific tissue distribution of the gRNA or sgRNA.

In certain embodiments, the at least one moiety is selected from the group consisting of fatty acids, steroids, secosteroids, lipids, gangliosides analogs, nucleoside analogs, endocannabinoids, vitamins, receptor ligands, peptides, aptamers, and alkyl chains. In certain embodiments, the at least one moiety is selected from the group consisting of cholesterol, docosahexaenoic acid (DHA), docosanoic acid (DCA), lithocholic acid (LA), GalNAc, amphiphilic block copolymer (ABC), hydrophilic block copolymer (HBC), poloxamer, Cy5, and Cy3.

In certain embodiments, the at least one moiety is conjugated to the gRNA or sgRNA via a linker. In certain embodiments, the linker is selected from the group consisting of an ethylene glycol chain, an alkyl chain, a polypeptide, a polysaccharide, and a block copolymer.

In certain embodiments, the at least one moiety is a modified lipid. In certain embodiments, the modified lipid is a branched lipid. In certain embodiments, the modified lipid is a branched lipid of Formula I, Formula I: X-MC(═Y)M-Z-[L-MC(═Y)M-R]n, where X is a moiety that links the lipid to the gRNA or sgRNA, each Y is independently oxygen or sulfur, each M is independently CH₂, NH, O or S, Z is a branching group which allows two or three (“n”) chains to be joined to a chemically modified guide RNA, L is an optional linker moiety, and each R is independently a saturated, monounsaturated or polyunsaturated linear or branched moiety from 2 to 30 atoms in length, a sterol, or other hydrophobic group.

In certain embodiments, the modified lipid is a headgroup-modified lipid. In certain embodiments, the modified lipid is a headgroup-modified lipid of Formula II, Formula II: X-MC(═Y)M-Z[L-MC(═Y)M-R]n-L-K-J, where X is a moiety that links the lipid to the gRNA or sgRNA, each Y is independently oxygen or sulfur, each M is independently CH₂, NH, N-alkyl, O or S, Z is a branching group which allows two or three (“n”) chains to be joined to chemically modified guide RNA, each L is independently an optional linker moiety, and R is a saturated, monounsaturated or polyunsaturated linear or branched moiety from 2 to 30 atoms in length, a sterol, or other hydrophobic group, K is a phosphate, sulfate, or amide and J is an aminoalkane or quaternary aminoalkane group.

In certain embodiments, the guide RNA binds to a N. meningitidis Cas9 nuclease (NmeCas9) or variant thereof. In certain embodiments, the Nme Cas9 nuclease or variant thereof is Nme1Cas9, Nme2Cas9, or Nme3Cas9. In certain embodiments, the Nme Cas9 nuclease or variant thereof comprises an amino acid sequence with at least 80% identity to an amino acid sequence set forth in SEQ ID NO: 1, 2, or 3.

In certain embodiments, the NmeCas9 further comprises one or more nuclear localization signal (NLS) polypeptides. In certain embodiments, the NLS polypeptide comprises one or both of a nucleoplasmin NLS and an SV40 NLS.

In certain embodiments, the NmeCas9 is a variant NmeCas9. In certain embodiments, the variant NmeCas9 comprises one or both of a D16A mutation and a H588A mutation.

In certain embodiments, the NmeCas9 or variant NmeCas9 is fused to a nucleotide deaminase. In certain embodiments, the nucleotide deaminase is a cytidine deaminase. In certain embodiments, the nucleotide deaminase is an adenosine deaminase. In certain embodiments, the NmeCas9 or variant NmeCas9 further comprises a uracil glycosylase inhibitor.

In certain embodiments, Cas9 off-target activity is reduced relative to an unmodified gRNA or sgRNA.

In certain embodiments, Cas9 on-target activity is increased relative to an unmodified gRNA or sgRNA.

In certain embodiments, the chemically modified Nme gRNA or sgRNA comprises at least about 50% activity relative to an unmodified gRNA or sgRNA.

In one aspect, the disclosure provides a method of altering expression of a target gene in a cell, comprising contacting the cell with a genome editing system comprising: the chemically modified Nme gRNA or sgRNA described above; and an RNA-guided nuclease or a polynucleotide encoding an RNA-guided nuclease.

In certain embodiments, the target gene is in a cell in an organism. In certain embodiments, the contacting occurs in vivo. In certain embodiments, the contacting occurs ex vivo or in vitro.

In certain embodiments, expression of the target gene is knocked out or knocked down.

In certain embodiments, the sequence of the target gene is modified, edited, corrected or enhanced.

In certain embodiments, the guide RNA and the RNA-guided nuclease comprise a ribonucleoprotein (RNP) complex.

In certain embodiments, the RNA-guided nuclease is an N. meningitidis Cas9 (NmeCas9). In certain embodiments, the Nme Cas9 is Nme1 Cas9, Nme2Cas9, or Nme3Cas9.

In certain embodiments, the NmeCas9 further comprises one or more nuclear localization signal (NLS) polypeptides. In certain embodiments, the NLS polypeptide comprises one or both of a nucleoplasmin NLS and an SV40 NLS.

In certain embodiments, the NmeCas9 is a variant NmeCas9. In certain embodiments, the variant NmeCas9 comprises one or both of a D16A mutation and a H588A mutation.

In certain embodiments, the NmeCas9 or variant NmeCas9 is fused to a nucleotide deaminase. In certain embodiments, the nucleotide deaminase is a cytidine deaminase. In certain embodiments, the nucleotide deaminase is an adenosine deaminase. In certain embodiments, the NmeCas9 or variant NmeCas9 further comprises a uracil glycosylase inhibitor.

In certain embodiments, the polynucleotide encoding an RNA-guided nuclease comprises a vector.

In certain embodiments, the vector further comprises a polynucleotide encoding a crRNA portion or a tracrRNA portion.

In certain embodiments, the vector is a viral vector. In certain embodiments, the viral vector is an adeno-associated virus (AAV) vector, an adenovirus vector, or a lentivirus (LV) vector.

In certain embodiments, the polynucleotide encoding an RNA-guided nuclease comprises an mRNA.

In certain embodiments, the mRNA is formulated in a lipid nanoparticle (LNP). In certain embodiments, the LNP comprises at least one cationic lipid. In certain embodiments, the LNP further comprises a PEGylated lipid, a helper lipid, and cholesterol or derivative thereof.

In certain embodiments, expression of the target gene is reduced by at least about 20%.

In one aspect, the disclosure provides a CRISPR genome editing system comprising: a chemically modified gRNA or sgRNA described above; and an RNA-guided nuclease or a polynucleotide encoding an RNA-guided nuclease.

In one aspect, the disclosure provides a chemically modified Neisseria meningitidis (Nme) guide RNA (gRNA) comprising: (a) a crRNA portion comprising (i) a guide sequence capable of hybridizing to a target polynucleotide sequence, and (ii) a repeat sequence; and (b) a tracrRNA portion comprising an anti-repeat nucleotide sequence that is complementary to the repeat sequence, wherein the crRNA portion comprises at least 40% modified nucleotides, and wherein the gRNA is capable of binding to an Nme2Cas9 nuclease.

In one aspect, the disclosure provides a ribonucleoprotein (RNP) complex comprising: i) a Neisseria meningitidis (Nme) Cas9 nuclease; and ii) a chemically modified Nme guide RNA (gRNA), wherein the Nme gRNA comprises: (a) a crRNA portion comprising (i) a guide sequence capable of hybridizing to a target polynucleotide sequence, and (ii) a repeat sequence; and (b) a tracrRNA portion comprising an anti-repeat nucleotide sequence that is complementary to the repeat sequence, wherein the crRNA portion comprises at least 40% modified nucleotides.

In certain embodiments, the gRNA is capable of binding to the Nme Cas9 nuclease or variant thereof. In certain embodiments, the Nme Cas9 or variant thereof is Nme1 Cas9, Nme2Cas9, or Nme3Cas9.

### BRIEF DESCRIPTION OF THE DRAWINGS

The foregoing and other features and advantages of the present disclosure will be more fully understood from the following detailed description of illustrative embodiments taken in conjunction with the accompanying drawings. The patent or application file contains at least one drawing executed in color. Copies of this patent or patent application publication with color drawing(s) will be provided by the Office upon request and payment of the necessary fee.

FIG. 1 depicts editing efficiencies of several 48-nucleotide long Nme chemically modified crRNAs (CR48-2, CR48-154, CR48-155, CR48-190 to CR48-205, and CR48-213 to CR48-227). An unmodified Nme tracrRNA was paired with each crRNA. The Traffic Light Reporter Multi-Cas Variant 1 (TLR-MCV1) reporter cell line was used and transfected with an RNP comprising Nme2Cas9 and the gRNAs. The graphs show the percentages of red fluorescent (RF) cells obtained by fluorescence activated cell sorting (FACS) analysis. Data are mean values of three biological replicates and error bars represent s.e.m.

FIG. 2 depicts editing efficiencies of several 42-nucleotide long Nme chemically modified crRNAs (CR42-2, CR42-4, CR42-154 to CR42-161, CR42-167 to CR42-189). An unmodified Nme tracrRNA was paired with each crRNA. The TLR-MCV1 reporter cell line was used and transfected with an RNP comprising Nme2Cas9 and the gRNAs. The graphs show the percentages of RF cells obtained by FACS analysis. Data are mean values of three biological replicates and error bars represent s.e.m.

FIG. 3 depicts editing efficiencies of several 48-nucleotide long Nme chemically modified crRNAs (CR48-199 V1.1 to CR48-199 V1.36). An unmodified Nme tracrRNA was paired with each crRNA. The TLR-MCV1 reporter cell line was used and transfected with an RNP comprising Nme2Cas9 and the gRNAs. The graphs show the percentages of RF cells obtained by FACS analysis. Data are mean values of three biological replicates and error bars represent s.e.m.

FIG. 4 depicts editing efficiencies of several 48-nucleotide long Nme chemically modified crRNAs (CR48-203 V1.7, CR48-203 V1.10, CR48-203 V1.12, CR48-203 V1.18, CR48-203 V1.19, CR48-203 V1.22. CR48-203 V1.24, CR48-203 V1.30, CR48-203 V1.31, CR48-203 V1.34, CR48-203 V1.36, CR48-203 V1.31*). An unmodified Nme tracrRNA was paired with each crRNA. The TLR-MCV1 reporter cell line was used and transfected with an RNP comprising Nme2Cas9 and the gRNAs. The graphs show the percentages of RF cells obtained by FACS analysis. Data are mean values of three biological replicates and error bars represent s.e.m.

FIG. 5 depicts editing efficiencies of several 93-nucleotide long Nme chemically modified tracrRNAs (TR-0, TR-1, and TR-2). A minimally modified Nme crRNA (CR48-2) was paired with each tracrRNA. The TLR-MCV1 reporter cell line was used and transfected with an RNP comprising Nme2Cas9 and the gRNAs. The graphs show the percentages of RF cells obtained by FACS analysis. Data are mean values of three biological replicates and error bars represent s.e.m.

FIG. 6 depicts editing efficiencies of several 103-nucleotide long Nme chemically modified single guide RNAs (sgRNAs) (SG-0 to SG-14). The TLR-MCV1 reporter cell line was used and transfected with an RNP comprising Nme2Cas9 and the gRNAs. The graphs show the percentages of RF cells obtained by FACS analysis. Data are mean values of three biological replicates and error bars represent s.e.m.

FIG. 7 depicts editing efficiencies of several sgRNAs (SG-1 and SG-5) when co-delivered with an AAV expressing Nme2Cas9. The TLR-MCV1 reporter cell line was used and transduced with the AAV. 50 pmole of the chemically modified gRNAs were co-delivered. The graphs show the percentages of RF cells obtained by FACS analysis. Data are mean values of three biological replicates and error bars represent s.e.m.

FIG. 8 depicts in vivo editing with Nme2Cas9-SG-5 RNPs. RNPs with a shuttle peptide delivered to the mouse striatum via a bilateral injection targeted the TLR-MCV1 reporter. Immunohistochemical staining of mCherry was performed 1 week after RNP injections.

FIG. 9 depicts the codelivery of mRNA and sgRNA using 103-nucleotide long Nme chemically modified sgRNAs (SG103-0) vs 121-nucleotide long Nme chemically modified sgRNAs (SG121-0). Nme2Cas9 mRNA was kept constant and end-modified synthetic sgRNA was titrated. The bars represent activity in the TLR-MCV cell line. Data are mean values of two biological replicates and error bars represent s.e.m.

FIG. 10 depicts the activity of several SG121 modifications (SG121-0, SG121-2, SG121-3, and SG121-4) in targeting human and mouse genomic loci. Human HEK293T cells and mouse Hepa1-6 cells were used for this experiment. SG121 modified guides were electroporated into cells with Nme2Cas9 mRNA. SG121-0, SG121-2, SG121-3, and SG121-4 were used to target human TLR-MCV reporter cell line, Linc01588, LSP1, FancF, and SG121-0, SG121-3 were used to target murine Ttr.
FIG. 11 depicts the editing activity of several SG121 modified sgRNA with various NMe2 adenosine base editors (ABE) at genomic locus Linc01588. The max editing rate observed within the adenine within the target was plotted.

FIG. 12 displays editing activity of select heavily modified CR48 variants identified via TLR-MCV screening (CR48-0, CR48-4, CR48-199, CR48-203, CR48-199, 7, CR48-199, 18, and CR48-199.31) along with SG121 guides (SG121-0 and SG121-3). The guides target mouse TTR in Hepa1-6 cells.

### DETAILED DESCRIPTION

Provided herewith are novel chemically modified crRNAs and tracrRNAs, including heavily or fully chemically modified crRNAs and tracrRNAs. In certain embodiments, crRNAs and tracrRNAs with 5′ and/or 3′ conjugated moieties are provided. In yet other embodiments, crRNAs and tracrRNAs with modifications in the repeat region of the crRNA or the anti-repeat region of the tracrRNA are provided. Methods of using the crRNAs and tracrRNAs of the disclosure for genome editing with a CRISPR nuclease and kits for performing the same are also provided.

Unless otherwise defined herein, nomenclature used in connection with cell and tissue culture, molecular biology, immunology, microbiology, genetics and protein and nucleic acid chemistry and hybridization described herein are those well-known and commonly used in the art. The methods and techniques provided herein are usually performed according to conventional methods well known in the art and as described in various general and more specific references that are cited and discussed throughout the present specification unless otherwise indicated. Enzymatic reactions and purification techniques are performed according to manufacturer's specifications unless otherwise specified, as commonly accomplished in the art or as described herein. The nomenclature used in connection with, and the laboratory procedures and techniques of, analytical chemistry, synthetic organic chemistry, and medicinal and pharmaceutical chemistry described herein are those well-known and commonly used in the art, unless otherwise specified. Standard techniques are used for chemical syntheses, chemical analyses, pharmaceutical preparation, formulation, and delivery, and treatment of patients.

Unless otherwise defined herein, scientific and technical terms used herein have the meanings that are commonly understood by those of ordinary skill in the art. In the event of any latent ambiguity, definitions provided herein take precedent over any dictionary or extrinsic definition. Unless otherwise required by context, singular terms shall include pluralities and plural terms shall include the singular. The use of “or” means “and/or” unless stated otherwise. The use of the term “including,” as well as other forms, such as “includes” and “included,” is not limiting.

So that the disclosure may be more readily understood, certain terms are first defined.

As used herein, the term “guide RNA” or “gRNA” refer to any nucleic acid that promotes the specific association (or “targeting”) of an RNA-guided nuclease such as a Cas9 to a target sequence (e.g., a genomic or episomal sequence) in a cell.

As used herein, a “modular” or “dual RNA” guide comprises more than one, and typically two, separate RNA molecules, such as a CRISPR RNA (crRNA) and a trans-activating crRNA (tracrRNA), which are usually associated with one another, for example by duplexing. gRNAs and their component parts are described throughout the literature (see, e.g., Briner et al. Mol. Cell, 56(2), 333-339 (2014), which is incorporated by reference).

As used herein, a “unimolecular gRNA,” “chimeric gRNA,” or “single guide RNA (sgRNA)” comprises a single RNA molecule. The sgRNA may be a crRNA and tracrRNA linked together. For example, the 3′ end of the crRNA may be linked to the 5′ end of the tracrRNA. A crRNA and a tracrRNA may be joined into a single unimolecular or chimeric gRNA, for example, by means of a four nucleotide (e.g., GAAA) “tetraloop” or “linker” sequence bridging complementary regions of the crRNA (at its 3′ end) and the tracrRNA (at its 5′ end).

As used herein, a “repeat” sequence or region is a nucleotide sequence at or near the 3′ end of the crRNA which is complementary to an anti-repeat sequence of a tracrRNA.

As used herein, an “anti-repeat” sequence or region is a nucleotide sequence at or near the 5′ end of the tracrRNA which is complementary to the repeat sequence of a crRNA.

Additional details regarding guide RNA structure and function, including the gRNA/Cas9 complex for genome editing may be found in, at least, Mali et al. Science, 339(6121), 823-826 (2013); Jiang et al. Nat. Biotechnol. 31(3). 233-239 (2013); Jinek et al. Science, 337(6096), 816-821 (2012); and Sun et al. Mol. Cell, 76, 938-952 (2019), each of which are incorporated herein by reference.

As used herein, a “guide sequence” or “targeting sequence” refers to the nucleotide sequence of a gRNA, whether unimolecular or modular, that is fully or partially complementary to a target domain or target polynucleotide within a DNA sequence in the genome of a cell where editing is desired. Guide sequences are typically 10-30 nucleotides in length, preferably 16-26 nucleotides in length (for example, 16, 17, 18, 19, 20, 21, 22, 23, 24, 25, or 26 nucleotides in length), and are at or near the 5′ terminus of a Cas9 gRNA.

As used herein, a “target domain” or “target polynucleotide sequence” is the DNA sequence in a genome of a cell that is complementary to the guide sequence of the gRNA.

In addition to the targeting domains, gRNAs typically include a plurality of domains that influence the formation or activity of gRNA/Cas9 complexes. For example, as mentioned above, the duplexed structure formed by first and secondary complementarity domains of a gRNA (also referred to as a repeat: anti-repeat duplex) interacts with the recognition (REC) lobe of Cas9 and may mediate the formation of Cas9/gRNA complexes (Nishimasu et al. Cell 156: 935-949 (2014); Nishimasu et al. Cell 162(2), 1113-1126 (2015); Sun et al., supra, each incorporated by reference herein). It should be noted that the first and/or second complementarity domains can contain one or more poly-U tracts, which can be recognized by RNA polymerases as a termination signal. The sequence of the first and second complementarity domains are, therefore, optionally modified to eliminate these tracts and promote the complete in vitro transcription of gRNAs, for example through the use of A-G swaps as described in Briner 2014, or A-U swaps. These and other similar modifications to the first and second complementarity domains are within the scope of the present disclosure.

Along with the first and second complementarity domains, Cas9 gRNAs typically include two or more additional duplexed regions that are necessary for nuclease activity in vivo but not necessarily in vitro (Nishimasu 2015, supra; Sun et al., supra). A first stem-loop near the 3′ portion of the second complementarity domain is referred to variously as the “proximal domain,” “stem loop 1” (Nishimasu 2014, supra; Nishimasu 2015, supra; Sun et al., supra) and the “nexus” (Briner 2014, supra). One or more additional stem loop structures are generally present near the 3′ end of the gRNA, with the number varying by species: N. meningitidis gRNAs typically include two 3′ stem loops (for a total of four stem loop structures including the repeat: anti-repeat duplex), while S. aureus and other species have only one (for a total of three). A description of conserved stem loop structures (and gRNA structures more generally) organized by species is provided in Briner 2014, which is incorporated herein by reference. Additional details regarding guide RNAs generally may be found in WO2018026976A1, which is incorporated herein by reference.

Chemically Modified N. meningitidis Guide RNA

The chemically modified Nme guide RNAs of the disclosure possess improved in vivo stability, improved genome editing efficacy, and/or reduced immunotoxicity relative to unmodified or minimally modified guide RNAs.

Chemically modified guide RNAs of the disclosure contain one or more modified nucleotides comprising a modification in a ribose group, a phosphate group, a nucleobase, or a combination thereof.

Chemical modifications to the ribose group may include, but are not limited to, 2′-O-methyl, 2′-fluoro, 2′-deoxy, 2′-O-(2-methoxy ethyl) (MOE), 2′-NH₂ (2′-amino), 4′-thio, 2′-O-Allyl, 2′-O-Ethylamine, 2′-O-Cyanoethyl, 2′-O-Acetalester, or a bicyclic nucleotide, such as locked nucleic acid (LNA), 2′-(S)-constrained ethyl (S-cEt), constrained MOE, or 2′-O,4′-C-aminomethylene bridged nucleic acid (2′,4′-BNA^{NC}).

The term “4′-thio” as used herein corresponds to a ribose group modification where the sugar ring oxygen of the ribose is replaced with a sulfur.

Chemical modifications to the phosphate group may include, but are not limited to, a phosphorothioate, phosphonoacetate (PACE), thiophosphonoacetate (thioPACE), amide, triazole, phosphonate, or phosphotriester modification.

In an embodiment, the crRNA portion of the chemically modified guide RNA comprises between 1 and 20 phosphorothioate modifications (i.e., 1, 2, 3, 4, 5, 6, 7, 8, 9, 10, 11, 12, 13, 14, 15, 16, 17, 18, 19, or 20 phosphorothioate modifications). In an embodiment, the crRNA portion of the chemically modified guide RNA comprises between 1 and 20 phosphorothioate modifications (i.e., 1, 2, 3, 4, 5, 6, 7, 8, 9, 10, 11, 12, 13, 14, 15, 16, 17, 18, 19, or 20 phosphorothioate modifications) and comprises at least about 50% activity relative to a guide RNA that does not comprise phosphorothioate modifications (e.g., 50% activity, 60% activity, 70% activity, 80% activity, 90% activity, 95% activity, or 100% activity, relative to a guide RNA that does not comprise phosphorothioate modifications).

Chemical modifications to the nucleobase may include, but are not limited to, 2-thiouridine, 4-thiouridine, N⁶-methyladenosine, pseudouridine, 2,6-diaminopurine, inosine, thymidine, 5-methylcytosine, 5-substituted pyrimidine, isoguanine, isocytosine, or halogenated aromatic groups.

The chemically modified guide RNAs may have one or more chemical modifications in the crRNA portion and/or the tracrRNA portion for a modular or dual RNA guide. The chemically modified guide RNAs may also have one or more chemical modifications in the single guide RNA for the unimolecular guide RNA.

The chemically modified guide RNAs may comprise at least about 40% to about 100% chemically modified nucleotides, at least about 50% to about 100% chemically modified nucleotides, at least about 60% to about 100% chemically modified nucleotides, at least about 70% to about 100% chemically modified nucleotides, at least about 80% to about 100% chemically modified nucleotides, at least about 90% to about 100% chemically modified nucleotides, and at least about 95% to about 100% chemically modified nucleotides.

The chemically modified guide RNAs may comprise at least about 40% chemically modified nucleotides, at least about 50% chemically modified nucleotides, at least about 60% chemically modified nucleotides, at least about 70% chemically modified nucleotides, at least about 80% chemically modified nucleotides, at least about 90% chemically modified nucleotides, at least about 95% chemically modified nucleotides, at least about 99% chemically modified, or 100% (fully) chemically modified nucleotides.

The chemically modified guide RNAs may comprise about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or 100% chemically modified nucleotides.

The chemically modified guide RNAs may comprise at least about 80%, 81%, 82%, 83%, 84%, 85%, 86%, 87%, 88%, 89%, 90%, 91%, 92%, 93%, 94%, 95%, 96%, 97%, 98%, 99%, or 100% chemically modified nucleotides.

Guide RNAs that comprise at least about 40% chemically modified nucleotides to at least about 99% chemically modified nucleotides are considered “heavily” modified, as used herein.

Guide RNAs that comprise 100% chemically modified nucleotides are considered “fully” modified, as used herein.

In certain exemplary embodiments, the chemically modified guide RNAs may comprise a chemically modified ribose group at about 40% of the guide RNA nucleotides to about 100% of the guide RNA nucleotides, at about 50% of the guide RNA nucleotides to about 100% of the guide RNA nucleotides, at about 60% of the guide RNA nucleotides to about 100% of the guide RNA nucleotides, at about 70% of the guide RNA nucleotides to about 100% of the guide RNA nucleotides, at about 80% of the guide RNA nucleotides to about 100% of the guide RNA nucleotides, at about 90% of the guide RNA nucleotides to about 100% of the guide RNA nucleotides, and at about 95% of the guide RNA nucleotides to about 100% of the guide RNA nucleotides

In certain exemplary embodiments, the chemically modified guide RNAs may comprise a chemically modified ribose group at about 40% of the guide RNA nucleotides, at about 50% of the guide RNA nucleotides, at about 60% of the guide RNA nucleotides, at about 70% of the guide RNA nucleotides, at about 80% of the guide RNA nucleotides, at about 90% of the guide RNA nucleotides, at about 95% of the guide RNA nucleotides, at about 99% of the guide RNA nucleotides, or at 100% of the guide RNA nucleotides.

In certain exemplary embodiments, the chemically modified guide RNAs may comprise a chemically modified ribose group at about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or 100% of the guide RNA nucleotides.

Guide RNAs that have at least about 40% of the ribose groups chemically modified to at least about 99% of the ribose groups chemically modified are also considered “heavily” modified, as used herein.

Guide RNAs that have 100% of the ribose groups chemically modified are also considered “fully” modified, as used herein.

In certain exemplary embodiments, the chemically modified guide RNAs may comprise a chemically modified phosphate group at about 50% of the guide RNA nucleotides to about 100% of the guide RNA nucleotides, at about 60% of the guide RNA nucleotides to about 100% of the guide RNA nucleotides, at about 70% of the guide RNA nucleotides to about 100% of the guide RNA nucleotides, at about 80% of the guide RNA nucleotides to about 100% of the guide RNA nucleotides, at about 90% of the guide RNA nucleotides to about 100% of the guide RNA nucleotides, and at about 95% of the guide RNA nucleotides to about 100% of the guide RNA nucleotides

In certain exemplary embodiments, the chemically modified guide RNAs may comprise a chemically modified phosphate group at about 50% of the guide RNA nucleotides, at about 60% of the guide RNA nucleotides, at about 70% of the guide RNA nucleotides, at about 80% of the guide RNA nucleotides, at about 90% of the guide RNA nucleotides, at about 95% of the guide RNA nucleotides, at about 99% of the guide RNA nucleotides, or at 100% of the guide RNA nucleotides.

In certain exemplary embodiments, the chemically modified guide RNAs may comprise a chemically modified phosphate group at about 80%, 81%, 82%, 83%, 84%, 85%, 86%, 87%, 88%, 89%, 90%, 91%, 92%, 93%, 94%, 95%, 96%, 97%, 98%, 99%, or 100% of the guide RNA nucleotides.

Guide RNAs that have at least about 80% of the phosphate groups chemically modified to at least about 99% of the phosphate groups chemically modified are also considered “heavily” modified, as used herein.

Guide RNAs that have 100% of the phosphate groups chemically modified are also considered “fully” modified, as used herein.

In certain exemplary embodiments, the chemically modified guide RNAs may comprise a chemically modified nucleobase at about 40% of the guide RNA nucleotides to about 100% of the guide RNA nucleotides, at about 50% of the guide RNA nucleotides to about 100% of the guide RNA nucleotides, at about 60% of the guide RNA nucleotides to about 100% of the guide RNA nucleotides, at about 70% of the guide RNA nucleotides to about 100% of the guide RNA nucleotides, at about 80% of the guide RNA nucleotides to about 100% of the guide RNA nucleotides, at about 90% of the guide RNA nucleotides to about 100% of the guide RNA nucleotides, and at about 95% of the guide RNA nucleotides to about 100% of the guide RNA nucleotides.

In certain exemplary embodiments, the chemically modified guide RNAs may comprise a chemically modified nucleobase at about 40% of the guide RNA nucleotides, at about 50% of the guide RNA nucleotides, at about 60% of the guide RNA nucleotides, at about 70% of the guide RNA nucleotides, at about 80% of the guide RNA nucleotides, at about 90% of the guide RNA nucleotides, at about 95% of the guide RNA nucleotides, at about 99% of the guide RNA nucleotides, or at 100% of the guide RNA nucleotides.

In certain exemplary embodiments, the chemically modified guide RNAs may comprise a chemically modified nucleobase at about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or 100% of the guide RNA nucleotides.

Guide RNAs that have at least about 40% of the nucleobases chemically modified to at least about 99% of the nucleobases chemically modified are also considered “heavily” modified, as used herein.

Guide RNAs that have 100% of the nucleobases chemically modified are also considered “fully” modified, as used herein.

In certain exemplary embodiments, the chemically modified guide RNAs may comprise any combination of chemically modified ribose groups, chemically modified phosphate groups, and chemically modified nucleobases at about 40% of the guide RNA nucleotides to about 100% of the guide RNA nucleotides, at about 50% of the guide RNA nucleotides to about 100% of the guide RNA nucleotides, at about 60% of the guide RNA nucleotides to about 100% of the guide RNA nucleotides, at about 70% of the guide RNA nucleotides to about 100% of the guide RNA nucleotides, at about 80% of the guide RNA nucleotides to about 100% of the guide RNA nucleotides, at about 90% of the guide RNA nucleotides to about 100% of the guide RNA nucleotides, and at about 95% of the guide RNA nucleotides to about 100% of the guide RNA nucleotides.

In certain exemplary embodiments, the chemically modified guide RNAs may comprise any combination of chemically modified ribose groups, chemically modified phosphate groups, and chemically modified nucleobases at about 40% of the guide RNA nucleotides, at about 50% of the guide RNA nucleotides, at about 60% of the guide RNA nucleotides, at about 70% of the guide RNA nucleotides, at about 80% of the guide RNA nucleotides, at about 90% of the guide RNA nucleotides, at about 95% of the guide RNA nucleotides, at about 99% of the guide RNA nucleotides, or at 100% of the guide RNA nucleotides.

In certain exemplary embodiments, the chemically modified guide RNAs may comprise any combination of chemically modified ribose groups, chemically modified phosphate groups, and chemically modified nucleobases at about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or 100% of the guide RNA nucleotides.

Guide RNAs that have at least about 80% of any combination of the ribose groups, the phosphate groups, and the nucleobases chemically modified to at least about 99% of the nucleobases chemically modified are also considered “heavily” modified, as used herein.

Guide RNAs that have 100% of any combination of the ribose groups, the phosphate groups, and the nucleobases chemically modified are also considered “fully” modified, as used herein.

The heavily and fully chemically modified guide RNAs of the disclosure possess several advantages over the minimally modified guide RNAs in the art. Heavily and fully chemically modified guide RNAs are expected to ease chemical synthesis, further enhance in vivo stability, and provide a scaffold for terminally appended chemical functionalities that facilitate delivery and efficacy during clinical applications to genome editing.

The chemical modification pattern used in the guide RNA is such that activity of the guide RNA is maintained when paired with an RNA-guided DNA endonuclease, e.g., Nme Cas9.

In an embodiment, the chemically modified guide RNAs of the disclosure comprise at least about 50% activity relative to an unmodified guide RNA (e.g., 50% activity, 60% activity, 70% activity, 80% activity, 90% activity, 95% activity, or 100% activity, relative to an unmodified guide RNA).

The activity of a guide RNA can be readily determined by any means known in the art. In an embodiment, % activity is measured with the traffic light reporter (TLR) Multi-Cas Variant 1 system (TLR-MCV1), described below. The TLR-MCV1 system will provide a % fluorescent cells which is a measure of % activity.

Exemplary chemical modification patterns are described in Table 1 and Table 2 below.

TABLE 1
Exemplary chemical modification patterns for crRNAs

| | | SEQ |
|---|---|---|
| Name | Sequence | ID NO: |
| CR48-2 | (mN)#(mN)#(mN)#(mN)(mN)(rN)(rN)(rN)(rN)(rN)(rN)(rN)(rN)(rN)(rN)(rN)(rN)(rN) | 13 |
| | (rN)(rN)(rN)(rN)(rN)(rN)(rG)(rU)(rU)(rG)(rU)(rA)(rG)(rC)(rU)(rC)(rC)(rC)(rU) | |
| | (mU)(mU)(mC)(mU)(mC)(mA)(mU)(mU)#(mU)#(mC)#(mG) | |
| CR48-154 | (mN)#(mN)#(mN)#(mN)(mN)(mN)(dN)#(mN)(mN)(mN)(mN)(fN)(mN)(fN)(mN)(fN) | 14 |
| | (mN)(mN)(rN)(rN)(rN)(mN)(rN)(rN)(mG)(rU)(rU)(rG)(mU)(mA)(mG)(mC)(rU)(rC) | |
| | (rC)(rC)(rU)(mU)(mU)(mC)(mU)(mC)(mA)(mU)(mU)#(mU)#(mC)#(mG) | |
| CR48-155 | (mN)#(mN)#(mN)#(mN)(mN)(mN)(dN)#(mN)(mN)(mN)(mN)(mN)(mN)(fN)(mN)(fN) | 15 |
| | (mN)(mN)(rN)(rN)(rN)(mN)(rN)(rN)(mG)(rU)(rU)(rG)(mU)(mA)(mG)(mC)(rU)(rC) | |
| | (rC)(rC)(rU)(mU)(mU)(mC)(mU)(mC)(mA)(mU)(mU)#(mU)#(mC)#(mG) | |
| CR48-190 | (mN)#(mN)#(mN)#(mN)(mN)(mN)(dN)#(mN)(mN)(mN)(mN)(fN)(mN)(fN)(mN)(fN) | 16 |
| | (mN)(mN)(rN)(fN)(mN)(mN)(mN)(mN)(mG)(rU)(rU)(rG)(mU)(mA)(mG)(mC)(rU)(rC) | |
| | (rC)(rC)(rU)(mU)(mU)(mC)(mU)(mC)(mA)(mU)(mU)#(mU)#(mC)#(mG) | |
| CR48-191 | (mN)#(mN)#(mN)#(mN)(mN)(mN)(dN)#(mN)(mN)(mN)(mN)(fN)(mN)(fN)(mN)(fN) | 17 |
| | (mN)(mN)(rN)(mN)(fN)(mN)(mN)(mN)(mG)(rU)(rU)(rG)(mU)(mA)(mG)(mC)(rU)(rC) | |
| | (rC)(rC)(rU)(mU)(mU)(mC)(mU)(mC)(mA)(mU)(mU)#(mU)#(mC)#(mG) | |
| CR48-192 | (mN)#(mN)#(mN)#(mN)(mN)(mN)(dN)#(mN)(mN)(mN)(mN)(mN)(mN)(fN)(mN)(fN) | 18 |
| | (mN)(mN)(rN)(fN)(mN)(mN)(mN)(mN)(mG)(rU)(rU)(rG)(mU)(mA)(mG)(mC)(rU) | |
| | (rC)(rC)(rC)(rU)(mU)(mU)(mC)(mU)(mC)(mA)(mU)(mU)#(mU)#(mC)#(mG) | |
| CR48-193 | (mN)#(mN)#(mN)#(mN)(mN)(mN)(dN)#(mN)(mN)(mN)(mN)(mN)(mN)(fN)(mN)(fN) | 19 |
| | (mN)(mN)(rN)(mN)(fN)(mN)(mN)(mN)(mG)(rU)(rU)(rG)(mU)(mA)(mG)(mC)(rU) | |
| | (rC)(rC)(rC)(rU)(mU)(mU)(mC)(mU)(mC)(mA)(mU)(mU)#(mU)#(mC)#(mG) | |
| CR48-194 | (mN)#(mN)#(mN)#(mN)(mN)(mN)(dN)#(mN)(mN)(mN)(mN)(fN)(mN)(fN)(mN)(fN) | 20 |
| | (mN)(mN)(rN)#(fN)(mN)(mN)(mN)(mN)(mG)(rU)(rU)(rG)(mU)(mA)(mG)(mC)(rU) | |
| | (rC)(rC)(rC)(rU)(mU)(mU)(mC)(mU)(mC)(mA)(mU)(mU)#(mU)#(mC)#(mG) | |
| CR48-195 | (mN)#(mN)#(mN)#(mN)(mN)(mN)(dN)#(mN)(mN)(mN)(mN)(fN)(mN)(fN)(mN)(fN) | 21 |
| | (mN)(mN)(rN)#(mN)(fN)(mN)(mN)(mN)(mG)(rU)(rU)(rG)(mU)(mA)(mG)(mC)(rU) | |
| | (rC)(rC)(rC)(rU)(mU)(mU)(mC)(mU)(mC)(mA)(mU)(mU)#(mU)#(mC)#(mG) | |
| CR48-196 | (mN)#(mN)#(mN)#(mN)(mN)(mN)(dN)#(mN)(mN)(mN)(mN)(mN)(mN)(fN)(mN)(fN) | 22 |
| | (mN)(mN)(rN)#(fN)(mN)(mN)(mN)(mN)(mG)(rU)(rU)(rG)(mU)(mA)(mG)(mC)(rU) | |
| | (rC)(rC)(rC)(rU)(mU)(mU)(mC)(mU)(mC)(mA)(mU)(mU)#(mU)#(mC)#(mG) | |
| CR48-197 | (mN)#(mN)#(mN)#(mN)(mN)(mN)(dN)#(mN)(mN)(mN)(mN)(mN)(mN)(fN)(mN)(fN) | 23 |
| | (mN)(mN)(rN)#(mN)(fN)(mN)(mN)(mN)(mG)(rU)(rU)(rG)(mU)(mA)(mG)(mC)(rU) | |
| | (rC)(rC)(rC)(rU)(mU)(mU)(mC)(mU)(mC)(mA)(mU)(mU)#(mU)#(mC)#(mG) | |
| CR48-198 | (mN)#(mN)#(mN)#(mN)(mN)(mN)(dN)#(mN)(mN)(mN)(mN)(fN)(mN)(fN)(mN)(fN) | 24 |
| | (mN)(mN)(mN)(fN)(mN)(mN)(mN)(mN)(mG)(rU)(rU)(rG)(mU)(mA)(mG)(mC)(rU) | |
| | (rC)(rC)(rC)(rU)(mU)(mU)(mC)(mU)(mC)(mA)(mU)(mU)#(mU)#(mC)#(mG) | |
| CR48-199 | (mN)#(mN)#(mN)#(mN)(mN)(mN)(dN)#(mN)(mN)(mN)(mN)(fN)(mN)(fN)(mN)(fN) | 25 |
| | (mN)(mN)(mN)(mN)(fN)(mN)(mN)(mN)(mG)(rU)(rU)(rG)(mU)(mA)(mG)(mC)(rU) | |
| | (rC)(rC)(rC)(rU)(mU)(mU)(mC)(mU)(mC)(mA)(mU)(mU)#(mU)#(mC)#(mG) | |
| CR48-200 | (mN)#(mN)#(mN)#(mN)(mN)(mN)(dN)#(mN)(mN)(mN)(mN)(mN)(mN)(fN)(mN)(fN) | 26 |
| | (mN)(mN)(mN)(fN)(mN)(mN)(mN)(mN)(mG)(rU)(rU)(rG)(mU)(mA)(mG)(mC)(rU) | |
| | (rC)(rC)(rC)(rU)(mU)(mU)(mC)(mU)(mC)(mA)(mU)(mU)#(mU)#(mC)#(mG) | |
| CR48-201 | (mN)#(mN)#(mN)#(mN)(mN)(mN)(dN)#(mN)(mN)(mN)(mN)(mN)(mN)(fN)(mN)(fN) | 27 |
| | (mN)(mN)(mN)(mN)(fN)(mN)(mN)(mN)(mG)(rU)(rU)(rG)(mU)(mA)(mG)(mC)(rU) | |
| | (rC)(rC)(rC)(rU)(mU)(mU)(mC)(mU)(mC)(mA)(mU)(mU)#(mU)#(mC)#(mG) | |
| CR48-202 | (mN)#(mN)#(mN)#(mN)(mN)(mN)(dN)#(mN)(mN)(mN)(mN)(fN)(mN)(fN)(mN)(fN) | 28 |
| | (mN)(mN)(fN)(fN)(mN)(mN)(mN)(mN)(mG)(rU)(rU)(rG)(mU)(mA)(mG)(mC)(rU)(rC) | |
| | (rC)(rC)(rU)(mU)(mU)(mC)(mU)(mC)(mA)(mU)(mU)#(mU)#(mC)#(mG) | |
| CR48-203 | (mN)#(mN)#(mN)#(mN)(mN)(mN)(dN)#(mN)(mN)(mN)(mN)(fN)(mN)(fN)(mN)(fN) | 29 |
| | (mN)(mN)(fN)(mN)(fN)(mN)(mN)(mN)(mG)(rU)(rU)(rG)(mU)(mA)(mG)(mC)(rU) | |
| | (rC)(rC)(rC)(rU)(mU)(mU)(mC)(mU)(mC)(mA)(mU)(mU)#(mU)#(mC)#(mG) | |
| CR48-204 | (mN)#(mN)#(mN)#(mN)(mN)(mN)(dN)#(mN)(mN)(mN)(mN)(mN)(mN)(fN)(mN)(fN) | 30 |
| | (mN)(mN)(fN)(fN)(mN)(mN)(mN)(mN)(mG)(rU)(rU)(rG)(mU)(mA)(mG)(mC)(rU) | |
| | (rC)(rC)(rC)(rU)(mU)(mU)(mC)(mU)(mC)(mA)(mU)(mU)#(mU)#(mC)#(mG) | |
| CR48-205 | (mN)#(mN)#(mN)#(mN)(mN)(mN)(dN)#(mN)(mN)(mN)(mN)(mN)(mN)(fN)(mN)(fN) | 31 |
| | (mN)(mN)(fN)(mN)(fN)(mN)(mN)(mN)(mG)(rU)(rU)(rG)(mU)(mA)(mG)(mC)(rU) | |
| | (rC)(rC)(rC)(rU)(mU)(mU)(mC)(mU)(mC)(mA)(mU)(mU)#(mU)#(mC)#(mG) | |
| CR48-213 | (mN)#(mN)#(mN)#(mN)(mN)(mN)(dN)#(mN)(mN)(mN)(mN)(mN)(mN)(fN)(mN)(fN) | 32 |
| | (mN)(mN)(rN)(rN)(rN)(mN)(rN)(rN)(mG)(dT)#(rU)(mG)(mU)(mA)(mG)(mC)(rU) | |
| | (rC)(rC)(rC)(rU)(mU)(mU)(mC)(mU)(mC)(mA)(mU)(mU)#(mU)#(mC)#(mG) | |
| CR48-214 | (mN)#(mN)#(mN)#(mN)(mN)(mN)(dN)#(mN)(mN)(mN)(mN)(mN)(mN)(fN)(mN)(fN) | 33 |
| | (mN)(mN)(rN)(rN)(rN)(mN)(rN)(rN)(mG)(dT)#(rU)(mG)(mU)(mA)(mG)(mC)(rU) | |
| | (rC)(mC)(rC)(rU)(mU)(mU)(mC)(mU)(mC)(mA)(mU)(mU)#(mU)#(mC)#(mG) | |
| CR48-215 | (mN)#(mN)#(mN)#(mN)(mN)(mN)(dN)#(mN)(mN)(mN)(mN)(mN)(mN)(fN)(mN)(fN) | 34 |
| | (mN)(mN)(rN)(rN)(rN)(mN)(rN)(rN)(mG)(dT)#(rU)(mG)(mU)(mA)(mG)(mC)(rU) | |
| | (rC)(rC)(mC)(rU)(mU)(mU)(mC)(mU)(mC)(mA)(mU)(mU)#(mU)#(mC)#(mG) | |
| CR48-216 | (mN)#(mN)#(mN)#(mN)(mN)(mN)(dN)#(mN)(mN)(mN)(mN)(mN)(mN)(fN)(mN)(fN) | 35 |
| | (mN)(mN)(rN)(rN)(rN)(mN)(rN)(rN)(mG)(dT)#(rU)(mG)(mU)(mA)(mG)(mC)(rU) | |
| | (rC)(mC)(mC)(rU)(mU)(mU)(mC)(mU)(mC)(mA)(mU)(mU)#(mU)#(mC)#(mG) | |
| CR48-217 | (mN)#(mN)#(mN)#(mN)(mN)(mN)(dN)#(mN)(mN)(mN)(mN)(mN)(mN)(fN)(mN)(fN) | 36 |
| | (mN)(mN)(rN)(rN)(rN)(mN)(rN)(rN)(mG)(rU)(rU)(rG)(mU)(mA)(mG)(mC)(dT)#(rC) | |
| | (rC)(rC)(rU)(mU)(mU)(mC)(mU)(mC)(mA)(mU)(mU)#(mU)#(mC)#(mG) | |
| CR48-218 | (mN)#(mN)#(mN)#(mN)(mN)(mN)(dN)#(mN)(mN)(mN)(mN)(mN)(mN)(fN)(mN)(fN) | 37 |
| | (mN)(mN)(rN)(rN)(rN)(mN)(rN)(rN)(mG)(rU)(rU)(rG)(mU)(mA)(mG)(mC)(rU)(dC) | |
| | #(rC)(rC)(rU)(mU)(mU)(mC)(mU)(mC)(mA)(mU)(mU)#(mU)#(mC)#(mG) | |
| CR48-219 | (mN)#(mN)#(mN)#(mN)(mN)(mN)(dN)#(mN)(mN)(mN)(mN)(mN)(mN)(fN)(mN)(fN) | 38 |
| | (mN)(mN)(rN)(rN)(rN)(mN)(rN)(rN)(mG)(rU)(rU)(rG)(mU)(mA)(mG)(mC)(rU)(rC) | |
| | (dC)#(rC)(rU)(mU)(mU)(mC)(mU)(mC)(mA)(mU)(mU)#(mU)#(mC)#(mG) | |
| CR48-220 | (mN)#(mN)#(mN)#(mN)(mN)(mN)(dN)#(mN)(mN)(mN)(mN)(mN)(mN)(fN)(mN)(fN) | 39 |
| | (mN)(mN)(rN)(rN)(rN)(mN)(rN)(rN)(mG)(rU)(rU)(rG)(mU)(mA)(mG)(mC)(rU)(rC) | |
| | (rC)(dC)#(rU)(mU)(mU)(mC)(mU)(mC)(mA)(mU)(mU)#(mU)#(mC)#(mG) | |
| CR48-221 | (mN)#(mN)#(mN)#(mN)(mN)(mN)(dN)#(mN)(mN)(mN)(mN)(mN)(mN)(fN)(mN)(fN) | 40 |
| | (mN)(mN)(rN)(rN)(rN)(mN)(rN)(rN)(mG)(rU)(rU)(rG)(mU)(mA)(mG)(mC)(rU)(rC) | |
| | (rC)(rC)(dT)#(mU)(mU)(mC)(mU)(mC)(mA)(mU)(mU)#(mU)#(mC)#(mG) | |
| CR48-222 | (mN)#(mN)#(mN)#(mN)(mN)(mN)(dN)#(mN)(mN)(mN)(mN)(mN)(mN)(fN)(mN)(fN) | 41 |
| | (mN)(mN)(rN)(rN)(rN)(mN)(rN)(rN)(mG)(rU)(rU)(rG)(mU)(mA)(mG)(mC)(dT)#(rC) | |
| | (dC)#(rC)(dT)#(mU)(mU)(mC)(mU)(mC)(mA)(mU)(mU)#(mU)#(mC)#(mG) | |
| CR48-223 | (mN)#(mN)#(mN)#(mN)(mN)(mN)(dN)#(mN)(mN)(mN)(mN)(mN)(mN)(fN)(mN)(fN) | 42 |
| | (mN)(mN)(rN)(rN)(rN)(mN)(rN)(rN)(mG)(dT)#(rU)(rG)(mU)(mA)(mG)(mC)(dT)# | |
| | (dC)#(dC)#(rC)(dT)#(mU)(mU)(mC)(mU)(mC)(mA)(mU)(mU)#(mU)#(mC)#(mG) | |
| CR48-224 | (mN)#(mN)#(mN)#(mN)(mN)(mN)(dN)#(mN)(mN)(mN)(mN)(mN)(mN)(fN)(mN)(fN) | 43 |
| | (mN)(mN)(rN)(rN)(rN)(mN)(rN)(rN)(mG)(dT)#(rU)(mG)(mU)(mA)(mG)(mC)(dT)# | |
| | (rC)(mC)(rC)(dT)#(mU)(mU)(mC)(mU)(mC)(mA)(mU)(mU)#(mU)#(mC)#(mG) | |
| CR48-225 | (mN)#(mN)#(mN)#(mN)(mN)(mN)(dN)#(mN)(mN)(mN)(mN)(mN)(mN)(fN)(mN)(fN) | 44 |
| | (mN)(mN)(rN)(rN)(rN)(mN)(rN)(rN)(mG)(dT)#(rU)(mG)(mU)(mA)(mG)(mC)(dT)# | |
| | (rC)(dC)#(mC)(dT)#(mU)(mU)(mC)(mU)(mC)(mA)(mU)(mU)#(mU)#(mC)#(mG) | |
| CR48-226 | (mN)#(mN)#(mN)#(mN)(mN)(mN)(dN)#(mN)(mN)(mN)(mN)(mN)(mN)(fN)(mN)(fN) | 45 |
| | (mN)(mN)(rN)(rN)(rN)(mN)(rN)(rN)(mG)(dT)#(rU)(mG)(mU)(mA)(mG)(mC)(dT)# | |
| | (rC)(mC)(mC)(dT)#(mU)(mU)(mC)(mU)(mC)(mA)(mU)(mU)#(mU)#(mC)#(mG) | |
| CR48-227 | (mN)#(mN)#(mN)#(mN)(mN)(mN)(dN)#(mN)(mN)(mN)(mN)(mN)(mN)(fN)(mN)(fN) | 46 |
| | (mN)(mN)(rN)(rN)(rN)(mN)(rN)(rN)(mG)(dT)#(rU)(mG)(mU)(mA)(mG)(mC)(dT)# | |
| | (dC)#(mC)(mC)(dT)#(mU)(mU)(mC)(mU)(mC)(mA)(mU)(mU)#(mU)#(mC)#(mG) | |
| CR48-199- | (mN)#(mN)#(mN)#(mN)(mN)(mN)(dN)#(mN)(mN)(mN)(mN)(fN)(mN)(fN)(mN)(fN) | 47 |
| V1.1 | (mN)(mN)(mN)(mN)(fN)(mN)(mN)(mN)(mG)(rU)(rU)(mG)(mU)(mA)(mG)(mC)(rU) | |
| | (rC)(rC)(dC)#(rU)(mU)(mU)(mC)(mU)(mC)(mA)(mU)(mU)#(mU)#(mC)#(mG) | |
| CR48-199- | (mN)#(mN)#(mN)#(mN)(mN)(mN)(dN)#(mN)(mN)(mN)(mN)(fN)(mN)(fN)(mN)(fN) | 48 |
| V1.2 | (mN)(mN)(mN)(mN)(fN)(mN)(mN)(mN)(mG)(rU)(rU)(mG)(mU)(mA)(mG)(mC)(rU) | |
| | (rC)(dC)#(rC)(rU)(mU)(mU)(mC)(mU)(mC)(mA)(mU)(mU)#(mU)#(mC)#(mG) | |
| CR48-199- | (mN)#(mN)#(mN)#(mN)(mN)(mN)(dN)#(mN)(mN)(mN)(mN)(fN)(mN)(fN)(mN)(fN) | 49 |
| V1.3 | (mN)(mN)(mN)(mN)(fN)(mN)(mN)(mN)(mG)(rU)(rU)(mG)(mU)(mA)(mG)(mC)(rU) | |
| | (rC)(rC)(rC)(dT)#(mU)(mU)(mC)(mU)(mC)(mA)(mU)(mU)#(mU)#(mC)#(mG) | |
| CR48-199- | (mN)#(mN)#(mN)#(mN)(mN)(mN)(dN)#(mN)(mN)(mN)(mN)(fN)(mN)(fN)(mN)(fN) | 50 |
| V1.4 | (mN)(mN)(mN)(mN)(fN)(mN)(mN)(mN)(mG)(rU)(rU)(mG)(mU)(mA)(mG)(mC)(rU) | |
| | (rC)(mC)(rC)(rU)(mU)(mU)(mC)(mU)(mC)(mA)(mU)(mU)#(mU)#(mC)#(mG) | |
| CR48-199- | (mN)#(mN)#(mN)#(mN)(mN)(mN)(dN)#(mN)(mN)(mN)(mN)(N)(mN)(fN)(mN)(fN) | 51 |
| V1.5 | (mN)(mN)(mN)(mN)(fN)(mN)(mN)(mN)(mG)(rU)(rU)(mG)(mU)(mA)(mG)(mC)(rU) | |
| | (rC)(dC)#(dC)#(rU)(mU)(mU)(mC)(mU)(mC)(mA)(mU)(mU)#(mU)#(mC)#(mG) | |
| CR48-199- | (mN)#(mN)#(mN)#(mN)(mN)(mN)(dN)#(mN)(mN)(mN)(mN)(fN)(mN)(fN)(mN)(fN) | 52 |
| V1.6 | (mN)(mN)(mN)(mN)(fN)(mN)(mN)(mN)(mG)(rU)(rU)(mG)(mU)(mA)(mG)(mC)(rU) | |
| | (rC)(dC)#(rC)(dT)#(mU)(mU)(mC)(mU)(mC)(mA)(mU)(mU)#(mU)#(mC)#(mG) | |
| CR48-199- | (mN)#(mN)#(mN)#(mN)(mN)(mN)(dN)#(mN)(mN)(mN)(mN)(fN)(mN)(fN)(mN)(fN) | 53 |
| V1.7 | (mN)(mN)(mN)(mN)(fN)(mN)(mN)(mN)(mG)(rU)(rU)(mG)(mU)(mA)(mG)(mC)(rU) | |
| | (rC)(dC)#(dC)#(dT)#(mU)(mU)(mC)(mU)(mC)(mA)(mU)(mU)#(mU)#(mC)#(mG) | |
| CR48-199- | (mN)#(mN)#(mN)#(mN)(mN)(mN)(dN)#(mN)(mN)(mN)(mN)(fN)(mN)(fN)(mN)(fN) | 54 |
| V1.8 | (mN)(mN)(mN)(mN)(fN)(mN)(mN)(mN)(mG)(rU)(rU)(mG)(mU)(mA)(mG)(mC)(rU) | |
| | (rC)(mC)(dC)#(rU)(mU)(mU)(mC)(mU)(mC)(mA)(mU)(mU)#(mU)#(mC)#(mG) | |
| CR48-199- | (mN)#(mN)#(mN)#(mN)(mN)(mN)(dN)#(mN)(mN)(mN)(mN)(fN)(mN)(fN)(mN)(fN) | 55 |
| V1.9 | (mN)(mN)(mN)(mN)(fN)(mN)(mN)(mN)(mG)(rU)(rU)(mG)(mU)(mA)(mG)(mC)(rU) | |
| | (rC)(mC)(rC)(dT)#(mU)(mU)(mC)(mU)(mC)(mA)(mU)(mU)#(mU)#(mC)#(mG) | |
| CR48-199- | (mN)#(mN)#(mN)#(mN)(mN)(mN)(dN)#(mN)(mN)(mN)(mN)(fN)(mN)(fN)(mN)(fN) | 56 |
| V1.10 | (mN)(mN)(mN)(mN)(fN)(mN)(mN)(mN)(mG)(rU)(rU)(mG)(mU)(mA)(mG)(mC)(rU) | |
| | (rC)(mC)(dC)#(dT)#(mU)(mU)(mC)(mU)(mC)(mA)(mU)(mU)#(mU)#(mC)#(mG) | |
| CR48-199- | (mN)#(mN)#(mN)#(mN)(mN)(mN)(dN)#(mN)(mN)(mN)(mN)(fN)(mN)(fN)(mN)(fN) | 57 |
| V1.11 | (mN)(mN)(mN)(mN)(fN)(mN)(mN)(mN)(mG)(rU)(rU)(mG)(mU)(mA)(mG)(mC)(rU) | |
| | (rC)(mC)(mC)(rU)(mU)(mU)(mC)(mU)(mC)(mA)(mU)(mU)#(mU)#(mC)#(mG) | |
| CR48-199- | (mN)#(mN)#(mN)#(mN)(mN)(mN)(dN)#(mN)(mN)(mN)(mN)(fN)(mN)(fN)(mN)(fN) | 58 |
| V1.12 | (mN)(mN)(mN)(mN)(fN)(mN)(mN)(mN)(mG)(rU)(rU)(mG)(mU)(mA)(mG)(mC)(rU) | |
| | (rC)(mC)(mC)(dT)#(mU)(mU)(mC)(mU)(mC)(mA)(mU)(mU)#(mU)#(mC)#(mG) | |
| CR48-199- | (mN)#(mN)#(mN)#(mN)(mN)(mN)(dN)#(mN)(mN)(mN)(mN)(fN)(mN)(fN)(mN)(fN) | 59 |
| V1.13 | (mN)(mN)(mN)(mN)(fN)(mN)(mN)(mN)(mG)(rU)(rU)(mG)(mU)(mA)(mG)(mC)(dT)# | |
| | (rC)(rC)(dC)#(rU)(mU)(mU)(mC)(mU)(mC)(mA)(mU)(mU)#(mU)#(mC)#(mG) | |
| CR48-199- | (mN)#(mN)#(mN)#(mN)(mN)(mN)(dN)#(mN)(mN)(mN)(mN)(fN)(mN)(fN)(mN)(fN) | 60 |
| V1.14 | (mN)(mN)(mN)(mN)(fN)(mN)(mN)(mN)(mG)(rU)(rU)(mG)(mU)(mA)(mG)(mC)(dT)# | |
| | (rC)(dC)#(rC)(rU)(mU)(mU)(mC)(mU)(mC)(mA)(mU)(mU)#(mU)#(mC)#(mG) | |
| CR48-199- | (mN)#(mN)#(mN)#(mN)(mN)(mN)(dN)#(mN)(mN)(mN)(mN)(fN)(mN)(fN)(mN)(fN) | 61 |
| V1.15 | (mN)(mN)(mN)(mN)(fN)(mN)(mN)(mN)(mG)(rU)(rU)(mG)(mU)(mA)(mG)(mC)(dT)# | |
| | (rC)(rC)(rC)(dT)#(mU)(mU)(mC)(mU)(mC)(mA)(mU)(mU)#(mU)#(mC)#(mG) | |
| CR48-199- | (mN)#(mN)#(mN)#(mN)(mN)(mN)(dN)#(mN)(mN)(mN)(mN)(fN)(mN)(fN)(mN)(fN) | 62 |
| V1.16 | (mN)(mN)(mN)(mN)(fN)(mN)(mN)(mN)(mG)(rU)(rU)(mG)(mU)(mA)(mG)(mC)(dT)# | |
| | (rC)(mC)(rC)(rU)(mU)(mU)(mC)(mU)(mC)(mA)(mU)(mU)#(mU)#(mC)#(mG) | |
| CR48-199- | (mN)#(mN)#(mN)#(mN)(mN)(mN)(dN)#(mN)(mN)(mN)(mN)(fN)(mN)(fN)(mN)(fN) | 63 |
| V1.17 | (mN)(mN)(mN)(mN)(fN)(mN)(mN)(mN)(mG)(rU)(rU)(mG)(mU)(mA)(mG)(mC)(dT)# | |
| | (rC)(dC)#(dC)#(rU)(mU)(mU)(mC)(mU)(mC)(mA)(mU)(mU)#(mU)#(mC)#(mG) | |
| CR48-199- | (mN)#(mN)#(mN)#(mN)(mN)(mN)(dN)#(mN)(mN)(mN)(mN)(fN)(mN)(fN)(mN)(fN) | 64 |
| V1.18 | (mN)(mN)(mN)(mN)(fN)(mN)(mN)(mN)(mG)(rU)(rU)(mG)(mU)(mA)(mG)(mC)(dT)# | |
| | (rC)(dC)#(rC)(dT)#(mU)(mU)(mC)(mU)(mC)(mA)(mU)(mU)#(mU)#(mC)#(mG) | |
| CR48-199- | (mN)#(mN)#(mN)#(mN)(mN)(mN)(dN)#(mN)(mN)(mN)(mN)(fN)(mN)(fN)(mN)(fN) | 65 |
| V1.19 | (mN)(mN)(mN)(mN)(fN)(mN)(mN)(mN)(mG)(rU)(rU)(mG)(mU)(mA)(mG)(mC)(dT)# | |
| | (rC)(dC)#(dC)#(dT)#(mU)(mU)(mC)(mU)(mC)(mA)(mU)(mU)#(mU)#(mC)#(mG) | |
| CR48-199- | (mN)#(mN)#(mN)#(mN)(mN)(mN)(dN)#(mN)(mN)(mN)(mN)(fN)(mN)(fN)(mN)(fN) | 66 |
| V1.20 | (mN)(mN)(mN)(mN)(fN)(mN)(mN)(mN)(mG)(rU)(rU)(mG)(mU)(mA)(mG)(mC)(dT)# | |
| | (rC)(mC)(dC)#(rU)(mU)(mU)(mC)(mU)(mC)(mA)(mU)(mU)#(mU)#(mC)#(mG) | |
| CR48-199- | (mN)#(mN)#(mN)#(mN)(mN)(mN)(dN)#(mN)(mN)(mN)(mN)(fN)(mN)(fN)(mN)(fN) | 67 |
| V1.21 | (mN)(mN)(mN)(mN)(fN)(mN)(mN)(mN)(mG)(rU)(rU)(mG)(mU)(mA)(mG)(mC)(dT)# | |
| | (rC)(mC)(rC)(dT)#(mU)(mU)(mC)(mU)(mC)(mA)(mU)(mU)#(mU)#(mC)#(mG) | |
| CR48-199- | (mN)#(mN)#(mN)#(mN)(mN)(mN)(dN)#(mN)(mN)(mN)(mN)(fN)(mN)(fN)(mN)(fN) | 68 |
| V1.22 | (mN)(mN)(mN)(mN)(fN)(mN)(mN)(mN)(mG)(rU)(rU)(mG)(mU)(mA)(mG)(mC)(dT)# | |
| | (rC)(mC)(dC)#(dT)#(mU)(mU)(mC)(mU)(mC)(mA)(mU)(mU)#(mU)#(mC)#(mG) | |
| CR48-199- | (mN)#(mN)#(mN)#(mN)(mN)(mN)(dN)#(mN)(mN)(mN)(mN)(N)(mN)(fN)(mN)(fN) | 69 |
| V1.23 | (mN)(mN)(mN)(mN)(fN)(mN)(mN)(mN)(mG)(rU)(rU)(mG)(mU)(mA)(mG)(mC)(dT)# | |
| | (rC)(mC)(mC)(rU)(mU)(mU)(mC)(mU)(mC)(mA)(mU)(mU)#(mU)#(mC)#(mG) | |
| CR48-199- | (mN)#(mN)#(mN)#(mN)(mN)(mN)(dN)#(mN)(mN)(mN)(mN)(fN)(mN)(fN)(mN)(fN) | 70 |
| V1.24 | (mN)(mN)(mN)(mN)(fN)(mN)(mN)(mN)(mG)(rU)(rU)(mG)(mU)(mA)(mG)(mC)(dT)# | |
| | (rC)(mC)(mC)(dT)#(mU)(mU)(mC)(mU)(mC)(mA)(mU)(mU)#(mU)#(mC)#(mG) | |
| CR48-199- | (mN)#(mN)#(mN)#(mN)(mN)(mN)(dN)#(mN)(mN)(mN)(mN)(fN)(mN)(fN)(mN)(fN) | 71 |
| V1.25 | (mN)(mN)(mN)(mN)(IN)(mN)(mN)(mN)(mG)(dT)#(rU)(mG)(mU)(mA)(mG)(mC)(dT) | |
| | #(rC)(rC)(dC)#(rU)(mU)(mU)(mC)(mU)(mC)(mA)(mU)(mU)#(mU)#(mC)#(mG) | |
| CR48-199- | (mN)#(mN)#(mN)#(mN)(mN)(mN)(dN)#(mN)(mN)(mN)(mN)(fN)(mN)(fN)(mN)(fN) | 72 |
| V1.26 | (mN)(mN)(mN)(mN)(IN)(mN)(mN)(mN)(mG)(dT)#(rU)(mG)(mU)(mA)(mG)(mC)(dT) | |
| | #(rC)(dC)#(rC)(rU)(mU)(mU)(mC)(mU)(mC)(mA)(mU)(mU)#(mU)#(mC)#(mG) | |
| CR48-199- | (mN)#(mN)#(mN)#(mN)(mN)(mN)(dN)#(mN)(mN)(mN)(mN)(fN)(mN)(fN)(mN)(fN) | 73 |
| V1.27 | (mN)(mN)(mN)(mN)(IN)(mN)(mN)(mN)(mG)(dT)#(rU)(mG)(mU)(mA)(mG)(mC)(dT) | |
| | #(rC)(rC)(rC)(dT)#(mU)(mU)(mC)(mU)(mC)(mA)(mU)(mU)#(mU)#(mC)#(mG) | |
| CR48-199- | (mN)#(mN)#(mN)#(mN)(mN)(mN)(dN)#(mN)(mN)(mN)(mN)(fN)(mN)(fN)(mN)(fN) | 74 |
| V1.28 | (mN)(mN)(mN)(mN)(IN)(mN)(mN)(mN)(mG)(dT)#(rU)(mG)(mU)(mA)(mG)(mC)(dT) | |
| | #(rC)(mC)(rC)(rU)(mU)(mU)(mC)(mU)(mC)(mA)(mU)(mU)#(mU)#(mC)#(mG) | |
| CR48-199- | (mN)#(mN)#(mN)#(mN)(mN)(mN)(dN)#(mN)(mN)(mN)(mN)(fN)(mN)(fN)(mN)(fN) | 75 |
| V1.29 | (mN)(mN)(mN)(mN)(IN)(mN)(mN)(mN)(mG)(dT)#(rU)(mG)(mU)(mA)(mG)(mC)(dT) | |
| | #(rC)(dC)#(dC)#(rU)(mU)(mU)(mC)(mU)(mC)(mA)(mU)(mU)#(mU)#(mC)#(mG) | |
| CR48-199- | (mN)#(mN)#(mN)#(mN)(mN)(mN)(dN)#(mN)(mN)(mN)(mN)(fN)(mN)(fN)(mN)(fN) | 76 |
| V1.30 | (mN)(mN)(mN)(mN)(IN)(mN)(mN)(mN)(mG)(dT)#(rU)(mG)(mU)(mA)(mG)(mC)(dT) | |
| | #(rC)(dC)#(rC)(dT)#(mU)(mU)(mC)(mU)(mC)(mA)(mU)(mU)#(mU)#(mC)#(mG) | |
| CR48-199- | (mN)#(mN)#(mN)#(mN)(mN)(mN)(dN)#(mN)(mN)(mN)(mN)(fN)(mN)(fN)(mN)(fN) | 77 |
| V1.31 | (mN)(mN)(mN)(mN)(IN)(mN)(mN)(mN)(mG)(dT)#(rU)(mG)(mU)(mA)(mG)(mC)(dT) | |
| | #(rC)(dC)#(dC)#(dT)#(mU)(mU)(mC)(mU)(mC)(mA)(mU)(mU)#(mU)#(mC)#(mG) | |
| CR48-199- | (mN)#(mN)#(mN)#(mN)(mN)(mN)(dN)#(mN)(mN)(mN)(mN)(fN)(mN)(fN)(mN)(fN) | 78 |
| V1.32 | (mN)(mN)(mN)(mN)(IN)(mN)(mN)(mN)(mG)(dT)#(rU)(mG)(mU)(mA)(mG)(mC)(dT) | |
| | #(rC)(mC)(dC)#(rU)(mU)(mU)(mC)(mU)(mC)(mA)(mU)(mU)#(mU)#(mC)#(mG) | |
| CR48-199- | (mN)#(mN)#(mN)#(mN)(mN)(mN)(dN)#(mN)(mN)(mN)(mN)(fN)(mN)(fN)(mN)(fN) | 79 |
| V1.33 | (mN)(mN)(mN)(mN)(IN)(mN)(mN)(mN)(mG)(dT)#(rU)(mG)(mU)(mA)(mG)(mC)(dT) | |
| | #(rC)(mC)(rC)(dT)#(mU)(mU)(mC)(mU)(mC)(mA)(mU)(mU)#(mU)#(mC)#(mG) | |
| CR48-199- | (mN)#(mN)#(mN)#(mN)(mN)(mN)(dN)#(mN)(mN)(mN)(mN)(fN)(mN)(fN)(mN)(fN) | 80 |
| V1.34 | (mN)(mN)(mN)(mN)(IN)(mN)(mN)(mN)(mG)(dT)#(rU)(mG)(mU)(mA)(mG)(mC)(dT) | |
| | #(rC)(mC)(dC)#(dT)#(mU)(mU)(mC)(mU)(mC)(mA)(mU)(mU)#(mU)#(mC)#(mG) | |
| CR48-199- | (mN)#(mN)#(mN)#(mN)(mN)(mN)(dN)#(mN)(mN)(mN)(mN)(fN)(mN)(fN)(mN)(fN) | 81 |
| V1.35 | (mN)(mN)(mN)(mN)(IN)(mN)(mN)(mN)(mG)(dT)#(rU)(mG)(mU)(mA)(mG)(mC)(dT) | |
| | #(rC)(mC)(mC)(rU)(mU)(mU)(mC)(mU)(mC)(mA)(mU)(mU)#(mU)#(mC)#(mG) | |
| CR48-199- | (mN)#(mN)#(mN)#(mN)(mN)(mN)(dN)#(mN)(mN)(mN)(mN)(fN)(mN)(fN)(mN)(fN) | 82 |
| V1.36 | (mN)(mN)(mN)(mN)(IN)(mN)(mN)(mN)(mG)(dT)#(rU)(mG)(mU)(mA)(mG)(mC)(dT) | |
| | #(rC)(mC)(mC)(dT)#(mU)(mU)(mC)(mU)(mC)(mA)(mU)(mU)#(mU)#(mC)#(mG) | |
| CR48-203- | (mN)#(mN)#(mN)#(mN)(mN)(mN)(dN)#(mN)(mN)(mN)(mN)(fN)(mN)(fN)(mN)(fN) | 83 |
| V1.7 | (mN)(mN)(fN)(mN)(fN)(mN)(mN)(mN)(mG)(rU)(rU)(mG)(mU)(mA)(mG)(mC)(rU) | |
| | (rC)(dC)#(dC)#(dT)#(mU)(mU)(mC)(mU)(mC)(mA)(mU)(mU)#(mU)#(mC)#(mG) | |
| CR48-203- | (mN)#(mN)#(mN)#(mN)(mN)(mN)(dN)#(mN)(mN)(mN)(mN)(fN)(mN)(fN)(mN)(fN) | 84 |
| V1.10 | (mN)(mN)(fN)(mN)(fN)(mN)(mN)(mN)(mG)(rU)(rU)(mG)(mU)(mA)(mG)(mC)(rU) | |
| | (rC)(mC)(dC)#(dT)#(mU)(mU)(mC)(mU)(mC)(mA)(mU)(mU)#(mU)#(mC)#(mG) | |
| CR48-203- | (mN)#(mN)#(mN)#(mN)(mN)(mN)(dN)#(mN)(mN)(mN)(mN)(fN)(mN)(fN)(mN)(fN) | 85 |
| V1.12 | (mN)(mN)(fN)(mN)(fN)(mN)(mN)(mN)(mG)(rU)(rU)(mG)(mU)(mA)(mG)(mC)(rU) | |
| | (rC)(mC)(mC)(dT)#(mU)(mU)(mC)(mU)(mC)(mA)(mU)(mU)#(mU)#(mC)#(mG) | |
| CR48-203- | (mN)#(mN)#(mN)#(mN)(mN)(mN)(dN)#(mN)(mN)(mN)(mN)(fN)(mN)(fN)(mN)(fN) | 86 |
| V1.18 | (mN)(mN)(fN)(mN)(fN)(mN)(mN)(mN)(mG)(rU)(rU)(mG)(mU)(mA)(mG)(mC)(dT)# | |
| | (rC)(dC)#(rC)(dT)#(mU)(mU)(mC)(mU)(mC)(mA)(mU)(mU)#(mU)#(mC)#(mG) | |
| CR48-203- | (mN)#(mN)#(mN)#(mN)(mN)(mN)(dN)#(mN)(mN)(mN)(mN)(N)(mN)(fN)(mN)(fN) | 87 |
| V1.19 | (mN)(mN)(fN)(mN)(fN)(mN)(mN)(mN)(mG)(rU)(rU)(mG)(mU)(mA)(mG)(mC)(dT)# | |
| | (rC)(dC)#(dC)#(dT)#(mU)(mU)(mC)(mU)(mC)(mA)(mU)(mU)#(mU)#(mC)#(mG) | |
| CR48-203- | (mN)#(mN)#(mN)#(mN)(mN)(mN)(dN)#(mN)(mN)(mN)(mN)(fN)(mN)(fN)(mN)(fN) | 88 |
| V1.22 | m(N)(mN)(fN)(mN)(fN)(mN)(mN)(mN)(mG)(rU)(rU)(mG)(mU)(mA)(mG)(mC)(dT)# | |
| | (rC)(mC)(dC)#(dT)#(mU)(mU)(mC)(mU)(mC)(mA)(mU)(mU)#(mU)#(mC)#(mG) | |
| CR48-203- | (mN)#(mN)#(mN)#(mN)(mN)(mN)(dN)#(mN)(mN)(mN)(mN)(fN)(mN)(fN)(mN)(fN) | 89 |
| V1.24 | (mN)(mN)(fN)(mN)(fN)(mN)(mN)(mN)(mG)(rU)(rU)(mG)(mU)(mA)(mG)(mC)(dT)# | |
| | (rC)(mC)(mC)(dT)#(mU)(mU)(mC)(mU)(mC)(mA)(mU)(mU)#(mU)#(mC)#(mG) | |
| CR48-203- | (mN)#(mN)#(mN)#(mN)(mN)(mN)(dN)#(mN)(mN)(mN)(mN)(fN)(mN)(fN)(mN)(fN) | 90 |
| V1.30 | (mN)(mN)(fN)(mN)(fN)(mN)(mN)(mN)(mG)(dT)#(rU)(mG)(mU)(mA)(mG)(mC)(dT)# | |
| | (rC)(dC)#(rC)(dT)#(mU)(mU)(mC)(mU)(mC)(mA)(mU)(mU)#(mU)#(mC)#(mG) | |
| CR48-203- | (mN)#(mN)#(mN)#(mN)(mN)(mN)(dN)#(mN)(mN)(mN)(mN)(fN)(mN)(fN)(mN)(fN) | 91 |
| V1.31 | (mN)(mN)(fN)(mN)(fN)(mN)(mN)(mN)(mG)(dT)#(rU)(mG)(mU)(mA)(mG)(mC)(dT)# | |
| | (rC)(dC)#(dC)#(dT)#(mU)(mU)(mC)(mU)(mC)(mA)(mU)(mU)#(mU)#(mC)#(mG) | |
| CR48-203- | (mN)#(mN)#(mN)#(mN)(mN)(mN)(dN)#(mN)(mN)(mN)(mN)(fN)(mN)(fN)(mN)(fN) | 92 |
| V1.34 | (mN)(mN)(fN)(mN)(fN)(mN)(mN)(mN)(mG)(dT)#(rU)(mG)(mU)(mA)(mG)(mC)(dT)# | |
| | (rC)(mC)(dC)#(dT)#(mU)(mU)(mC)(mU)(mC)(mA)(mU)(mU)#(mU)#(mC)#(mG) | |
| CR48-203- | (mN)#(mN)#(mN)#(mN)(mN)(mN)(dN)#(mN)(mN)(mN)(mN)(fN)(mN)(fN)(mN)(fN) | 93 |
| V1.36 | (mN)(mN)(fN)(mN)(fN)(mN)(mN)(mN)(mG)(dT)#(rU)(mG)(mU)(mA)(mG)(mC)(dT)# | |
| | (rC)(mC)(mC)(dT)#(mU)(mU)(mC)(mU)(mC)(mA)(mU)(mU)#(mU)#(mC)#(mG) | |

KEY:
rN = RNA, mN = 2′-O-methyl RNA, fN = 2’-fluoro RNA, dN = 2’-deoxy RNA, N = A, U, G, or C
N#N = phosphorothioate linkage

TABLE 2
Exemplary chemical modification patterns for tracrRNAs

| | | SEQ |
|---|---|---|
| Name | Sequence | ID NO: |
| TR93-0 | (mC)#(mG)#(mA)#(rA)(rA)(rU)(rG)(rA)(rG)(rA)(rA)(rC)(rC)(rG)(rU) | 94 |
| | (rU)(rG)(rC)(rU)(rA)(rC)(rA)(rA)(rU)(rA)(rA)(rG)(rG)(rC)(rC)(rG)(rU) | |
| | (rC)(rU)(rG)(rA)(rA)(rA)(rA)(rG)(rA)(rU)(rG)(rU)(rG)(rC)(rC)(rG)(rC) | |
| | (rA)(rA)(rC)(rG)(rC)(rU)(rC)(rU)(rG)(rC)(rC)(rC)(rC)(rU)(rU)(rA)(rA) | |
| | (rA)(rG)(rC)(rU)(rC)(rC)(rU)(rG)(rC)(rU)(rU)(rU)(rA)(rA)(rG)(rG)(rG) | |
| | (rG)(rC)(rA)(rU)(rC)(rG)(rU)#(mU)#(mU)#(mA) | |
| TR93-1 | (mC)#(mG)#(mA)#(rA)(rA)(rU)(rG)(rA)(rG)(rA)(rA)(rC)(rC)(rG)(rU) | 95 |
| | (rU)(rG)(rC)(rU)(rA)(rC)(rA)(rA)(rU)(rA)(rA)(rG)(rG)(rC)(rC)(rG)(rU) | |
| | (rC)(rU)(mG)(mA)(mA)(mA)(rA)(rG)(rA)(rU)(rG)(rU)(rG)(rC)(rC)(rG) | |
| | (rC)(rA)(rA)(rC)(rG)(rC)(rU)(rC)(rU)(rG)(rC)(rC)(rC)(rC)(rU)(rU)(rA) | |
| | (rA)(rA)(rG)(rC)(mU)(mC)(mC)(mU)(rG)(rC)(rU)(rU)(rU)(rA)(rA)(rG) | |
| | (rG)(rG)(rG)(rC)(rA)(rU)(rC)(rG)(rU)#(mU)#(mU)#(mA) | |
| TR93-2 | (mC)#(mG)#(mA)#(mA)(mA)(mU)(mG)(mA)(mG)(mA)(mA)(mC)(rC) | 96 |
| | (rG)(rU)(rU)(rG)(rC)(rU)(rA)(rC)(rA)(rA)(rU)(rA)(rA)(rG)(rG)(rC)(rC) | |
| | (rG)(mU)(mC)(mU)(mG)(mA)(mA)(mA)(mA)(mG)(rA)(rU)(rG)(rU)(rG) | |
| | (rC)(rC)(rG)(rC)(rA)(rA)(rC)(rG)(rC)(rU)(rC)(rU)(rG)(rC)(rC)(mC) | |
| | (mC)(mU)(mU)(mA)(mA)(mA)(mG)(mC)(mU)(mC)(mC)(mU)(mG) | |
| | (mC)(mU)(mU)(mU)(mA)(mA)(mG)(mG)(rG)(rG)(rC)(rA)(rU)(mC)(mG) | |
| | (mU)#(mU)#(mU)#(mA) | |

KEY: rN = RNA, mN = 2’-O-methyl RNA, fN = 2’-fluoro RNA, dN = 2’-deoxy RNA, N = A, U, G, or C
N#N = phosphorothioate linkage

It will be understood to those of skill in the art that the base sequence of the first 24 nucleotides of the exemplary crRNAs recited in Table 1 above are directed to a specific target. This 24-nucleotide base sequence may be changed based on the target nucleic acid, however the chemical modifications remain the same. An exemplary unmodified Nme crRNA sequence, from 5′ to 3′, is (N)ₓ GUUGUAGCUCCCUUUCUC (CR42) (SEQ ID NO: 8); or (N)ₓ GUUGUAGCUCCCUUUCUCAUUUCG (CR48) (SEQ ID NO: 9), where “N” corresponds to any nucleotide (e.g., A, U, G, or C) and x corresponds to any integer between 18 and 26. As used herein, “(N)ₓ” corresponds to the guide sequence of a crRNA or sgRNA.

An exemplary unmodified Nme tracrRNA sequence, from 5′ to 3′, is CGAAAUGAGAACCGUUGCUACAAUAAGGCCGUCUGAAAAGAUGUGCCG CAACGCUCUGCCCCUUAAAGCUUCUGCUUUAAGGGGCAUCGUUUA (TR93) (SEQ ID NO: 10) or AGAACCGUUGCUACAAUAAGGCCGUCUGAAAAGAUGUGCCGCAACGCU CUGCCCCUUAAAGCUCCUGCUUUAAGGGGCAUCGUUUA (TR87) (SEQ ID NO: 11).

It will be further understood to those of skill in the art that the guide sequence may be 10-30 nucleotides in length, preferably 16-26 nucleotides in length (for example, 16, 17, 18, 19, 20, 21, 22, 23, 24, 25, or 26 nucleotides in length), and is at or near the 5′ terminus of a Cas9 gRNA.

Chemically Modified N. meningitidis Single Guide RNA

The chemically modified Nme guide RNAs of the disclosure may be in a single guide RNA (sgRNA) format, as described above.

In certain embodiments, the chemically modified Nme gRNA described above further comprises a nucleotide or non-nucleotide loop or linker linking the 3′ end of the crRNA portion to the 5′ end of the tracrRNA portion.

In certain embodiments, the nucleotide loop is chemically modified. In certain embodiments, the nucleotide loop comprises the nucleotide sequence of GAAA. In certain embodiments, the nucleotide loop comprises the nucleotide sequence of (mG)(mA)(mA)(mA), wherein mN corresponds to a 2′-O-methyl RNA and N corresponds to any nucleotide.

In certain embodiments, the non-nucleotide linker comprises an azide linker, an ethylene glycol oligomer, a tetrazine linker, an alkyl chain, a peptide, an amide, or a carbamate (see, e.g., Pils et al. Nucleic Acids Res. 28(9): 1859-1863 (2000)).

In one aspect, the disclosure provides a chemically modified Neisseria meningitidis (Nme) single guide RNA (sgRNA) comprising one or more chemical modifications.

In certain embodiments, the sgRNA is between 99 and 145 nucleotides in length. In certain embodiments, the sgRNA is 145 nucleotides in length, 121 nucleotides in length, 111 nucleotides in length, 107 nucleotides in length, 105 nucleotides in length, 103 nucleotides in length, 102 nucleotides in length, 101 nucleotides in length, 100 nucleotides in length, or 99 nucleotides in length.

In certain embodiments, the chemically modified Nme sgRNA comprises between 2 and 100 modified nucleotides.

In certain embodiments, the chemically modified Nme sgRNA comprises at least one chemical modification in the nucleotide sequence of any one of:

(SEQ ID NO: 97)
(N)ₓGUUGUAGCUCCCUUUCUCAUUUCGGAAACGAAAUGA
GAACCGUUGCUACAAUAAGGCCGUCUGAAAAGAUGUGCCGCAACGCUC
UGCCCCUUAAAGCUUCUGCUUUAAGGGGCAUCGUUUA (SG-145NT);
(SEQ ID NO: 98)
(N)ₓGUUGUAGCUCCCGAAACGUUGCUACAAUAAGGCCGU
CUGAAAAGAUGUGCCGCAACGCUCUGCCCCUUAAAGCUUCUGCUUUAAG
GGGCAUCGUU (SG-121NTv1);
(SEQ ID NO: 99)
(N)ₓGUUGUAGCUCCCGAAACGUUGCUAC AAUAAGGCCGU
CUGAAAAGAUGUGCCGCAACGCUCUGCCCCUUAAAGCUUCUGCUUUAAG
GGGCAUCGUUUA (SG-121NTv2);
(SEQ ID NO: 100)
(N)ₓGUUGUAGCUCCCGAAACGUUGCUACAAUAAGGCCGU
CUGAAAAGAUGUGCCGCAACGCUCUGCCCCUUUUCUAAGGGGCAUCGUU
UA (SG-111NT);
(SEQ ID NO: 101)
(N)ₓGUUGUAGCUCCCGAAACGUUGCUACAAUAAGGCCGU
CUGAAAAGAUGUGCCGCAACGCUCUGCCCCUUUCUAGGGGCAUCGUU
(SG-107NT);
(SEQ ID NO: 102)
(N)ₓGUUGUAGCUCCCGAAACGUUGCUACAAUAAGGCCGU
CUGAAAAGAUGUGCCGCAACGCUCUGCCCCUUCUGGGGCAUCGUU
(SG-105NTv1);
(SEQ ID NO: 103)
(N)ₓGUUGUAGCUCCCGAAACGUUGCUACAAUAAGGCCGU
CUGAAAAGAUGUGCCGCAACGCUCUGCCCCUUUUCUAAGGGGCAU
(SG-105NTv2);
(SEQ ID NO: 104)
(N)ₓGUUGUAGCUCCCGAAACGUUGCUACAAUAAGGCCGU
CUGAAAAGAUGUGCCGCAACGCUCUGCCUUCUGGCAUCGUUUA
(SG-103NTv1);
(SEQ ID NO: 105)
(N)ₓGUUGUAGCUCCCGAAACGUUGCUACAAUAAGGCCGU
CUGAAAAGAUGUGCCGCAACGCUCUGCCCUUCUGGGCAUCGUU
(SG-103NTv2);
(SEQ ID NO: 106)
(N)ₓGUUGUAGCUCCCGAAACGUUGCUACAAUAAGGCCGU
CUGAAAAGAUGUGCCGCAACGCUCUGCUUCUGCAUCGUUUA
(SG-101NTv1);
or
(SEQ ID NO: 107)
(N)ₓGUUGUAGCUCCCGAAACGUUGCUACAAUAAGGCCGU
CUGAAAAGAUGUGCCGCAACGCUCUGCUUCUGCAUCGUU
(SG-99NT),

wherein N corresponds to any nucleotide and x corresponds to any integer between 18 and 26.

In another aspect, the disclosure provides a chemically modified Neisseria meningitidis (Nme) single guide RNA (sgRNA) comprising a chemical modification pattern selected from anyone of:

(SEQ ID NO: 108)
(mN)#(mN)#(mN)#(rN)(rN)(rN)(rN)(rN)(rN)(rN)(rN)(rN)(rN)(rN)(rN)
(rN)(rN)(rN)(rN)(rN)(rN)(rN)(rN)(rN)(rG)(rU)(rU)(rG)(rU)(rA)(rG)(rC)(rU)(rC)(rC)
(rC)(mG)(mA)(mA)(mA)(rC)(rG)(rU)(rU)(rG)(rC)(rU)(rA)(rC)(rA)(rA)(rU)(rA)(rA)
(rG)(rG)(rC)(rC)(rG)(rU)(mC)(mU)(mG)(mA)(mA)(mA)(mA)(mG)(rA)(rU)(rG)(rU)
(rG)(rC)(rC)(rG)(rC)(rA)(rA)(rC)(rG)(rC)(rU)(rC)(rU)(rG)(rC)(rC)(mU)(mU)(mC)
(mU)(rG)(rG)(rC)(rA)(rU)(rC)(rG)(rU)#(mU)#(mU)#(mA) (SG-1);
(SEQ ID NO: 109)
(mN)#(mN)#(mN)#(rN)(rN)(rN)(rN)(rN)(rN)(rN)(rN)(rN)(rN)(rN)(rN)
(rN)(rN)(rN)(rN)(rN)(rN)(rN)(rN)(rN)(rG)(rU)(rU)(rG)(rU)(mA)(mG)(mC)(rU)(rC)
(rC)(rC)(mG)(mA)(mA)(mA)(rC)(rG)(rU)(rU)(rG)(rC)(rU)(rA)(rC)(rA)(rA)(rU)(rA)
(rA)(rG)(rG)(mC)(mC)(rG)(mU)(mC)(mU)(mG)(mA)(mA)(mA)(mA)(mG)(rA)(rU)
(rG)(rU)(rG)(rC)(rC)(rG)(rC)(rA)(rA)(rC)(rG)(rC)(mU)(mC)(mU)(mG)(rC)(rC)(mU)
(mU)(mC)(mU)(rG)(rG)(rC)(rA)(rU)(mC)(mG)(mU)#(mU)#(mU)#(mA)
(SG-2);
(SEQ ID NO: 110)
(mN)#(mN)#(mN)#(mN)(mN)(rN)(rN)(rN)(mN)(mN)(mN)(rN)(mN)
(rN)(rN)(rN)(rN)(mN)(rN)(rN)(rN)(mN)(rN)(rN)(mG)(rU)(rU)(rG)(mU)(mA)(mG)
(mC)(rU)(rC)(rC)(rC)(mG)(mA)(mA)(mA)(mC)(rG)(mU)(rU)(mG)(mC)(rU)(mA)
(mC)(rA)(rA)(rU)(rA)(mA)(rG)(mG)(mC)(mC)(mG)(mU)(mC)(mU)(mG)(mA)(mA)
(mA)(mA)(mG)(mA)(mU)(rG)(mU)(mG)(mC)(mC)(mG)(mC)(mA)(mA)(mC)(mG)
(mC)(mU)(mC)(mU)(mG)(mC)(mC)(mU)(mU)(mC)(mU)(rG)(mG)(rC)(mA)(mU)(mC)
(mG)(mU)#(mU)#(mU)#(mA) (SG-3);
(SEQ ID NO: 111)
(mN)#(mN)#(mN)#(mN)(mN)(rN)(rN)(rN)(mN)(mN)(mN)(rN)(mN)
(rN)(rN)(mN)(rN)(mN)(rN)(rN)(rN)(mN)(rN)(rN)(mG)(rU)(rU)(rG)(mU)(mA)(mG)
(mC)(rU)(rC)(rC)(rC)(mG)(mA)(mA)(mA)(mC)(rG)(mU)(rU)(mG)(mC)(rU)(mA)
(mC)(rA)(rA)(rU)(rA)(mA)(rG)(mG)(fC)(mC)(fG)(mU)(fC)(mU)(fG)(mA)(fA)(mA)
(fA)(mG)(fA)(mU)(rG)(mU)(fG)(mC)(fC)(mG)(fC)(mA)(fA)(mC)(fG)(mC)
(fU)(mC)(fU)(mG)(fC)(mC)(fU)(mU)(fC)(mU)(rG)(mG)(rC)(mA)(fU)(mC)(fG)
(mU)#(mU)#(mU)#(mA) (SG-4);
(SEQ ID NO: 112)
(mN)#(mN)#(mN)#(mN)(mN)(rN)(rN)(rN)(mN)(mN)(mN)(rN)(mN)
(rN)(rN)(rN)(rN)(mN)(rN)(rN)(rN)(mN)(rN)(rN)(mG)(rU)(rU)(rG)(mU)(mA)(mG)
(mC)(rU)(rC)(rC)(rC)(mG)(mA)(mA)(mA)(rC)(rG)(rU)(rU)(rG(rC)(rU)(rA)(rC)(rA)
(rA)(rU)(rA)(rA)(rG)(rG)(mC)(mC)(rG)(mU)(mC(mU)(mG)(mA)(mA)(mA)(mA)
(mG)(rA)(rU)(rG)(rU)(rG)(rC)(rC)(rG)(rC)(rA(rA)(rC)(rG)(rC)(mU)(mC)(mU)(mG)(rC)
(rC)(mU)(mU)(mC)(mU)(rG)(rG)(rC)(rA)(rU)(mC)(mG)(mU)#(mU)#(mU)#(mA)
(SG-5);
(SEQ ID NO: 113)
(mN)#(mN)#(mN)#(rN)(mN)(rN)(rN)(rN)(mN)(mN)(rN)(rN)(mN)
(rN)(rN)(rN)(rN)(mN)(rN)(rN)(rN)(mN)(rN)(rN)(mG)(rU)(rU)(rG)(rU)(mA)(mG)
(mC)(rU)(rC)(rC)(rC)(mG)(mA)(mA)(mA)(rC)(rG)(rU)(rU)(rG)(rC)(rU)(rA)(rC)(rA)(rA)
(rU)(rA)(rA)(rG)(rG)(mC)(mC)(rG)(mU)(mC)(mU)(mG)(mA)(mA)(mA)(mA)(mG)
(rA)(rU)(rG)(rU)(rG)(rC)(rC)(rG)(rC)(rA)(rA)(rC)(rG)(rC)(mU)(mC)(mU)(mG)(rC)
(rC)(mU)(mU)(mC)(mU)(rG)(rG)(rC)(rA)(rU)(mC)(mG)(mU)#(mU)#(mU)#(mA)
(SG-6);
(SEQ ID NO: 114)
(mN)#(mN)#(mN)#(rN)(rN)(rN)(rN)(rN)(rN)(rN)(rN)(rN)(rN)(rN)
(rN)(rN)(rN)(mN)(rN)(rN)(rN)(mN)(rN)(rN)(rG)(rU)(rU)(rG)(rU)(mA)(mG)(mC)(rU)
(rC)(rC)(rC)(mG)(mA)(mA)(mA)(rC)(rG)(rU)(rU)(rG)(rC)(rU)(rA)(rC)(rA)(rA)(rU)
(rA)(rA)(rG)(rG)(mC)(mC)(rG)(mU)(mC)(mU)(mG)(mA)(mA)(mA)(mA)(mG)(rA)
(rU)(rG)(rU)(rG)(rC)(rC)(rG)(rC)(rA)(rA)(rC)(rG)(rC)(mU)(mC)(mU)(mG)(rC)(rC)
(mU)(mU)(mC)(mU)(rG)(rG)(rC)(rA)(rU)(mC)(mG)(mU)#(mU)#(mU)#(mA)
(SG-7);
(SEQ ID NO: 115)
(mN)#(mN)#(mN)#(mN)(mN)(mN)(mN)(mN)(mN)(mN)(mN)(mN)
(mN)(mN)(rN)(rN)(rN)(rN)(rN)(rN)(rN)(rN)(rN)(rN)(rG)(rU)(rU)(rG)(rU)(mA)(mG)
(mC)(rU)(rC)(rC)(rC)(mG)(mA)(mA)(mA)(rC)(rG)(rU)(rU)(rG)(rC)(rU)(rA)(rC)
(rA(rA)(rU)(rA)(rA)(rG)(rG)(mC)(mC)(rG)(mU)(mC)(mU)(mG)(mA)(mA)(mA)(mA)
(mG)(rA)(rU)(rG)(rU)(rG)(rC)(rC)(rG)(rC)(rA)(rA)(rC)(rG)(rC)(mU)(mC)(mU)(mG)
(rC)(rC)(mU)(mU)(mC)(mU)(rG)(rG)(rC)(rA)(rU)(mC)(mG)(mU)#(mU)#(mU)#(mA)
(SG-8);
(SEQ ID NO: 116)
(mN)#(mN)#(mN)#(mN)(mN)(mN)(mN)(mN)(mN)(mN)(mN)(mN)
(mN)(mN)(rN)(rN)(rN)(mN)(rN)(rN)(rN)(mN)(rN)(rN)(mG)(rU)(rU)(rG)(mU)(mA)
(mG)(mC)(rU)(rC)(rC)(rC)(mG)(mA)(mA)(mA)(rC)(rG)(rU)(rU)(rG)(rC)(rU)(rA)
(rC)(rA)(rA)(rU)(rA)(rA)(rG)(rG)(mC)(mC)(rG)(mU)(mC)(mU)(mG)(mA)(mA)(mA)
(mA)(mG)(rA)(rU)(rG)(rU)(rG)(rC)(rC)(rG)(rC)(rA)(rA)(rC)(rG)(rC)(mU)(mC)(mU)
(mG)(rC)(rC)(mU)(mU)(mC)(mU)(rG)(rG)(rC)(rA)(rU)(mC)(mG)(mU)#(mU)#(mU)#
(mA) (SG-9);
(SEQ ID NO: 117)
(mN)#(mN)#(mN)#(mN)(mN)(rN)(rN)(rN)(mN)(mN)(mN)(rN)(mN)
(rN)(rN)(rN)(rN)(mN)(rN)(rN)(rN)(mN)(rN)(rN)(mG)(rU)(rU)(rG)(mU)(mA)(mG)
(mC)(rU)(rC)(rC)(rC)(mG)(mA)(mA)(mA)(rC)(rG)(rU)(rU)(rG)(rC)(rU)(rA)(rC)(rA)
(rA)(rU)(rA)(mA)(rG)(mG)(mC)(mC)(mG)(mU)(mC)(mU)(mG)(mA)(mA)(mA)(mA)
(mG)(mA)(mU)(rG)(rU)(mG)(mC)(mC)(rG)(rC)(rA)(rA)(rC)(rG)(rC)(mU)(mC)(mU)
(mG)(rC)(rC)(mU)(mU)(mC)(mU)(rG)(rG)(rC)(rA)(rU)(mC)(mG)(mU)#(mU)#(mU)
#(mA) (SG-10);
(SEQ ID NO: 118)
(mN)#(mN)#(mN)#(mN)(mN)(rN)(rN)(rN)(mN)(mN)(mN)(rN)(mN)
(rN)(rN)(rN)(rN)(mN)(rN)(rN)(rN)(mN)(rN)(rN)(mG)(rU)(rU)(rG)(mU)(mA)(mG)
(mC)(rU)(rC)(rC)(rC)(mG)(mA)(mA)(mA)(rC)(rG)(rU)(rU)(rG)(rC)(rU)(rA)(rC)(rA)
(rA)(rU)(rA)(rA)(rG)(rG)(mC)(mC)(rG)(mU)(mC)(mU)(mG)(mA)(mA)(mA)(mA)
(mG)(rA)(rU)(rG)(rU)(rG)(rC)(rC)(mG)(rC)(mA)(mA)(mC)(mG)(mC)(mU)(mC)(mU)
(mG)(mC)(mC)(mU)(mU)(mC)(mU)(rG)(mG)(rC)(mA)(rU)(mC)(mG)(mU)#(mU)#
(mU)#(mA) (SG-11);
(SEQ ID NO: 119)
(mN)#(mN)#(mN)#(mN)(mN)(rN)(rN)(rN)(mN)(mN)(mN)(rN)(mN)
(rN)(rN)(rN)(rN)(mN)(rN)(rN)(rN)(mN)(rN)(rN)(fG)(rU)(rU)(rG)(fU)(fA)(fG)(fC)
(rU)(rC)(rC)(rC)(mG)(mA)(mA)(mA)(rC)(rG)(rU)(rU)(rG)(rC)(rU)(rA)(rC)(rA)(rA)
(rU)(rA)(rA)(rG)(rG)(fC)(fC)(rG)(fU)(fC)(fU)(mG)(mA)(mA)(mA)(fA)(fG)(rA)(rU)
(rG)(rU)(rG)(rC)(rC)(rG)(rC)(rA)(rA)(rC)(rG)(rC)(mU)(mC)(fU)(fG)(rC)(rC)(mU)
(mU)(mC)(mU)(rG)(rG)(rC)(rA)(rU)(fC)(fG)(fU)#(fU)#(mU)#(mA)
(SG-12);
(SEQ ID NO: 120)
(mN)#(mN)#(mN)#(mN)(mN)(rN)(rN)(rN)(mN)(mN)(mN)(rN)(mN)
(rN)(rN)(rN)(rN)(mN)(rN)(rN)(rN)(mN)(rN)(rN)(fG)(rU)(rU)(rG)(fU)(fA)(fG)(fC)
(rU)(rC)(rC)(rC)(mG)(mA)(mA)(mA)(mC)(rG)(mU)(rU)(fG)(fC)(rU)(fA)(fC)(rA)(rA)
(rU)(rA)(rA)(rG)(rG)(mC)(mC)(rG)(mU)(mC)(mU)(mG)(mA)(mA)(mA)(mA)(mG)
(rA)(rU)(rG)(rU)(rG)(rC)(rC)(rG)(rC)(rA)(rA)(rC)(rG)(rC)(mU)(mC)(mU)(mG)(rC)
(rC)(mU)(mU)(mC)(mU)(rG)(rG)(rC)(rA)(rU)(mC)(mG)(mU)#(mU)#(mU)#(mA)
(SG-13);
(SEQ ID NO: 121)
(mN)#(mN)#(mN)#(mN)(mN)(rN)(rN)(rN)(mN)(mN)(mN)(rN)(mN)
(rN)(rN)(rN)(rN)(mN)(rN)(rN)(rN)(mN)(rN)(rN)(fG)(rU)(rU)(rG)(fU)(fA)(fG)(fC)
(rU)(rC)(rC)(rC)(mG)(mA)(mA)(mA)(mC)(rG)(mU)(rU)(fG)(fC)(rU)(fA)(fC)(rA)(rA)
(rU)(rA)(rA)(rG)(rG)(fC)(fC)(rG)(fU)(fC)(fU)(mG)(mA)(mA)(mA)(fA)(fG)(rA)(rU)
(rG)(rU)(rG)(rC)(rC)(rG)(rC)(rA)(rA)(rC)(rG)(rC)(mU)(mC)(fU)(fG)(rC)(rC)(mU)
(mU)(mC)(mU)(rG)(rG)(rC)(rA)(rU)(fC)(fG)(fU)#(fU)#(mU)#(mA)
(SG-14);
(SEQ ID NO: 122)
mN#mN#mN#rNrNrNrNrNrNrNrNrNrNrNrNrNrNrNrNrNrNrNrNr
NrGrUrUrGrUrArGrCrUrCrCrCrGrArArArCrGrUrUrGrCrUrArCrArArUrArArGrGr
CrCrGrUrCrUrGrArArArArGrArUrGrUrGrCrCrGrCrArArCrGrCrUrCrUrGrCrCrCr
CrUrUrArArArGrCrUrCrCrUrGrCrUrUrUrArArGrGrGrGrCrArUrCrGrUmU#mU#
mA (SG121-0);
(SEQ ID NO: 123)
mN#mN#mN#mNmNrNrNrNrNrNrNrNrNrNrNrNrNrNrNrNrNrNr
NrNrGrUrUrGrUrArGrCrUrCrCrCmGmAmAmArCrGrUrUrGrCrUrArCrArArUrAr
ArGrGrCrCrGmUmCmUmGmAmAmAmAmGrArUrGrUrGrCrCrGrCrArArCrGrCr
UrCrUrGrCrCmCmCmUmUmAmAmAmGmCmUmCmCmUmGmCmUmUmUmA
mAmGmGrGrGrCrArUmCmGmU#mU#mU#mA (SG121-2);
(SEQ ID NO: 124)
mN#mN#mN#mNmNrNrNrNmNmNmNrNmNrNrNrNrNmNrNrNr
NmNrNrNmGrUrUrGmUmAmGmCrUrCrCrCmGmAmAmArCrGrUrUrGrCrUrArC
rArArUrArArGrGrCrCrGmUmCmUmGmAmAmAmAmGrArUrGrUrGrCrCrGrCrAr
ArCrGrCrUrCrUrGrCrCmCmCmUmUmAmAmAmGmCmUmCmCmUmGmCmUm
UmUmAmAmGmGrGrGrCrArUmCmGmU#mU#mU#mA
(SG121-3);
and
(SEQ ID NO: 125)
mN#mN#mN#mNmNmNmNmNmNmNmNmNmNmNrNrNrNmNr
NrNrNmNrNrNmGrUrUrGmUmAmGmCrUrCrCrCmGmAmAmArCrGrUrUrGrCrU
rArCrArArUrArArGrGrCrCrGmUmCmUmGmAmAmAmAmGrArUrGrUrGrCrCrGr
CrArArCrGrCrUrCrUrGrCrCmCmCmUmUmAmAmAmGmCmUmCmCmUmGmC
mUmUmUmAmAmGmGrGrGrCrArUmCmGmU#mU#mU#mA
(SG121-4),

wherein rN=RNA, mN=2′-O-methyl RNA, fN=2′-fluoro RNA, N #N=phosphorothioate linkage, and N=any nucleotide.

### Guide RNA Conjugates

The chemically modified Nme guide RNAs of the disclosure may be modified with terminally conjugated moieties. As used herein, a “terminally conjugated moiety” or “moiety” refers to a compound which may be linked or attached to the 5′ and/or 3′ end of the crRNA and/or tracrRNA of a guide RNA. Terminally conjugated moieties can provide increased stability, increased ability to penetrate cell membranes, increase cellular uptake, increase circulation time in vivo, act as a cell-specific directing reagent, and/or provide a means to monitor cellular or tissue-specific uptake.

In certain embodiments, the terminally conjugated moiety is conjugated to the 5′ end of the crRNA portion of a guide RNA. In certain embodiments, the terminally conjugated moiety is conjugated to the 3′ end of the crRNA portion of a guide RNA. In certain embodiments, the terminally conjugated moiety is conjugated to the 5′ end of the tracrRNA portion of a guide RNA. In certain embodiments, the terminally conjugated moiety is conjugated to the 3′ end of the tracrRNA portion of a guide RNA.

In certain exemplary embodiments, a terminally conjugated moiety includes, but is not limited to, fatty acid, steroid, secosteroid, lipid, ganglioside analog, nucleoside analogs, endocannabinoid, vitamin, receptor ligand, peptide, aptamer, alkyl chain, fluorophore, antibody, nuclear localization signal, and the like.

In certain exemplary embodiments, a terminally conjugated moiety includes, but is not limited to, cholesterol, cholesterol-triethylene glycol (TEGChol), docosahexaenoic acid (DHA), docosanoic acid (DCA), lithocholic acid (LA), GalNAc, amphiphilic block copolymer (ABC), hydrophilic block copolymer (HBC), poloxamer, Cy5, Cy3, and the like.

In certain exemplary embodiments, the at least one terminally conjugated moiety is a modified lipid, including a branched lipid (such as the structure shown in Formula I) or a headgroup-modified lipid (such as the structure shown in Formula II).

X-MC(═Y)M-Z[L-MC(═Y)M-R]n Formula I:

where X is a moiety that links the lipid to the guide RNA, each Y is independently oxygen or sulfur, each M is independently CH₂, NH, O or S, Z is a branching group which allows two or three (“n”) chains to be joined to the rest of the structure, L is an optional linker moiety, and each R is independently a saturated, monounsaturated or polyunsaturated linear or branched moiety from 2 to 30 atoms in length, a sterol, or other hydrophobic group.

X-MC(═Y)M-Z-[L-MC(═Y)M-R]n-L-K-J Formula II:

where X is a moiety that links the lipid to the guide RNA, each Y is independently oxygen or sulfur, each M is independently CH₂, NH, N-alkyl, O or S, Z is a branching group which allows two or three (“n”) chains to be joined to the rest of the structure, each L is independently an optional linker moiety, and R is a saturated, monounsaturated or polyunsaturated linear or branched moiety from 2 to 30 atoms in length, a sterol, or other hydrophobic group, K is a phosphate, sulfate, or amide and J is an aminoalkane or quaternary aminoalkane group.

The moieties may be attached to the terminal nucleotides of the guide RNA via a linker. Exemplary linkers include, but are not limited to, an ethylene glycol chain, an alkyl chain, a polypeptide, a polysaccharide, a block copolymer, and the like.

In certain embodiments, the moiety is conjugated to the 5′ end and/or 3′ end of any one of the crRNAs recited in Table 1.

In certain embodiments, the moiety is conjugated to the 5′ end and/or 3′ end of any one of tracrRNAs recited in Table 2.

In certain embodiments, the moiety is conjugated to the 5′ end and/or 3′ end of any one of sgRNA SG-1 to SG-14 (i.e., SG-1, SG-2, SG-3, SG-4, SG-5, SG-6, SG-7, SG-8, SG-9, SG-10, SG-11, SG-12, SG-13, or SG-14).

In certain embodiments, the moiety is conjugated to the 5′ end and/or 3′ end of any one of sgRNA SG121-0, SG121-2, SG121-3, or SG121-4.

N. meningitidis Cas9 RNA-Guided Nucleases

N. meningitidis RNA-guided nucleases (e.g., Nme Cas9 or NmCas9) according to the present disclosure include, without limitation, any Cas9 nuclease obtained from N. meningitidis (e.g., Nme1Cas9, Nme2Cas9, or Nme3Cas9), as well as other Cas9 nucleases derived or obtained therefrom. In functional terms, N. meningitidis RNA-guided nucleases are defined as those nucleases that: (a) interact with (e.g., complex with) a gRNA; and (b) together with the gRNA, associate with, and optionally cleave or modify, a target region of a DNA that includes (i) a sequence complementary to the targeting domain of the gRNA and, optionally, (ii) an additional sequence referred to as a “protospacer adjacent motif,” or “PAM,” which is described in greater detail below. As the following examples will illustrate, RNA-guided nucleases can be defined, in broad terms, by their PAM specificity and cleavage activity, even though variations may exist between individual RNA-guided nucleases that share the same PAM specificity or cleavage activity. Skilled artisans will appreciate that some aspects of the present disclosure relate to systems, methods and compositions that can be implemented using any suitable RNA-guided nuclease having a certain PAM specificity and/or cleavage activity. For this reason, unless otherwise specified, the term RNA-guided nuclease should be understood as a generic term, and not limited to any particular type (e.g., Nme1Cas9, Nme2Cas9, or Nme3Cas9), or variation (e.g., full-length vs. truncated or split; naturally-occurring PAM specificity vs. engineered PAM specificity).

N. meningitidis Cas9 nucleases belong to the Type II-C Cas9 nucleases, which are generally less than 1,100 amino acids in length and are capable of genome editing, including genome editing in mammalian cells.

In certain embodiments, the chemically modified Nme gRNAs of the disclosure are capable of binding to an Nme Cas9 nuclease or a variant thereof.

In certain embodiments, the Nme Cas9 nuclease is Nme1Cas9, Nme2Cas9, or Nme3Cas9.

Exemplary Nme Cas9 amino acid and nucleic acid sequences are recited below in Table 3.

TABLE 3
Exemplary Nme Cas9 amino acid and nucleic acid sequences

| Description | Sequence |
|---|---|
| Nme1Cas9 | AAFKPNSINYILGLDIGIASVGWAMVEIDEEENPIRLIDLGVRV |
| Amino Acid | FERAEVPKTGDSLAMARRLARSVRRLTRRRAHRLLRTRRLLK |
| | REGVLQAANFDENGLIKSLPNTPWQLRAAALDRKLTPLEWS |
| | AVLLHLIKHRGYLSQRKNEGETADKELGALLKGVAGNAHAL |
| | QTGDFRTPAELALNKFEKESGHIRNQRSDYSHTFSRKDLQAE |
| | LILLFEKQKEFGNPHVSGGLKEGIETLLMTQRPALSGDAVQK |
| | MLGHCTFEPAEPKAAKNTYTAERFIWLTKLNNLRILEQGSER |
| | PLTDTERATLMDEPYRKSKLTYAQARKLLGLEDTAFFKGLRY |
| | GKDNAEASTLMEMKAYHAISRALEKEGLKDKKSPLNLSPEL |
| | QDEIGTAFSLFKTDEDITGRLKDRIQPEILEALLKHISFDKFVQI |
| | SLKALRRIVPLMEQGKRYDEACAEIYGDHYGKKNTEEKIYLP |
| | PIPADEIRNPVVLRALSQARKVINGVVRRYGSPARIHIETAREV |
| | GKSFKDRKEIEKRQEENRKDREKAAAKFREYFPNFVGEPKSK |
| | DILKLRLYEQQHGKCLYSGKEINLGRLNEKGYVEIDHALPFSR |
| | TWDDSFNNKVLVLGSENQNKGNQTPYEYFNGKDNSREWQE |
| | FKARVETSRFPRSKKQRILLQKFDEDGFKERNLNDTRYVNRF |
| | LCQFVADRMRLTGKGKKRVFASNGQITNLLRGFWGLRKVRA |
| | ENDRHHALDAVVVACSTVAMQQKITRFVRYKEMNAFDGKTI |
| | DKETGEVLHQKTHFPQPWEFFAQEVMIRVFGKPDGKPEFEEA |
| | DTLEKLRTLLAEKLSSRPEAVHEYVTPLFVSRAPNRKMSGQG |
| | HMETVKSAKRLDEGVSVLRVPLTQLKLKDLEKMVNREREPK |
| | LYEALKARLEAHKDDPAKAFAEPFYKYDKAGNRTQQVKAV |
| | RVEQVQKTGVWVRNHNGIADNATMVRVDVFEKGDKYYLV |
| | PIYSWQVAKGILPDRAVVQGKDEEDWQLIDDSFNFKFSLHPN |
| | DLVEVITKKARMFGYFASCHRGTGNINIRIHDLDHKIGKNGIL |
| | EGIGVKTALSFQKYQIDELGKEIRPCRLKKRPPVR (SEQ ID NO: |
| | 1) |
| Nme2Cas9 | AAFKPNPINYILGLDIGIASVGWAMVEIDEEENPIRLIDLGVRV |
| Amino Acid | FERAEVPKTGDSLAMARRLARSVRRLTRRRAHRLLRARRLL |
| | KREGVLQAADFDENGLIKSLPNTPWQLRAAALDRKLTPLEW |
| | SAVLLHLIKHRGYLSQRKNEGETADKELGALLKGVANNAHA |
| | LQTGDFRTPAELALNKFEKESGHIRNQRGDYSHTFSRKDLQA |
| | ELILLFEKQKEFGNPHVSGGLKEGIETLLMTQRPALSGDAVQ |
| | KMLGHCTFEPAEPKAAKNTYTAERFIWLTKLNNLRILEQGSE |
| | RPLTDTERATLMDEPYRKSKLTYAQARKLLGLEDTAFFKGLR |
| | YGKDNAEASTLMEMKAYHAISRALEKEGLKDKKSPLNLSSE |
| | LQDEIGTAFSLFKTDEDITGRLKDRVQPEILEALLKHISFDKFV |
| | QISLKALRRIVPLMEQGKRYDEACAEIYGDHYGKKNTEEKIY |
| | LPPIPADEIRNPVVLRALSQARKVINGVVRRYGSPARIHIETAR |
| | EVGKSFKDRKEIEKRQEENRKDREKAAAKFREYFPNFVGEPK |
| | SKDILKLRLYEQQHGKCLYSGKEINLVRLNEKGYVEIDHALP |
| | FSRTWDDSFNNKVLVLGSENQNKGNQTPYEYFNGKDNSRE |
| | WQEFKARVETSRFPRSKKQRILLQKFDEDGFKECNLNDTRYV |
| | NRFLCQFVADHILLTGKGKRRVFASNGQITNLLRGFWGLRKV |
| | RAENDRHHALDAVVVACSTVAMQQKITRFVRYKEMNAFDG |
| | KTIDKETGKVLHQKTHFPQPWEFFAQEVMIRVFGKPDGKPEF |
| | EEADTPEKLRTLLAEKLSSRPEAVHEYVTPLFVSRAPNRKMS |
| | GAHKDTLRSAKRFVKHNEKISVKRVWLTEIKLADLENMVNY |
| | KNGREIELYEALKARLEAYGGNAKQAFDPKDNPFYKKGGQL |
| | VKAVRVEKTQESGVLLNKKNAYTIADNGDMVRVDVFCKVD |
| | KKGKNQYFIVPIYAWQVAENILPDIDCKGYRIDDSYTFCFSLH |
| | KYDLIAFQKDEKSKVEFAYYINCDSSNGRFYLAWHDKGSKE |
| | QQFRISTQNLVLIQKYQVNELGKEIRPCRLKKRPPVR (SEQ ID |
| | NO: 2) |
| Nme3Cas9 | AAFKPNPINYILGLDIGIASVGWAMVEIDEEENPIRLIDLGVRV |
| Amino Acid | FERAEVPKTGDSLAMARRLARSVRRLTRRRAHRLLRARRLL |
| | KREGVLQAADFDENGLIKSLPNTPWQLRAAALDRKLTPLEW |
| | SAVLLHLIKHRGYLSQRKNEGETADKELGALLKGVADNAHA |
| | LQTGDFRTPAELALNKFEKECGHIRNQRGDYSHTFSRKDLQA |
| | ELNLLFEKQKEFGNPHVSGGLKEGIETLLMTQRPALSGDAVQ |
| | KMLGHCTFEPAEPKAAKNTYTAERFIWLTKLNNLRILEQGSE |
| | RPLTDTERATLMDEPYRKSKLTYAQARKLLSLEDTAFFKGLR |
| | YGKDNAEASTLMEMKAYHTISRALEKEGLKDKKSPLNLSPEL |
| | QDEIGTAFSLFKTDEDITGRLKDRIQPEILEALLKHISFDKFVQI |
| | SLKALRRIVPLMEQGKRYDEACAEIYGDHYGKKNTEEKIYLP |
| | PIPADEIRNPVVLRALSQARKVINGVVRRYGSPARIHIETAREV |
| | GKSFKDRKEIEKRQEENRKDREKAAAKFREYFPNFVGEPKSK |
| | DILKLRLYEQQHGKCLYSGKEINLGRLNEKGYVEIDHALPFSR |
| | TWDDSFNNKVLVLGSENQNKGNQTPYEYFNGKDNSREWQE |
| | FKARVETSRFPRSKKQRILLQKFDEDGFKERNLNDTRYVNRF |
| | LCQFVADRMRLTGKGKKRVFASNGQITNLLRGFWGLRKVRA |
| | ENDRHHALDAVVVACSTVAMQQKITRFVRYKEMNAFDGKTI |
| | DKETGEVLHQKTHFPQPWEFFAQEVMIRVFGKPDGKPEFEEA |
| | DTPEKLRTLLAEKLSSRPEAVHEYVTPLFVSRAPNRKMSGQG |
| | HMETVKSAKRLDEGVSVLRVPLTQLKLKDLEKMVNREREPK |
| | LYEALKARLEAHKDDPAKAFAEPFYKYDKAGNRTQQVKAV |
| | RVEQVQKTGVWVRNHNGIADNATMVRVDVFEKGDKYYLV |
| | PIYSWQVAKGILPDRAVVAYADEEDWTVIDESFRFKFVLYSN |
| | DLIKVQLKKDSFLGYFSGLDRATGAISLREHDLEKSKGKDGM |
| | HRIGVKTALSFQKYQIDEMGKEIRPCRLKKRPPVR (SEQ ID |
| | NO: 3) |
| Nme1Cas9 | ATGGCTGCCTTCAAACCTAATTCAATCAACTACATCCTCGG |
| Nucleic Acid | CCTCGATATCGGCATCGCATCCGTCGGCTGGGCGATGGTA |
| | GAAATTGACGAAGAAGAAAACCCCATCCGCCTGATTGATT |
| | TGGGCGTGCGCGTATTTGAGCGTGCCGAAGTACCGAAAAC |
| | AGGCGACTCCCTTGCCATGGCAAGGCGTTTGGCGCGCAGT |
| | GTTCGCCGCCTGACCCGCCGTCGCGCCCACCGCCTGCTTCG |
| | GACCCGCCGCCTATTGAAACGCGAAGGCGTATTACAAGCC |
| | GCCAATTTTGACGAAAACGGCTTGATTAAATCCTTACCGA |
| | ATACACCATGGCAACTTCGCGCAGCCGCATTAGACCGCAA |
| | ACTGACGCCTTTAGAGTGGTCGGCAGTCTTGTTGCATTTAA |
| | TCAAACATCGCGGCTATTTATCGCAACGGAAAAACGAGGG |
| | CGAAACTGCCGATAAGGAGCTTGGCGCTTTGCTTAAAGGC |
| | GTAGCCGGCAATGCCCATGCCTTACAGACAGGCGATTTCC |
| | GCACACCGGCCGAATTGGCTTTAAATAAATTTGAGAAAGA |
| | AAGCGGCCATATCCGCAATCAGCGCAGCGATTATTCGCAT |
| | ACGTTCAGCCGCAAAGATTTACAGGCGGAGCTGATTTTGC |
| | TGTTTGAAAAACAAAAAGAATTTGGCAATCCGCATGTTTC |
| | AGGCGGCCTTAAAGAAGGTATTGAAACCCTACTGATGACG |
| | CAACGCCCTGCCCTGTCCGGCGATGCCGTTCAAAAAATGT |
| | TGGGGCATTGCACCTTCGAACCGGCAGAGCCGAAAGCCGC |
| | TAAAAACACCTACACAGCCGAACGTTTCATCTGGCTGACC |
| | AAGCTGAACAACCTGCGTATTTTAGAGCAAGGCAGCGAGC |
| | GGCCATTGACCGATACCGAACGCGCCACGCTTATGGACGA |
| | GCCATACAGAAAATCCAAACTGACTTACGCACAAGCCCGT |
| | AAGCTGCTGGGTTTAGAAGATACCGCCTTTTTCAAAGGCTT |
| | GCGCTATGGTAAAGACAATGCCGAAGCCTCAACATTGATG |
| | GAAATGAAGGCCTACCATGCCATCAGCCGTGCACTGGAAA |
| | AAGAAGGATTGAAAGACAAAAAATCCCCATTAAACCTTTC |
| | TCCCGAATTACAAGACGAAATCGGCACGGCATTCTCCCTG |
| | TTCAAAACCGATGAAGACATTACAGGCCGTCTGAAAGACC |
| | GTATACAGCCCGAAATCTTAGAAGCGCTGTTGAAACACAT |
| | CAGCTTCGATAAGTTCGTCCAAATTTCCTTGAAAGCATTGC |
| | GCCGAATTGTGCCTCTAATGGAACAAGGCAAACGTTACGA |
| | TGAAGCCTGCGCCGAAATCTACGGAGACCATTACGGCAAG |
| | AAGAATACGGAAGAAAAGATTTATCTGCCGCCGATTCCCG |
| | CCGACGAAATCCGCAACCCCGTCGTCTTGCGCGCCTTATCT |
| | CAAGCACGTAAGGTCATTAACGGCGTGGTACGCCGTTACG |
| | GCTCCCCAGCTCGTATCCATATTGAAACTGCAAGGGAAGT |
| | AGGTAAATCGTTTAAAGACCGCAAAGAAATTGAGAAACGC |
| | CAAGAAGAAAACCGCAAAGACCGGGAAAAAGCCGCCGCC |
| | AAATTCCGAGAGTATTTCCCCAATTTTGTCGGAGAACCCA |
| | AATCCAAAGATATTCTGAAACTGCGCCTGTACGAGCAACA |
| | ACACGGCAAATGCCTGTATTCGGGCAAAGAAATCAACTTA |
| | GGCCGTCTGAACGAAAAAGGCTATGTCGAAATCGACCATG |
| | CCCTGCCGTTCTCGCGCACATGGGACGACAGTTTCAACAA |
| | TAAAGTACTGGTATTGGGCAGCGAAAACCAAAACAAAGG |
| | CAATCAAACCCCTTACGAATACTTCAACGGCAAAGACAAC |
| | AGCCGCGAATGGCAGGAATTTAAAGCGCGTGTCGAAACCA |
| | GCCGTTTCCCGCGCAGTAAAAAACAACGGATTCTGCTGCA |
| | AAAATTCGATGAAGACGGCTTTAAAGAACGCAATCTGAAC |
| | GACACGCGCTACGTCAACCGTTTCCTGTGTCAATTTGTTGC |
| | CGACCGTATGCGGCTGACAGGTAAAGGCAAGAAACGTGTC |
| | TTTGCATCCAACGGACAAATTACCAATCTGTTGCGCGGCTT |
| | TTGGGGATTGCGCAAAGTGCGTGCGGAAAACGACCGCCAT |
| | CACGCCTTGGACGCCGTCGTCGTTGCCTGCTCGACCGTTGC |
| | CATGCAGCAGAAAATTACCCGTTTTGTACGCTATAAAGAG |
| | ATGAACGCGTTTGACGGTAAAACCATAGACAAAGAAACA |
| | GGAGAAGTGCTGCATCAAAAAACACACTTCCCACAACCTT |
| | GGGAATTTTTCGCACAAGAAGTCATGATTCGCGTCTTCGGC |
| | AAACCGGACGGCAAACCCGAATTCGAAGAAGCCGATACC |
| | CTAGAAAAACTGCGCACGTTGCTTGCCGAAAAATTATCAT |
| | CTCGCCCCGAAGCCGTACACGAATACGTTACGCCACTGTTT |
| | GTTTCACGCGCGCCCAATCGGAAGATGAGCGGGCAAGGGC |
| | ATATGGAGACCGTCAAATCCGCCAAACGACTGGACGAAGG |
| | CGTCAGCGTGTTGCGCGTACCGCTGACACAGTTAAAACTG |
| | AAAGACTTGGAAAAAATGGTCAATCGGGAGCGCGAACCT |
| | AAGCTATACGAAGCACTGAAAGCACGGCTGGAAGCACAT |
| | AAAGACGATCCTGCCAAAGCCTTTGCCGAGCCGTTTTACA |
| | AATACGATAAAGCAGGCAACCGCACCCAACAGGTAAAAG |
| | CCGTACGCGTAGAGCAAGTACAGAAAACCGGCGTATGGGT |
| | GCGCAACCATAACGGTATTGCCGACAACGCAACCATGGTG |
| | CGCGTAGATGTGTTTGAGAAAGGCGACAAGTATTATCTGG |
| | TACCGATTTACAGTTGGCAGGTAGCGAAAGGGATTTTGCC |
| | GGATAGGGCTGTTGTACAAGGAAAAGATGAAGAAGATTG |
| | GCAACTTATTGATGATAGTTTCAACTTTAAATTCTCATTAC |
| | ACCCTAATGATTTAGTCGAGGTTATAACAAAAAAAGCTAG |
| | AATGTTTGGTTACTTTGCCAGCTGCCATCGAGGCACAGGTA |
| | ATATCAATATACGCATTCATGATCTTGATCATAAAATTGGC |
| | AAAAATGGAATACTGGAAGGTATCGGCGTCAAAACCGCCC |
| | TTTCATTCCAAAAATACCAAATTGACGAACTGGGCAAAGA |
| | AATCAGACCATGCCGTCTGAAAAAACGCCCGCCTGTCCGT |
| | TAA (SEQ ID NO: 4) |
| Nme2Cas9 | ATGGCCGCCTTCAAGCCTAACCCAATCAATTACATCCTGG |
| Nucleic Acid | GACTGGACATCGGAATCGCATCCGTGGGATGGGCTATGGT |
| Bacterial | GGAGATCGACGAGGAGGAGAATCCTATCCGGCTGATCGAT |
| Expression | CTGGGCGTGAGAGTGTTTGAGAGGGCCGAGGTGCCAAAGA |
| | CCGGCGATTCTCTGGCTATGGCCCGGAGACTGGCACGGAG |
| | CGTGAGGCGCCTGACACGGAGAAGGGCACACAGGCTGCT |
| | GAGGGCACGCCGGCTGCTGAAGAGAGAGGGCGTGCTGCA |
| | GGCAGCAGACTTCGATGAGAATGGCCTGATCAAGAGCCTG |
| | CCAAACACCCCCTGGCAGCTGAGAGCAGCCGCCCTGGACA |
| | GGAAGCTGACACCACTGGAGTGGTCTGCCGTGCTGCTGCA |
| | CCTGATCAAGCACCGCGGCTACCTGAGCCAGCGGAAGAAC |
| | GAGGGAGAGACAGCAGACAAGGAGCTGGGCGCCCTGCTG |
| | AAGGGAGTGGCCAACAATGCCCACGCCCTGCAGACCGGCG |
| | ATTTCAGGACACCTGCCGAGCTGGCCCTGAATAAGTTTGA |
| | GAAGGAGTCCGGCCACATCAGAAACCAGAGGGGCGACTA |
| | TAGCCACACCTTCTCCCGCAAGGATCTGCAGGCCGAGCTG |
| | ATCCTGCTGTTCGAGAAGCAGAAGGAGTTTGGCAATCCAC |
| | ACGTGAGCGGAGGCCTGAAGGAGGGAATCGAGACCCTGC |
| | TGATGACACAGAGGCCTGCCCTGTCCGGCGACGCAGTGCA |
| | GAAGATGCTGGGACACTGCACCTTCGAGCCTGCAGAGCCA |
| | AAGGCCGCCAAGAACACCTACACAGCCGAGCGGTTTATCT |
| | GGCTGACAAAGCTGAACAATCTGAGAATCCTGGAGCAGGG |
| | ATCCGAGAGGCCACTGACCGACACAGAGAGGGCCACCCTG |
| | ATGGATGAGCCTTACCGGAAGTCTAAGCTGACATATGCCC |
| | AGGCCAGAAAGCTGCTGGGCCTGGAGGACACCGCCTTCTT |
| | TAAGGGCCTGAGATACGGCAAGGATAATGCCGAGGCCTCC |
| | ACACTGATGGAGATGAAGGCCTATCACGCCATCTCTCGCG |
| | CCCTGGAGAAGGAGGGCCTGAAGGACAAGAAGTCCCCCCT |
| | GAACCTGAGCTCCGAGCTGCAGGATGAGATCGGCACCGCC |
| | TTCTCTCTGTTTAAGACCGACGAGGATATCACAGGCCGCCT |
| | GAAGGACAGGGTGCAGCCTGAGATCCTGGAGGCCCTGCTG |
| | AAGCACATCTCTTTCGATAAGTTTGTGCAGATCAGCCTGAA |
| | GGCCCTGAGAAGGATCGTGCCACTGATGGAGCAGGGCAA |
| | GCGGTACGACGAGGCCTGCGCCGAGATCTACGGCGATCAC |
| | TATGGCAAGAAGAACACAGAGGAGAAGATCTATCTGCCCC |
| | CTATCCCTGCCGACGAGATCAGAAATCCTGTGGTGCTGAG |
| | GGCCCTGTCCCAGGCAAGAAAAGTGATCAACGGAGTGGTG |
| | CGCCGGTACGGATCTCCAGCCCGGATCCACATCGAGACCG |
| | CCAGAGAAGTGGGCAAGAGCTTCAAGGACCGGAAGGAGA |
| | TCGAGAAGAGACAGGAGGAGAATCGCAAGGATCGGGAGA |
| | AGGCCGCCGCCAAGTTTAGGGAGTACTTCCCTAACTTTGTG |
| | GGCGAGCCAAAGTCTAAGGACATCCTGAAGCTGCGCCTGT |
| | ACGAGCAGCAGCACGGCAAGTGTCTGTATAGCGGCAAGG |
| | AGATCAATCTGGTGCGGCTGAACGAGAAGGGCTATGTGGA |
| | GATCGATCACGCCCTGCCTTTCTCCAGAACCTGGGACGATT |
| | CTTTTAACAATAAGGTGCTGGTGCTGGGCAGCGAGAACCA |
| | GAATAAGGGCAATCAGACACCATACGAGTATTTCAATGGC |
| | AAGGACAACTCCAGGGAGTGGCAGGAGTTCAAGGCCCGC |
| | GTGGAGACCTCTAGATTTCCCAGGAGCAAGAAGCAGCGGA |
| | TCCTGCTGCAGAAGTTCGACGAGGATGGCTTTAAGGAGTG |
| | CAACCTGAATGACACCAGATACGTGAACCGGTTCCTGTGC |
| | CAGTTTGTGGCCGATCACATCCTGCTGACCGGCAAGGGCA |
| | AGAGAAGGGTGTTCGCCTCTAATGGCCAGATCACAAACCT |
| | GCTGAGGGGATTTTGGGGACTGAGGAAGGTGCGGGCAGA |
| | GAATGACAGACACCACGCACTGGATGCAGTGGTGGTGGCA |
| | TGCAGCACCGTGGCAATGCAGCAGAAGATCACAAGATTCG |
| | TGAGGTATAAGGAGATGAACGCCTTTGACGGCAAGACCAT |
| | CGATAAGGAGACAGGCAAGGTGCTGCACCAGAAGACCCA |
| | CTTCCCCCAGCCTTGGGAGTTCTTTGCCCAGGAAGTGATGA |
| | TCCGGGTGTTCGGCAAGCCAGACGGCAAGCCTGAGTTTGA |
| | GGAGGCCGATACCCCAGAGAAGCTGAGGACACTGCTGGC |
| | AGAGAAGCTGTCTAGCAGGCCAGAGGCAGTGCACGAGTA |
| | CGTGACCCCACTGTTCGTGTCCAGGGCACCCAATCGGAAG |
| | ATGTCTGGCGCCCACAAGGACACACTGAGAAGCGCCAAGA |
| | GGTTTGTGAAGCACAACGAGAAGATCTCCGTGAAGAGAGT |
| | GTGGCTGACCGAGATCAAGCTGGCCGATCTGGAGAACATG |
| | GTGAATTACAAGAACGGCAGGGAGATCGAGCTGTATGAG |
| | GCCCTGAAGGCAAGGCTGGAGGCCTACGGAGGAAATGCC |
| | AAGCAGGCCTTCGACCCAAAGGATAACCCCTTTTATAAGA |
| | AGGGAGGACAGCTGGTGAAGGCCGTGCGGGTGGAGAAGA |
| | CCCAGGAGAGCGGCGTGCTGCTGAATAAGAAGAACGCCTA |
| | CACAATCGCCGACAATGGCGATATGGTGAGAGTGGACGTG |
| | TTCTGTAAGGTGGATAAGAAGGGCAAGAATCAGTACTTTA |
| | TCGTGCCTATCTATGCCTGGCAGGTGGCCGAGAACATCCT |
| | GCCAGACATCGATTGCAAGGGCTACAGAATCGACGATAGC |
| | TATACATTCTGTTTTTCCCTGCACAAGTATGACCTGATCGC |
| | CTTCCAGAAGGATGAGAAGTCCAAGGTGGAGTTTGCCTAC |
| | TATATCAATTGCGACTCCTCTAACGGCAGGTTCTACCTGGC |
| | CTGGCACGATAAGGGCAGCAAGGAGCAGCAGTTTCGCATC |
| | TCCACCCAGAATCTGGTGCTGATCCAGAAGTATCAGGTGA |
| | ACGAGCTGGGCAAGGAGATCAGGCCATGTCGGCTGAAGA |
| | AGCGCCCACCCGTGCGG (SEQ ID NO: 5) |
| Nme2Cas9 | ATGGCCGCCTTCAAGCCTAACCCAATCAATTACATCCTGG |
| Nucleic Acid- | GACTGGACATCGGAATCGCATCCGTGGGATGGGCTATGGT |
| Humanized | GGAGATCGACGAGGAGGAGAATCCTATCCGGCTGATCGAT |
| | CTGGGCGTGAGAGTGTTTGAGAGGGCCGAGGTGCCAAAGA |
| | CCGGCGATTCTCTGGCTATGGCCCGGAGACTGGCACGGAG |
| | CGTGAGGCGCCTGACACGGAGAAGGGCACACAGGCTGCT |
| | GAGGGCACGCCGGCTGCTGAAGAGAGAGGGCGTGCTGCA |
| | GGCAGCAGACTTCGATGAGAATGGCCTGATCAAGAGCCTG |
| | CCAAACACCCCCTGGCAGCTGAGAGCAGCCGCCCTGGACA |
| | GGAAGCTGACACCACTGGAGTGGTCTGCCGTGCTGCTGCA |
| | CCTGATCAAGCACCGCGGCTACCTGAGCCAGCGGAAGAAC |
| | GAGGGAGAGACAGCAGACAAGGAGCTGGGCGCCCTGCTG |
| | AAGGGAGTGGCCAACAATGCCCACGCCCTGCAGACCGGCG |
| | ATTTCAGGACACCTGCCGAGCTGGCCCTGAATAAGTTTGA |
| | GAAGGAGTCCGGCCACATCAGAAACCAGAGGGGCGACTA |
| | TAGCCACACCTTCTCCCGCAAGGATCTGCAGGCCGAGCTG |
| | ATCCTGCTGTTCGAGAAGCAGAAGGAGTTTGGCAATCCAC |
| | ACGTGAGCGGAGGCCTGAAGGAGGGAATCGAGACCCTGC |
| | TGATGACACAGAGGCCTGCCCTGTCCGGCGACGCAGTGCA |
| | GAAGATGCTGGGACACTGCACCTTCGAGCCTGCAGAGCCA |
| | AAGGCCGCCAAGAACACCTACACAGCCGAGCGGTTTATCT |
| | GGCTGACAAAGCTGAACAATCTGAGAATCCTGGAGCAGGG |
| | ATCCGAGAGGCCACTGACCGACACAGAGAGGGCCACCCTG |
| | ATGGATGAGCCTTACCGGAAGTCTAAGCTGACATATGCCC |
| | AGGCCAGAAAGCTGCTGGGCCTGGAGGACACCGCCTTCTT |
| | TAAGGGCCTGAGATACGGCAAGGATAATGCCGAGGCCTCC |
| | ACACTGATGGAGATGAAGGCCTATCACGCCATCTCTCGCG |
| | CCCTGGAGAAGGAGGGCCTGAAGGACAAGAAGTCCCCCCT |
| | GAACCTGAGCTCCGAGCTGCAGGATGAGATCGGCACCGCC |
| | TTCTCTCTGTTTAAGACCGACGAGGATATCACAGGCCGCCT |
| | GAAGGACAGGGTGCAGCCTGAGATCCTGGAGGCCCTGCTG |
| | AAGCACATCTCTTTCGATAAGTTTGTGCAGATCAGCCTGAA |
| | GGCCCTGAGAAGGATCGTGCCACTGATGGAGCAGGGCAA |
| | GCGGTACGACGAGGCCTGCGCCGAGATCTACGGCGATCAC |
| | TATGGCAAGAAGAACACAGAGGAGAAGATCTATCTGCCCC |
| | CTATCCCTGCCGACGAGATCAGAAATCCTGTGGTGCTGAG |
| | GGCCCTGTCCCAGGCAAGAAAAGTGATCAACGGAGTGGTG |
| | CGCCGGTACGGATCTCCAGCCCGGATCCACATCGAGACCG |
| | CCAGAGAAGTGGGCAAGAGCTTCAAGGACCGGAAGGAGA |
| | TCGAGAAGAGACAGGAGGAGAATCGCAAGGATCGGGAGA |
| | AGGCCGCCGCCAAGTTTAGGGAGTACTTCCCTAACTTTGTG |
| | GGCGAGCCAAAGTCTAAGGACATCCTGAAGCTGCGCCTGT |
| | ACGAGCAGCAGCACGGCAAGTGTCTGTATAGCGGCAAGG |
| | AGATCAATCTGGTGCGGCTGAACGAGAAGGGCTATGTGGA |
| | GATCGATCACGCCCTGCCTTTCTCCAGAACCTGGGACGATT |
| | CTTTTAACAATAAGGTGCTGGTGCTGGGCAGCGAGAACCA |
| | GAATAAGGGCAATCAGACACCATACGAGTATTTCAATGGC |
| | AAGGACAACTCCAGGGAGTGGCAGGAGTTCAAGGCCCGC |
| | GTGGAGACCTCTAGATTTCCCAGGAGCAAGAAGCAGCGGA |
| | TCCTGCTGCAGAAGTTCGACGAGGATGGCTTTAAGGAGTG |
| | CAACCTGAATGACACCAGATACGTGAACCGGTTCCTGTGC |
| | CAGTTTGTGGCCGATCACATCCTGCTGACCGGCAAGGGCA |
| | AGAGAAGGGTGTTCGCCTCTAATGGCCAGATCACAAACCT |
| | GCTGAGGGGATTTTGGGGACTGAGGAAGGTGCGGGCAGA |
| | GAATGACAGACACCACGCACTGGATGCAGTGGTGGTGGCA |
| | TGCAGCACCGTGGCAATGCAGCAGAAGATCACAAGATTCG |
| | TGAGGTATAAGGAGATGAACGCCTTTGACGGCAAGACCAT |
| | CGATAAGGAGACAGGCAAGGTGCTGCACCAGAAGACCCA |
| | CTTCCCCCAGCCTTGGGAGTTCTTTGCCCAGGAAGTGATGA |
| | TCCGGGTGTTCGGCAAGCCAGACGGCAAGCCTGAGTTTGA |
| | GGAGGCCGATACCCCAGAGAAGCTGAGGACACTGCTGGC |
| | AGAGAAGCTGTCTAGCAGGCCAGAGGCAGTGCACGAGTA |
| | CGTGACCCCACTGTTCGTGTCCAGGGCACCCAATCGGAAG |
| | ATGTCTGGCGCCCACAAGGACACACTGAGAAGCGCCAAGA |
| | GGTTTGTGAAGCACAACGAGAAGATCTCCGTGAAGAGAGT |
| | GTGGCTGACCGAGATCAAGCTGGCCGATCTGGAGAACATG |
| | GTGAATTACAAGAACGGCAGGGAGATCGAGCTGTATGAG |
| | GCCCTGAAGGCAAGGCTGGAGGCCTACGGAGGAAATGCC |
| | AAGCAGGCCTTCGACCCAAAGGATAACCCCTTTTATAAGA |
| | AGGGAGGACAGCTGGTGAAGGCCGTGCGGGTGGAGAAGA |
| | CCCAGGAGAGCGGCGTGCTGCTGAATAAGAAGAACGCCTA |
| | CACAATCGCCGACAATGGCGATATGGTGAGAGTGGACGTG |
| | TTCTGTAAGGTGGATAAGAAGGGCAAGAATCAGTACTTTA |
| | TCGTGCCTATCTATGCCTGGCAGGTGGCCGAGAACATCCT |
| | GCCAGACATCGATTGCAAGGGCTACAGAATCGACGATAGC |
| | TATACATTCTGTTTTTCCCTGCACAAGTATGACCTGATCGC |
| | CTTCCAGAAGGATGAGAAGTCCAAGGTGGAGTTTGCCTAC |
| | TATATCAATTGCGACTCCTCTAACGGCAGGTTCTACCTGGC |
| | CTGGCACGATAAGGGCAGCAAGGAGCAGCAGTTTCGCATC |
| | TCCACCCAGAATCTGGTGCTGATCCAGAAGTATCAGGTGA |
| | ACGAGCTGGGCAAGGAGATCAGGCCATGTCGGCTGAAGA |
| | AGCGCCCACCCGTGCGG (SEQ ID NO: 6) |
| Nme3Cas9 | ATGGCTGCCTTCAAACCTAATCCAATCAACTACATCCTCGG |
| Nucleic Acid | CCTCGATATCGGCATCGCATCCGTCGGCTGGGCGATGGTA |
| Bacterial | GAAATTGACGAAGAAGAAAACCCCATCCGCCTGATTGATT |
| Expression | TGGGCGTGCGCGTATTTGAGCGTGCCGAAGTACCGAAAAC |
| | AGGCGACTCCCTTGCCATGGCAAGGCGTTTGGCGCGCAGT |
| | GTCCGCCGCCTGACCCGCCGTCGCGCCCATCGCCTGCTTCG |
| | GGCCCGCCGCCTATTGAAACGCGAAGGCGTATTACAAGCC |
| | GCTGATTTTGACGAAAACGGCTTGATCAAATCCTTACCGA |
| | ATACACCATGGCAACTTCGCGCAGCCGCATTAGACCGCAA |
| | ACTGACGCCTTTAGAGTGGTCGGCAGTCTTATTGCATTTAA |
| | TCAAACACCGCGGTTATTTGTCGCAAAGAAAAAACGAGGG |
| | CGAAACTGCCGATAAAGAGCTTGGCGCTTTGCTCAAAGGC |
| | GTAGCCGACAATGCCCATGCCTTACAGACAGGCGATTTCC |
| | GCACACCGGCCGAATTGGCTTTAAATAAATTTGAGAAAGA |
| | ATGCGGCCATATCCGCAACCAACGCGGCGATTATTCGCAT |
| | ACGTTCAGCCGCAAAGATTTGCAGGCGGAGCTGAATTTAC |
| | TGTTTGAAAAACAAAAAGAATTCGGCAATCCGCATGTTTC |
| | AGGCGGCCTTAAAGAAGGTATTGAAACCCTACTGATGACG |
| | CAACGCCCTGCCCTGTCCGGCGATGCCGTTCAAAAAATGT |
| | TGGGGCATTGCACCTTCGAACCGGCAGAGCCGAAAGCCGC |
| | TAAAAACACCTACACAGCCGAACGTTTCATCTGGCTGACC |
| | AAGCTGAACAACCTGCGTATTTTAGAGCAAGGCAGCGAGC |
| | GGCCATTGACCGATACCGAACGCGCCACGCTTATGGACGA |
| | GCCATACAGAAAATCCAAACTGACTTACGCACAAGCCCGT |
| | AAGCTGCTGAGTTTAGAAGATACCGCCTTTTTCAAAGGCTT |
| | GCGCTATGGTAAAGACAATGCCGAAGCCTCAACATTGATG |
| | GAAATGAAGGCCTACCATACCATCAGCCGTGCACTGGAAA |
| | AAGAAGGATTGAAAGACAAAAAATCCCCATTAAACCTTTC |
| | TCCCGAATTACAAGACGAAATCGGCACGGCATTCTCCCTG |
| | TTCAAAACCGATGAAGACATTACAGGCCGTCTGAAAGACC |
| | GTATACAGCCCGAAATCTTAGAAGCGCTGTTGAAACACAT |
| | CAGCTTCGATAAGTTCGTCCAAATTTCCTTGAAAGCATTGC |
| | GCCGAATTGTGCCTCTAATGGAACAAGGCAAACGTTACGA |
| | TGAAGCCTGCGCCGAAATCTACGGAGACCATTACGGCAAG |
| | AAGAATACGGAAGAAAAGATTTATCTGCCGCCGATTCCCG |
| | CCGACGAAATCCGCAACCCCGTCGTCTTGCGCGCCTTATCT |
| | CAAGCACGTAAGGTCATTAACGGCGTGGTACGCCGTTACG |
| | GCTCCCCAGCTCGTATCCATATTGAAACTGCAAGGGAAGT |
| | AGGTAAATCGTTTAAAGACCGCAAAGAAATTGAGAAACGC |
| | CAAGAAGAAAACCGCAAAGACAGGGAAAAAGCCGCCGCC |
| | AAATTCCGAGAGTATTTCCCCAATTTTGTCGGAGAACCCA |
| | AATCCAAAGATATTCTGAAACTGCGCCTGTACGAGCAACA |
| | ACACGGCAAATGCCTGTATTCGGGCAAAGAAATCAACTTA |
| | GGCCGTCTGAACGAAAAAGGCTATGTCGAAATCGACCATG |
| | CCCTGCCGTTCTCGCGCACATGGGACGACAGTTTCAACAA |
| | TAAAGTACTGGTATTGGGCAGCGAAAACCAAAACAAAGG |
| | CAATCAAACCCCTTACGAATACTTCAACGGCAAAGACAAC |
| | AGCCGCGAATGGCAGGAATTTAAAGCGCGTGTCGAAACCA |
| | GCCGTTTCCCGCGCAGTAAAAAACAACGGATTCTGCTGCA |
| | AAAATTCGATGAAGACGGCTTTAAAGAACGCAATCTGAAC |
| | GACACGCGCTACGTCAACCGTTTCCTGTGTCAATTTGTTGC |
| | CGACCGTATGCGGCTGACAGGTAAAGGCAAGAAACGTGTC |
| | TTTGCATCCAACGGACAAATTACCAATCTGTTGCGCGGCTT |
| | TTGGGGATTGCGCAAAGTGCGTGCGGAAAACGACCGCCAT |
| | CACGCCTTGGACGCCGTCGTCGTTGCCTGCTCGACCGTTGC |
| | CATGCAGCAGAAAATTACCCGTTTTGTACGCTATAAAGAG |
| | ATGAACGCGTTTGACGGTAAAACCATAGACAAAGAAACA |
| | GGAGAAGTGCTGCATCAAAAAACACACTTCCCACAACCTT |
| | GGGAATTTTTCGCACAAGAAGTCATGATTCGCGTCTTCGGC |
| | AAACCGGACGGCAAACCCGAATTCGAAGAAGCCGATACC |
| | CCAGAAAAACTGCGCACGTTGCTTGCCGAAAAATTATCAT |
| | CTCGCCCCGAAGCCGTACACGAATACGTTACGCCACTGTTT |
| | GTTTCACGCGCGCCCAATCGGAAGATGAGCGGGCAAGGGC |
| | ATATGGAGACCGTCAAATCCGCCAAACGACTGGACGAAGG |
| | CGTCAGCGTGTTGCGCGTACCGTTAACACAGTTAAAACTG |
| | AAAGACTTGGAAAAAATGGTCAATCGGGAGCGCGAACCT |
| | AAGCTATACGAAGCACTGAAAGCACGGCTGGAAGCACAT |
| | AAAGACGATCCTGCCAAAGCCTTTGCCGAGCCGTTTTACA |
| | AATACGATAAAGCAGGCAACCGCACCCAACAGGTAAAAG |
| | CCGTACGCGTAGAGCAAGTACAGAAAACCGGCGTATGGGT |
| | GCGCAACCATAACGGTATTGCCGACAACGCAACCATGGTG |
| | CGCGTAGATGTGTTTGAGAAAGGCGACAAGTATTATCTGG |
| | TACCGATTTACAGTTGGCAGGTAGCGAAAGGGATTTTGCC |
| | GGATAGGGCTGTTGTTGCTTATGCAGATGAAGAGGACTGG |
| | ACTGTAATAGATGAAAGCTTCAGATTTAAATTTGTACTTTA |
| | TTCGAATGATTTAATTAAAGTTCAACTTAAAAAAGACAGC |
| | TTTTTAGGTTACTTTTCAGGATTAGATCGAGCCACTGGAGC |
| | CATTTCTCTTCGGGAGCATGATTTGGAAAAATCAAAAGGT |
| | AAAGACGGTATGCATCGTATCGGCGTTAAAACCGCCCTTT |
| | CCTTTCAAAAATACCAAATCGACGAAATGGGCAAAGAAAT |
| | CAGACCATGCCGTCTGAAAAAACGCCCGCCTGTCCGTTAA |
| | (SEQ ID NO: 7) |

In certain embodiments, the chemically modified Nme gRNAs of the disclosure are capable of binding to an Nme Cas9 nuclease or variant thereof comprising an amino acid sequence with at least 80% identity to an amino acid sequence set forth in SEQ ID NO: 1, 2, or 3.

In certain embodiments, the chemically modified Nme gRNAs of the disclosure are capable of binding to an Nme Cas9 nuclease or variant thereof comprising an amino acid sequence with 80%, 81%, 82%, 83%, 84%, 85%, 86%, 87%, 88%, 89%, 90%, 91%, 92%, 93%, 94%, 95%, 96%, 97%, 98%, 99%, or 100%, identity to an amino acid sequence set forth in SEQ ID NO: 1, 2, or 3.

In certain embodiments, the crRNA portion of the chemically modified Nme gRNAs of the disclosure bind to an Nme Cas9 nuclease or variant thereof through one or more nucleobases at positions 2, 11, 12, 14, 17, 20, 34, 35, and 36 from the 5′ end of the crRNA portion.

In certain embodiments, the repeat sequence of the crRNA portion of the chemically modified Nme gRNAs of the disclosure bind to an Nme Cas9 nuclease or variant thereof through one or more nucleobases at positions 10, 11, and 12 from the 5′ end of the repeat sequence of the crRNA portion.

In certain embodiments, the guide sequence of the crRNA portion of the chemically modified Nme gRNAs of the disclosure bind to an Nme Cas9 nuclease or variant thereof through one or more nucleobases at positions 5, 8, 11, 13, 14, and 23 from the 3′ end of the guide sequence of the crRNA portion.

Nme Cas9 nucleases are described in further detail in Esvelt et al. (Nat. Methods. 10: 1116-1121. 2013); Hou et al. (PNAS. 110: 15644-15649. 2013); Lee et al. (Mol. Thera. 24: 645-654. 2016); Amrani et al. (Genome Biol. 19: 214. 2018); Edraki et al. (Mol. Cell. 73: 714-726. 2019); U.S. Patent Publication 2014/0349405; U.S. Pat. No. 10,190,106; U.S. Patent Publication 2018/0355331; and U.S. Patent Publication 2019/0338308, each of which is incorporated herein by reference.

PAM sequences recognized by the Nme Cas9 nucleases of the disclosure may vary depending on Nme Cas9 type (e.g., Nme1Cas9, Nme2Cas9, or Nme3Cas9).

For example, but in no way limiting, Nme1Cas9 is capable of recognizing any one of PAM sequences N₄GAYW, N₄GYTT, or N₄GTCT, where “Y” corresponds to nucleotides C or T; “R” corresponds to nucleotides A or G; “M” corresponds to nucleotides A or C; “W” corresponds to nucleotides A or T; and “N” corresponds to nucleotides A, T, G, or C (see, Esvelt et al., supra; Hou et al., supra; Lee et al., supra; Amrani et al., supra).

In certain embodiments, Nme2Cas9 is capable of recognizing a PAM sequence of N₄CC (see, Sun et al., supra; Edraki et al., supra).

In certain embodiments, Nme3Cas9 is capable of recognizing a PAM sequence of N₄CAAA (see, Edraki et al., supra).

In certain embodiments, the NmeCas9 further comprises one or more nuclear localization signal (NLS) polypeptides. In certain embodiments, the NLS polypeptide comprises one or both of a nucleoplasmin NLS and an SV40 NLS.

In certain embodiments, the NmeCas9 is a variant NmeCas9. In certain embodiments, the variant NmeCas9 is a NmeCas9 nickase. In certain embodiments, the variant NmeCas9 comprises one or both of a D16A mutation and a H588A mutation.

In certain embodiments, the NmeCas9 or variant NmeCas9 is fused to a nucleotide deaminase. In certain embodiments, the nucleotide deaminase is a cytidine deaminase. In certain embodiments, the nucleotide deaminase is an adenosine deaminase. In certain embodiments, NmeCas9 or variant NmeCas9 further comprises a uracil glycosylase inhibitor.

The RNA-guided nucleases may be combined with the chemically modified guide RNAs of the present disclosure to form a genome-editing system. The RNA-guided nucleases may be combined with the chemically modified guide RNAs to form an RNP complex that may be delivered to a cell where genome-editing is desired. The RNA-guided nucleases may be expressed in a cell where genome-editing is desired with the chemically modified guide RNAs delivered separately. For example, the RNA-guided nucleases may be expressed from a polynucleotide such as a vector or a synthetic mRNA. The vector may be a viral vector, including, but not limited to, an adeno-associated virus (AAV) vector, an adenovirus vector, or a lentivirus (LV) vector.

It will be readily apparent to those skilled in the art that other suitable modifications and adaptations of the methods described herein may be made using suitable equivalents without departing from the scope of the embodiments disclosed herein. Having now described certain embodiments in detail, the same will be more clearly understood by reference to the following examples, which are included for purposes of illustration only and are not intended to be limiting.

### EXAMPLES

### Example 1—Synthesis of Chemically Modified crRNA and tracrRNA

crRNAs and tracrRNAs were synthesized at 1 μmole scale on a Dr. Oligo 48 DNA/RNA synthesizer. BTT (0.25 M in acetonitrile, ChemGenes) was used as activator. 0.05 M iodine in pyridine:water (9:1) (TEDIA) was used as oxidizer. DDTT (0.1 M, ChemGenes) was used as sulfurizing agent. 3% TCA in DCM (TEDIA) was used as deblock solution. RNAs were grown on 1000 Å CPG functionalized with Unylinker (˜42 μmol/g). Phosphoramidites (ChemGenes) were dissolved in acetonitrile to 0.15 M; the total contact time during coupling steps was approximately 10 min for each base; the phosphoramidite and activator were delivered in two aliquots during this time. After synthesis, the oligonucleotides were cleaved from solid support and nucleobases were deprotected with a 3:1 NH₄OH:EtOH solution for 48 hours at room temperature. Deprotection of the TBDMS group was achieved with DMSO:NEt3·3HF (4:1) solution (500 μL) at 65° C. for 3 hours. RNA oligonucleotides were then recovered by precipitation in 3M NaOAc (25 μL) and n-BuOH (1 mL), and the pellet was washed with cold 70% EtOH and resuspended in 1 mL RNase-free water.

Purification of the crRNAs and tracrRNAs were carried out by high performance liquid chromatography using a 1260 infinity system with an Agilent PL-SAX 1000 Å column (150×7.5 mm, 8 μm). Buffer A: 30% acetonitrile in water; Buffer B: 30% acetonitrile in 1M NaClO₄ (aq). Excess salt was removed with a Sephadex Nap-10 column.

crRNAs and tracrRNAs were analyzed on an Agilent 6530 Q-TOF LC/MS system with electrospray ionization and time of flight ion separation in negative ionization mode. The data were analyzed using Agilent Mass Hunter software. Buffer A: 100 mM hexafluoroisopropanol with 9 mM triethylamine in water; Buffer B: 100 mM hexafluoroisopropanol with 9 mM trimethylamine in methanol.

The crRNAs used in the Examples are recited below in Table 4. The sgRNAs used in the Examples are recited below in Table 5. Table 2 above recites tracrRNAs used in the Examples.

TABLE 4
Exemplary crRNAs.

| | | SEQ ID |
|---|---|---|
| Name | Sequence | NO: |
| CR42- | (mC)(mU)(mG)(rA)(rA)(rC)(rU)(rU)(rG)(rU)(rG)(rG)(rC)(rC)(rG)(rU)(rU) | 126 |
| 1_MCV | (rU)(rA)(rC)(rG)(rU)(rC)(rG)(rG)(rU)(rU)(rG)(rU)(rA)(rG)(rC)(rU)(rC) | |
| | (rC)(rC)(rU)(mU)(mU)(mC)(mU)(mC) | |
| CR42- | (mC)#(mU)#(mG)#(rA)(rA)(rC)(rU)(rU)(rG)(rU)(rG)(rG)(rC)(rC)(rG) | 127 |
| 2_MCV | (rU)(rU)(rU)(rA)(rC)(rG)(rU)(rC)(rG)(rG)(rU)(rU)(rG)(rU)(rA)(rG)(rC)(rU) | |
| | (rC)(rC)(rC)(rU)(mU)(mU)#(mC)#(mU)#(mC) | |
| CR42- | (mC)#(mU)(mG)(rA)(rA)(rC)(rU)(rU)(rG)(rU)(rG)(rG)(rC)(rC)(rG)(rU) | 128 |
| 3_MCV | (rU)(rU)(rA)(rC)(rG)(rU)(rC)(rG)(rG)(rU)(rU)(rG)(rU)(rA)(rG)(rC)(rU) | |
| | (rC)(rC)(rC)(rU)(mU)(mU)#(mC)#(mU)#(mC) | |
| CR42- | (mC)#(mU)#(mG)#(mA)(mA)(rC)(rU)(rU)(mG)(mU)(mG)(rG)(mC)(rC) | 129 |
| 4_MCV | (rG)(rU)(rU)(mU)(rA)(rC)(rG)(mU)(rC)(rG)(mG)(rU)(rU)(rG)(mU)(mA) | |
| | (mG)(mC)(rU)(rC)(rC)(rC)(rU)(mU)(mU)#(mC)#(mU)#(mC) | |
| CR42- | (mC)#(mU)#(mG)#(mA)(mA)(mC)(rU)(rU)(mG)(mU)(mG)(rG)(mC)(rC) | 130 |
| 5_MCV | (rG)(rU)(rU)(mU)(rA)(rC)(rG)(mU)(rC)(rG)(mG)(rU)(rU)(rG)(mU)(mA) | |
| | (mG)(mC)(rU)(rC)(rC)(rC)(rU)(mU)(mU)#(mC)#(mU)#(mC) | |
| CR42- | (mC)#(mU)#(mG)#(mA)(mA)(rC)(mU)(rU)(mG)(mU)(mG)(rG)(mC)(rC) | 131 |
| 6_MCV | (rG)(rU)(rU)(mU)(rA)(rC)(rG)(mU)(rC)(rG)(mG)(rU)(rU)(rG)(mU)(mA) | |
| | (mG)(mC)(rU)(rC)(rC)(rC)(rU)(mU)(mU)#(mC)#(mU)#(mC) | |
| CR42- | (mC)#(mU)#(mG)#(mA)(mA)(rC)(rU)(mU)(mG)(mU)(mG)(rG)(mC)(rC) | 132 |
| 7_MCV | (rG)(rU)(rU)(mU)(rA)(rC)(rG)(mU)(rC)(rG)(mG)(rU)(rU)(rG)(mU)(mA) | |
| | (mG)(mC)(rU)(rC)(rC)(rC)(rU)(mU)(mU)#(mC)#(mU)#(mC) | |
| CR42- | (mC)#(mU)#(mG)#(mA)(mA)(rC)(rU)(rU)(mG)(mU)(mG)(mG)(mC)(rC) | 133 |
| 8_MCV | (rG)(rU)(rU)(mU)(rA)(rC)(rG)(mU)(rC)(rG)(mG)(rU)(rU)(rG)(mU)(mA) | |
| | (mG)(mC)(rU)(rC)(rC)(rC)(rU)(mU)(mU)#(mC)#(mU)#(mC) | |
| CR42- | (mC)#(mU)#(mG)#(mA)(mA)(rC)(rU)(rU)(mG)(mU)(mG)(rG)(mC)(mC) | 134 |
| 9_MCV | (rG)(rU)(rU)(mU)(rA)(rC)(rG)(mU)(rC)(rG)(mG)(rU)(rU)(rG)(mU)(mA) | |
| | (mG)(mC)(rU)(rC)(rC)(rC)(rU)(mU)(mU)#(mC)#(mU)#(mC) | |
| CR42- | (mC)#(mU)#(mG)#(mA)(mA)(rC)(rU)(rU)(mG)(mU)(mG)(rG)(mC)(rC) | 135 |
| 10_MCV | (mG)(rU)(rU)(mU)(rA)(rC)(rG)(mU)(rC)(rG)(mG)(rU)(rU)(rG)(mU)(mA) | |
| | (mG)(mC)(rU)(rC)(rC)(rC)(rU)(mU)(mU)#(mC)#(mU)#(mC) | |
| CR42- | (mC)#(mU)#(mG)#(mA)(mA)(rC)(rU)(rU)(mG)(mU)(mG)(rG)(mC)(rC) | 136 |
| 11_MCV | (rG)(mU)(rU)(mU)(rA)(rC)(rG)(mU)(rC)(rG)(mG)(rU)(rU)(rG)(mU)(mA) | |
| | (mG)(mC)(rU)(rC)(rC)(rC)(rU)(mU)(mU)#(mC)#(mU)#(mC) | |
| CR42- | (mC)#(mU)#(mG)#(mA)(mA)(rC)(rU)(rU)(mG)(mU)(mG)(rG)(mC)(rC) | 137 |
| 12_MCV | (rG)(rU)(mU)(mU)(rA)(rC)(rG)(mU)(rC)(rG)(mG)(rU)(rU)(rG)(mU)(mA) | |
| | (mG)(mC)(rU)(rC)(rC)(rC)(rU)(mU)(mU)#(mC)#(mU)#(mC) | |
| CR42- | (mC)#(mU)#(mG)#(mA)(mA)(rC)(rU)(rU)(mG)(mU)(mG)(rG)(mC)(rC) | 138 |
| 13_MCV | (rG)(rU)(rU)(mU)(mA)(rC)(rG)(mU)(rC)(rG)(mG)(rU)(rU)(rG)(mU)(mA) | |
| | (mG)(mC)(rU)(rC)(rC)(rC)(rU)(mU)(mU)#(mC)#(mU)#(mC) | |
| CR42- | (mC)#(mU)#(mG)#(mA)(mA)(rC)(rU)(rU)(mG)(mU)(mG)(rG)(mC)(rC) | 139 |
| 14_MCV | (rG)(rU)(rU)(mU)(rA)(mC)(rG)(mU)(rC)(rG)(mG)(rU)(rU)(rG)(mU)(mA) | |
| | (mG)(mC)(rU)(rC)(rC)(rC)(rU)(mU)(mU)#(mC)#(mU)#(mC) | |
| CR42- | (mC)#(mU)#(mG)#(mA)(mA)(rC)(rU)(rU)(mG)(mU)(mG)(rG)(mC)(rC) | 140 |
| 15_MCV | (rG)(rU)(rU)(mU)(rA)(rC)(mG)(mU)(rC)(rG)(mG)(rU)(rU)(rG)(mU)(mA) | |
| | (mG)(mC)(rU)(rC)(rC)(rC)(rU)(mU)(mU)#(mC)#(mU)#(mC) | |
| CR42- | (mC)#(mU)#(mG)#(mA)(mA)(rC)(rU)(rU)(mG)(mU)(mG)(rG)(mC)(rC) | 141 |
| 16_MCV | (rG)(rU)(rU)(mU)(rA)(rC)(rG)(mU)(mC)(rG)(mG)(rU)(rU)(rG)(mU)(mA) | |
| | (mG)(mC)(rU)(rC)(rC)(rC)(rU)(mU)(mU)#(mC)#(mU)#(mC) | |
| CR42- | (mC)#(mU)#(mG)#(mA)(mA)(rC)(rU)(rU)(mG)(mU)(mG)(rG)(mC)(rC) | 142 |
| 17_MCV | (rG)(rU)(rU)(mU)(rA)(rC)(rG)(mU)(rC)(mG)(mG)(rU)(rU)(rG)(mU)(mA) | |
| | (mG)(mC)(rU)(rC)(rC)(rC)(rU)(mU)(mU)#(mC)#(mU)#(mC) | |
| CR42- | (mC)#(mU)#(mG)#(mA)(mA)(rC)(rU)(rU)(mG)(mU)(mG)(rG)(mC)(rC) | 143 |
| 18_MCV | (rG)(rU)(rU)(mU)(rA)(rC)(rG)(mU)(rC)(rG)(mG)(mU)(rU)(rG)(mU)(mA) | |
| | (mG)(mC)(rU)(rC)(rC)(rC)(rU)(mU)(mU)#(mC)#(mU)#(mC) | |
| CR42- | (mC)#(mU)#(mG)#(mA)(mA)(rC)(rU)(rU)(mG)(mU)(mG)(rG)(mC)(rC) | 144 |
| 19_MCV | (rG)(rU)(rU)(mU)(rA)(rC)(rG)(mU)(rC)(rG)(mG)(rU)(mU)(rG)(mU)(mA) | |
| | (mG)(mC)(rU)(rC)(rC)(rC)(rU)(mU)(mU)#(mC)#(mU)#(mC) | |
| CR42- | (mC)#(mU)#(mG)#(mA)(mA)(rC)(rU)(rU)(mG)(mU)(mG)(rG)(mC)(rC) | 145 |
| 20_MCV | (rG)(rU)(rU)(mU)(rA)(rC)(rG)(mU)(rC)(rG)(mG)(rU)(rU)(mG)(mU)(mA) | |
| | (mG)(mC)(rU)(rC)(rC)(rC)(rU)(mU)(mU)#(mC)#(mU)#(mC) | |
| CR42- | (mC)#(mU)#(mG)#(mA)(mA)(rC)(rU)(rU)(mG)(mU)(mG)(rG)(mC)(rC) | 146 |
| 21_MCV | (rG)(rU)(rU)(mU)(rA)(rC)(rG)(mU)(rC)(rG)(mG)(rU)(rU)(rG)(mU)(mA) | |
| | (mG)(mC)(mU)(rC)(rC)(rC)(rU)(mU)(mU)#(mC)#(mU)#(mC) | |
| CR42- | (mC)#(mU)#(mG)#(mA)(mA)(rC)(rU)(rU)(mG)(mU)(mG)(rG)(mC)(rC) | 147 |
| 22_MCV | (rG)(rU)(rU)(mU)(rA)(rC)(rG)(mU)(rC)(rG)(mG)(rU)(rU)(rG)(mU)(mA) | |
| | (mG)(mC)(rU)(mC)(rC)(rC)(rU)(mU)(mU)#(mC)#(mU)#(mC) | |
| CR42- | (mC)#(mU)#(mG)#(mA)(mA)(rC)(rU)(rU)(mG)(mU)(mG)(rG)(mC)(rC) | 148 |
| 23_MCV | (rG)(rU)(rU)(mU)(rA)(rC)(rG)(mU)(rC)(rG)(mG)(rU)(rU)(rG)(mU)(mA) | |
| | (mG)(mC)(rU)(rC)(mC)(rC)(rU)(mU)(mU)#(mC)#(mU)#(mC) | |
| CR42- | (mC)#(mU)#(mG)#(mA)(mA)(rC)(rU)(rU)(mG)(mU)(mG)(rG)(mC)(rC) | 149 |
| 24_MCV | (rG)(rU)(rU)(mU)(rA)(rC)(rG)(mU)(rC)(rG)(mG)(rU)(rU)(rG)(mU)(mA) | |
| | (mG)(mC)(rU)(rC)(rC)(mC)(rU)(mU)(mU)#(mC)#(mU)#(mC) | |
| CR42- | (mC)#(mU)#(mG)#(mA)(mA)(rC)(rU)(rU)(mG)(mU)(mG)(rG)(mC)(rC) | 150 |
| 25_MCV | (rG)(rU)(rU)(mU)(rA)(rC)(rG)(mU)(rC)(rG)(mG)(rU)(rU)(rG)(mU)(mA) | |
| | (mG)(mC)(rU)(rC)(rC)(rC)(mU)(mU)(mU)#(mC)#(mU)#(mC) | |
| CR42- | (mC)#(mU)#(mG)#(mA)(mA)(fC)(rU)(rU)(mG)(mU)(mG)(rG)(mC)(rC) | 151 |
| 26_MCV | (rG)(rU)(rU)(mU)(rA)(rC)(rG)(mU)(rC)(rG)(mG)(rU)(rU)(rG)(mU)(mA) | |
| | (mG)(mC)(rU)(rC)(rC)(rC)(rU)(mU)(mU)#(mC)#(mU)#(mC) | |
| CR42- | (mC)#(mU)#(mG)#(mA)(mA)(rC)(fU)(rU)(mG)(mU)(mG)(rG)(mC)(rC) | 152 |
| 27_MCV | (rG)(rU)(rU)(mU)(rA)(rC)(rG)(mU)(rC)(rG)(mG)(rU)(rU)(rG)(mU)(mA) | |
| | (mG)(mC)(rU)(rC)(rC)(rC)(rU)(mU)(mU)#(mC)#(mU)#(mC) | |
| CR42- | (mC)#(mU)#(mG)#(mA)(mA)(rC)(rU)(fU)(mG)(mU)(mG)(rG)(mC)(rC) | 153 |
| 28_MCV | (rG)(rU)(rU)(mU)(rA)(rC)(rG)(mU)(rC)(rG)(mG)(rU)(rU)(rG)(mU)(mA) | |
| | (mG)(mC)(rU)(rC)(rC)(rC)(rU)(mU)(mU)#(mC)#(mU)#(mC) | |
| CR42- | (mC)#(mU)#(mG)#(mA)(mA)(rC)(rU)(rU)(mG)(mU)(mG)(fG)(mC)(rC) | 154 |
| 29_MCV | (rG)(rU)(rU)(mU)(rA)(rC)(rG)(mU)(rC)(rG)(mG)(rU)(rU)(rG)(mU)(mA) | |
| | (mG)(mC)(rU)(rC)(rC)(rC)(rU)(mU)(mU)#(mC)#(mU)#(mC) | |
| CR42- | (mC)#(mU)#(mG)#(mA)(mA)(rC)(rU)(rU)(mG)(mU)(mG)(rG)(mC)(fC) | 155 |
| 30_MCV | (rG)(rU)(rU)(mU)(rA)(rC)(rG)(mU)(rC)(rG)(mG)(rU)(rU)(rG)(mU)(mA) | |
| | (mG)(mC)(rU)(rC)(rC)(rC)(rU)(mU)(mU)#(mC)#(mU)#(mC) | |
| CR42- | (mC)#(mU)#(mG)#(mA)(mA)(rC)(rU)(rU)(mG)(mU)(mG)(rG)(mC)(rC) | 156 |
| 31_MCV | (fG)(rU)(rU)(mU)(rA)(rC)(rG)(mU)(rC)(rG)(mG)(rU)(rU)(rG)(mU)(mA) | |
| | (mG)(mC)(rU)(rC)(rC)(rC)(rU)(mU)(mU)#(mC)#(mU)#(mC) | |
| CR42- | (mC)#(mU)#(mG)#(mA)(mA)(rC)(rU)(rU)(mG)(mU)(mG)(rG)(mC)(rC) | 157 |
| 32_MCV | (rG)(fU)(rU)(mU)(rA)(rC)(rG)(mU)(rC)(rG)(mG)(rU)(rU)(rG)(mU)(mA) | |
| | (mG)(mC)(rU)(rC)(rC)(rC)(rU)(mU)(mU)#(mC)#(mU)#(mC) | |
| CR42- | (mC)#(mU)#(mG)#(mA)(mA)(rC)(rU)(rU)(mG)(mU)(mG)(rG)(mC)(rC) | 158 |
| 33_MCV | (rG)(rU)(fU)(mU)(rA)(rC)(rG)(mU)(rC)(rG)(mG)(rU)(rU)(rG)(mU)(mA) | |
| | (mG)(mC)(rU)(rC)(rC)(rC)(rU)(mU)(mU)#(mC)#(mU)#(mC) | |
| CR42- | (mC)#(mU)#(mG)#(mA)(mA)(rC)(rU)(rU)(mG)(mU)(mG)(rG)(mC)(rC) | 159 |
| 34_MCV | (rG)(rU)(rU)(mU)(fA)(rC)(rG)(mU)(rC)(rG)(mG)(rU)(rU)(rG)(mU)(mA) | |
| | (mG)(mC)(rU)(rC)(rC)(rC)(rU)(mU)(mU)#(mC)#(mU)#(mC) | |
| CR42- | (mC)#(mU)#(mG)#(mA)(mA)(rC)(rU)(rU)(mG)(mU)(mG)(rG)(mC)(rC) | 160 |
| 35_MCV | (rG)(rU)(rU)(mU)(rA)(fC)(rG)(mU)(rC)(rG)(mG)(rU)(rU)(rG)(mU)(mA) | |
| | (mG)(mC)(rU)(rC)(rC)(rC)(rU)(mU)(mU)#(mC)#(mU)#(mC) | |
| CR42- | (mC)#(mU)#(mG)#(mA)(mA)(rC)(rU)(rU)(mG)(mU)(mG)(rG)(mC)(rC) | 161 |
| 36_MCV | (rG)(rU)(rU)(mU)(rA)(rC)(fG)(mU)(rC)(rG)(mG)(rU)(rU)(rG)(mU)(mA) | |
| | (mG)(mC)(rU)(rC)(rC)(rC)(rU)(mU)(mU)#(mC)#(mU)#(mC) | |
| CR42- | (mC)#(mU)#(mG)#(mA)(mA)(rC)(rU)(rU)(mG)(mU)(mG)(rG)(mC)(rC) | 162 |
| 37_MCV | (rG)(rU)(rU)(mU)(rA)(rC)(rG)(mU)(fC)(rG)(mG)(rU)(rU)(rG)(mU)(mA) | |
| | (mG)(mC)(rU)(rC)(rC)(rC)(rU)(mU)(mU)#(mC)#(mU)#(mC) | |
| CR42- | (mC)#(mU)#(mG)#(mA)(mA)(rC)(rU)(rU)(mG)(mU)(mG)(rG)(mC)(rC) | 163 |
| 38_MCV | (rG)(rU)(rU)(mU)(rA)(rC)(rG)(mU)(rC)(fG)(mG)(rU)(rU)(rG)(mU)(mA) | |
| | (mG)(mC)(rU)(rC)(rC)(rC)(rU)(mU)(mU)#(mC)#(mU)#(mC) | |
| CR42- | (mC)#(mU)#(mG)#(mA)(mA)(rC)(rU)(rU)(mG)(mU)(mG)(rG)(mC)(rC) | 164 |
| 39_MCV | (rG)(rU)(rU)(mU)(rA)(rC)(rG)(mU)(rC)(rG)(mG)(fU)(rU)(rG)(mU)(mA) | |
| | (mG)(mC)(rU)(rC)(rC)(rC)(rU)(mU)(mU)#(mC)#(mU)#(mC) | |
| CR42- | (mC)#(mU)#(mG)#(mA)(mA)(rC)(rU)(rU)(mG)(mU)(mG)(rG)(mC)(rC) | 165 |
| 40_MCV | (rG)(rU)(rU)(mU)(rA)(rC)(rG)(mU)(rC)(rG)(mG)(rU)(fU)(rG)(mU)(mA) | |
| | (mG)(mC)(rU)(rC)(rC)(rC)(rU)(mU)(mU)#(mC)#(mU)#(mC) | |
| CR42- | (mC)#(mU)#(mG)#(mA)(mA)(rC)(rU)(rU)(mG)(mU)(mG)(rG)(mC)(rC) | 166 |
| 41_MCV | (rG)(rU)(rU)(mU)(rA)(rC)(rG)(mU)(rC)(rG)(mG)(rU)(rU)(fG)(mU)(mA) | |
| | (mG)(mC)(rU)(rC)(rC)(rC)(rU)(mU)(mU)#(mC)#(mU)#(mC) | |
| CR42- | (mC)#(mU)#(mG)#(mA)(mA)(rC)(rU)(rU)(mG)(mU)(mG)(rG)(mC)(rC) | 167 |
| 42_MCV | (rG)(rU)(rU)(mU)(rA)(rC)(rG)(mU)(rC)(rG)(mG)(rU)(rU)(rG)(mU)(mA) | |
| | (mG)(mC)(fU)(rC)(rC)(rC)(rU)(mU)(mU)#(mC)#(mU)#(mC) | |
| CR42- | (mC)#(mU)#(mG)#(mA)(mA)(rC)(rU)(rU)(mG)(mU)(mG)(rG)(mC)(rC) | 168 |
| 43_MCV | (rG)(rU)(rU)(mU)(rA)(rC)(rG)(mU)(rC)(rG)(mG)(rU)(rU)(rG)(mU)(mA) | |
| | (mG)(mC)(rU)(fC)(rC)(rC)(rU)(mU)(mU)#(mC)#(mU)#(mC) | |
| CR42- | (mC)#(mU)#(mG)#(mA)(mA)(rC)(rU)(rU)(mG)(mU)(mG)(rG)(mC)(rC) | 169 |
| 44_MCV | (rG)(rU)(rU)(mU)(rA)(rC)(rG)(mU)(rC)(rG)(mG)(rU)(rU)(rG)(mU)(mA) | |
| | (mG)(mC)(rU)(rC)(fC)(rC)(rU)(mU)(mU)#(mC)#(mU)#(mC) | |
| CR42- | (mC)#(mU)#(mG)#(mA)(mA)(rC)(rU)(rU)(mG)(mU)(mG)(rG)(mC)(rC) | 170 |
| 45_MCV | (rG)(rU)(rU)(mU)(rA)(rC)(rG)(mU)(rC)(rG)(mG)(rU)(rU)(rG)(mU)(mA) | |
| | (mG)(mC)(rU)(rC)(rC)(fC)(rU)(mU)(mU)#(mC)#(mU)#(mC) | |
| CR42- | (mC)#(mU)#(mG)#(mA)(mA)(rC)(rU)(rU)(mG)(mU)(mG)(rG)(mC)(rC) | 171 |
| 46_MCV | (rG)(rU)(rU)(mU)(rA)(rC)(rG)(mU)(rC)(rG)(mG)(rU)(rU)(rG)(mU)(mA) | |
| | (mG)(mC)(rU)(rC)(rC)(rC)(fU)(mU)(mU)#(mC)#(mU)#(mC) | |
| CR42- | (mC)#(mU)#(mG)#(mA)(mA)(mC)(mU)(mU)(mG)(mU)(mG)(mG)(mC) | 172 |
| 47_MCV | (mC)(rG)(rU)(rU)(mU)(rA)(rC)(rG)(mU)(rC)(rG)(mG)(rU)(rU)(rG)(mU) | |
| | (mA)(mG)(mC)(rU)(rC)(rC)(rC)(rU)(mU)(mU)#(mC)#(mU)#(mC) | |
| CR42- | (mC)#(mU)#(mG)#(mA)(mA)(fC)(fU)(fU)(mG)(mU)(mG)(mG)(mC)(mC) | 173 |
| 48_MCV | (rG)(rU)(rU)(mU)(rA)(rC)(rG)(mU)(rC)(rG)(mG)(rU)(rU)(rG)(mU)(mA) | |
| | (mG)(mC)(rU)(rC)(rC)(rC)(rU)(mU)(mU)#(mC)#(mU)#(mC) | |
| CR42- | (mC)#(mU)#(mG)#(mA)(mA)(dC)(rU)(rU)(mG)(mU)(mG)(rG)(mC)(rC) | 174 |
| 49_MCV | (rG)(rU)(rU)(mU)(rA)(rC)(rG)(mU)(rC)(rG)(mG)(rU)(rU)(rG)(mU)(mA) | |
| | (mG)(mC)(rU)(rC)(rC)(rC)(rU)(mU)(mU)#(mC)#(mU)#(mC) | |
| CR42- | (mC)#(mU)#(mG)#(mA)(mA)(rC)(dT)(rU)(mG)(mU)(mG)(rG)(mC)(rC) | 175 |
| 50_MCV | (rG)(rU)(rU)(mU)(rA)(rC)(rG)(mU)(rC)(rG)(mG)(rU)(rU)(rG)(mU)(mA) | |
| | (mG)(mC)(rU)(rC)(rC)(rC)(rU)(mU)(mU)#(mC)#(mU)#(mC) | |
| CR42- | (mC)#(mU)#(mG)#(mA)(mA)(rC)(rU)(dT)(mG)(mU)(mG)(rG)(mC)(rC) | 176 |
| 51_MCV | (rG)(rU)(rU)(mU)(rA)(rC)(rG)(mU)(rC)(rG)(mG)(rU)(rU)(rG)(mU)(mA) | |
| | (mG)(mC)(rU)(rC)(rC)(rC)(rU)(mU)(mU)#(mC)#(mU)#(mC) | |
| CR42- | (mC)#(mU)#(mG)#(mA)(mA)(rC)(rU)(rU)(mG)(mU)(mG)(dG)(mC)(rC) | 177 |
| 52_MCV | (rG)(rU)(rU)(mU)(rA)(rC)(rG)(mU)(rC)(rG)(mG)(rU)(rU)(rG)(mU)(mA) | |
| | (mG)(mC)(rU)(rC)(rC)(rC)(rU)(mU)(mU)#(mC)#(mU)#(mC) | |
| CR42- | (mC)#(mU)#(mG)#(mA)(mA)(rC)(rU)(rU)(mG)(mU)(mG)(rG)(mC)(dC) | 178 |
| 53_MCV | (rG)(rU)(rU)(mU)(rA)(rC)(rG)(mU)(rC)(rG)(mG)(rU)(rU)(rG)(mU)(mA) | |
| | (mG)(mC)(rU)(rC)(rC)(rC)(rU)(mU)(mU)#(mC)#(mU)#(mC) | |
| CR42- | (mC)#(mU)#(mG)#(mA)(mA)(rC)(rU)(rU)(mG)(mU)(mG)(rG)(mC)(rC) | 179 |
| 54_MCV | (dG)(rU)(rU)(mU)(rA)(rC)(rG)(mU)(rC)(rG)(mG)(rU)(rU)(rG)(mU)(mA) | |
| | (mG)(mC)(rU)(rC)(rC)(rC)(rU)(mU)(mU)#(mC)#(mU)#(mC) | |
| CR42- | (mC)#(mU)#(mG)#(mA)(mA)(rC)(rU)(rU)(mG)(mU)(mG)(rG)(mC)(rC) | 180 |
| 55_MCV | (rG)(dT)(rU)(mU)(rA)(rC)(rG)(mU)(rC)(rG)(mG)(rU)(rU)(rG)(mU)(mA) | |
| | (mG)(mC)(rU)(rC)(rC)(rC)(rU)(mU)(mU)#(mC)#(mU)#(mC) | |
| CR42- | (mC)#(mU)#(mG)#(mA)(mA)(rC)(rU)(rU)(mG)(mU)(mG)(rG)(mC)(rC) | 181 |
| 56_MCV | (rG)(rU)(dT)(mU)(rA)(rC)(rG)(mU)(rC)(rG)(mG)(rU)(rU)(rG)(mU)(mA) | |
| | (mG)(mC)(rU)(rC)(rC)(rC)(rU)(mU)(mU)#(mC)#(mU)#(mC) | |
| CR42- | (mC)#(mU)#(mG)#(mA)(mA)(rC)(rU)(rU)(mG)(mU)(mG)(rG)(mC)(rC) | 182 |
| 57_MCV | (rG)(rU)(rU)(mU)(dA)(rC)(rG)(mU)(rC)(rG)(mG)(rU)(rU)(rG)(mU)(mA) | |
| | (mG)(mC)(rU)(rC)(rC)(rC)(rU)(mU)(mU)#(mC)#(mU)#(mC) | |
| CR42- | (mC)#(mU)#(mG)#(mA)(mA)(rC)(rU)(rU)(mG)(mU)(mG)(rG)(mC)(rC) | 183 |
| 58_MC | (rG)(rU)(rU)(mU)(rA)(dC)(rG)(mU)(rC)(rG)(mG)(rU)(rU)(rG)(mU)(mA) | |
| | (mG)(mC)(rU)(rC)(rC)(rC)(rU)(mU)(mU)#(mC)#(mU)#(mC) | |
| CR42- | (mC)#(mU)#(mG)#(mA)(mA)(rC)(rU)(rU)(mG)(mU)(mG)(rG)(mC)(rC) | 184 |
| 59_MCV | (rG)(rU)(rU)(mU)(rA)(rC)(dG)(mU)(rC)(rG)(mG)(rU)(rU)(rG)(mU)(mA) | |
| | (mG)(mC)(rU)(rC)(rC)(rC)(rU)(mU)(mU)#(mC)#(mU)#(mC) | |
| CR42- | (mC)#(mU)#(mG)#(mA)(mA)(rC)(rU)(rU)(mG)(mU)(mG)(rG)(mC)(rC) | 185 |
| 60_MCV | (rG)(rU)(rU)(mU)(rA)(rC)(rG)(mU)(dC)(rG)(mG)(rU)(rU)(rG)(mU)(mA) | |
| | (mG)(mC)(rU)(rC)(rC)(rC)(rU)(mU)(mU)#(mC)#(mU)#(mC) | |
| CR42- | (mC)#(mU)#(mG)#(mA)(mA)(rC)(rU)(rU)(mG)(mU)(mG)(rG)(mC)(rC) | 186 |
| 61_MCV | (rG)(rU)(rU)(mU)(rA)(rC)(rG)(mU)(rC)(dG)(mG)(rU)(rU)(rG)(mU)(mA) | |
| | (mG)(mC)(rU)(rC)(rC)(rC)(rU)(mU)(mU)#(mC)#(mU)#(mC) | |
| CR42- | (mC)#(mU)#(mG)#(mA)(mA)(rC)(rU)(rU)(mG)(mU)(mG)(rG)(mC)(rC) | 187 |
| 62_MCV | (rG)(rU)(rU)(mU)(rA)(rC)(rG)(mU)(rC)(rG)(mG)(dT)(rU)(rG)(mU)(mA) | |
| | (mG)(mC)(rU)(rC)(rC)(rC)(rU)(mU)(mU)#(mC)#(mU)#(mC) | |
| CR42- | (mC)#(mU)#(mG)#(mA)(mA)(rC)(rU)(rU)(mG)(mU)(mG)(rG)(mC)(rC) | 188 |
| 63_MCV | (rG)(rU)(rU)(mU)(rA)(rC)(rG)(mU)(rC)(rG)(mG)(rU)(dT)(rG)(mU)(mA) | |
| | (mG)(mC)(rU)(rC)(rC)(rC)(rU)(mU)(mU)#(mC)#(mU)#(mC) | |
| CR42- | (mC)#(mU)#(mG)#(mA)(mA)(rC)(rU)(rU)(mG)(mU)(mG)(rG)(mC)(rC) | 189 |
| 64_MCV | (rG)(rU)(rU)(mU)(rA)(rC)(rG)(mU)(rC)(rG)(mG)(rU)(rU)(dG)(mU)(mA) | |
| | (mG)(mC)(rU)(rC)(rC)(rC)(rU)(mU)(mU)#(mC)#(mU)#(mC) | |
| CR42- | (mC)#(mU)#(mG)#(mA)(mA)(rC)(rU)(rU)(mG)(mU)(mG)(rG)(mC)(rC) | 190 |
| 65_MCV | (rG)(rU)(rU)(mU)(rA)(rC)(rG)(mU)(rC)(rG)(mG)(rU)(rU)(rG)(mU)(mA) | |
| | (mG)(mC)(dT)(rC)(rC)(rC)(rU)(mU)(mU)#(mC)#(mU)#(mC) | |
| CR42- | (mC)#(mU)#(mG)#(mA)(mA)(rC)(rU)(rU)(mG)(mU)(mG)(rG)(mC)(rC) | 191 |
| 66_MCV | (rG)(rU)(rU)(mU)(rA)(rC)(rG)(mU)(rC)(rG)(mG)(rU)(rU)(rG)(mU)(mA) | |
| | (mG)(mC)(rU)(dC)(rC)(rC)(rU)(mU)(mU)#(mC)#(mU)#(mC) | |
| CR42- | (mC)#(mU)#(mG)#(mA)(mA)(rC)(rU)(rU)(mG)(mU)(mG)(rG)(mC)(rC) | 192 |
| 67_MCV | (rG)(rU)(rU)(mU)(rA)(rC)(rG)(mU)(rC)(rG)(mG)(rU)(rU)(rG)(mU)(mA) | |
| | (mG)(mC)(rU)(rC)(dC)(rC)(rU)(mU)(mU)#(mC)#(mU)#(mC) | |
| CR42- | (mC)#(mU)#(mG)#(mA)(mA)(rC)(rU)(rU)(mG)(mU)(mG)(rG)(mC)(rC) | 193 |
| 68_MCV | (rG)(rU)(rU)(mU)(rA)(rC)(rG)(mU)(rC)(rG)(mG)(rU)(rU)(rG)(mU)(mA) | |
| | (mG)(mC)(rU)(rC)(rC)(dC)(rU)(mU)(mU)#(mC)#(mU)#(mC) | |
| CR42- | (mC)#(mU)#(mG)#(mA)(mA)(rC)(rU)(rU)(mG)(mU)(mG)(rG)(mC)(rC) | 194 |
| 69_MCV | (rG)(rU)(rU)(mU)(rA)(rC)(rG)(mU)(rC)(rG)(mG)(rU)(rU)(rG)(mU)(mA) | |
| | (mG)(mC)(rU)(rC)(rC)(rC)(dT)(mU)(mU)#(mC)#(mU)#(mC) | |
| CR42- | (mC)#(mU)#(mG)#(mA)(mA)(rC)#(rU)(rU)(mG)(mU)(mG)(rG)(mC)(rC) | 195 |
| 70_MCV | (rG)(rU)(rU)(mU)(rA)(rC)(rG)(mU)(rC)(rG)(mG)(rU)(rU)(rG)(mU)(mA) | |
| | (mG)(mC)(rU)(rC)(rC)(rC)(rU)(mU)(mU)#(mC)#(mU)#(mC) | |
| CR42- | (mC)#(mU)#(mG)#(mA)(mA)(rC)(rU)#(rU)(mG)(mU)(mG)(rG)(mC)(rC) | 196 |
| 71_MCV | (rG)(rU)(rU)(mU)(rA)(rC)(rG)(mU)(rC)(rG)(mG)(rU)(rU)(rG)(mU)(mA) | |
| | (mG)(mC)(rU)(rC)(rC)(rC)(rU)(mU)(mU)#(mC)#(mU)#(mC) | |
| CR42- | (mC)#(mU)#(mG)#(mA)(mA)(rC)(rU)(rU)#(mG)(mU)(mG)(rG)(mC)(rC) | 197 |
| 72_MCV | (rG)(rU)(rU)(mU)(rA)(rC)(rG)(mU)(rC)(rG)(mG)(rU)(rU)(rG)(mU)(mA) | |
| | (mG)(mC)(rU)(rC)(rC)(rC)(rU)(mU)(mU)#(mC)#(mU)#(mC) | |
| CR42- | (mC)#(mU)#(mG)#(mA)(mA)(rC)(rU)(rU)(mG)(mU)(mG)(rG)#(mC)(rC) | 198 |
| 73_MCV | (rG)(rU)(rU)(mU)(rA)(rC)(rG)(mU)(rC)(rG)(mG)(rU)(rU)(rG)(mU)(mA) | |
| | (mG)(mC)(rU)(rC)(rC)(rC)(rU)(mU)(mU)#(mC)#(mU)#(mC) | |
| CR42- | (mC)#(mU)#(mG)#(mA)(mA)(rC)(rU)(rU)(mG)(mU)(mG)(rG)(mC)(rC)# | 199 |
| 74_MCV | (rG)(rU)(rU)(mU)(rA)(rC)(rG)(mU)(rC)(rG)(mG)(rU)(rU)(rG)(mU)(mA) | |
| | (mG)(mC)(rU)(rC)(rC)(rC)(rU)(mU)(mU)#(mC)#(mU)#(mC) | |
| CR42- | (mC)#(mU)#(mG)#(mA)(mA)(rC)(rU)(rU)(mG)(mU)(mG)(rG)(mC)(rC) | 200 |
| 75_MCV | (rG)#(rU)(rU)(mU)(rA)(rC)(rG)(mU)(rC)(rG)(mG)(rU)(rU)(rG)(mU)(mA) | |
| | (mG)(mC)(rU)(rC)(rC)(rC)(rU)(mU)(mU)#(mC)#(mU)#(mC) | |
| CR42- | (mC)#(mU)#(mG)#(mA)(mA)(rC)(rU)(rU)(mG)(mU)(mG)(rG)(mC)(rC) | 201 |
| 76_MCV | (rG)(rU)#(rU)(mU)(rA)(rC)(rG)(mU)(rC)(rG)(mG)(rU)(rU)(rG)(mU)(mA) | |
| | (mG)(mC)(rU)(rC)(rC)(rC)(rU)(mU)(mU)#(mC)#(mU)#(mC) | |
| CR42- | (mC)#(mU)#(mG)#(mA)(mA)(rC)(rU)(rU)(mG)(mU)(mG)(rG)(mC)(rC) | 202 |
| 77_MCV | (rG)(rU)(rU)#(mU)(rA)(rC)(rG)(mU)(rC)(rG)(mG)(rU)(rU)(rG)(mU)(mA) | |
| | (mG)(mC)(rU)(rC)(rC)(rC)(rU)(mU)(mU)#(mC)#(mU)#(mC) | |
| CR42- | (mC)#(mU)#(mG)#(mA)(mA)(rC)(rU)(rU)(mG)(mU)(mG)(rG)(mC)(rC) | 203 |
| 78_MCV | (rG)(rU)(rU)(mU)(rA)#(rC)(rG)(mU)(rC)(rG)(mG)(rU)(rU)(rG)(mU)(mA) | |
| | (mG)(mC)(rU)(rC)(rC)(rC)(rU)(mU)(mU)#(mC)#(mU)#(mC) | |
| CR42- | (mC)#(mU)#(mG)#(mA)(mA)(rC)(rU)(rU)(mG)(mU)(mG)(rG)(mC)(rC) | 204 |
| 79_MCV | (rG)(rU)(rU)(mU)(rA)(rC)#(rG)(mU)(rC)(rG)(mG)(rU)(rU)(rG)(mU)(mA) | |
| | (mG)(mC)(rU)(rC)(rC)(rC)(rU)(mU)(mU)#(mC)#(mU)#(mC) | |
| CR42- | (mC)#(mU)#(mG)#(mA)(mA)(rC)(rU)(rU)(mG)(mU)(mG)(rG)(mC)(rC) | 205 |
| 80_MCV | (rG)(rU)(rU)(mU)(rA)(rC)(rG)#(mU)(rC)(rG)(mG)(rU)(rU)(rG)(mU)(mA) | |
| V | (mG)(mC)(rU)(rC)(rC)(rC)(rU)(mU)(mU)#(mC)#(mU)#(mC) | |
| CR42- | (mC)#(mU)#(mG)#(mA)(mA)(rC)(rU)(rU)(mG)(mU)(mG)(rG)(mC)(rC) | 206 |
| 81_MCV | (rG)(rU)(rU)(mU)(rA)(rC)(rG)(mU)(rC)#(rG)(mG)(rU)(rU)(rG)(mU)(mA) | |
| | (mG)(mC)(rU)(rC)(rC)(rC)(rU)(mU)(mU)#(mC)#(mU)#(mC) | |
| CR42- | (mC)#(mU)#(mG)#(mA)(mA)(rC)(rU)(rU)(mG)(mU)(mG)(rG)(mC)(rC) | 207 |
| 82_MCV | (rG)(rU)(rU)(mU)(rA)(rC)(rG)(mU)(rC)(rG)#(mG)(rU)(rU)(rG)(mU)(mA) | |
| | (mG)(mC)(rU)(rC)(rC)(rC)(rU)(mU)(mU)#(mC)#(mU)#(mC) | |
| CR42- | (mC)#(mU)#(mG)#(mA)(mA)(rC)(rU)(rU)(mG)(mU)(mG)(rG)(mC)(rC) | 208 |
| 83_MCV | (rG)(rU)(rU)(mU)(rA)(rC)(rG)(mU)(rC)(rG)(mG)(rU)#(rU)(rG)(mU)(mA) | |
| | (mG)(mC)(rU)(rC)(rC)(rC)(rU)(mU)(mU)#(mC)#(mU)#(mC) | |
| CR42- | (mC)#(mU)#(mG)#(mA)(mA)(rC)(rU)(rU)(mG)(mU)(mG)(rG)(mC)(rC) | 209 |
| 84_MCV | (rG)(rU)(rU)(mU)(rA)(rC)(rG)(mU)(rC)(rG)(mG)(rU)(rU)#(rG)(mU)(mA) | |
| | (mG)(mC)(rU)(rC)(rC)(rC)(rU)(mU)(mU)#(mC)#(mU)#(mC) | |
| CR42- | (mC)#(mU)#(mG)#(mA)(mA)(rC)(rU)(rU)(mG)(mU)(mG)(rG)(mC)(rC) | 210 |
| 85_MCV | (rG)(rU)(rU)(mU)(rA)(rC)(rG)(mU)(rC)(rG)(mG)(rU)(rU)(rG)#(mU)(mA) | |
| | (mG)(mC)(rU)(rC)(rC)(rC)(rU)(mU)(mU)#(mC)#(mU)#(mC) | |
| CR42- | (mC)#(mU)#(mG)#(mA)(mA)(rC)(rU)(rU)(mG)(mU)(mG)(rG)(mC)(rC) | 211 |
| 86_MCV | (rG)(rU)(rU)(mU)(rA)(rC)(rG)(mU)(rC)(rG)(mG)(rU)(rU)(rG)(mU)(mA) | |
| | (mG)(mC)(rU)#(rC)(rC)(rC)(rU)(mU)(mU)#(mC)#(mU)#(mC) | |
| CR42- | (mC)#(mU)#(mG)#(mA)(mA)(rC)(rU)(rU)(mG)(mU)(mG)(rG)(mC)(rC) | 212 |
| 87_MCV | (rG)(rU)(rU)(mU)(rA)(rC)(rG)(mU)(rC)(rG)(mG)(rU)(rU)(rG)(mU)(mA) | |
| | (mG)(mC)(rU)(rC)#(rC)(rC)(rU)(mU)(mU)#(mC)#(mU)#(mC) | |
| CR42- | (mC)#(mU)#(mG)#(mA)(mA)(rC)(rU)(rU)(mG)(mU)(mG)(rG)(mC)(rC) | 213 |
| 88_MCV | (rG)(rU)(rU)(mU)(rA)(rC)(rG)(mU)(rC)(rG)(mG)(rU)(rU)(rG)(mU)(mA) | |
| | (mG)(mC)(rU)(rC)(rC)#(rC)(rU)(mU)(mU)#(mC)#(mU)#(mC) | |
| CR42- | (mC)#(mU)#(mG)#(mA)(mA)(rC)(rU)(rU)(mG)(mU)(mG)(rG)(mC)(rC) | 214 |
| 89_MCV | (rG)(rU)(rU)(mU)(rA)(rC)(rG)(mU)(rC)(rG)(mG)(rU)(rU)(rG)(mU)(mA) | |
| | (mG)(mC)(rU)(rC)(rC)(rC)#(rU)(mU)(mU)#(mC)#(mU)#(mC) | |
| CR42- | (mC)#(mU)#(mG)#(mA)(mA)(rC)(rU)(rU)(mG)(mU)(mG)(rG)(mC)(rC) | 215 |
| 90_MCV | (rG)(rU)(rU)(mU)(rA)(rC)(rG)(mU)(rC)(rG)(mG)(rU)(rU)(rG)(mU)(mA) | |
| | (mG)(mC)(rU)(rC)(rC)(rC)(rU)#(mU)(mU)#(mC)#(mU)#(mC) | |
| CR42- | (mC)#(mU)#(mG)#(mA)(mA)(dC)(dT)(dT)(mG)(mU)(mG)(mG)(mC) | 216 |
| 91_MCV | (mC)(rG)(rU)(rU)(mU)(rA)(rC)(rG)(mU)(rC)(rG)(mG)(rU)(rU)(rG)(mU) | |
| | (mA)(mG)(mC)(rU)(rC)(rC)(rC)(rU)(mU)(mU)#(mC)#(mU)#(mC) | |
| CR42- | (mC)#(mU)#(mG)#(mA)(mA)(rC)#(rU)#(rU)#(mG)(mU)(mG)(mG)(mC) | 217 |
| 92_MCV | (mC)(rG)(rU)(rU)(mU)(rA)(rC)(rG)(mU)(rC)(rG)(mG)(rU)(rU)(rG)(mU) | |
| | (mA)(mG)(mC)(rU)(rC)(rC)(rC)(rU)(mU)(mU)#(mC)#(mU)#(mC) | |
| CR42- | (mC)#(mU)#(mG)#(mA)(mA)(dC)#(dT)#(dT)#(mG)(mU)(mG)(mG)(mC) | 218 |
| 93_MCV | (mC)(rG)(rU)(rU)(mU)(rA)(rC)(rG)(mU)(rC)(rG)(mG)(rU)(rU)(rG)(mU) | |
| | (mA)(mG)(mC)(rU)(rC)(rC)(rC)(rU)(mU)(mU)#(mC)#(mU)#(mC) | |
| CR42- | (mC)#(mU)(mG)(mA)(mA)(rC)(rU)(rU)(mG)(mU)#(mG)(rG)(mC)(rC)# | 219 |
| 94_MCV | (rG)(rU)(rU)(mU)(rA)(rC)(rG)(mU)(rC)(rG)#(mG)(rU)(rU)(rG)(mU)#(mA) | |
| | #(mG)#(mC)#(rU)#(rC)#(rC)(rC)#(rU)#(mU)#(mU)#(mC)#(mU)#(mC) | |
| CR42- | (mC)#(mU)#(mG)#(mA)(mA)(rC)(rU)(rU)(mG)(mU)#(mG)(rG)(mC)(rC) | 220 |
| 95_MCV | #(rG)(rU)(rU)(mU)(rA)(rC)(rG)(mU)(rC)(rG)#(mG)(rU)(rU)(rG)(mU)# | |
| | (mA)#(mG)#(mC)#(rU)#(rC)#(rC)(rC)#(rU)#(mU)#(mU)#(mC)#(mU)#(mC) | |
| CR42- | (mC)#(mU)#(mG)#(mA)(mA)(rC)#(rU)#(rU)#(mG)(mU)#(mG)(rG)(mC) | 221 |
| 96_MCV | (rC)#(rG)(rU)(rU)(mU)(rA)(rC)(rG)(mU)(rC)(rG)#(mG)(rU)(rU)(rG)(mU) | |
| | #(mA)#(mG)#(mC)#(rU)#(rC)#(rC)(rC)#(rU)#(mU)#(mU)#(mC)#(mU)# | |
| | (mC) | |
| CR42- | (mC)#(mU)#(mG)#(mA)(mA)(rC)(rU)(rU)(mG)(mU)(mG)(rG)(mC)(rC) | 222 |
| 97_MCV | (rG)(rU)(rU)(mU)(rA)(rC)(rG)(mU)(rC)(rG)(mG)(dU)#(dU)#(mG)(mU) | |
| | (mA)(mG)(mC)(rU)(rC)(rC)(rC)(rU)(mU)(mU)#(mC)#(mU)#(mC) | |
| CR42- | (mC)#(mU)#(mG)#(mA)(mA)(rC)(rU)(rU)(mG)(mU)(mG)(rG)(mC)(rC) | 223 |
| 98_MCV | (rG)(rU)(rU)(mU)(rA)(rC)(rG)(mU)(rC)(rG)(mG)(dU)#(dU)#(dG)#(mU) | |
| | (mA)(mG)(mC)(rU)(rC)(rC)(rC)(rU)(mU)(mU)#(mC)#(mU)#(mC) | |
| CR42- | (mC)#(mU)#(mG)#(mA)(mA)(rC)(rU)(rU)(mG)(mU)(mG)(rG)(mC)(rC) | 224 |
| 99_MCV | (rG)(rU)(rU)(mU)(rA)(rC)(rG)(mU)(rC)(rG)(mG)(rU)(rU)(rG)(mU)(mA) | |
| | (mG)(mC)(dU)#(dC)#(dC)#(rC)(dU)#(mU)(mU)#(mC)#(mU)#(mC) | |
| CR42- | (mC)#(mU)#(mG)#(mA)(mA)(rC)(rU)(rU)(mG)(mU)(mG)(rG)(mC)(rC) | 225 |
| 100_MCV | (rG)(rU)(rU)(mU)(rA)(rC)(rG)(mU)(rC)(rG)(mG)(rU)(rU)(rG)(mU)(mA) | |
| | (mG)(mC)(dU)#(dC)#(dC)#(mC)(dU)#(mU)(mU)#(mC)#(mU)#(mC) | |
| CR42- | (mC)#(mU)#(mG)#(mA)(mA)(rC)(rU)(rU)(mG)(mU)(mG)(rG)(mC)(rC) | 226 |
| 101_MCV | (rG)(rU)(rU)(mU)(rA)(rC)(rG)(mU)(rC)(rG)(mG)(rU)(rU)(rG)(mU)(mA) | |
| | (mG)(mC)(mU)(dC)#(dC)#(rC)(dU)#(mU)(mU)#(mC)#(mU)#(mC) | |
| CR42- | (mC)#(mU)#(mG)#(mA)(mA)(rC)(rU)(rU)(mG)(mU)(mG)(rG)(mC)(rC) | 227 |
| 102_MCV | (rG)(rU)(rU)(mU)(rA)(rC)(rG)(mU)(rC)(rG)(mG)(rU)(rU)(rG)(mU)(mA) | |
| | (mG)(mC)(dU)#(mC)(dC)#(rC)(dU)#(mU)(mU)#(mC)#(mU)#(mC) | |
| CR42- | (mC)#(mU)#(mG)#(mA)(mA)(rC)(rU)(rU)(mG)(mU)(mG)(rG)(mC)(rC) | 228 |
| 103_MCV | (rG)(rU)(rU)(mU)(rA)(rC)(rG)(mU)(rC)(rG)(mG)(rU)(rU)(rG)(mU)(mA) | |
| | (mG)(mC)(dU)#(dC)#(mC)(rC)(dU)#(mU)(mU)#(mC)#(mU)#(mC) | |
| CR42- | (mC)#(mU)#(mG)#(mA)(mA)(rC)(rU)(rU)(mG)(mU)(mG)(rG)(mC)(rC) | 229 |
| 104_MCV | (rG)(rU)(rU)(mU)(rA)(rC)(rG)(mU)(rC)(rG)(mG)(rU)(rU)(rG)(mU)(mA) | |
| | (mG)(mC)(mU)(mC)(dC)#(rC)(dU)#(mU)(mU)#(mC)#(mU)#(mC) | |
| CR42- | (mC)#(mU)#(mG)#(mA)(mA)(rC)(rU)(rU)(mG)(mU)(mG)(rG)(mC)(rC) | 230 |
| 105_MCV | (rG)(rU)(rU)(mU)(rA)(rC)(rG)(mU)(rC)(rG)(mG)(rU)(rU)(rG)(mU)(mA) | |
| | (mG)(mC)(mU)(dC)#(mC)(rC)(dU)#(mU)(mU)#(mC)#(mU)#(mC) | |
| CR42- | (mC)#(mU)#(mG)#(mA)(mA)(rC)(rU)(rU)(mG)(mU)(mG)(rG)(mC)(rC) | 231 |
| 106_MCV | (rG)(rU)(rU)(mU)(rA)(rC)(rG)(mU)(rC)(rG)(mG)(rU)(rU)(rG)(mU)(mA) | |
| | (mG)(mC)(dU)#(mC)(mC)(rC)(dU)#(mU)(mU)#(mC)#(mU)#(mC) | |
| CR42- | (mC)#(mU)#(mG)#(mA)(mA)(rC)(rU)(rU)(mG)(mU)(mG)(rG)(mC)(rC) | 232 |
| 107_MCV | (rG)(rU)(rU)(mU)(rA)(rC)(rG)(mU)(rC)(rG)(mG)(rU)(rU)(rG)(mU)(mA) | |
| | (mG)(mC)(mU)(mC)(mC)(rC)(dU)#(mU)(mU)#(mC)#(mU)#(mC) | |
| CR42- | (mC)#(mU)#(mG)#(mA)(mA)(rC)(rU)(rU)(mG)(mU)(mG)(rG)(mC)(rC) | 233 |
| 108_MCV | (rG)(rU)(rU)(mU)(rA)(rC)(rG)(mU)(rC)(rG)(mG)(dU)#(dU)#(mG)(mU) | |
| | (mA)(mG)(mC)(dU)#(dC)#(dC)#(rC)(dU)#(mU)(mU)#(mC)#(mU)#(mC) | |
| CR42- | (mC)#(mU)#(mG)#(mA)(mA)(rC)(rU)(rU)(mG)(mU)(mG)(rG)(mC)(rC) | 234 |
| 109_MCV | (rG)(rU)(rU)(mU)(rA)(rC)(rG)(mU)(rC)(rG)(mG)(dU)#(dU)#(mG)(mU) | |
| | (mA)(mG)(mC)(dU)#(dC)#(dC)#(mC)(dU)#(mU)(mU)#(mC)#(mU)#(mC) | |
| CR42- | (mC)#(mU)#(mG)#(mA)(mA)(rC)(rU)(rU)(mG)(mU)(mG)(rG)(mC)(rC) | 235 |
| 110_MCV | (rG)(rU)(rU)(mU)(rA)(rC)(rG)(mU)(rC)(rG)(mG)(dU)#(dU)#(mG)(mU) | |
| | (mA)(mG)(mC)(mU)(dC)#(dC)#(rC)(dU)#(mU)(mU)#(mC)#(mU)#(mC) | |
| CR42- | (mC)#(mU)#(mG)#(mA)(mA)(rC)(rU)(rU)(mG)(mU)(mG)(rG)(mC)(rC) | 236 |
| 111_MCV | (rG)(rU)(rU)(mU)(rA)(rC)(rG)(mU)(rC)(rG)(mG)(dU)#(dU)#(mG)(mU) | |
| | (mA)(mG)(mC)(dU)#(mC)(dC)#(rC)(dU)#(mU)(mU)#(mC)#(mU)#(mC) | |
| CR42- | (mC)#(mU)#(mG)#(mA)(mA)(rC)(rU)(rU)(mG)(mU)(mG)(rG)(mC)(rC) | 237 |
| 112_MCV | (rG)(rU)(rU)(mU)(rA)(rC)(rG)(mU)(rC)(rG)(mG)(dU)#(dU)#(mG)(mU) | |
| | (mA)(mG)(mC)(dU)#(dC)#(mC)(rC)(dU)#(mU)(mU)#(mC)#(mU)#(mC) | |
| CR42- | (mC)#(mU)#(mG)#(mA)(mA)(rC)(rU)(rU)(mG)(mU)(mG)(rG)(mC)(rC) | 238 |
| 113_MCV | (rG)(rU)(rU)(mU)(rA)(rC)(rG)(mU)(rC)(rG)(mG)(dU)#(dU)#(mG)(mU) | |
| | (mA)(mG)(mC)(mU)(mC)(dC)#(rC)(dU)#(mU)(mU)#(mC)#(mU)#(mC) | |
| CR42- | (mC)#(mU)#(mG)#(mA)(mA)(rC)(rU)(rU)(mG)(mU)(mG)(rG)(mC)(rC) | 239 |
| 114_MCV | (rG)(rU)(rU)(mU)(rA)(rC)(rG)(mU)(rC)(rG)(mG)(dU)#(dU)#(mG)(mU) | |
| | (mA)(mG)(mC)(mU)(dC)#(mC)(rC)(dU)#(mU)(mU)#(mC)#(mU)#(mC) | |
| CR42- | (mC)#(mU)#(mG)#(mA)(mA)(rC)(rU)(rU)(mG)(mU)(mG)(rG)(mC)(rC) | 240 |
| 115_MCV | (rG)(rU)(rU)(mU)(rA)(rC)(rG)(mU)(rC)(rG)(mG)(dU)#(dU)#(mG)(mU) | |
| | (mA)(mG)(mC)(dU)#(mC)(mC)(rC)(dU)#(mU)(mU)#(mC)#(mU)#(mC) | |
| CR42- | (mC)#(mU)#(mG)#(mA)(mA)(rC)(rU)(rU)(mG)(mU)(mG)(rG)(mC)(rC) | 241 |
| 116_MCV | (rG)(rU)(rU)(mU)(rA)(rC)(rG)(mU)(rC)(rG)(mG)(dU)#(dU)#(mG)(mU) | |
| | (mA)(mG)(mC)(mU)(mC)(mC)(rC)(dU)#(mU)(mU)#(mC)#(mU)#(mC) | |
| CR42- | (mC)#(mU)#(mG)#(mA)(mA)(rC)(rU)(rU)(mG)(mU)(mG)(rG)(mC)(rC) | 242 |
| 117_MCV | (rG)(rU)(rU)(mU)(rA)(rC)(rG)(mU)(rC)(rG)(mG)(dU)#(dU)#(dG)#(mU) | |
| | (mA)(mG)(mC)(dU)#(dC)#(dC)#(rC)(dU)#(mU)(mU)#(mC)#(mU)#(mC) | |
| CR42- | (mC)#(mU)#(mG)#(mA)(mA)(rC)(rU)(rU)(mG)(mU)(mG)(rG)(mC)(rC) | 243 |
| 118_MCV | (rG)(rU)(rU)(mU)(rA)(rC)(rG)(mU)(rC)(rG)(mG)(dU)#(dU)#(dG)#(mU) | |
| | (mA)(mG)(mC)(dU)#(dC)#(dC)#(mC)(dU)#(mU)(mU)#(mC)#(mU)#(mC) | |
| CR42- | (mC)#(mU)#(mG)#(mA)(mA)(rC)(rU)(rU)(mG)(mU)(mG)(rG)(mC)(rC) | 244 |
| 119_MCV | (rG)(rU)(rU)(mU)(rA)(rC)(rG)(mU)(rC)(rG)(mG)(dU)#(dU)#(dG)#(mU) | |
| | (mA)(mG)(mC)(mU)(dC)#(dC)#(rC)(dU)#(mU)(mU)#(mC)#(mU)#(mC) | |
| CR42- | (mC)#(mU)#(mG)#(mA)(mA)(rC)(rU)(rU)(mG)(mU)(mG)(rG)(mC)(rC) | 245 |
| 120_MCV | (rG)(rU)(rU)(mU)(rA)(rC)(rG)(mU)(rC)(rG)(mG)(dU)#(dU)#(dG)#(mU) | |
| | (mA)(mG)(mC)(dU)#(mC)(dC)#(rC)(dU)#(mU)(mU)#(mC)#(mU)#(mC) | |
| CR42- | (mC)#(mU)#(mG)#(mA)(mA)(rC)(rU)(rU)(mG)(mU)(mG)(rG)(mC)(rC) | 246 |
| 121_MCV | (rG)(rU)(rU)(mU)(rA)(rC)(rG)(mU)(rC)(rG)(mG)(dU)#(dU)#(dG)#(mU) | |
| | (mA)(mG)(mC)(dU)#(dC)#(mC)(rC)(dU)#(mU)(mU)#(mC)#(mU)#(mC) | |
| CR42- | (mC)#(mU)#(mG)#(mA)(mA)(rC)(rU)(rU)(mG)(mU)(mG)(rG)(mC)(rC) | 247 |
| 122_MCV | (rG)(rU)(rU)(mU)(rA)(rC)(rG)(mU)(rC)(rG)(mG)(dU)#(dU)#(dG)#(mU) | |
| | (mA)(mG)(mC)(mU)(mC)(dC)#(rC)(dU)#(mU)(mU)#(mC)#(mU)#(mC) | |
| CR42- | (mC)#(mU)#(mG)#(mA)(mA)(rC)(rU)(rU)(mG)(mU)(mG)(rG)(mC)(rC) | 248 |
| 123_MCV | (rG)(rU)(rU)(mU)(rA)(rC)(rG)(mU)(rC)(rG)(mG)(dU)#(dU)#(dG)#(mU) | |
| | (mA)(mG)(mC)(mU)(dC)#(mC)(rC)(dU)#(mU)(mU)#(mC)#(mU)#(mC) | |
| CR42- | (mC)#(mU)#(mG)#(mA)(mA)(rC)(rU)(rU)(mG)(mU)(mG)(rG)(mC)(rC) | 249 |
| 124_MCV | (rG)(rU)(rU)(mU)(rA)(rC)(rG)(mU)(rC)(rG)(mG)(dU)#(dU)#(dG)#(mU) | |
| | (mA)(mG)(mC)(dU)#(mC)(mC)(rC)(dU)#(mU)(mU)#(mC)#(mU)#(mC) | |
| CR42- | (mC)#(mU)#(mG)#(mA)(mA)(rC)(rU)(rU)(mG)(mU)(mG)(rG)(mC)(rC) | 250 |
| 125_MCV | (rG)(rU)(rU)(mU)(rA)(rC)(rG)(mU)(rC)(rG)(mG)(dU)#(dU)#(dG)#(mU) | |
| | (mA)(mG)(mC)(mU)(mC)(mC)(rC)(dU)#(mU)(mU)#(mC)#(mU)#(mC) | |
| CR42- | (mC)#(mU)#(mG)#(mA)(mA)(mC)(fU)(mU)(mG)(mU)(mG)(rG)(mC)(rC) | 251 |
| 126_MCV | (rG)(rU)(rU)(mU)(rA)(rC)(rG)(mU)(rC)(rG)(mG)(rU)(rU)(rG)(mU)(mA) | |
| | (mG)(mC)(rU)(rC)(rC)(rC)(rU)(mU)(mU)#(mC)#(mU)#(mC) | |
| CR42- | (mC)#(mU)#(mG)#(mA)(mA)(mC)(fU)(dU)#(mG)(mU)(mG)(rG)(mC) | 252 |
| 127_MCV | (rC)(rG)(rU)(rU)(mU)(rA)(rC)(rG)(mU)(rC)(rG)(mG)(rU)(rU)(rG)(mU) | |
| | (mA)(mG)(mC)(rU)(rC)(rC)(rC)(rU)(mU)(mU)#(mC)#(mU)#(mC) | |
| CR42- | (mC)#(mU)#(mG)#(mA)(mA)(mC)(dU)#(mU)(mG)(mU)(mG)(rG)(mC) | 253 |
| 128_MCV | (rC)(rG)(rU)(rU)(mU)(rA)(rC)(rG)(mU)(rC)(rG)(mG)(rU)(rU)(rG)(mU) | |
| | (mA)(mG)(mC)(rU)(rC)(rC)(rC)(rU)(mU)(mU)#(mC)#(mU)#(mC) | |
| CR42- | (mC)#(mU)#(mG)#(mA)(mA)(mC)(dU)#(dU)#(mG)(mU)(mG)(rG)(mC) | 254 |
| 129_MCV | (rC)(rG)(rU)(rU)(mU)(rA)(rC)(rG)(mU)(rC)(rG)(mG)(rU)(rU)(rG)(mU) | |
| | (mA)(mG)(mC)(rU)(rC)(rC)(rC)(rU)(mU)(mU)#(mC)#(mU)#(mC) | |
| CR42- | (mC)#(mU)#(mG)#(mA)(mA)(fC)(fU)(mU)(mG)(mU)(mG)(rG)(mC)(rC) | 255 |
| 130_MCV | (rG)(rU)(rU)(mU)(rA)(rC)(rG)(mU)(rC)(rG)(mG)(rU)(rU)(rG)(mU)(mA) | |
| | (mG)(mC)(rU)(rC)(rC)(rC)(rU)(mU)(mU)#(mC)#(mU)#(mC) | |
| CR42- | (mC)#(mU)#(mG)#(mA)(mA)(fC)(fU)(dU)#(mG)(mU)(mG)(rG)(mC)(rC) | 256 |
| 131_MCV | (rG)(rU)(rU)(mU)(rA)(rC)(rG)(mU)(rC)(rG)(mG)(rU)(rU)(rG)(mU)(mA) | |
| | (mG)(mC)(rU)(rC)(rC)(rC)(rU)(mU)(mU)#(mC)#(mU)#(mC) | |
| CR42- | (mC)#(mU)#(mG)#(mA)(mA)(fC)(dU)#(mU)(mG)(mU)(mG)(rG)(mC) | 257 |
| 132_MCV | (rC)(rG)(rU)(rU)(mU)(rA)(rC)(rG)(mU)(rC)(rG)(mG)(rU)(rU)(rG)(mU) | |
| | (mA)(mG)(mC)(rU)(rC)(rC)(rC)(rU)(mU)(mU)#(mC)#(mU)#(mC) | |
| CR42- | (mC)#(mU)#(mG)#(mA)(mA)(fC)(dU)#(dU)#(mG)(mU)(mG)(rG)(mC) | 258 |
| 133_MCV | (rC)(rG)(rU)(rU)(mU)(rA)(rC)(rG)(mU)(rC)(rG)(mG)(rU)(rU)(rG)(mU) | |
| | (mA)(mG)(mC)(rU)(rC)(rC)(rC)(rU)(mU)(mU)#(mC)#(mU)#(mC) | |
| CR42- | (mC)#(mU)#(mG)#(mA)(mA)(rC)(rU)(rU)(mG)(mU)(mG)(rG)(mC)(fC) | 259 |
| 134_MCV | (fG)(fU)(fU)(mU)(rA)(rC)(rG)(mU)(rC)(rG)(mG)(rU)(rU)(rG)(mU)(mA) | |
| | (mG)(mC)(rU)(rC)(rC)(rC)(rU)(mU)(mU)#(mC)#(mU)#(mC) | |
| CR42- | (mC)#(mU)#(mG)#(mA)(mA)(rC)(rU)(rU)(mG)(mU)(mG)(rG)(mC)(fC) | 260 |
| 135_MCV | (mG)(fU)(fU)(mU)(rA)(rC)(rG)(mU)(rC)(rG)(mG)(rU)(rU)(rG)(mU)(mA) | |
| | (mG)(mC)(rU)(rC)(rC)(rC)(rU)(mU)(mU)#(mC)#(mU)#(mC) | |
| CR42- | (mC)#(mU)#(mG)#(mA)(mA)(rC)(rU)(rU)(mG)(mU)(mG)(rG)(mC)(fC) | 261 |
| 136_MCV | (fG)(mU)(fU)(mU)(rA)(rC)(rG)(mU)(rC)(rG)(mG)(rU)(rU)(rG)(mU)(mA) | |
| | (mG)(mC)(rU)(rC)(rC)(rC)(rU)(mU)(mU)#(mC)#(mU)#(mC) | |
| CR42- | (mC)#(mU)#(mG)#(mA)(mA)(rC)(rU)(rU)(mG)(mU)(mG)(rG)(mC)(fC) | 262 |
| 137_MCV | (fG)(fU)(mU)(mU)(rA)(rC)(rG)(mU)(rC)(rG)(mG)(rU)(rU)(rG)(mU)(mA) | |
| | (mG)(mC)(rU)(rC)(rC)(rC)(rU)(mU)(mU)#(mC)#(mU)#(mC) | |
| CR42- | (mC)#(mU)#(mG)#(mA)(mA)(rC)(rU)(rU)(mG)(mU)(mG)(rG)(mC)(fC) | 263 |
| 138_MCV | (mG)(mU)(fU)(mU)(rA)(rC)(rG)(mU)(rC)(rG)(mG)(rU)(rU)(rG)(mU) | |
| | (mA)(mG)(mC)(rU)(rC)(rC)(rC)(rU)(mU)(mU)#(mC)#(mU)#(mC) | |
| CR42- | (mC)#(mU)#(mG)#(mA)(mA)(rC)(rU)(rU)(mG)(mU)(mG)(rG)(mC)(fC) | 264 |
| 139_MCV | (fG)(mU)(mU)(mU)(rA)(rC)(rG)(mU)(rC)(rG)(mG)(rU)(rU)(rG)(mU)(mA) | |
| | (mG)(mC)(rU)(rC)(rC)(rC)(rU)(mU)(mU)#(mC)#(mU)#(mC) | |
| CR42- | (mC)#(mU)#(mG)#(mA)(mA)(rC)(rU)(rU)(mG)(mU)(mG)(rG)(mC)(fC) | 265 |
| 140_MCV | (mG)(fU)(mU)(mU)(rA)(rC)(rG)(mU)(rC)(rG)(mG)(rU)(rU)(rG)(mU)(mA) | |
| | (mG)(mC)(rU)(rC)(rC)(rC)(rU)(mU)(mU)#(mC)#(mU)#(mC) | |
| CR42- | (mC)#(mU)#(mG)#(mA)(mA)(rC)(rU)(rU)(mG)(mU)(mG)(rG)(mC)(fC) | 266 |
| 141_MCV | (mG)(mU)(mU)(mU)(rA)(rC)(rG)(mU)(rC)(rG)(mG)(rU)(rU)(rG)(mU) | |
| | (mA)(mG)(mC)(rU)(rC)(rC)(rC)(rU)(mU)(mU)#(mC)#(mU)#(mC) | |
| CR42- | (mC)#(mU)#(mG)#(mA)(mA)(mC)(dU)#(mU)(mG)(mU)(mG)(rG)(mC) | 267 |
| 142_MCV | (fC)(mG)(fU)(mU)(mU)(rA)(rC)(rG)(mU)(rC)(rG)(mG)(rU)(rU)(rG)(mU) | |
| | (mA)(mG)(mC)(rU)(rC)(rC)(rC)(rU)(mU)(mU)#(mC)#(mU)#(mC) | |
| CR42- | (mC)#(mU)#(mG)#(mA)(mA)(mC)(dU)#(mU)(mG)(mU)(mG)(rG)(mC) | 268 |
| 143_MCV | (fC)(mG)(mU)(mU)(mU)(rA)(rC)(rG)(mU)(rC)(rG)(mG)(rU)(rU)(rG)(mU) | |
| | (mA)(mG)(mC)(rU)(rC)(rC)(rC)(rU)(mU)(mU)#(mC)#(mU)#(mC) | |
| CR42- | (mC)#(mU)#(mG)#(mA)(mA)(fC)(dU)#(mU)(mG)(mU)(mG)(rG)(mC) | 269 |
| 144_MCV | (fC)(mG)(fU)(mU)(mU)(rA)(rC)(rG)(mU)(rC)(rG)(mG)(rU)(rU)(rG)(mU) | |
| | (mA)(mG)(mC)(rU)(rC)(rC)(rC)(rU)(mU)(mU)#(mC)#(mU)#(mC) | |
| CR42- | (mC)#(mU)#(mG)#(mA)(mA)(fC)(dU)#(mU)(mG)(mU)(mG)(rG)(mC) | 270 |
| 145_MCV | (fC)(mG)(mU)(mU)(mU)(rA)(rC)(rG)(mU)(rC)(rG)(mG)(rU)(rU)(rG)(mU) | |
| | (mA)(mG)(mC)(rU)(rC)(rC)(rC)(rU)(mU)(mU)#(mC)#(mU)#(mC) | |
| CR42- | (mC)#(mU)#(mG)#(mA)(mA)(mC)(dU)#(mU)(mG)(mU)(mG)(fG)(mC) | 271 |
| 146_MCV | (rC)(rG)(rU)(rU)(mU)(rA)(rC)(rG)(mU)(rC)(rG)(mG)(rU)(rU)(rG)(mU) | |
| | (mA)(mG)(mC)(rU)(rC)(rC)(rC)(rU)(mU)(mU)#(mC)#(mU)#(mC) | |
| CR42- | (mC)#(mU)#(mG)#(mA)(mA)(mC)(dU)#(mU)(mG)(mU)(mG)(mG)(mC) | 272 |
| 147_MCV | (rC)(rG)(rU)(rU)(mU)(rA)(rC)(rG)(mU)(rC)(rG)(mG)(rU)(rU)(rG)(mU) | |
| | (mA)(mG)(mC)(rU)(rC)(rC)(rC)(rU)(mU)(mU)#(mC)#(mU)#(mC) | |
| CR42- | (mC)#(mU)#(mG)#(mA)(mA)(fC)(dU)#(mU)(mG)(mU)(mG)(fG)(mC) | 273 |
| 148_MCV | (rC)(rG)(rU)(rU)(mU)(rA)(rC)(rG)(mU)(rC)(rG)(mG)(rU)(rU)(rG)(mU) | |
| | (mA)(mG)(mC)(rU)(rC)(rC)(rC)(rU)(mU)(mU)#(mC)#(mU)#(mC) | |
| CR42- | (mC)#(mU)#(mG)#(mA)(mA)(fC)(dU)#(mU)(mG)(mU)(mG)(mG)(mC) | 274 |
| 149_MCV | (rC)(rG)(rU)(rU)(mU)(rA)(rC)(rG)(mU)(rC)(rG)(mG)(rU)(rU)(rG)(mU) | |
| | (mA)(mG)(mC)(rU)(rC)(rC)(rC)(rU)(mU)(mU)#(mC)#(mU)#(mC) | |
| CR42- | (mC)#(mU)#(mG)#(mA)(mA)(rC)(rU)(rU)(mG)(mU)(mG)(fG)(mC)(fC) | 275 |
| 150_MCV | (mG)(fU)(mU)(mU)(rA)(rC)(rG)(mU)(rC)(rG)(mG)(rU)(rU)(rG)(mU) | |
| | (mA)(mG)(mC)(rU)(rC)(rC)(rC)(rU)(mU)(mU)#(mC)#(mU)#(mC) | |
| CR42- | (mC)#(mU)#(mG)#(mA)(mA)(rC)(rU)(rU)(mG)(mU)(mG)(mG)(mC)(fC) | 276 |
| 151_MCV | (mG)(fU)(mU)(mU)(rA)(rC)(rG)(mU)(rC)(rG)(mG)(rU)(rU)(rG)(mU) | |
| | (mA)(mG)(mC)(rU)(rC)(rC)(rC)(rU)(mU)(mU)#(mC)#(mU)#(mC) | |
| CR42- | (mC)#(mU)#(mG)#(mA)(mA)(rC)(rU)(rU)(mG)(mU)(mG)(fG)(mC)(fC) | 277 |
| 152_MCV | (mG)(mU)(mU)(mU)(rA)(rC)(rG)(mU)(rC)(rG)(mG)(rU)(rU)(rG)(mU) | |
| | (mA)(mG)(mC)(rU)(rC)(rC)(rC)(rU)(mU)(mU)#(mC)#(mU)#(mC) | |
| CR42- | (mC)#(mU)#(mG)#(mA)(mA)(rC)(rU)(rU)(mG)(mU)(mG)(mG)(mC)(fC) | 278 |
| 153_MCV | (mG)(mU)(mU)(mU)(rA)(rC)(rG)(mU)(rC)(rG)(mG)(rU)(rU)(rG)(mU) | |
| | (mA)(mG)(mC)(rU)(rC)(rC)(rC)(rU)(mU)(mU)#(mC)#(mU)#(mC) | |
| CR42- | (mC)#(mU)#(mG)#(mA)(mA)(mC)(dU)#(mU)(mG)(mU)(mG)(fG)(mC) | 279 |
| 154_MCV | (fC)(mG)(fU)(mU)(mU)(rA)(rC)(rG)(mU)(rC)(rG)(mG)(rU)(rU)(rG)(mU) | |
| | (mA)(mG)(mC)(rU)(rC)(rC)(rC)(rU)(mU)(mU)#(mC)#(mU)#(mC) | |
| CR42- | (mC)#(mU)#(mG)#(mA)(mA)(mC)(dU)#(mU)(mG)(mU)(mG)(mG)(mC) | 280 |
| 155_MCV | (fC)(mG)(fU)(mU)(mU)(rA)(rC)(rG)(mU)(rC)(rG)(mG)(rU)(rU)(rG)(mU) | |
| | (mA)(mG)(mC)(rU)(rC)(rC)(rC)(rU)(mU)(mU)#(mC)#(mU)#(mC) | |
| CR42- | (mC)#(mU)#(mG)#(mA)(mA)(fC)(dU)#(mU)(mG)(mU)(mG)(fG)(mC) | 281 |
| 156_MCV | (fC)(mG)(fU)(mU)(mU)(rA)(rC)(rG)(mU)(rC)(rG)(mG)(rU)(rU)(rG)(mU) | |
| | (mA)(mG)(mC)(rU)(rC)(rC)(rC)(rU)(mU)(mU)#(mC)#(mU)#(mC) | |
| CR42- | (mC)#(mU)#(mG)#(mA)(mA)(fC)(dU)#(mU)(mG)(mU)(mG)(mG)(mC) | 282 |
| 157_MCV | (fC)(mG)(fU)(mU)(mU)(rA)(rC)(rG)(mU)(rC)(rG)(mG)(rU)(rU)(rG)(mU) | |
| | (mA)(mG)(mC)(rU)(rC)(rC)(rC)(rU)(mU)(mU)#(mC)#(mU)#(mC) | |
| CR42- | (mC)#(mU)#(mG)#(mA)(mA)(mC)(dU)#(mU)(mG)(mU)(mG)(fG)(mC) | 283 |
| 158_MCV | (fC)(mG)(mU)(mU)(mU)(rA)(rC)(rG)(mU)(rC)(rG)(mG)(rU)(rU)(rG)(mU) | |
| | (mA)(mG)(mC)(rU)(rC)(rC)(rC)(rU)(mU)(mU)#(mC)#(mU)#(mC) | |
| CR42- | (mC)#(mU)#(mG)#(mA)(mA)(mC)(dU)#(mU)(mG)(mU)(mG)(mG)(mC) | 284 |
| 159_MCV | (fC)(mG)(mU)(mU)(mU)(rA)(rC)(rG)(mU)(rC)(rG)(mG)(rU)(rU)(rG) | |
| | (mU)(mA)(mG)(mC)(rU)(rC)(rC)(rC)(rU)(mU)(mU)#(mC)#(mU)#(mC) | |
| CR42- | (mC)#(mU)#(mG)#(mA)(mA)(fC)(dU)#(mU)(mG)(mU)(mG)(fG)(mC) | 285 |
| 160_MCV | (fC)(mG)(mU)(mU)(mU)(rA)(rC)(rG)(mU)(rC)(rG)(mG)(rU)(rU)(rG)(mU) | |
| | (mA)(mG)(mC)(rU)(rC)(rC)(rC)(rU)(mU)(mU)#(mC)#(mU)#(mC) | |
| CR42- | (mC)#(mU)#(mG)#(mA)(mA)(fC)(dU)#(mU)(mG)(mU)(mG)(mG)(mC) | 286 |
| 161_MCV | (fC)(mG)(mU)(mU)(mU)(rA)(rC)(rG)(mU)(rC)(rG)(mG)(rU)(rU)(rG)(mU) | |
| | (mA)(mG)(mC)(rU)(rC)(rC)(rC)(rU)(mU)(mU)#(mC)#(mU)#(mC) | |
| CR42- | (mC)#(mU)#(mG)#(mA)(mA)(rC)(rU)(rU)(mG)(mU)(mG)(rG)(mC)(rC) | 287 |
| 162_MCV | (rG)(rU)(rU)(mU)(rA)(fC)(mG)(mU)(rC)(rG)(mG)(rU)(rU)(rG)(mU)(mA) | |
| | (mG)(mC)(rU)(rC)(rC)(rC)(rU)(mU)(mU)#(mC)#(mU)#(mC) | |
| CR42- | (mC)#(mU)#(mG)#(mA)(mA)(rC)(rU)(rU)(mG)(mU)(mG)(rG)(mC)(rC) | 288 |
| 163_MCV | (rG)(rU)(rU)(mU)(rA)(fC)(fG)(mU)(rC)(rG)(mG)(rU)(rU)(rG)(mU)(mA) | |
| | (mG)(mC)(rU)(rC)(rC)(rC)(rU)(mU)(mU)#(mC)#(mU)#(mC) | |
| CR42- | (mC)#(mU)#(mG)#(mA)(mA)(rC)(rU)(rU)(mG)(mU)(mG)(rG)(mC)(rC) | 289 |
| 164_MCV | (rG)(rU)(rU)(mU)(rA)(mC)(mG)(mU)(rC)(rG)(mG)(rU)(rU)(rG)(mU)(mA) | |
| | (mG)(mC)(rU)(rC)(rC)(rC)(rU)(mU)(mU)#(mC)#(mU)#(mC) | |
| CR42- | (mC)#(mU)#(mG)#(mA)(mA)(rC)(rU)(rU)(mG)(mU)(mG)(rG)(mC)(rC) | 290 |
| 165_MCV | (rG)(rU)(rU)(mU)(rA)(mC)(fG)(mU)(rC)(rG)(mG)(rU)(rU)(rG)(mU)(mA) | |
| | (mG)(mC)(rU)(rC)(rC)(rC)(rU)(mU)(mU)#(mC)#(mU)#(mC) | |
| CR42- | (mC)#(mU)#(mG)#(mA)(mA)(rC)(rU)(rU)(mG)(mU)(mG)(rG)(mC)(rC) | 291 |
| 166_MCV | (rG)(rU)(rU)(mU)(rA)(rC)(rG)(mU)(mC)(mG)(mG)(rU)(rU)(rG)(mU)(mA) | |
| | (mG)(mC)(rU)(rC)(rC)(rC)(rU)(mU)(mU)#(mC)#(mU)#(mC) | |
| CR42- | (mC)#(mU)#(mG)#(mA)(mA)(rC)(rU)(rU)(mG)(mU)(mG)(rG)(mC)(rC) | 292 |
| 167_MCV | (rG)(rU)(rU)(mU)(rA)(fC)(mG)(mU)(mC)(mG)(mG)(rU)(rU)(rG)(mU) | |
| | (mA)(mG)(mC)(rU)(rC)(rC)(rC)(rU)(mU)(mU)#(mC)#(mU)#(mC) | |
| CR42- | (mC)#(mU)#(mG)#(mA)(mA)(rC)(rU)(rU)(mG)(mU)(mG)(rG)(mC)(rC) | 293 |
| 168_MCV | (rG)(rU)(rU)(mU)(rA)(fC)(fG)(mU)(mC)(mG)(mG)(rU)(rU)(rG)(mU)(mA) | |
| | (mG)(mC)(rU)(rC)(rC)(rC)(rU)(mU)(mU)#(mC)#(mU)#(mC) | |
| CR42- | (mC)#(mU)#(mG)#(mA)(mA)(rC)(rU)(rU)(mG)(mU)(mG)(rG)(mC)(rC) | 294 |
| 169_MCV | (rG)(rU)(rU)(mU)(rA)(mC)(mG)(mU)(mC)(mG)(mG)(rU)(rU)(rG)(mU) | |
| | (mA)(mG)(mC)(rU)(rC)(rC)(rC)(rU)(mU)(mU)#(mC)#(mU)#(mC) | |
| CR42- | (mC)#(mU)#(mG)#(mA)(mA)(rC)(rU)(rU)(mG)(mU)(mG)(rG)(mC)(rC) | 295 |
| 170_MCV | (rG)(rU)(rU)(mU)(rA)(mC)(fG)(mU)(mC)(mG)(mG)(rU)(rU)(rG)(mU) | |
| | (mA)(mG)(mC)(rU)(rC)(rC)(rC)(rU)(mU)(mU)#(mC)#(mU)#(mC) | |
| CR42- | (mC)#(mU)#(mG)#(mA)(mA)(rC)(rU)(rU)(mG)(mU)(mG)(rG)(mC)(rC) | 296 |
| 171_MCV | (rG)(rU)(rU)(mU)(fA)(rC)(rG)(mU)(mC)(mG)(mG)(rU)(rU)(rG)(mU)(mA) | |
| | (mG)(mC)(rU)(rC)(rC)(rC)(rU)(mU)(mU)#(mC)#(mU)#(mC) | |
| CR42- | (mC)#(mU)#(mG)#(mA)(mA)(rC)(rU)(rU)(mG)(mU)(mG)(rG)(mC)(rC) | 297 |
| 172_MCV | (rG)(rU)(rU)(mU)(dA)#(rC)(rG)(mU)(mC)(mG)(mG)(rU)(rU)(rG)(mU) | |
| | (mA)(mG)(mC)(rU)(rC)(rC)(rC)(rU)(mU)(mU)#(mC)#(mU)#(mC) | |
| CR42- | (mC)#(mU)#(mG)#(mA)(mA)(rC)(rU)(rU)(mG)(mU)(mG)(rG)(mC)(rC) | 298 |
| 173_MCV | (rG)(rU)(rU)(mU)(mA)(rC)(rG)(mU)(mC)(mG)(mG)(rU)(rU)(rG)(mU) | |
| | (mA)(mG)(mC)(rU)(rC)(rC)(rC)(rU)(mU)(mU)#(mC)#(mU)#(mC) | |
| CR42- | (mC)#(mU)#(mG)#(mA)(mA)(rC)(rU)(rU)(mG)(mU)(mG)(rG)(mC)(rC) | 299 |
| 174_MCV | (rG)(rU)(rU)(mU)(rA)#(rC)(rG)(mU)(mC)(mG)(mG)(rU)(rU)(rG)(mU) | |
| | (mA)(mG)(mC)(rU)(rC)(rC)(rC)(rU)(mU)(mU)#(mC)#(mU)#(mC) | |
| CR42- | (mC)#(mU)#(mG)#(mA)(mA)(rC)(rU)(rU)(mG)(mU)(mG)(rG)(mC)(rC) | 300 |
| 175_MCV | (rG)(rU)(rU)(mU)(rA)(rC)(rG)(mU)(rC)(rG)(mG)(rU)#(rU)#(rG)#(mU) | |
| | (mA)(mG)(mC)(rU)(rC)(rC)(rC)(rU)(mU)(mU)#(mC)#(mU)#(mC) | |
| CR42- | (mC)#(mU)#(mG)#(mA)(mA)(rC)(rU)(rU)(mG)(mU)(mG)(rG)(mC)(rC) | 301 |
| 176_MCV | (rG)(rU)(rU)(mU)(rA)(rC)(rG)(mU)(rC)(rG)(mG)(rU)#(rU)#(mG)(mU) | |
| | (mA)(mG)(mC)(rU)(rC)(rC)(rC)(rU)(mU)(mU)#(mC)#(mU)#(mC) | |
| CR42- | (mC)#(mU)#(mG)#(mA)(mA)(rC)(rU)(rU)(mG)(mU)(mG)(rG)(mC)(rC) | 302 |
| 177_MCV | (rG)(rU)(rU)(mU)(rA)(rC)(rG)(mU)(rC)(rG)(mG)(rU)#(rU)(rG)#(mU)(mA) | |
| | (mG)(mC)(rU)(rC)(rC)(rC)(rU)(mU)(mU)#(mC)#(mU)#(mC) | |
| CR42- | (mC)#(mU)#(mG)#(mA)(mA)(rC)(rU)(rU)(mG)(mU)(mG)(rG)(mC)(rC) | 303 |
| 178_MCV | (rG)(rU)(rU)(mU)(rA)(rC)(rG)(mU)(rC)(rG)(mG)(rU)#(rU)(mG)(mU)(mA) | |
| | (mG)(mC)(rU)(rC)(rC)(rC)(rU)(mU)(mU)#(mC)#(mU)#(mC) | |
| CR42- | (mC)#(mU)#(mG)#(mA)(mA)(rC)(rU)(rU)(mG)(mU)(mG)(rG)(mC)(rC) | 304 |
| 179_MCV | (rG)(rU)(rU)(mU)(rA)(rC)(rG)(mU)(rC)(rG)(mG)(rU)(rU)(rG)(mU)(mA) | |
| | (mG)(mC)(rU)#(rC)#(rC)#(rC)(rU)#(mU)(mU)#(mC)#(mU)#(mC) | |
| CR42- | (mC)#(mU)#(mG)#(mA)(mA)(rC)(rU)(rU)(mG)(mU)(mG)(rG)(mC)(rC) | 305 |
| 180_MCV | (rG)(rU)(rU)(mU)(rA)(rC)(rG)(mU)(rC)(rG)(mG)(rU)(rU)(rG)(mU)(mA) | |
| | (mG)(mC)(mU)(rC)#(rC)#(rC)(rU)#(mU)(mU)#(mC)#(mU)#(mC) | |
| CR42- | (mC)#(mU)#(mG)#(mA)(mA)(rC)(rU)(rU)(mG)(mU)(mG)(rG)(mC)(rC) | 306 |
| 181_MCV | (rG)(rU)(rU)(mU)(rA)(rC)(rG)(mU)(rC)(rG)(mG)(rU)(rU)(rG)(mU)(mA) | |
| | (mG)(mC)(rU)#(mC)(rC)#(rC)(rU)#(mU)(mU)#(mC)#(mU)#(mC) | |
| CR42- | (mC)#(mU)#(mG)#(mA)(mA)(rC)(rU)(rU)(mG)(mU)(mG)(rG)(mC)(rC) | 307 |
| 182_MCV | (rG)(rU)(rU)(mU)(rA)(rC)(rG)(mU)(rC)(rG)(mG)(rU)(rU)(rG)(mU)(mA) | |
| | (mG)(mC)(rU)#(rC)#(mC)(rC)(rU)#(mU)(mU)#(mC)#(mU)#(mC) | |
| CR42- | (mC)#(mU)#(mG)#(mA)(mA)(rC)(rU)(rU)(mG)(mU)(mG)(rG)(mC)(rC) | 308 |
| 183_MCV | (rG)(rU)(rU)(mU)(rA)(rC)(rG)(mU)(rC)(rG)(mG)(rU)(rU)(rG)(mU)(mA) | |
| | (mG)(mC)(rU)#(rC)#(rC)(rC)(rU)#(mU)(mU)#(mC)#(mU)#(mC) | |
| CR42- | (mC)#(mU)#(mG)#(mA)(mA)(rC)(rU)(rU)(mG)(mU)(mG)(rG)(mC)(rC) | 309 |
| 184_MCV | (rG)(rU)(rU)(mU)(rA)(rC)(rG)(mU)(rC)(rG)(mG)(rU)(rU)(rG)(mU)(mA) | |
| | (mG)(mC)(mU)(rC)#(rC)(rC)(rU)#(mU)(mU)#(mC)#(mU)#(mC) | |
| CR42- | (mC)#(mU)#(mG)#(mA)(mA)(rC)(rU)(rU)(mG)(mU)(mG)(rG)(mC)(rC) | 310 |
| 185_MCV | (rG)(rU)(rU)(mU)(rA)(rC)(rG)(mU)(rC)(rG)(mG)(rU)(rU)(rG)(mU)(mA) | |
| | (mG)(mC)(rU)#(mC)(rC)(rC)(rU)#(mU)(mU)#(mC)#(mU)#(mC) | |
| CR42- | (mC)#(mU)#(mG)#(mA)(mA)(rC)(rU)(rU)(mG)(mU)(mG)(rG)(mC)(rC) | 311 |
| 186_MCV | (rG)(rU)(rU)(mU)(rA)(rC)(rG)(mU)(rC)(rG)(mG)(rU)(rU)(rG)(mU)(mA) | |
| | (mG)(mC)(rU)#(rC)(rC)#(rC)(rU)#(mU)(mU)#(mC)#(mU)#(mC) | |
| CR42- | (mC)#(mU)#(mG)#(mA)(mA)(rC)(rU)(rU)(mG)(mU)(mG)(rG)(mC)(rC) | 312 |
| 187_MCV | (rG)(rU)(rU)(mU)(rA)(rC)(rG)(mU)(rC)(rG)(mG)(rU)(rU)(rG)(mU)(mA) | |
| | (mG)(mC)(rU)#(rC)(mC)(rC)(rU)#(mU)(mU)#(mC)#(mU)#(mC) | |
| CR42- | (mC)#(mU)#(mG)#(mA)(mA)(rC)(rU)(rU)(mG)(mU)(mG)(rG)(mC)(rC) | 304 |
| 188_MCV | (rG)(rU)(rU)(mU)(rA)(rC)(rG)(mU)(rC)(rG)(mG)(rU)(rU)(rG)(mU)(mA) | |
| | (mG)(mC)(rU)#(rC)#(rC)#(rC)(rU)#(mU)(mU)#(mC)#(mU)#(mC) | |
| CR42- | (mC)#(mU)#(mG)#(mA)(mA)(rC)(rU)(rU)(mG)(mU)(mG)(rG)(mC)(rC) | 313 |
| 189_MCV | (rG)(rU)(rU)(mU)(rA)(rC)(rG)(mU)(rC)(rG)(mG)(rU)#(rU)(rG)(mU)(mA) | |
| | (mG)(mC)(rU)#(rC)#(rC)#(rC)(rU)#(mU)(mU)#(mC)#(mU)#(mC) | |
| CR48- | (mC)#(mU)#(mG)#(rA)(rA)(rC)(rU)(rU)(rG)(rU)(rG)(rG)(rC)(rC)(rG)(rU) | 314 |
| 0_MCV | (rU)(rU)(rA)(rC)(rG)(rU)(rC)(rG)(rG)(rU)(rU)(rG)(rU)(rA)(rG)(rC)(rU) | |
| | (rC)(rC)(rC)(rU)(rU)(rU)(rC)(rU)(rC)(rA)(rU)(rU)#(mU)#(mC)#(mG) | |
| CR48- | (mC)#(mU)#(mG)#(mA)(mA)(rC)(rU)(rU)(rG)(rU)(rG)(rG)(rC)(rC)(rG) | 315 |
| 2_MCV | (rU)(rU)(rU)(rA)(rC)(rG)(rU)(rC)(rG)(rG)(rU)(rU)(rG)(rU)(rA)(rG)(rC) | |
| | (rU)(rC)(rC)(rC)(rU)(mU)(mU)(mC)(mU)(mC)(mA)(mU)(mU)#(mU)#(mC)# | |
| | (mG) | |
| CR48- | (mC)#(mU)#(mG)#(mA)(mA)(rC)(rU)(rU)(mG)(mU)(mG)(rG)(mC)(rC) | 316 |
| 4_MCV | (rG)(rU)(rU)(mU)(rA)(rC)(rG)(mU)(rC)(rG)(mG)(rU)(rU)(rG)(mU)(mA) | |
| | (mG)(mC)(rU)(rC)(rC)(rC)(rU)(mU)(mU)(mC)(mU)(mC)(mA)(mU)(mU) | |
| | #(mU)#(mC)#(mG) | |
| CR48- | (mC)#(mU)#(mG)#(mA)(mA)(mC)(dT)#(mU)(mG)(mU)(mG)(rG)(mC) | 317 |
| 128_MCV | (rC)(rG)(rU)(rU)(mU)(rA)(rC)(rG)(mU)(rC)(rG)(mG)(rU)(rU)(rG)(mU) | |
| | (mA)(mG)(mC)(rU)(rC)(rC)(rC)(rU)(mU)(mU)(mC)(mU)(mC)(mA)(mU) | |
| | (mU)#(mU)#(mC)#(mG) | |
| CR48- | (mC)#(mU)#(mG)#(mA)(mA)(rC)(rU)(rU)(mG)(mU)(mG)(rG)(mC)(fC) | 318 |
| 140_MCV | (mG)(fU)(mU)(mU)(rA)(rC)(rG)(mU)(rC)(rG)(mG)(rU)(rU)(rG)(mU) | |
| | (mA)(mG)(mC)(rU)(rC)(rC)(rC)(rU)(mU)(mU)(mC)(mU)(mC)(mA)(mU) | |
| | (mU#(mU)#(mC)#(mG) | |
| CR48- | (mC)#(mU)#(mG)#(mA)(mA)(mC)(dT)#(mU)(mG)(mU)(mG)(rG)(mC) | 319 |
| 142_MCV | (fC)(mG)(fU)(mU)(mU)(rA)(rC)(rG)(mU)(rC)(rG)(mG)(rU)(rU)(rG)(mU) | |
| | (mA)(mG)(mC)(rU)(rC)(rC)(rC)(rU)(mU)(mU)(mC)(mU)(mC)(mA)(mU) | |
| | (mU)#(mU)#(mC)#(mG) | |
| CR48- | (mC)#(mU)#(mG)#(mA)(mA)(mC)(dT)#(mU)(mG)(mU)(mG)(mG)(mC) | 320 |
| 155_MCV | (fC)(mG)(fU)(mU)(mU)(rA)(rC)(rG)(mU)(rC)(rG)(mG)(rU)(rU)(rG)(mU) | |
| | (mA)(mG)(mC)(rU)(rC)(rC)(rC)(rU)(mU)(mU)(mC)(mU)(mC)(mA)(mU) | |
| | (mU)#(mU)#(mC)#(mG) | |
| CR48- | (mC)#(mU)#(mG)#(mA)(mA)(rC)(rU)(rU)(mG)(mU)(mG)(rG)(mC)(rC) | 321 |
| 165_MCV | (rG)(rU)(rU)(mU)(rA)(mC)(fG)(mU)(rC)(rG)(mG)(rU)(rU)(rG)(mU)(mA) | |
| | (mG)(mC)(rU)(rC)(rC)(rC)(rU)(mU)(mU)(mC)(mU)(mC)(mA)(mU)(mU) | |
| | #(mU)#(mC)#(mG) | |
| CR48- | (mC)#(mU)#(mG)#(mA)(mA)(rC)(rU)(rU)(mG)(mU)(mG)(rG)(mC)(rC) | 322 |
| 170_MCV | (rG)(rU)(rU)(mU)(rA)(mC)(fG)(mU)(mC)(mG)(mG)(rU)(rU)(rG)(mU)(mA) | |
| | (mG)(mC)(rU)(rC)(rC)(rC)(rU)(mU)(mU)(mC)(mU)(mC)(mA)(mU)(mU) | |
| | #(mU)#(mC)#(mG) | |
| CR48- | (mC)#(mU)#(mG)#(mA)(mA)(rC)(rU)(rU)(mG)(mU)(mG)(rG)(mC)(rC) | 323 |
| 177_MCV | (rG)(rU)(rU)(mU)(rA)(rC)(rG)(mU)(rC)(rG)(mG)(rU)#(rU)(rG)#(mU)(mA) | |
| | (mG)(mC)(rU)(rC)(rC)(rC)(rU)(mU)(mU)(mC)(mU)(mC)(mA)(mU)(mU) | |
| | #(mU)#(mC)#(mG) | |
| CR48- | (mC)#(mU)#(mG)#(mA)(mA)(rC)(rU)(rU)(mG)(mU)(mG)(rG)(mC)(rC) | 324 |
| 186_MCV | (rG)(rU)(rU)(mU)(rA)(rC)(rG)(mU)(rC)(rG)(mG)(rU)(rU)(rG)(mU)(mA) | |
| | (mG)(mC)(rU)#(rC)(rC)#(rC)(rU)#(mU)(mU)(mC)(mU)(mC)(mA)(mU) | |
| | (mU)#(mU)#(mC)#(mG) | |
| CR48- | (mC)#(mU)#(mG)#(mA)(mA)(rC)(rU)(rU)(mG)(mU)(mG)(rG)(mC)(rC) | 325 |
| 189_MCV | (rG)(rU)(rU)(mU)(rA)(rC)(rG)(mU)(rC)(rG)(mG)(rU)#(rU)(rG)(mU)(mA) | |
| | (mG)(mC)(rU)#(rC)#(rC)#(rC)(rU)#(mU)(mU)(mC)(mU)(mC)(mA)(mU) | |
| | (mU)#(mU)#(mC)#(mG) | |
| CR48- | (mC)#(mU)#(mG)#(mA)(mA)(mC)(dT)#(mU)(mG)(mU)(mG)(fG)(mC) | 326 |
| 154_MCV | (fC)(mG)(fU)(mU)(mU)(rA)(rC)(rG)(mU)(rC)(rG)(mG)(rU)(rU)(rG)(mU) | |
| | (mA)(mG)(mC)(rU)(rC)(rC)(rC)(rU)(mU)(mU)(mC)(mU)(mC)(mA)(mU) | |
| | (mU)#(mU)#(mC)#(mG) | |
| CR48- | (mC)#(mU)#(mG)#(mA)(mA)(mC)(dT)#(mU)(mG)(mU)(mG)(fG)(mC) | 327 |
| 190_MCV | (fC)(mG)(fU)(mU)(mU)(rA)(fC)(mG)(mU)(mC)(mG)(mG)(rU)(rU)(rG) | |
| | (mU)(mA)(mG)(mC)(rU)(rC)(rC)(rC)(rU)(mU)(mU)(mC)(mU)(mC)(mA) | |
| | (mU)(mU)#(mU)#(mC)#(mG) | |
| CR48- | (mC)#(mU)#(mG)#(mA)(mA)(mC)(dT)#(mU)(mG)(mU)(mG)(fG)(mC) | 328 |
| 191_MCV | (fC)(mG)(fU)(mU)(mU)(rA)(mC)(fG)(mU)(mC)(mG)(mG)(rU)(rU)(rG) | |
| | (mU)(mA)(mG)(mC)(rU)(rC)(rC)(rC)(rU)(mU)(mU)(mC)(mU)(mC)(mA) | |
| | (mU)(mU)#(mU)#(mC)#(mG) | |
| CR48- | (mC)#(mU)#(mG)#(mA)(mA)(mC)(dT)#(mU)(mG)(mU)(mG)(mG)(mC) | 329 |
| 192_MCV | (fC)(mG)(fU)(mU)(mU)(rA)(fC)(mG)(mU)(mC)(mG)(mG)(rU)(rU)(rG) | |
| | (mU)(mA)(mG)(mC)(rU)(rC)(rC)(rC)(rU)(mU)(mU)(mC)(mU)(mC)(mA) | |
| | (mU)(mU)#(mU)#(mC)#(mG) | |
| CR48- | (mC)#(mU)#(mG)#(mA)(mA)(mC)(dT)#(mU)(mG)(mU)(mG)(mG)(mC) | 330 |
| 193_MCV | (fC)(mG)(fU)(mU)(mU)(rA)(mC)(fG)(mU)(mC)(mG)(mG)(rU)(rU)(rG) | |
| | (mU)(mA)(mG)(mC)(rU)(rC)(rC)(rC)(rU)(mU)(mU)(mC)(mU)(mC)(mA) | |
| | (mU)(mU)#(mU)#(mC)#(mG) | |
| CR48- | (mC)#(mU)#(mG)#(mA)(mA)(mC)(dT)#(mU)(mG)(mU)(mG)(fG)(mC) | 331 |
| 194_MCV | (fC)(mG)(fU)(mU)(mU)(rA)#(fC)(mG)(mU)(mC)(mG)(mG)(rU)(rU)(rG) | |
| | (mU)(mA)(mG)(mC)(rU)(rC)(rC)(rC)(rU)(mU)(mU)(mC)(mU)(mC)(mA) | |
| | (mU)(mU)#(mU)#(mC)#(mG) | |
| CR48- | (mC)#(mU)#(mG)#(mA)(mA)(mC)(dT)#(mU)(mG)(mU)(mG)(fG)(mC) | 332 |
| 195_MCV | (fC)(mG)(fU)(mU)(mU)(rA)#(mC)(fG)(mU)(mC)(mG)(mG)(rU)(rU)(rG) | |
| | (mU)(mA)(mG)(mC)(rU)(rC)(rC)(rC)(rU)(mU)(mU)(mC)(mU)(mC)(mA) | |
| | (mU)(mU)#(mU)#(mC)#(mG) | |
| CR48- | (mC)#(mU)#(mG)#(mA)(mA)(mC)(dT)#(mU)(mG)(mU)(mG)(mG)(mC) | 333 |
| 196_MCV | (fC)(mG)(fU)(mU)(mU)(rA)#(fC)(mG)(mU)(mC)(mG)(mG)(rU)(rU)(rG) | |
| | (mU)(mA)(mG)(mC)(rU)(rC)(rC)(rC)(rU)(mU)(mU)(mC)(mU)(mC)(mA) | |
| | (mU)(mU)#(mU)#(mC)#(mG) | |
| CR48- | (mC)#(mU)#(mG)#(mA)(mA)(mC)(dT)#(mU)(mG)(mU)(mG)(mG)(mC) | 334 |
| 197_MCV | (fC)(mG)(fU)(mU)(mU)(rA)#(mC)(fG)(mU)(mC)(mG)(mG)(rU)(rU)(rG) | |
| | (mU)(mA)(mG)(mC)(rU)(rC)(rC)(rC)(rU)(mU)(mU)(mC)(mU)(mC)(mA) | |
| | (mU)(mU)#(mU)#(mC)#(mG) | |
| CR48- | (mC)#(mU)#(mG)#(mA)(mA)(mC)(dT)#(mU)(mG)(mU)(mG)(fG)(mC) | 335 |
| 198_MCV | (fC)(mG)(fU)(mU)(mU)(mA)(fC)(mG)(mU)(mC)(mG)(mG)(rU)(rU)(rG) | |
| | (mU)(mA)(mG)(mC)(rU)(rC)(rC)(rC)(rU)(mU)(mU)(mC)(mU)(mC)(mA) | |
| | (mU)(mU)#(mU)#(mC)#(mG) | |
| CR48- | (mC)#(mU)#(mG)#(mA)(mA)(mC)(dT)#(mU)(mG)(mU)(mG)(fG)(mC) | 336 |
| 199_MCV | (fC)(mG)(fU)(mU)(mU)(mA)(mC)(fG)(mU)(mC)(mG)(mG)(rU)(rU)(rG) | |
| | (mU)(mA)(mG)(mC)(rU)(rC)(rC)(rC)(rU)(mU)(mU)(mC)(mU)(mC)(mA) | |
| | (mU)(mU)#(mU)#(mC)#(mG) | |
| CR48- | (mC)#(mU)#(mG)#(mA)(mA)(mC)(dT)#(mU)(mG)(mU)(mG)(mG)(mC) | 337 |
| 200_MCV | (fC)(mG)(fU)(mU)(mU)(mA)(fC)(mG)(mU)(mC)(mG)(mG)(rU)(rU)(rG) | |
| | (mU)(mA)(mG)(mC)(rU)(rC)(rC)(rC)(rU)(mU)(mU)(mC)(mU)(mC)(mA) | |
| | (mU)(mU)#(mU)#(mC)#(mG) | |
| CR48- | (mC)#(mU)#(mG)#(mA)(mA)(mC)(dT)#(mU)(mG)(mU)(mG)(mG)(mC) | 338 |
| 201_MCV | (fC)(mG)(fU)(mU)(mU)(mA)(mC)(fG)(mU)(mC)(mG)(mG)(rU)(rU)(rG) | |
| | (mU)(mA)(mG)(mC)(rU)(rC)(rC)(rC)(rU)(mU)(mU)(mC)(mU)(mC)(mA) | |
| | (mU)(mU)#(mU)#(mC)#(mG) | |
| CR48- | (mC)#(mU)#(mG)#(mA)(mA)(mC)(dT)#(mU)(mG)(mU)(mG)(fG)(mC) | 339 |
| 202_MCV | (fC)(mG)(fU)(mU)(mU)(fA)(fC)(mG)(mU)(mC)(mG)(mG)(rU)(rU)(rG) | |
| | (mU)(mA)(mG)(mC)(rU)(rC)(rC)(rC)(rU)(mU)(mU)(mC)(mU)(mC)(mA) | |
| | (mU)(mU)#(mU)#(mC)#(mG) | |
| CR48- | (mC)#(mU)#(mG)#(mA)(mA)(mC)(dT)#(mU)(mG)(mU)(mG)(fG)(mC) | 340 |
| 203_MCV | (fC)(mG)(fU)(mU)(mU)(fA)(mC)(fG)(mU)(mC)(mG)(mG)(rU)(rU)(rG) | |
| | (mU)(mA)(mG)(mC)(rU)(rC)(rC)(rC)(rU)(mU)(mU)(mC)(mU)(mC)(mA) | |
| | (mU)(mU)#(mU)#(mC)#(mG) | |
| CR48- | (mC)#(mU)#(mG)#(mA)(mA)(mC)(dT)#(mU)(mG)(mU)(mG)(mG)(mC) | 341 |
| 204_MCV | (fC)(mG)(fU)(mU)(mU)(fA)(fC)(mG)(mU)(mC)(mG)(mG)(rU)(rU)(rG) | |
| | (mU)(mA)(mG)(mC)(rU)(rC)(rC)(rC)(rU)(mU)(mU)(mC)(mU)(mC)(mA) | |
| | (mU)(mU)#(mU)#(mC)#(mG) | |
| CR48- | (mC)#(mU)#(mG)#(mA)(mA)(mC)(dT)#(mU)(mG)(mU)(mG)(mG)(mC) | 342 |
| 205_MCV | (fC)(mG)(fU)(mU)(mU)(fA)(mC)(fG)(mU)(mC)(mG)(mG)(rU)(rU)(rG) | |
| | (mU)(mA)(mG)(mC)(rU)(rC)(rC)(rC)(rU)(mU)(mU)(mC)(mU)(mC)(mA) | |
| | (mU)(mU)#(mU)#(mC)#(mG) | |
| CR48- | (mC)#(mU)#(mG)#(mA)(mA)(mC)(dT)#(mU)(mG)(mU)(mG)(mG)(mC) | 343 |
| 206_MCV | (fC)(mG)(fU)(mU)(mU)(rA)(rC)(rG)(mU)(rC)(rG)(mG)(rU)(rU)(mG) | |
| | (mU)(mA)(mG)(mC)(rU)(rC)(mC)(rC)(rU)(mU)(mU)(mC)(mU)(mC)(mA) | |
| | (mU)(mU)#(mU)#(mC)#(mG) | |
| CR48- | (mC)#(mU)#(mG)#(mA)(mA)(mC)(dT)#(mU)(mG)(mU)(mG)(mG)(mC) | 344 |
| 207_MCV | (fC)(mG)(fU)(mU)(mU)(rA)(rC)(rG)(mU)(rC)(rG)(mG)(rU)(rU)(mG) | |
| | (mU)(mA)(mG)(mC)(rU)(rC)(rC)(mC)(rU)(mU)(mU)(mC)(mU)(mC)(mA) | |
| | (mU)(mU)#(mU)#(mC)#(mG) | |
| CR48- | (mC)#(mU)#(mG)#(mA)(mA)(mC)(dT)#(mU)(mG)(mU)(mG)(mG)(mC) | 345 |
| 208_MCV | (fC)(mG)(fU)(mU)(mU)(rA)(rC)(rG)(mU)(rC)(rG)(mG)(rU)(rU)(rG)(mU) | |
| | (mA)(mG)(mC)(rU)(rC)(mC)(mC)(rU)(mU)(mU)(mC)(mU)(mC)(mA) | |
| | (mU)(mU)#(mU)#(mC)#(mG) | |
| CR48- | (mC)#(mU)#(mG)#(mA)(mA)(mC)(dT)#(mU)(mG)(mU)(mG)(mG)(mC) | 346 |
| 209_MCV | (fC)(mG)(fU)(mU)(mU)(rA)(rC)(rG)(mU)(rC)(rG)(mG)(rU)(rU)(mG) | |
| | (mU)(mA)(mG)(mC)(rU)(rC)(mC)(mC)(rU)(mU)(mU)(mC)(mU)(mC)(mA) | |
| | (mU)(mU)#(mU)#(mC)#(mG) | |
| CR48- | (mC)#(mU)#(mG)#(mA)(mA)(mC)(dT)#(mU)(mG)(mU)(mG)(mG)(mC) | 347 |
| 210_MCV | (fC)(mG)(fU)(mU)(mU)(rA)(rC)(rG)(mU)(rC)(rG)(mG)(rU)#(rU)(mG) | |
| | (mU)(mA)(mG)(mC)(rU)#(rC)(mC)(rC)(rU)#(mU)(mU)(mC)(mU)(mC) | |
| | (mA)(mU)(mU)#(mU)#(mC)#(mG) | |
| CR48- | (mC)#(mU)#(mG)#(mA)(mA)(mC)(dT)#(mU)(mG)(mU)(mG)(mG)(mC) | 348 |
| 211_MCV | (fC)(mG)(fU)(mU)(mU)(rA)(rC)(rG)(mU)(rC)(rG)(mG)(rU)#(rU)(mG) | |
| | (mU)(mA)(mG)(mC)(rU)#(rC)#(mC)(rC)(rU)#(mU)(mU)(mC)(mU)(mC) | |
| | (mA)(mU)(mU)#(mU)#(mC)#(mG) | |
| CR48- | (mC)#(mU)#(mG)#(mA)(mA)(mC)(dT)#(mU)(mG)(mU)(mG)(mG)(mC) | 349 |
| 212_MCV | (fC)(mG)(fU)(mU)(mU)(rA)(rC)(rG)(mU)(rC)(rG)(mG)(rU)#(rU)(mG) | |
| | (mU)(mA)(mG)(mC)(rU)#(rC)#(mC)(mC)(rU)#(mU)(mU)(mC)(mU)(mC) | |
| | (mA)(mU)(mU)#(mU)#(mC)#(mG) | |
| CR48- | (mC)#(mU)#(mG)#(mA)(mA)(mC)(dT)#(mU)(mG)(mU)(mG)(mG)(mC) | 350 |
| 213_MCV | (fC)(mG)(fU)(mU)(mU)(rA)(rC)(rG)(mU)(rC)(rG)(mG)(dT)#(rU)(mG) | |
| | (mU)(mA)(mG)(mC)(rU)(rC)(rC)(rC)(rU)(mU)(mU)(mC)(mU)(mC)(mA) | |
| | (mU)(mU)#(mU)#(mC)#(mG) | |
| CR48- | (mC)#(mU)#(mG)#(mA)(mA)(mC)(dT)#(mU)(mG)(mU)(mG)(mG)(mC) | 351 |
| 214_MCV | (fC)(mG)(fU)(mU)(mU)(rA)(rC)(rG)(mU)(rC)(rG)(mG)(dT)#(rU)(mG) | |
| | (mU)(mA)(mG)(mC)(rU)(rC)(mC)(rC)(rU)(mU)(mU)(mC)(mU)(mC)(mA) | |
| | (mU)(mU)#(mU)#(mC)#(mG) | |
| CR48- | (mC)#(mU)#(mG)#(mA)(mA)(mC)(dT)#(mU)(mG)(mU)(mG)(mG)(mC) | 352 |
| 215_MCV | (fC)(mG)(fU)(mU)(mU)(rA)(rC)(rG)(mU)(rC)(rG)(mG)(dT)#(rU)(mG) | |
| | (mU)(mA)(mG)(mC)(rU)(rC)(rC)(mC)(rU)(mU)(mU)(mC)(mU)(mC)(mA) | |
| | (mU)(mU)#(mU)#(mC)#(mG) | |
| CR48- | (mC)#(mU)#(mG)#(mA)(mA)(mC)(dT)#(mU)(mG)(mU)(mG)(mG)(mC) | 353 |
| 216_MCV | (fC)(mG)(fU)(mU)(mU)(rA)(rC)(rG)(mU)(rC)(rG)(mG)(dT)#(rU)(mG) | |
| | (mU)(mA)(mG)(mC)(rU)(rC)(mC)(mC)(rU)(mU)(mU)(mC)(mU)(mC)(mA) | |
| | (mU)(mU)#(mU)#(mC)#(mG) | |
| CR48- | (mC)#(mU)#(mG)#(mA)(mA)(mC)(dT)#(mU)(mG)(mU)(mG)(mG)(mC) | 354 |
| 217_MCV | (fC)(mG)(fU)(mU)(mU)(rA)(rC)(rG)(mU)(rC)(rG)(mG)(rU)(rU)(rG)(mU) | |
| | (mA)(mG)(mC)(dT)#(rC)(rC)(rC)(rU)(mU)(mU)(mC)(mU)(mC)(mA)(mU) | |
| | (mU)#(mU)#(mC)#(mG) | |
| CR48- | (mC)#(mU)#(mG)#(mA)(mA)(mC)(dT)#(mU)(mG)(mU)(mG)(mG)(mC) | 355 |
| 218_MCV | (fC)(mG)(fU)(mU)(mU)(rA)(rC)(rG)(mU)(rC)(rG)(mG)(rU)(rU)(rG)(mU) | |
| | (mA)(mG)(mC)(rU)(dC)#(rC)(rC)(rU)(mU)(mU)(mC)(mU)(mC)(mA)(mU) | |
| | (mU)#(mU)#(mC)#(mG) | |
| CR48- | (mC)#(mU)#(mG)#(mA)(mA)(mC)(dT)#(mU)(mG)(mU)(mG)(mG)(mC) | 356 |
| 219_MCV | (fC)(mG)(fU)(mU)(mU)(rA)(rC)(rG)(mU)(rC)(rG)(mG)(rU)(rU)(rG)(mU) | |
| | (mA)(mG)(mC)(rU)(rC)(dC)#(rC)(rU)(mU)(mU)(mC)(mU)(mC)(mA)(mU) | |
| | (mU)#(mU)#(mC)#(mG) | |
| CR48- | (mC)#(mU)#(mG)#(mA)(mA)(mC)(dT)#(mU)(mG)(mU)(mG)(mG)(mC) | 357 |
| 220_MCV | (fC)(mG)(fU)(mU)(mU)(rA)(rC)(rG)(mU)(rC)(rG)(mG)(rU)(rU)(rG)(mU) | |
| | (mA)(mG)(mC)(rU)(rC)(rC)(dC)#(rU)(mU)(mU)(mC)(mU)(mC)(mA)(mU) | |
| | (mU)#(mU)#(mC)#(mG) | |
| CR48- | (mC)#(mU)#(mG)#(mA)(mA)(mC)(dT)#(mU)(mG)(mU)(mG)(mG)(mC) | 358 |
| 221_MCV | (fC)(mG)(fU)(mU)(mU)(rA)(rC)(rG)(mU)(rC)(rG)(mG)(rU)(rU)(rG)(mU) | |
| | (mA)(mG)(mC)(rU)(rC)(rC)(rC)(dT)#(mU)(mU)(mC)(mU)(mC)(mA)(mU) | |
| | (mU)#(mU)#(mC)#(mG) | |
| CR48- | (mC)#(mU)#(mG)#(mA)(mA)(mC)(dT)#(mU)(mG)(mU)(mG)(mG)(mC) | 359 |
| 222_MCV | (fC)(mG)(fU)(mU)(mU)(rA)(rC)(rG)(mU)(rC)(rG)(mG)(rU)(rU)(rG)(mU) | |
| | (mA)(mG)(mC)(dT)#(rC)(dC)#(rC)(dT)#(mU)(mU)(mC)(mU)(mC)(mA) | |
| | (mU)(mU)#(mU)#(mC)#(mG) | |
| CR48- | (mC)#(mU)#(mG)#(mA)(mA)(mC)(dT)#(mU)(mG)(mU)(mG)(mG)(mC) | 360 |
| 223_MCV | (fC)(mG)(fU)(mU)(mU)(rA)(rC)(rG)(mU)(rC)(rG)(mG)(dT)#(rU)(rG)(mU) | |
| | (mA)(mG)(mC)(dT)#(dC)#(dC)#(rC)(dT)#(mU)(mU)(mC)(mU)(mC)(mA) | |
| | (mU)(mU)#(mU)#(mC)#(mG) | |
| CR48- | (mC)#(mU)#(mG)#(mA)(mA)(mC)(dT)#(mU)(mG)(mU)(mG)(mG)(mC) | 361 |
| 224_MCV | (fC)(mG)(fU)(mU)(mU)(rA)(rC)(rG)(mU)(rC)(rG)(mG)(dT)#(rU)(mG) | |
| | (mU)(mA)(mG)(mC)(dT)#(rC)(mC)(rC)(dT)#(mU)(mU)(mC)(mU)(mC)(mA) | |
| | (mU)(mU)#(mU)#(mC)#(mG) | |
| CR48- | (mC)#(mU)#(mG)#(mA)(mA)(mC)(dT)#(mU)(mG)(mU)(mG)(mG)(mC) | 362 |
| 225_MCV | (fC)(mG)(fU)(mU)(mU)(rA)(rC)(rG)(mU)(rC)(rG)(mG)(dT)#(rU)(mG) | |
| | (mU)(mA)(mG)(mC)(dT)#(rC)(dC)#(mC)(dT)#(mU)(mU)(mC)(mU)(mC) | |
| | (mA)(mU)(mU)#(mU)#(mC)#(mG) | |
| CR48- | (mC)#(mU)#(mG)#(mA)(mA)(mC)(dT)#(mU)(mG)(mU)(mG)(mG)(mC) | 363 |
| 226_MCV | (fC)(mG)(fU)(mU)(mU)(rA)(rC)(rG)(mU)(rC)(rG)(mG)(dT)#(rU)(mG) | |
| | (mU)(mA)(mG)(mC)(dT)#(rC)(mC)(mC)(dT)#(mU)(mU)(mC)(mU)(mC) | |
| | (mA)(mU)(mU)#(mU)#(mC)#(mG) | |
| CR48- | (mC)#(mU)#(mG)#(mA)(mA)(mC)(dT)#(mU)(mG)(mU)(mG)(mG)(mC) | 364 |
| 227_MCV | (fC)(mG)(fU)(mU)(mU)(rA)(rC)(rG)(mU)(rC)(rG)(mG)(dT)#(rU)(mG) | |
| | (mU)(mA)(mG)(mC)(dT)#(dC)#(mC)(mC)(dT)#(mU)(mU)(mC)(mU)(mC) | |
| | (mA)(mU)(mU)#(mU)#(mC)#(mG) | |
| CR48- | (mC)#(mU)#(mG)#(mA)(mA)(mC)(dT)#(mU)(mG)(mU)(mG)(fG)(mC) | 365 |
| 228_MCV | (fC)(mG)(fU)(mU)(mU)(rA)#(fC)(mG)(mU)(mC)(mG)(mG)(rU)#(rU)(mG) | |
| | (mU)(mA)(mG)(mC)(rU)#(rC)(rC)#(rC)(rU)#(mU)(mU)(mC)(mU)(mC) | |
| | (mA)(mU)(mU)#(mU)#(mC)#(mG) | |
| CR48- | (mC)#(mU)#(mG)#(mA)(mA)(mC)(dT)#(mU)(mG)(mU)(mG)(fG)(mC) | 366 |
| 229_MCV | (fC)(mG)(fU)(mU)(mU)(rA)#(mC)(fG)(mU)(mC)(mG)(mG)(rU)#(rU)(mG) | |
| | (mU)(mA)(mG)(mC)(rU)#(rC)(rC)#(rC)(rU)#(mU)(mU)(mC)(mU)(mC) | |
| | (mA)(mU)(mU)#(mU)#(mC)#(mG) | |
| CR48- | (mC)#(mU)#(mG)#(mA)(mA)(mC)(dT)#(mU)(mG)(mU)(mG)(mG)(mC) | 367 |
| 230_M | (fC)(mG)(fU)(mU)(mU)(rA)#(fC)(mG)(mU)(mC)(mG)(mG)(rU)#(rU) | |
| | (mG)(mU)(mA)(mG)(mC)(rU)#(rC)(rC)#(rC)(rU)#(mU)(mU)(mC)(mU)(mC) | |
| | (mA)(mU)(mU)#(mU)#(mC)#(mG) | |
| CR48- | (mC)#(mU)#(mG)#(mA)(mA)(mC)(dT)#(mU)(mG)(mU)(mG)(mG)(mC) | 368 |
| 231_MCV | (fC)(mG)(fU)(mU)(mU)(rA)#(mC)(fG)(mU)(mC)(mG)(mG)(rU)#(rU) | |
| | (mG)(mU)(mA)(mG)(mC)(rU)#(rC)(rC)#(rC)(rU)#(mU)(mU)(mC)(mU)(mC) | |
| | (mA)(mU)(mU)#(mU)#(mC)#(mG) | |
| CR48- | (mC)#(mU)#(mG)#(mA)(mA)(mC)(dT)#(mU)(mG)(mU)(mG)(fG)(mC) | 369 |
| 232_MCV | (fC)(mG)(fU)(mU)(mU)(rA)#(fC)(mG)(mU)(mC)(mG)(mG)(rU)#(rU)(mG) | |
| | (mU)(mA)(mG)(mC)(rU)#(rC)(mC)(rC)(rU)#(mU)(mU)(mC)(mU)(mC) | |
| | (mA)(mU)(mU)#(mU)#(mC)#(mG) | |
| CR48- | (mC)#(mU)#(mG)#(mA)(mA)(mC)(dT)#(mU)(mG)(mU)(mG)(fG)(mC) | 370 |
| 233_MCV | (fC)(mG)(fU)(mU)(mU)(rA)#(mC)(fG)(mU)(mC)(mG)(mG)(rU)#(rU)(mG) | |
| | (mU)(mA)(mG)(mC)(rU)#(rC)(mC)(rC)(rU)#(mU)(mU)(mC)(mU)(mC) | |
| | (mA)(mU)(mU)#(mU)#(mC)#(mG) | |
| CR48- | (mC)#(mU)#(mG)#(mA)(mA)(mC)(dT)#(mU)(mG)(mU)(mG)(mG)(mC) | 371 |
| 234_MCV | (fC)(mG)(fU)(mU)(mU)(rA)#(fC)(mG)(mU)(mC)(mG)(mG)(rU)#(rU) | |
| | (mG)(mU)(mA)(mG)(mC)(rU)#(rC)(mC)(rC)(rU)#(mU)(mU)(mC)(mU)(mC) | |
| | (mA)(mU)(mU)#(mU)#(mC)#(mG) | |
| CR48- | (mC)#(mU)#(mG)#(mA)(mA)(mC)(dT)#(mU)(mG)(mU)(mG)(mG)(mC) | 372 |
| 235_MCV | (fC)(mG)(fU)(mU)(mU)(rA)#(mC)(fG)(mU)(mC)(mG)(mG)(rU)#(rU)(mG) | |
| | (mU)(mA)(mG)(mC)(rU)#(rC)(mC)(rC)(rU)#(mU)(mU)(mC)(mU)(mC) | |
| | (mA)(mU)(mU)#(mU)#(mC)#(mG) | |
| CR48- | (mC)#(mU)#(mG)#(mA)(mA)(mC)(dT)#(mU)(mG)(mU)(mG)(mG)(mC) | 373 |
| 236_MCV | (fC)(mG)(fU)(mU)(mU)(rA)#(mC)(fG)(mU)(mC)(mG)(mG)(rU)#(rU)# | |
| | (mG)(mU)(mA)(mG)(mC)(rU)#(rC)#(mC)(rC)#(rU)#(mU)(mU)(mC)(mU) | |
| | (mC)(mA)(mU)(mU)#(mU)#(mC)#(mG) | |
| CR48- | (mC)#(mU)#(mG)#(mA)(mA)(mC)(dT)#(mU)(mG)(mU)(mG)(fG)(mC) | 374 |
| 199- | (fC)(mG)(fU)(mU)(mU)(mA)(mC)(fG)(mU)(mC)(mG)(mG)(rU)(rU)(mG) | |
| V1.1_MCV | (mU)(mA)(mG)(mC)(rU)(rC)(rC)(dC)#(rU)(mU)(mU)(mC)(mU)(mC) | |
| | (mA)(mU)(mU)#(mU)#(mC)#(mG) | |
| CR48- | (mC)#(mU)#(mG)#(mA)(mA)(mC)(dT)#(mU)(mG)(mU)(mG)(fG)(mC) | 375 |
| 199- | (fC)(mG)(fU)(mU)(mU)(mA)(mC)(fG)(mU)(mC)(mG)(mG)(rU)(rU)(mG) | |
| V1.2_MCV | (mU)(mA)(mG)(mC)(rU)(rC)(dC)#(rC)(rU)(mU)(mU)(mC)(mU)(mC) | |
| | (mA)(mU)(mU)#(mU)#(mC)#(mG) | |
| CR48- | (mC)#(mU)#(mG)#(mA)(mA)(mC)(dT)#(mU)(mG)(mU)(mG)(fG)(mC) | 376 |
| 199- | (fC)(mG)(fU)(mU)(mU)(mA)(mC)(fG)(mU)(mC)(mG)(mG)(rU)(rU)(mG) | |
| V1.3_MCV | (mU)(mA)(mG)(mC)(rU)(rC)(rC)(rC)(dT)#(mU)(mU)(mC)(mU)(mC) | |
| | (mA)(mU)(mU)#(mU)#(mC)#(mG) | |
| CR48- | (mC)#(mU)#(mG)#(mA)(mA)(mC)(dT)#(mU)(mG)(mU)(mG)(fG)(mC) | 377 |
| 199- | (fC)(mG)(fU)(mU)(mU)(mA)(mC)(fG)(mU)(mC)(mG)(mG)(rU)(rU)(mG) | |
| V1.4_MCV | (mU)(mA)(mG)(mC)(rU)(rC)(mC)(rC)(rU)(mU)(mU)(mC)(mU)(mC)(mA) | |
| | (mU)(mU)#(mU)#(mC)#(mG) | |
| CR48- | (mC)#(mU)#(mG)#(mA)(mA)(mC)(dT)#(mU)(mG)(mU)(mG)(fG)(mC) | 378 |
| 199- | (fC)(mG)(fU)(mU)(mU)(mA)(mC)(fG)(mU)(mC)(mG)(mG)(rU)(rU)(mG) | |
| V1.5_MCV | (mU)(mA)(mG)(mC)(rU)(rC)(dC)#(dC)#(rU)(mU)(mU)(mC)(mU)(mC) | |
| | (mA)(mU)(mU)#(mU)#(mC)#(mG) | |
| CR48- | (mC)#(mU)#(mG)#(mA)(mA)(mC)(dT)#(mU)(mG)(mU)(mG)(fG)(mC) | 379 |
| 199- | (fC)(mG)(fU)(mU)(mU)(mA)(mC)(fG)(mU)(mC)(mG)(mG)(rU)(rU)(mG) | |
| V1.6_MCV | (mU)(mA)(mG)(mC)(rU)(rC)(dC)#(rC)(dT)#(mU)(mU)(mC)(mU)(mC) | |
| | (mA)(mU)(mU)#(mU)#(mC)#(mG) | |
| CR48- | (mC)#(mU)#(mG)#(mA)(mA)(mC)(dT)#(mU)(mG)(mU)(mG)(fG)(mC) | 380 |
| 199- | (fC)(mG)(fU)(mU)(mU)(mA)(mC)(fG)(mU)(mC)(mG)(mG)(rU)(rU)(mG) | |
| V1.7_MCV | (mU)(mA)(mG)(mC)(rU)(rC)(dC)#(dC)#(dT)#(mU)(mU)(mC)(mU)(mC) | |
| | (mA)(mU)(mU)#(mU)#(mC)#(mG) | |
| CR48- | (mC)#(mU)#(mG)#(mA)(mA)(mC)(dT)#(mU)(mG)(mU)(mG)(fG)(mC) | 381 |
| 199- | (fC)(mG)(fU)(mU)(mU)(mA)(mC)(fG)(mU)(mC)(mG)(mG)(rU)(rU)(mG) | |
| V1.8_MCV | (mU)(mA)(mG)(mC)(rU)(rC)(mC)(dC)#(rU)(mU)(mU)(mC)(mU)(mC) | |
| | (mA)(mU)(mU)#(mU)#(mC)#(mG) | |
| CR48- | (mC)#(mU)#(mG)#(mA)(mA)(mC)(dT)#(mU)(mG)(mU)(mG)(fG)(mC) | 382 |
| 199- | (fC)(mG)(fU)(mU)(mU)(mA)(mC)(fG)(mU)(mC)(mG)(mG)(rU)(rU)(mG) | |
| V1.9_MCV | (mU)(mA)(mG)(mC)(rU)(rC)(mC)(rC)(dT)#(mU)(mU)(mC)(mU)(mC) | |
| | (mA)(mU)(mU)#(mU)#(mC)#(mG) | |
| CR48- | (mC)#(mU)#(mG)#(mA)(mA)(mC)(dT)#(mU)(mG)(mU)(mG)(fG)(mC) | 383 |
| 199- | (fC)(mG)(fU)(mU)(mU)(mA)(mC)(fG)(mU)(mC)(mG)(mG)(rU)(rU)(mG) | |
| V1.10_ | (mU)(mA)(mG)(mC)(rU)(rC)(mC)(dC)#(dT)#(mU)(mU)(mC)(mU)(mC) | |
| MCV | (mA)(mU)(mU)#(mU)#(mC)#(mG) | |
| CR48- | (mC)#(mU)#(mG)#(mA)(mA)(mC)(dT)#(mU)(mG)(mU)(mG)(fG)(mC) | 384 |
| 199- | (fC)(mG)(fU)(mU)(mU)(mA)(mC)(fG)(mU)(mC)(mG)(mG)(rU)(rU)(mG) | |
| V1.11_ | (mU)(mA)(mG)(mC)(rU)(rC)(mC)(mC)(rU)(mU)(mU)(mC)(mU)(mC) | |
| MCV | (mA)(mU)(mU)#(mU)#(mC)#(mG) | |
| CR48- | (mC)#(mU)#(mG)#(mA)(mA)(mC)(dT)#(mU)(mG)(mU)(mG)(fG)(mC) | 385 |
| 199- | (fC)(mG)(fU)(mU)(mU)(mA)(mC)(fG)(mU)(mC)(mG)(mG)(rU)(rU)(mG) | |
| V1.12_ | (mU)(mA)(mG)(mC)(rU)(rC)(mC)(mC)(dT)#(mU)(mU)(mC)(mU)(mC) | |
| MCV | (mA)(mU)(mU)#(mU)#(mC)#(mG) | |
| CR48- | (mC)#(mU)#(mG)#(mA)(mA)(mC)(dT)#(mU)(mG)(mU)(mG)(fG)(mC)(f | 386 |
| 199- | C)(mG)(fU)(mU)(mU)(mA)(mC)(fG)(mU)(mC)(mG)(mG)(rU)(rU)(mG)( | |
| V1.13_ | mU)(mA)(mG)(mC)(dT)#(rC)(rC)(dC)#(rU)(mU)(mU)(mC)(mU)(mC)(m | |
| MCV | A)(mU)(mU)#(mU)#(mC)#(mG) | |
| CR48- | (mC)#(mU)#(mG)#(mA)(mA)(mC)(dT)#(mU)(mG)(mU)(mG)(fG)(mC)(f | 387 |
| 199- | C)(mG)(fU)(mU)(mU)(mA)(mC)(fG)(mU)(mC)(mG)(mG)(rU)(rU)(mG)( | |
| V1.14_ | mU)(mA)(mG)(mC)(dT)#(rC)(dC)#(rC)(rU)(mU)(mU)(mC)(mU)(mC)(m | |
| MCV | A)(mU)(mU)#(mU)#(mC)#(mG) | |
| CR48- | (mC)#(mU)#(mG)#(mA)(mA)(mC)(dT)#(mU)(mG)(mU)(mG)(fG)(mC)(f | 388 |
| 199- | C)(mG)(fU)(mU)(mU)(mA)(mC)(fG)(mU)(mC)(mG)(mG)(rU)(rU)(mG)( | |
| V1.15_ | mU)(mA)(mG)(mC)(dT)#(rC)(rC)(rC)(dT)#(mU)(mU)(mC)(mU)(mC)(m | |
| MCV | A)(mU)(mU)#(mU)#(mC)#(mG) | |
| CR48- | (mC)#(mU)#(mG)#(mA)(mA)(mC)(dT)#(mU)(mG)(mU)(mG)(fG)(mC)(f | 389 |
| 199- | C)(mG)(fU)(mU)(mU)(mA)(mC)(fG)(mU)(mC)(mG)(mG)(rU)(rU)(mG)( | |
| V1.16_ | mU)(mA)(mG)(mC)(dT)#(rC)(mC)(rC)(rU)(mU)(mU)(mC)(mU)(mC)(m | |
| MCV | A)(mU)(mU)#(mU)#(mC)#(mG) | |
| CR48- | (mC)#(mU)#(mG)#(mA)(mA)(mC)(dT)#(mU)(mG)(mU)(mG)(fG)(mC)(f | 390 |
| 199- | C)(mG)(fU)(mU)(mU)(mA)(mC)(fG)(mU)(mC)(mG)(mG)(rU)(rU)(mG)( | |
| V1.17_ | mU)(mA)(mG)(mC)(dT)#(rC)(dC)#(dC)#(rU)(mU)(mU)(mC)(mU)(mC)( | |
| MCV | mA)(mU)(mU)#(mU)#(mC)#(mG) | |
| CR48- | (mC)#(mU)#(mG)#(mA)(mA)(mC)(dT)#(mU)(mG)(mU)(mG)(fG)(mC)(f | 391 |
| 199- | C)(mG)(fU)(mU)(mU)(mA)(mC)(fG)(mU)(mC)(mG)(mG)(rU)(rU)(mG)( | |
| V1.18_ | mU)(mA)(mG)(mC)(dT)#(rC)(dC)#(rC)(dT)#(mU)(mU)(mC)(mU)(mC)( | |
| MCV | mA)(mU)(mU)#(mU)#(mC)#(mG) | |
| CR48- | (mC)#(mU)#(mG)#(mA)(mA)(mC)(dT)#(mU)(mG)(mU)(mG)(fG)(mC)(f | 392 |
| 199- | C)(mG)(fU)(mU)(mU)(mA)(mC)(fG)(mU)(mC)(mG)(mG)(rU)(rU)(mG)( | |
| V1.19_ | mU)(mA)(mG)(mC)(dT)#(rC)(dC)#(dC)#(dT)#(mU)(mU)(mC)(mU)(mC) | |
| MCV | (mA)(mU)(mU)#(mU)#(mC)#(mG) | |
| CR48- | (mC)#(mU)#(mG)#(mA)(mA)(mC)(dT)#(mU)(mG)(mU)(mG)(fG)(mC)(f | 393 |
| 199- | C)(mG)(fU)(mU)(mU)(mA)(mC)(fG)(mU)(mC)(mG)(mG)(rU)(rU)(mG)( | |
| V1.20_ | mU)(mA)(mG)(mC)(dT)#(rC)(mC)(dC)#(rU)(mU)(mU)(mC)(mU)(mC)( | |
| MCV | mA)(mU)(mU)#(mU)#(mC)#(mG) | |
| CR48- | (mC)#(mU)#(mG)#(mA)(mA)(mC)(dT)#(mU)(mG)(mU)(mG)(fG)(mC)(f | 394 |
| 199- | C)(mG)(fU)(mU)(mU)(mA)(mC)(fG)(mU)(mC)(mG)(mG)(rU)(rU)(mG)( | |
| V1.21_ | mU)(mA)(mG)(mC)(dT)#(rC)(mC)(rC)(dT)#(mU)(mU)(mC)(mU)(mC)( | |
| MCV | mA)(mU)(mU)#(mU)#(mC)#(mG) | |
| CR48- | (mC)#(mU)#(mG)#(mA)(mA)(mC)(dT)#(mU)(mG)(mU)(mG)(fG)(mC)(f | 395 |
| 199- | C)(mG)(fU)(mU)(mU)(mA)(mC)(fG)(mU)(mC)(mG)(mG)(rU)(rU)(mG)( | |
| V1.22_ | mU)(mA)(mG)(mC)(dT)#(rC)(mC)(dC)#(dT)#(mU)(mU)(mC)(mU)(mC)( | |
| MCV | mA)(mU)(mU)#(mU)#(mC)#(mG) | |
| CR48- | (mC)#(mU)#(mG)#(mA)(mA)(mC)(dT)#(mU)(mG)(mU)(mG)(fG)(mC)(f | 396 |
| 199- | C)(mG)(fU)(mU)(mU)(mA)(mC)(fG)(mU)(mC)(mG)(mG)(rU)(rU)(mG)( | |
| V1.23_ | mU)(mA)(mG)(mC)(dT)#(rC)(mC)(mC)(rU)(mU)(mU)(mC)(mU)(mC)(m | |
| MCV | A)(mU)(mU)#(mU)#(mC)#(mG) | |
| CR48- | (mC)#(mU)#(mG)#(mA)(mA)(mC)(dT)#(mU)(mG)(mU)(mG)(fG)(mC)(f | 397 |
| 199- | C)(mG)(fU)(mU)(mU)(mA)(mC)(fG)(mU)(mC)(mG)(mG)(rU)(rU)(mG)( | |
| V1.24_ | mU)(mA)(mG)(mC)(dT)#(rC)(mC)(mC)(dT)#(mU)(mU)(mC)(mU)(mC)( | |
| MCV | mA)(mU)(mU)#(mU)#(mC)#(mG) | |
| CR48- | (mC)#(mU)#(mG)#(mA)(mA)(mC)(dT)#(mU)(mG)(mU)(mG)(fG)(mC)(f | 398 |
| 199- | C)(mG)(fU)(mU)(mU)(mA)(mC)(fG)(mU)(mC)(mG)(mG)(dT)#(rU)(mG) | |
| V1.25_ | (mU)(mA)(mG)(mC)(dT)#(rC)(rC)(dC)#(rU)(mU)(mU)(mC)(mU)(mC)( | |
| MCV | mA)(mU)(mU)#(mU)#(mC)#(mG) | |
| CR48- | (mC)#(mU)#(mG)#(mA)(mA)(mC)(dT)#(mU)(mG)(mU)(mG)(fG)(mC)(f | 399 |
| 199- | C)(mG)(fU)(mU)(mU)(mA)(mC)(fG)(mU)(mC)(mG)(mG)(dT)#(rU)(mG) | |
| V1.26_ | (mU)(mA)(mG)(mC)(dT)#(rC)(dC)#(rC)(rU)(mU)(mU)(mC)(mU)(mC)( | |
| MCV | mA)(mU)(mU)#(mU)#(mC)#(mG) | |
| CR48- | (mC)#(mU)#(mG)#(mA)(mA)(mC)(dT)#(mU)(mG)(mU)(mG)(fG)(mC)(f | 400 |
| 199- | C)(mG)(fU)(mU)(mU)(mA)(mC)(fG)(mU)(mC)(mG)(mG)(dT)#(rU)(mG) | |
| V1.27_ | (mU)(mA)(mG)(mC)(dT)#(rC)(rC)(rC)(dT)#(mU)(mU)(mC)(mU)(mC)(m | |
| MCV | A)(mU)(mU)#(mU)#(mC)#(mG) | |
| CR48- | (mC)#(mU)#(mG)#(mA)(mA)(mC)(dT)#(mU)(mG)(mU)(mG)(fG)(mC)(f | 401 |
| 199- | C)(mG)(fU)(mU)(mU)(mA)(mC)(fG)(mU)(mC)(mG)(mG)(dT)#(rU)(mG) | |
| V1.28_ | (mU)(mA)(mG)(mC)(dT)#(rC)(mC)(rC)(rU)(mU)(mU)(mC)(mU)(mC)(m | |
| MCV | A)(mU)(mU)#(mU)#(mC)#(mG) | |
| CR48- | (mC)#(mU)#(mG)#(mA)(mA)(mC)(dT)#(mU)(mG)(mU)(mG)(fG)(mC)(f | 402 |
| 199- | C)(mG)(fU)(mU)(mU)(mA)(mC)(fG)(mU)(mC)(mG)(mG)(dT)#(rU)(mG) | |
| V1.29_ | (mU)(mA)(mG)(mC)(dT)#(rC)(dC)#(dC)#(rU)(mU)(mU)(mC)(mU)(mC)( | |
| MCV | mA)(mU)(mU)#(mU)#(mC)#(mG) | |
| CR48- | (mC)#(mU)#(mG)#(mA)(mA)(mC)(dT)#(mU)(mG)(mU)(mG)(fG)(mC)(f | 403 |
| 199- | C)(mG)(fU)(mU)(mU)(mA)(mC)(fG)(mU)(mC)(mG)(mG)(dT)#(rU)(mG) | |
| V1.30_ | (mU)(mA)(mG)(mC)(dT)#(rC)(dC)#(rC)(dT)#(mU)(mU)(mC)(mU)(mC)( | |
| MCV | mA)(mU)(mU)#(mU)#(mC)#(mG) | |
| CR48- | (mC)#(mU)#(mG)#(mA)(mA)(mC)(dT)#(mU)(mG)(mU)(mG)(fG)(mC)(f | 404 |
| 199- | C)(mG)(fU)(mU)(mU)(mA)(mC)(fG)(mU)(mC)(mG)(mG)(dT)#(rU)(mG) | |
| V1.31_ | (mU)(mA)(mG)(mC)(dT)#(rC)(dC)#(dC)#(dT)#(mU)(mU)(mC)(mU)(mC | |
| MCV | )(mA)(mU)(mU)#(mU)#(mC)#(mG) | |
| CR48- | (mC)#(mU)#(mG)#(mA)(mA)(mC)(dT)#(mU)(mG)(mU)(mG)(fG)(mC)(f | 405 |
| 199- | C)(mG)(fU)(mU)(mU)(mA)(mC)(fG)(mU)(mC)(mG)(mG)(dT)#(rU)(mG) | |
| V1.32_ | (mU)(mA)(mG)(mC)(dT)#(rC)(mC)(dC)#(rU)(mU)(mU)(mC)(mU)(mC)( | |
| MCV | mA)(mU)(mU)#(mU)#(mC)#(mG) | |
| CR48- | (mC)#(mU)#(mG)#(mA)(mA)(mC)(dT)#(mU)(mG)(mU)(mG)(fG)(mC)(f | 406 |
| 199- | C)(mG)(fU)(mU)(mU)(mA)(mC)(fG)(mU)(mC)(mG)(mG)(dT)#(rU)(mG) | |
| V1.33_ | (mU)(mA)(mG)(mC)(dT)#(rC)(mC)(rC)(dT)#(mU)(mU)(mC)(mU)(mC)( | |
| MCV | mA)(mU)(mU)#(mU)#(mC)#(mG) | |
| CR48- | (mC)#(mU)#(mG)#(mA)(mA)(mC)(dT)#(mU)(mG)(mU)(mG)(fG)(mC)(f | 407 |
| 199- | C)(mG)(fU)(mU)(mU)(mA)(mC)(fG)(mU)(mC)(mG)(mG)(dT)#(rU)(mG) | |
| V1.34_ | (mU)(mA)(mG)(mC)(dT)#(rC)(mC)(dC)#(dT)#(mU)(mU)(mC)(mU)(mC) | |
| MCV | (mA)(mU)(mU)#(mU)#(mC)#(mG) | |
| CR48- | (mC)#(mU)#(mG)#(mA)(mA)(mC)(dT)#(mU)(mG)(mU)(mG)(fG)(mC)(f | 408 |
| 199- | C)(mG)(fU)(mU)(mU)(mA)(mC)(fG)(mU)(mC)(mG)(mG)(dT)#(rU)(mG) | |
| V1.35_ | (mU)(mA)(mG)(mC)(dT)#(rC)(mC)(mC)(rU)(mU)(mU)(mC)(mU)(mC)( | |
| MCV | mA)(mU)(mU)#(mU)#(mC)#(mG) | |
| CR48- | (mC)#(mU)#(mG)#(mA)(mA)(mC)(dT)#(mU)(mG)(mU)(mG)(fG)(mC)(f | 409 |
| 199- | C)(mG)(fU)(mU)(mU)(mA)(mC)(fG)(mU)(mC)(mG)(mG)(dT)#(rU)(mG) | |
| V1.36_ | (mU)(mA)(mG)(mC)(dT)#(rC)(mC)(mC)(dT)#(mU)(mU)(mC)(mU)(mC) | |
| MCV | (mA)(mU)(mU)#(mU)#(mC)#(mG) | |
| CR48- | (mC)#(mU)#(mG)#(mA)(mA)(mC)(dT)#(mU)(mG)(mU)(mG)(fG)(mC)(f | 410 |
| 203- | C)(mG)(fU)(mU)(mU)(fA)(mC)(fG)(mU)(mC)(mG)(mG)(rU)(rU)(mG)( | |
| V1.7_ | mU)(mA)(mG)(mC)(rU)(rC)(dC)#(dC)#(dT)#(mU)(mU)(mC)(mU)(mC)( | |
| MCV | mA)(mU)(mU)#(mU)#(mC)#(mG) | |
| CR48- | (mC)#(mU)#(mG)#(mA)(mA)(mC)(dT)#(mU)(mG)(mU)(mG)(fG)(mC)(f | 411 |
| 203- | C)(mG)(fU)(mU)(mU)(fA)(mC)(fG)(mU)(mC)(mG)(mG)(rU)(rU)(mG)( | |
| V1.10_ | mU)(mA)(mG)(mC)(rU)(rC)(mC)(dC)#(dT)#(mU)(mU)(mC)(mU)(mC)( | |
| MCV | mA)(mU)(mU)#(mU)#(mC)#(mG) | |
| CR48- | (mC)#(mU)#(mG)#(mA)(mA)(mC)(dT)#(mU)(mG)(mU)(mG)(fG)(mC)(f | 412 |
| 203- | C)(mG)(fU)(mU)(mU)(fA)(mC)(fG)(mU)(mC)(mG)(mG)(rU)(rU)(mG)( | |
| V1.12_ | mU)(mA)(mG)(mC)(rU)(rC)(mC)(mC)(dT)#(mU)(mU)(mC)(mU)(mC)(m | |
| MCV | A)(mU)(mU)#(mU)#(mC)#(mG) | |
| CR48- | (mC)#(mU)#(mG)#(mA)(mA)(mC)(dT)#(mU)(mG)(mU)(mG)(fG)(mC)(f | 413 |
| 203- | C)(mG)(fU)(mU)(mU)(fA)(mC)(fG)(mU)(mC)(mG)(mG)(rU)(rU)(mG)( | |
| V1.18_ | mU)(mA)(mG)(mC)(dT)#(rC)(dC)#(rC)(dT)#(mU)(mU)(mC)(mU)(mC)( | |
| MCV | mA)(mU)(mU)#(mU)#(mC)#(mG) | |
| CR48- | (mC)#(mU)#(mG)#(mA)(mA)(mC)(dT)#(mU)(mG)(mU)(mG)(fG)(mC)(f | 414 |
| 203- | C)(mG)(fU)(mU)(mU)(fA)(mC)(fG)(mU)(mC)(mG)(mG)(rU)(rU)(mG)( | |
| V1.19_ | mU)(mA)(mG)(mC)(dT)#(rC)(dC)#(dC)#(dT)#(mU)(mU)(mC)(mU)(mC) | |
| MCV | (mA)(mU)(mU)#(mU)#(mC)#(mG) | |
| CR48- | (mC)#(mU)#(mG)#(mA)(mA)(mC)(dT)#(mU)(mG)(mU)(mG)(fG)(mC)(f | 415 |
| 203- | C)(mG)(fU)(mU)(mU)(fA)(mC)(fG)(mU)(mC)(mG)(mG)(rU)(rU)(mG)( | |
| V1.22_ | mU)(mA)(mG)(mC)(dT)#(rC)(mC)(dC)#(dT)#(mU)(mU)(mC)(mU)(mC)( | |
| MCV | mA)(mU)(mU)#(mU)#(mC)#(mG) | |
| CR48- | (mC)#(mU)#(mG)#(mA)(mA)(mC)(dT)#(mU)(mG)(mU)(mG)(fG)(mC)(f | 416 |
| 203- | C)(mG)(fU)(mU)(mU)(fA)(mC)(fG)(mU)(mC)(mG)(mG)(rU)(rU)(mG)( | |
| V1.24_ | mU)(mA)(mG)(mC)(dT)#(rC)(mC)(mC)(dT)#(mU)(mU)(mC)(mU)(mC)( | |
| MCV | mA)(mU)(mU)#(mU)#(mC)#(mG) | |
| CR48- | (mC)#(mU)#(mG)#(mA)(mA)(mC)(dT)#(mU)(mG)(mU)(mG)(fG)(mC)(f | 417 |
| 203- | C)(mG)(fU)(mU)(mU)(fA)(mC)(fG)(mU)(mC)(mG)(mG)(dT)#(rU)(mG)( | |
| V1.30_ | mU)(mA)(mG)(mC)(dT)#(rC)(dC)#(rC)(dT)#(mU)(mU)(mC)(mU)(mC)( | |
| MCV | mA)(mU)(mU)#(mU)#(mC)#(mG) | |
| CR48- | (mC)#(mU)#(mG)#(mA)(mA)(mC)(dT)#(mU)(mG)(mU)(mG)(fG)(mC)(f | 418 |
| 203- | C)(mG)(fU)(mU)(mU)(fA)(mC)(fG)(mU)(mC)(mG)(mG)(dT)#(rU)(mG)( | |
| V1.31_ | mU)(mA)(mG)(mC)(dT)#(rC)(dC)#(dC)#(dT)#(mU)(mU)(mC)(mU)(mC) | |
| MCV | (mA)(mU)(mU)#(mU)#(mC)#(mG) | |
| CR48- | (mC)#(mU)#(mG)#(mA)(mA)(mC)(dT)#(mU)(mG)(mU)(mG)(fG)(mC)(f | 419 |
| 203- | C)(mG)(fU)(mU)(mU)(fA)(mC)(fG)(mU)(mC)(mG)(mG)(dT)#(rU)(mG)( | |
| V1.34_ | mU)(mA)(mG)(mC)(dT)#(rC)(mC)(dC)#(dT)#(mU)(mU)(mC)(mU)(mC)( | |
| MCV | mA)(mU)(mU)#(mU)#(mC)#(mG) | |
| CR48- | (mC)#(mU)#(mG)#(mA)(mA)(mC)(dT)#(mU)(mG)(mU)(mG)(fG)(mC)(f | 420 |
| 203- | C)(mG)(fU)(mU)(mU)(fA)(mC)(fG)(mU)(mC)(mG)(mG)(dT)#(rU)(mG)( | |
| V1.36_ | mU)(mA)(mG)(mC)(dT)#(rC)(mC)(mC)(dT)#(mU)(mU)(mC)(mU)(mC)( | |
| MCV | mA)(mU)(mU)#(mU)#(mC)#(mG) | |
| CR48- | (mC)#(mU)#(mG)#(mA)(mA)(mC)(dT)#(mU)(mG)(mU)(mG)(fG)(mC)(f | 421 |
| 203- | C)(mG)(fU)(mU)(mU)(fA)(mC)(fG)(mU)(mC)(mG)(mG)(dT)#(rU)(mG)( | |
| V1.31X_ | mU)(mA)(mG)(mC)(dT)#(dC)#(dC)#(dC)#(dT)#(mU)(mU)(mC)(mU)(m | |
| MCV | C)(mA)(mU)(mU)#(mU)#(mC)#(mG) | |
| CR48- | (mA)#(mG)#(mG)#(rC)(rA)(rA)(rA)(rG)(rG)(rA)(rG)(rG)(rA)(rA)(rG)(rA | 422 |
| 0_mTTR- | )(rG)(rU)(rC)(rG)(rA)(rA)(rG)(rG)(rG)(rU)(rU)(rG)(rU)(rA)(rG)(rC)(rU)( | |
| G1 | rC)(rC)(rC)(rU)(rU)(rU)(rC)(rU)(rC)(rA)(rU)(rU)#(mU)#(mC)#(mG) | |
| CR48- | (mA)#(mG)#(mG)#(mC)(mA)(rA)(rA)(rG)(mG)(mA)(mG)(rG)(mA)(rA)( | 423 |
| 4_mTTR- | rG)(rA)(rG)(mU)(rC)(rG)(rA)(mA)(rG)(rG)(mG)(rU)(rU)(rG)(mU)(mA)( | |
| G1 | mG)(mC)(rU)(rC)(rC)(rC)(rU)(mU)(mU)(mC)(mU)(mC)(mA)(mU)(mU) | |
| | #(mU)#(mC)#(mG) | |
| CR48- | (mA)#(mG)#(mG)#(mC)(mA)(mA)(dA)#(mG)(mG)(mA)(mG)(fG)(mA)(f | 424 |
| 199_ | A)(mG)(fA)(mG)(mU)(mC)(mG)(fA)(mA)(mG)(mG)(mG)(rU)(rU)(rG)( | |
| mTTR-G1 | mU)(mA)(mG)(mC)(rU)(rC)(rC)(rC)(rU)(mU)(mU)(mC)(mU)(mC)(mA)( | |
| | mU)(mU)#(mU)#(mC)#(mG) | |
| CR48- | (mA)#(mG)#(mG)#(mC)(mA)(mA)(dA)#(mG)(mG)(mA)(mG)(fG)(mA)(f | 425 |
| 203_ | A)(mG)(fA)(mG)(mU)(fC)(mG)(fA)(mA)(mG)(mG)(mG)(rU)(rU)(rG)(m | |
| mTTR-G1 | U)(mA)(mG)(mC)(rU)(rC)(rC)(rC)(rU)(mU)(mU)(mC)(mU)(mC)(mA)(m | |
| | U)(mU)#(mU)#(mC)#(mG) | |
| CR48- | (mA)#(mG)#(mG)#(mC)(mA)(mA)(dA)#(mG)(mG)(mA)(mG)(fG)(mA)(f | 426 |
| 199- | A)(mG)(fA)(mG)(mU)(mC)(mG)(fA)(mA)(mG)(mG)(mG)(rU)(rU)(mG)( | |
| V1.7_ | mU)(mA)(mG)(mC)(rU)(rC)(dC)#(dC)#(dT)#(mU)(mU)(mC)(mU)(mC)( | |
| mTTR- | mA)(mU)(mU)#(mU)#(mC)#(mG) | |
| G1 | | |
| CR199- | (mA)#(mG)#(mG)#(mC)(mA)(mA)(dA)#(mG)(mG)(mA)(mG)(fG)(mA)(f | 427 |
| V1.18_ | A)(mG)(fA)(mG)(mU)(mC)(mG)(fA)(mA)(mG)(mG)(mG)(rU)(rU)(mG)( | |
| mTTR- | mU)(mA)(mG)(mC)(dT)#(rC)(dC)#(rC)(dT)#(mU)(mU)(mC)(mU)(mC)( | |
| G1 | mA)(mU)(mU)#(mU)#(mC)#(mG) | |
| CR199- | (mA)#(mG)#(mG)#(mC)(mA)(mA)(dA)#(mG)(mG)(mA)(mG)(fG)(mA)(f | 428 |
| V1.31_ | A)(mG)(fA)(mG)(mU)(mC)(mG)(fA)(mA)(mG)(mG)(mG)(dT)#(rU)(mG | |
| mTTR- | )(mU)(mA)(mG)(mC)(dT)#(rC)(dC)#(dC)#(dT)#(mU)(mU)(mC)(mU)(m | |
| GI | C)(mA)(mU)(mU)#(mU)#(mC)#(mG) | |
| CR48- | (mG)#(mG)#(mG)#(rG)(rC)(rC)(rA)(rG)(rC)(rU)(rU)(rC)(rA)(rG)(rA)(rC | 429 |
| 0_ | )(rA)(rC)(rA)(rA)(rA)(rU)(rA)(rC)(rG)(rU)(rU)(rG)(rU)(rA)(rG)(rC)(rU) | |
| mTTR-G2 | (rC)(rC)(rC)(rU)(rU)(rU)(rC)(rU)(rC)(rA)(rU)(rU)#(mU)#(mC)#(mG) | |
| CR48- | (mG)#(mG)#(mG)#(mG)(mC)(rC)(rA)(rG)(mC)(mU)(mU)(rC)(mA)(rG)( | 430 |
| 4_ | mA)(rC)(rA)(mC)(rA)(rA)(rA)(mU)(rA)(rC)(mG)(rU)(rU)(rG)(mU)(mA)( | |
| mTTR-G2 | mG)(mC)(rU)(rC)(rC)(rC)(rU)(mU)(mU)(mC)(mU)(mC)(mA)(mU)(mU) | |
| | #(mU)#(mC)#(mG) | |
| CR48- | (mG)#(mG)#(mG)#(mG)(mC)(mC)(dA)#(mG)(mC)(mU)(mU)(fC)(mA)(f | 431 |
| 199_ | G)(mA)(mC)(mA)(mC)(mA)(mA)(fA)(mU)(mA)(mC)(mG)(rU)(rU)(rG)( | |
| mTTR-G2 | mU)(mA)(mG)(mC)(rU)(rC)(rC)(rC)(rU)(mU)(mU)(mC)(mU)(mC)(mA)( | |
| | mU)(mU)#(mU)#(mC)#(mG) | |
| CR48- | (mG)#(mG)#(mG)#(mG)(mC)(mC)(dA)#(mG)(mC)(mU)(mU)(fC)(mA)(f | 432 |
| 203_ | G)(mA)(mC)(mA)(mC)(fA)(mA)(fA)(mU)(mA)(mC)(mG)(rU)(rU)(rG)( | |
| mTTR-G2 | mU)(mA)(mG)(mC)(rU)(rC)(rC)(rC)(rU)(mU)(mU)(mC)(mU)(mC)(mA)( | |
| | mU)(mU)#(mU)#(mC)#(mG) | |
| CR48- | (mG)#(mG)#(mG)#(mG)(mC)(mC)(dA)#(mG)(mC)(mU)(mU)(fC)(mA)(f | 433 |
| 199- | G)(mA)(mC)(mA)(mC)(mA)(mA)(fA)(mU)(mA)(mC)(mG)(rU)(rU)(mG) | |
| V1.7_ | (mU)(mA)(mG)(mC)(rU)(rC)(dC)#(dC)#(dT)#(mU)(mU)(mC)(mU)(mC)( | |
| mTTR- | mA)(mU)(mU)#(mU)#(mC)#(mG) | |
| G2 | | |
| CR199- | (mG)#(mG)#(mG)#(mG)(mC)(mC)(dA)#(mG)(mC)(mU)(mU)(fC)(mA)(f | 434 |
| V1.18_ | G)(mA)(mC)(mA)(mC)(mA)(mA)(fA)(mU)(mA)(mC)(mG)(rU)(rU)(mG) | |
| mTTR- | (mU)(mA)(mG)(mC)(dT)#(rC)(dC)#(rC)(dT)#(mU)(mU)(mC)(mU)(mC)( | |
| G2 | mA)(mU)(mU)#(mU)#(mC)#(mG) | |
| CR199- | (mG)#(mG)#(mG)#(mG)(mC)(mC)(dA)#(mG)(mC)(mU)(mU)(fC)(mA)(f | 435 |
| V1.31_ | G)(mA)(mC)(mA)(mC)(mA)(mA)(fA)(mU)(mA)(mC)(mG)(dT)#(rU)(m | |
| mTTR- | G)(mU)(mA)(mG)(mC)(dT)#(rC)(dC)#(dC)#(dT)#(mU)(mU)(mC)(mU)( | |
| G2 | mC)(mA)(mU)(mU)#(mU)#(mC)#(mG) | |

KEY: rN = RNA, mN = 2’-O-methyl RNA, fN = 2’-fluoro RNA, dN = 2’-deoxy RNA, N = A, U, G, or C
N#N = phosphorothioate linkage

TABLE 5
Exemplary sgRNAs.
KEY: rN = RNA, mN = 2′-O-methyl RNA, fN = 2′-fluoro RNA, dN = 2′-deoxy RNA,
N = A, U, G, or C
N#N = phosphorothioate linkage

| | | SEQ |
|---|---|---|
| | | ID |
| Name | Sequence | NO: |
| SG-1 | (mC)#(mU)#(mG)#(rA)(rA)(rC)(rU)(rU)(rG)(rU)(rG)(rG)(rC)(rC)(rG)(rU)(rU) | 436 |
| TLR | (rU)(rA)(rC)(rG)(rU)(rC)(rG)(rG)(rU)(rU)(rG)(rU)(rA) | |
| MCV | (rG)(rC)(rU)(rC)(rC)(rC)(mG)(mA)(mA)(mA)(rC)(rG)(rU)(rU)(rG)(rC)(rU)(rA) | |
| | (rC)(rA)(rA)(rU)(rA)(rA)(rG)(rG)(rC)(rC)(rG) | |
| | (rU)(mC)(mU)(mG)(mA)(mA)(mA)(mA)(mG)(rA)(rU)(rG)(rU)(rG)(rC)(rC)(rG)(rC) | |
| | (rA)(rA)(rC)(rG)(rC)(rU)(rC)(rU)(rG)(rC)(rC) | |
| | (mU)(mU)(mC)(mU)(rG)(rG)(rC)(rA)(rU)(rC)(rG)(rU)#(mU)#(mU)#(mA) | |
| SG-2 | (mC)#(mU)#(mG)#(rA)(rA)(rC)(rU)(rU)(rG)(rU)(rG)(rG)(rC)(rC)(rG)(rU)(rU) | 437 |
| TLR | (rU)(rA)(rC)(rG)(rU)(rC)(rG)(rG)(rU)(rU)(rG)(rU)(mA)(mG)(mC)(rU)(rC)(rC) | |
| MCV | (rC)(mG)(mA)(mA)(mA)(rC)(rG)(rU)(rU)(rG)(rC)(rU)(rA)(rC)(rA)(rA)(rU)(rA) | |
| | ((rA)rG)(rG)(mC)(mC)(rG)(mU)(mC)(mU)(mG)(mA)(mA)(mA)(mA)(mG)(rA)(rU)(rG) | |
| | (rU)(rG)(rC)(rC)(rG)(rC)(rA)(rA)(rC)(rG)(rC)(mU)(mC)(mU)(mG)(rC)(rC)(mU) | |
| | (mU)(mC)(mU)(rG)(rG)(rC)(rA)(rU)(mC)(mG)(mU)#(mU)#(mU)#(mA) | |
| SG-3 | (mC)#(mU)#(mG)#(mA)(mA)(rC)(rU)(rU)(mG)(mU)(mG)(rG)(mC)(rC)(rG)(rU)(rU) | 438 |
| TLR | (mU)(rA)(rC)(rG)(mU)(rC)(rG)(mG)(rU)(rU)(rG)(mU)(mA)(mG)(mC)(rU)(rC) | |
| MCV | (rC)(rC)(mG)(mA)(mA)(mA)(mC)(rG)(mU)(rU)(mG)(mC)(rU)(mA)(mC)(rA)(rA) | |
| | (rU)(rA)(mA)(rG)(mG)(mC)(mC)(mG)(mU)(mC)(mU)(mG)(mA)(mA)(mA)(mA) | |
| | m(G)(mA)(mU)(rG)(mU)(mG)(mC)(mC)(mG)(mC)(mA)(mA)(mC)(mG)(mC)(mU) | |
| | (mC)(mU)(mG)(mC)(mC)(mU)(mU)(mC)(mU)(rG)(mG)(rC)(mA)(mU)(mC)(mG) | |
| | (mU)#(mU)#(mU)#(mA) | |
| SG-4 | (mC)#(mU)#(mG)#(mA)(mA)(rC)(rU)(rU)(mG)(mU)(mG)(rG)(mC)(rC)(rG)(mU) | 439 |
| TLR | (rU)(mU)(rA)(rC)(rG)(mU)(rC)(rG)(mG)(rU)(rU)(rG)(mU)(mA)(mG)(mC)(rU)(rC) | |
| MCV | (rC)(rC)(mG)(mA)(mA)(mA)(mC)(rG)(mU)(rU)(mG)(mC)(rU)(mA)(mC)(rA)(rA) | |
| | (rU)(rA)(mA)(rG)(mG)(fC)(mC)(fG)(mU)(fC)(mU)(fG)(mA)(fA)(mA)(fA)(mG) | |
| | (fA)(mU)(rG)(mU)(fG)(mC)(fC)(mG)(fC)(mA)(fA)(mC)(fG)(mC) | |
| | (fU)(mC)(fU)(mG)(fC)(mC)(fU)(mU)(fC)(mU)(rG)(mG)(rC)(mA)(fU)(mC)(fG) | |
| | (mU)#(mU)#(mU)#(mA) | |
| SG-5 | (mC)#(mU)#(mG)#(mA)(mA)(rC)(rU)(rU)(mG)(mU)(mG)(rG)(mC)(rC)(rG)(rU)(rU) | 440 |
| TLR | (mU)(rA)(rC)(rG)(mU)(rC)(rG)(mG)(rU)(rU)(rG)(mU)(mA)(mG)(mC)(rU)(rC) | |
| MCV | (rC)(rC)(mG)(mA)(mA)(mA)(rC)(rG)(rU)(rU)(rG(rC)(rU)(rA)(rC)(rA)(rA)(rU) | |
| | (rA)(rA)(rG)(rG)(mC)(mC)(rG)(mU)(mC(mU)(mG)(mA)(mA)(mA)(mA)(mG)(rA)(rU) | |
| | (rG)(rU)(rG)(rC)(rC)(rG)(rC)(rA(rA)(rC)(rG)(rC)(mU)(mC)(mU)(mG)(rC)(rC) | |
| | (mU)(mU)(mC)(mU)(rG)(rG)(rC)(rA)(rU)(mC)(mG)(mU)#(mU)#(mU)#(mA) | |
| SG-6 | (mC)#(mU)#(mG)#(rA)(mA)(rC)(rU)(rU)(mG)(mU)(rG)(rG)(mC)(rC)(rG)(rU)(rU) | 441 |
| TLR | (mU)(rA)(rC)(rG)(mU)(rC)(rG)(mG)(rU)(rU)(rG)(rU)(mA)(mG)(mC)(rU)(rC) | |
| MCV | (rC)(rC)(mG)(mA)(mA)(mA)(rC)(rG)(rU)(rU)(rG)(rC)(rU)(rA)(rC)(rA)(rA)(rU) | |
| | (rA)(rA)(rG)(rG)(mC)(mC)(rG)(mU)(mC)(mU)(mG)(mA)(mA)(mA)(mA)(mG)(rA)(rU) | |
| | (rG)(rU)(rG)(rC)(rC)(rG)(rC)(rA)(rA)(rC)(rG)(rC)(mU)(mC)(mU)(mG)(rC)(rC) | |
| | (mU)(mU)(mC)(mU)(rG)(rG)(rC)(rA)(rU)(mC)(mG)(mU)#(mU)#(mU)#(mA) | |
| SG-7 | (mC)#(mU)#(mG)#(rA)(rA)(rC)(rU)(rU)(rG)(rU)(rG)(rG)(rC)(rC)(rG)(rU)(rU) | 442 |
| TLR | (mU)(rA)(rC)(rG)(mU)(rC)(rG)(rG)(rU)(rU)(rG)(rU)(mA)(mG)(mC)(rU)(rC)(rC) | |
| MCV | (rC)(mG)(mA)(mA)(mA)(rC)(rG)(rU)(rU)(rG)(rC)(rU)(rA)(rC)(rA)(rA)(rU)(rA) | |
| | (rA)(rG)(rG)(mC)(mC)(rG)(mU)(mC)(mU)(mG)(mA)(mA)(mA)(mA)(mG)(rA)(rU)(rG) | |
| | (rU)(rG)(rC)(rC)(rG)(rC)(rA)(rA)(rC)(rG)(rC)(mU)(mC)(mU)(mG)(rC)(rC)(mU) | |
| | (mU)(mC)(mU)(rG)(rG)(rC)(rA)(rU)(mC)(mG)(mU)#(mU)#(mU)#(mA) | |
| SG-8 | (mC)#(mU)#(mG)#(mA)(mA)(mC)(mU)(mU)(mG)(mU)(mG)(mG)(mC)(mC)(rG) | 443 |
| TLR | (rU)(rU)(rU)(rA)(rC)(rG)(rU)(rC)(rG)(rG)(rU)(rU)(rG)(rU)(mA)(mG)(mC)(rU) | |
| MCV | (rC)(rC)(rC)(mG)(mA)(mA)(mA)(rC)(rG)(rU)(rU)(rG)(rC)(rU)(rA)(rC)(rA(rA) | |
| | (rU)(rA)(rA)(rG)(rG)(mC)(mC)(rG)(mU)(mC)(mU)(mG)(mA)(mA)(mA)(mA)(mG)(rA) | |
| | (rU)(rG)(rU)(rG)(rC)(rC)(rG)(rC)(rA)(rA)(rC)(rG)(rC)(mU)(mC)(mU)(mG)(rC) | |
| | (rC)(mU)(mU)(mC)(mU)(rG)(rG)(rC)(rA)(rU)(mC)(mG)(mU)#(mU)#(mU)#(mA) | |
| SG-9 | (mC)#(mU)#(mG)#(mA)(mA)(mC)(mU)(mU)(mG)(mU)(mG)(mG)(mC)(mC)(rG) | 444 |
| TLR | (rU)(rU)(mU)(rA)(rC)(rG)(mU)(rC)(rG)(mG)(rU)(rU)(rG)(mU)(mA)(mG)(mC)(rU) | |
| MCV | (rC)(rC)(rC)(mG)(mA)(mA)(mA)(rC)(rG)(rU)(rU)(rG)(rC)(rU)(rA)(rC)(rA)(rA) | |
| | (rU)(rA)(rA)(rG)(rG)(mC)(mC)(rG)(mU)(mC)(mU)(mG)(mA)(mA)(mA)(mA)(mG) | |
| | (rA)(rU)(rG)(rU)(rG)(rC)(rC)(rG)(rC)(rA)(rA)(rC)(rG)(rC)(mU)(mC)(mU)(mG) | |
| | (rC)(rC)(mU)(mU)(mC)(mU)(rG)(rG)(rC)(rA)(rU)(mC)(mG)(mU)#(mU)#(mU)#(mA) | |
| SG-10 | (mC)#(mU)#(mG)#(mA)(mA)(rC)(rU)(rU)(mG)(mU)(mG)(rG)(mC)(rC)(rG)(rU)(rU) | 445 |
| TLR | (mU)(rA)(rC)(rG)(mU)(rC)(rG)(mG)(rU)(rU)(rG)(mU)(mA)(mG)(mC)(rU)(rC)(rC) | |
| MCV | (rC)(mG)(mA)(mA)(mA)(rC)(rG)(rU)(rU)(rG)(rC)(rU)(rA)(rC)(rA)(rA)(rU)(rA) | |
| | (mA)(rG)(mG)(mC)(mC)(mG)(mU)(mC)(mU)(mG)(mA)(mA)(mA)(mA)(mG)(mA)(mU)(rG) | |
| | (rU)(mG)(mC)(mC)(rG)(rC)(rA)(rA)(rC)(rG)(rC)(mU)(mC)(mU)(mG)(rC)(rC)(mU) | |
| | (mU)(mC)(mU)(rG)(rG)(rC)(rA)(rU)(mC)(mG)(mU)#(mU)#(mU)#(mA) | |
| SG-11 | (mC)#(mU)#(mG)#(mA)(mA)(rC)(rU)(rU)(mG)(mU)(mG)(rG)(mC)(rC)(rG)(rU)(rU) | 446 |
| TLR | (mU)(rA)(rC)(rG)(mU)(rC)(rG)(mG)(rU)(rU)(rG)(mU)(mA)(mG)(mC)(rU)(rC)(rC) | |
| MCV | (rC)(mG)(mA)(mA)(mA)(rC)(rG)(rU)(rU)(rG)(rC)(rU)(rA)(rC)(rA)(rA)(rU)(rA) | |
| | (rA)(rG)(rG)(mC)(mC)(rG)(mU)(mC)(mU)(mG)(mA)(mA)(mA)(mA)(mG)(rA)(rU)(rG) | |
| | (rU)(rG)(rC)(rC)(mG)(rC)(mA)(mA)(mC)(mG)(mC)(mU)(mC)(mU)(mG)(mC)(mC)(mU) | |
| | (mU)(mC)(mU)(rG)(mG)(rC)(mA)(rU)(mC)(mG)(mU)#(mU)#(mU)#(mA) | |
| SG-12 | (mC)#(mU)#(mG)#(mA)(mA)(rC)(rU)(rU)(mG)(mU)(mG)(rG)(mC)(rC)(rG)(rU)(rU) | 447 |
| TLR | (mU)(rA)(rC)(rG)(mU)(rC)(rG)(fG)(rU)(rU)(rG)(fU)(fA)(fG)(fC)(rU)(rC)(rC) | |
| MCV | (rC)(mG)(mA)(mA)(mA)(rC)(rG)(rU)(rU)(rG)(rC)(rU)(rA)(rC)(rA)(rA)(rU)(rA) | |
| | (rA)(rG)(rG)(fC)(fC)(rG)(fU)(fC)(fU)(mG)(mA)(mA)(mA)(fA)(fG)(rA)(rU)(rG) | |
| | (rU)(rG)(rC)(rC)(rG)(rC)(rA)(rA)(rC)(rG)(rC)(mU)(mC)(fU)(fG)(rC)(rC)(mU) | |
| | (mU)(mC)(mU)(rG)(rG)(rC)(rA)(rU)(fC)(fG)(fU)#(fU)#(mU)#(mA) | |
| SG-13 | (mC)#(mU)#(mG)#(mA)(mA)(rC)(rU)(rU)(mG)(mU)(mG)(rG)(mC)(rC)(rG)(rU)(rU) | 448 |
| TLR | (mU)(rA)(rC)(rG)(mU)(rC)(rG)(fG)(rU)(rU)(rG)(fU)(fA)(fG)(fC)(rU)(rC)(rC) | |
| MCV | (rC)(mG)(mA)(mA)(mA)(mC)(rG)(mU)(rU)(fG)(fC)(rU)(fA)(fC)(rA)(rA)(rU)(rA) | |
| | (rA)(rG)(rG)(mC)(mC)(rG)(mU)(mC)(mU)(mG)(mA)(mA)(mA)(mA)(mG)(rA)(rU)(rG) | |
| | (rU)(rG)(rC)(rC)(rG)(rC)(rA)(rA)(rC)(rG)(rC)(mU)(mC)(mU)(mG)(rC)(rC)(mU) | |
| | (mU)(mC)(mU)(rG)(rG)(rC)(rA)(rU)(mC)(mG)(mU)#(mU)#(mU)#(mA) | |
| SG-14 | (mC)#(mU)#(mG)#(mA)(mA)(rC)(rU)(rU)(mG)(mU)(mG)(rG)(mC)(rC)(rG)(rU)(rU) | 449 |
| TLR | (mU)(rA)(rC)(rG)(mU)(rC)(rG)(fG)(rU)(rU)(rG)(fU)(fA)(fG)(fC)(rU)(rC)(rC) | |
| MCV | (rC)(mG)(mA)(mA)(mA)(mC)(rG)(mU)(rU)(fG)(fC)(rU)(fA)(fC)(rA)(rA)(rU)(rA) | |
| | (rA)(rG)(rG)(fC)(fC)(rG)(fU)(fC)(fU)(mG)(mA)(mA)(mA)(fA)(fG)(rA)(rU)(rG) | |
| | (rU)(rG)(rC)(rC)(rG)(rC)(rA)(rA)(rC)(rG)(rC)(mU)(mC)(fU)(fG)(rC)(rC) | |
| | (mU)(mU)(mC)(mU)(rG)(rG)(rC)(rA)(rU)(fC)(fG)(fU)#(fU)#(mU)#(mA) | |
| SG121- | mC#mU#mG#rArArCrUrUrGrUrGrGrCrCrGrUrUrUrArCrGrUrCrGrGrUrUrGrUr | 450 |
| 0_MC | ArGrCrUrCrCrCrGrArArArCrGrUrUrGrCrUrArCrArArUrArArGrGrCrCrGrUrCr | |
| V_N2T | UrGrArArArArGrArUrGrUrGrCrCrGrCrArArCrGrCrUrCrUrGrCrCrCrCrUrUrAr | |
| S126 | ArArGrCrUrCrCrUrGrCrUrUrUrArArGrGrGrGrCrArUrCrGrUmU#mU#mA | |
| SG121- | mC#mU#mG#mAmArCrUrUrGrUrGrGrCrCrGrUrUrUrArCrGrUrCrGrGrUrUrGr | 451 |
| 2_MC | UrArGrCrUrCrCrCmGmAmAmArCrGrUrUrGrCrUrArCrArArUrArArGrGrCrCr | |
| V_N2T | GmUmCmUmGmAmAmAmAmGrArUrGrUrGrCrCrGrCrArArCrGrCrUrCrUrGr | |
| S126 | CrCmCmCmUmUmAmAmAmGmCmUmCmCmUmGmCmUmUmUmAmAmGm | |
| | GrGrGrCrArUmCmGmU#mU#mU#mA | |
| SG121- | mC#mU#mG#mAmArCrUrUmGmUmGrGmCrCrGrUrUmUrArCrGmUrCrGmGr | 452 |
| 3_MC | UrUrGmUmAmGmCrUrCrCrCmGmAmAmArCrGrUrUrGrCrUrArCrArArUrArA | |
| V_N2T | rGrGrCrCrGmUmCmUmGmAmAmAmAmGrArUrGrUrGrCrCrGrCrArArCrGrCr | |
| S126 | UrCrUrGrCrCmCmCmUmUmAmAmAmGmCmUmCmCmUmGmCmUmUmUm | |
| | AmAmGmGrGrGrCrArUmCmGmU#mU#mU#mA | |
| SG121- | mC#mU#mG#mAmAmCmUmUmGmUmGmGmCmCrGrUrUmUrArCrGmUrCrG | 453 |
| 4_MC | mGrUrUrGmUmAmGmCrUrCrCrCmGmAmAmArCrGrUrUrGrCrUrArCrArArUr | |
| V_N2T | ArArGrGrCrCrGmUmCmUmGmAmAmAmAmGrArUrGrUrGrCrCrGrCrArArCr | |
| S126 | GrCrUrCrUrGrCrCmCmCmUmUmAmAmAmGmCmUmCmCmUmGmCmUmU | |
| | mUmAmAmGmGrGrGrCrArUmCmGmU#mU#mU#mA | |
| SG121- | mC#mC#mG#rCrArArArGrCrUrGrCrArUrCrCrArCrCrCrCrCrCrGrGrUrUrGrUr | 454 |
| 0_DS12 | ArGrCrUrCrCrCrGrArArArCrGrUrUrGrCrUrArCrArArUrArArGrGrCrCrGrUrCr | |
| | UrGrArArArArGrArUrGrUrGrCrCrGrCrArArCrGrCrUrCrUrGrCrCrCrCrUrUrAr | |
| | ArArGrCrUrCrCrUrGrCrUrUrUrArArGrGrGrGrCrArUrCrGrUmU#mU#mA | |
| SG121- | mC#mC#mG#mCmArArArGrCrUrGrCrArUrCrCrArCrCrCrCrCrCrGrGrUrUrGrU | 455 |
| 2_DS12 | rArGrCrUrCrCrCmGmAmAmArCrGrUrUrGrCrUrArCrArArUrArArGrGrCrCrG | |
| | mUmCmUmGmAmAmAmAmGrArUrGrUrGrCrCrGrCrArArCrGrCrUrCrUrGrCr | |
| | CmCmCmUmUmAmAmAmGmCmUmCmCmUmGmCmUmUmUmAmAmGmG | |
| | rGrGrCrArUmCmGmU#mU#mU#mA | |
| SG121- | mC#mC#mG#mCmArArArGmCmUmGrCmArUrCrCrAmCrCrCrCmCrCrGmGrU | 456 |
| 3_DS12 | rUrGmUmAmGmCrUrCrCrCmGmAmAmArCrGrUrUrGrCrUrArCrArArUrArAr | |
| | GrGrCrCrGmUmCmUmGmAmAmAmAmGrArUrGrUrGrCrCrGrCrArArCrGrCr | |
| | UrCrUrGrCrCmCmCmUmUmAmAmAmGmCmUmCmCmUmGmCmUmUmUm | |
| | AmAmGmGrGrGrCrArUmCmGmU#mU#mU#mA | |
| SG121- | mC#mC#mG#mCmAmAmAmGmCmUmGmCmAmUrCrCrAmCrCrCrCmCrCrG | 457 |
| 4_DS12 | mGrUrUrGmUmAmGmCrUrCrCrCmGmAmAmArCrGrUrUrGrCrUrArCrArArUr | |
| | ArArGrGrCrCrGmUmCmUmGmAmAmAmAmGrArUrGrUrGrCrCrGrCrArArCr | |
| | GrCrUrCrUrGrCrCmCmCmUmUmAmAmAmGmCmUmCmCmUmGmCmUmU | |
| | mUmAmAmGmGrGrGrCrArUmCmGmU#mU#mU#mA | |
| SG121- | mU#mA#mU#rGrUrUrCrCrArGrCrUrUrCrCrUrGrGrGrUrCrUrGrCrGrUrUrGrUr | 458 |
| 0_DS22 | ArGrCrUrCrCrCrGrArArArCrGrUrUrGrCrUrArCrArArUrArArGrGrCrCrGrUrCr | |
| | UrGrArArArArGrArUrGrUrGrCrCrGrCrArArCrGrCrUrCrUrGrCrCrCrCrUrUrAr | |
| | ArArGrCrUrCrCrUrGrCrUrUrUrArArGrGrGrGrCrArUrCrGrUmU#mU#mA | |
| SG121- | mU#mA#mU#mGmUrUrCrCrArGrCrUrUrCrCrUrGrGrGrUrCrUrGrCrGrUrUrGr | 459 |
| 2_DS22 | UrArGrCrUrCrCrCmGmAmAmArCrGrUrUrGrCrUrArCrArArUrArArGrGrCrCr | |
| | GmUmCmUmGmAmAmAmAmGrArUrGrUrGrCrCrGrCrArArCrGrCrUrCrUrGr | |
| | CrCmCmCmUmUmAmAmAmGmCmUmCmCmUmGmCmUmUmUmAmAmGm | |
| | GrGrGrCrArUmCmGmU#mU#mU#mA | |
| SG121- | mU#mA#mU#mGmUrUrCrCmAmGmCrUmUrCrCrUrGmGrGrUrCmUrGrCmGr | 460 |
| 3_DS22 | UrUrGmUmAmGmCrUrCrCrCmGmAmAmArCrGrUrUrGrCrUrArCrArArUrArA | |
| | rGrGrCrCrGmUmCmUmGmAmAmAmAmGrArUrGrUrGrCrCrGrCrArArCrGrCr | |
| | UrCrUrGrCrCmCmCmUmUmAmAmAmGmCmUmCmCmUmGmCmUmUmUm | |
| | AmAmGmGrGrGrCrArUmCmGmU#mU#mU#mA | |
| SG121- | mU#mA#mU#mGmUmUmCmCmAmGmCmUmUmCrCrUrGmGrGrUrCmUrGrC | 461 |
| 4_DS22 | mGrUrUrGmUmAmGmCrUrCrCrCmGmAmAmArCrGrUrUrGrCrUrArCrArArUr | |
| | ArArGrGrCrCrGmUmCmUmGmAmAmAmAmGrArUrGrUrGrCrCrGrCrArArCr | |
| | GrCrUrCrUrGrCrCmCmCmUmUmAmAmAmGmCmUmCmCmUmGmCmUmU | |
| | mUmAmAmGmGrGrGrCrArUmCmGmU#mU#mU#mA | |
| SG121- | mG#mA#mG#rGrCrArArGrArGrGrGrCrGrGrCrUrUrUrGrGrGrCrGrGrUrUrGrUr | 462 |
| 0_DS28 | ArGrCrUrCrCrCrGrArArArCrGrUrUrGrCrUrArCrArArUrArArGrGrCrCrGrUrCr | |
| | UrGrArArArArGrArUrGrUrGrCrCrGrCrArArCrGrCrUrCrUrGrCrCrCrCrUrUrAr | |
| | ArArGrCrUrCrCrUrGrCrUrUrUrArArGrGrGrGrCrArUrCrGrUmU#mU#mA | |
| SG121- | mG#mA#mG#mGmCrArArGrArGrGrGrCrGrGrCrUrUrUrGrGrGrCrGrGrUrUrGr | 463 |
| 2_DS28 | UrArGrCrUrCrCrCmGmAmAmArCrGrUrUrGrCrUrArCrArArUrArArGrGrCrCr | |
| | GmUmCmUmGmAmAmAmAmGrArUrGrUrGrCrCrGrCrArArCrGrCrUrCrUrGr | |
| | CrCmCmCmUmUmAmAmAmGmCmUmCmCmUmGmCmUmUmUmAmAmGm | |
| | GrGrGrCrArUmCmGmU#mU#mU#mA | |
| SG121- | mG#mA#mG#mGmCrArArGmAmGmGrGmCrGrGrCrUmUrUrGrGmGrCrGmGr | 464 |
| 3_DS28 | UrUrGmUmAmGmCrUrCrCrCmGmAmAmArCrGrUrUrGrCrUrArCrArArUrArA | |
| | rGrGrCrCrGmUmCmUmGmAmAmAmAmGrArUrGrUrGrCrCrGrCrArArCrGrCr | |
| | UrCrUrGrCrCmCmCmUmUmAmAmAmGmCmUmCmCmUmGmCmUmUmUm | |
| | AmAmGmGrGrGrCrArUmCmGmU#mU#mU#mA | |
| SG121- | mG#mA#mG#mGmCmAmAmGmAmGmGmGmCmGrGrCrUmUrUrGrGmGrCr | 465 |
| 4_DS28 | GmGrUrUrGmUmAmGmCrUrCrCrCmGmAmAmArCrGrUrUrGrCrUrArCrArAr | |
| | UrArArGrGrCrCrGmUmCmUmGmAmAmAmAmGrArUrGrUrGrCrCrGrCrArAr | |
| | CrGrCrUrCrUrGrCrCmCmCmUmUmAmAmAmGmCmUmCmCmUmGmCmUm | |
| | UmUmAmAmGmGrGrGrCrArUmCmGmU#mU#mU#mA | |

### Example 2—Genome Editing Efficiency of Chemically Modified Nme crRNA and Nme tracrRNA

Chemically Modified crRNA and tracrRNA Screening Methods

Cell Culture

Screening was performed in a HEK293T stable cell line expressing the traffic light reporter (TLR) Multi-Cas Variant 1 system (TLR-MCV1). The HEK293T cells were cultured in Dulbecco-modified Eagle's Minimum Essential Medium (DMEM; Life Technologies). DMEM was also supplemented with 10% Fetal Bovine Serum (FBS; Sigma). Cells were grown in a humidified 37° C., 5% CO₂ incubator.

Traffic Light Reporter (TLR) System

The traffic light reporter (TLR) system includes a GFP (containing an insertion), followed by an out-of-frame mCherry. Upon double stranded break induction, a subset of non-homologous end-joining (NHEJ) repair events generate indels that place mCherry in frame, leading to red fluorescence. Detection of the red fluorescence is therefore a readout of editing efficiency. This system was developed and further described in Certo et al. (Nat. Methods 8, 671 (2011)). This system was further developed for testing the modified crRNAs and tracrRNAs of the disclosure. The TLR Multi-Cas Variant 1 system (TLR-MCV1) was created to introduce protospacer adjacent motifs (PAMs) to multiple alternative CRISPR enzymes (Streptococcus pyogenes (SpyCas9), Neisseria meningitidis (Nme1Cas9 and Nme2Cas9), Campylobacter jejuni (CjeCas9), Staphylococcus aureus (SauCas9), Geobacillus stearothermophilus (GeoCas9), Lachnospiraceae bacterium ND2006 (LbaCas12a), Acidaminococcus sp. (AspCas12a), and Francisella novicida (FnoCas12)) (see Iyer et al. (BioRxiv. (2019)).

The gRNAs used herein contain a 24-nucleotide guide sequence of CUGAACUUGUGGCCGUUUACGUCG (SEQ ID NO: 12), for targeting the Nme2Cas9 PAM in the TLR-MCV1 system.

Expression and Purification of Nme2Cas9

A vector expressing Nme2Cas9 from Neisseria meningitidis was used. In this construct, the Cas9 also contains three nuclear localization signals (NLSs). Rosetta DE3 strain of Escherichia coli was transformed with the 3×NLS-Nme2Cas9 construct. For expression and purification of 3×NLS-Nme2Cas9, a previously described protocol was used (Jinek et al. Science, 337: 816 (2012)). The bacterial culture was grown at 37° C. until an OD600 of 0.6 was reached. Then, the bacterial culture was cooled to 18° C., and 1 mM Isopropyl β-D-1-thiogalactopyranoside (IPTG; Sigma) was added to induce protein expression. Cells were grown overnight for 16-20 hours.

The bacterial cells were harvested and resuspended in Lysis Buffer [50 mM Tris-HCl (pH 8.0), 5 mM imidazole]. 10 μg/mL of Lysozyme (Sigma) was then added to the mixture and incubated for 30 minutes at 4° C. This was followed by the addition of 1×HALT Protease Inhibitor Cocktail (ThermoFisher). The bacterial cells were then sonicated and centrifuged for 30 minutes at 18,000 rpm. The supernatant was then subjected to Nickel affinity chromatography. The elution fractions containing the Nme2Cas9 were then further purified using cation exchange chromatography using a 5 mL HiTrap S HP column (GE). This was followed by a final round of purification by size-exclusion chromatography using a Superdex-200 column (GE). The purified protein was concentrated and flash frozen for subsequent use.

Transfection of HEK293T Cells

The HEK293T cells were nucleofected using the Neon transfection system (ThermoFisher) according to the manufacturer's protocol. Briefly, 20 picomoles of 3×NLS-Nme2Cas9 was mixed with 25 picomoles of crRNA:tracrRNA in buffer R (ThermoFisher) and incubated at room temperature for 20-30 minutes. This Cas9 RNP complex was then mixed with approximately 100,000 cells which were already resuspended in buffer R. This mixture was nucleofected with a 104 Neon tip and then plated in 24-well plates containing 500 μL of DMEM and 10% FBS. The cells were stored in a humidified 37° C. and 5% CO2 incubator for 2-3 days.

Flow Cytometry Analysis

The nucleofected HEK293T cells were analyzed on MACSQuant® VYB from Miltenyi Biotec. For mCherry detection, the yellow laser (561 nm) was used for excitation and 615/20 nm filter used to detect emission. At least 20,000 events were recorded and the subsequent analysis was performed using FlowJo® v10.4.1. Cells were first sorted based on forward and side scattering (FSC-A vs SSC-A) to eliminate debris. Cells were then gated using FSC-A and FSC-H to select single cells. Finally, mCherry signal was used to select for mCherry-expressing cells. The percent of cells expressing mCherry was calculated and reported in this application as a measure of Cas9-based genome editing.

Indel Analysis by TIDE

The genomic DNA from HEK293T cells was harvested using DNeasy Blood and Tissue kit (Qiagen) as recommended by the manufacturer. Approximately 50 ng of genomic DNA was used to PCR-amplify a ˜700 base pair fragment that was subsequently purified using a QIAquick PCR Purification kit (Qiagen). The PCR fragment was then sequenced by Sanger sequencing and the trace files were subjected to indel analysis using the TIDE web tool (Brinkman et al. Nucleic Acids Research, 42: e168 (2014)). Results are reported as % Indel rate.

Screening of Chemical Modification Patterns

Structure-guided and systematic approaches were used to introduce 2′-OMe-RNA, 2′-F-RNA, 2′-deoxy, and PS modifications throughout Nme Cas9 nuclease-compatible gRNAs. These modifications were chosen because they have been shown to improve stability, efficacy, and immunotoxicity associated with RNA. The strategy described herein yielded active RNP complexes with both extensively modified versions of crRNAs and tracrRNAs.

CRISPR RNA (crRNA) Chemical Modifications

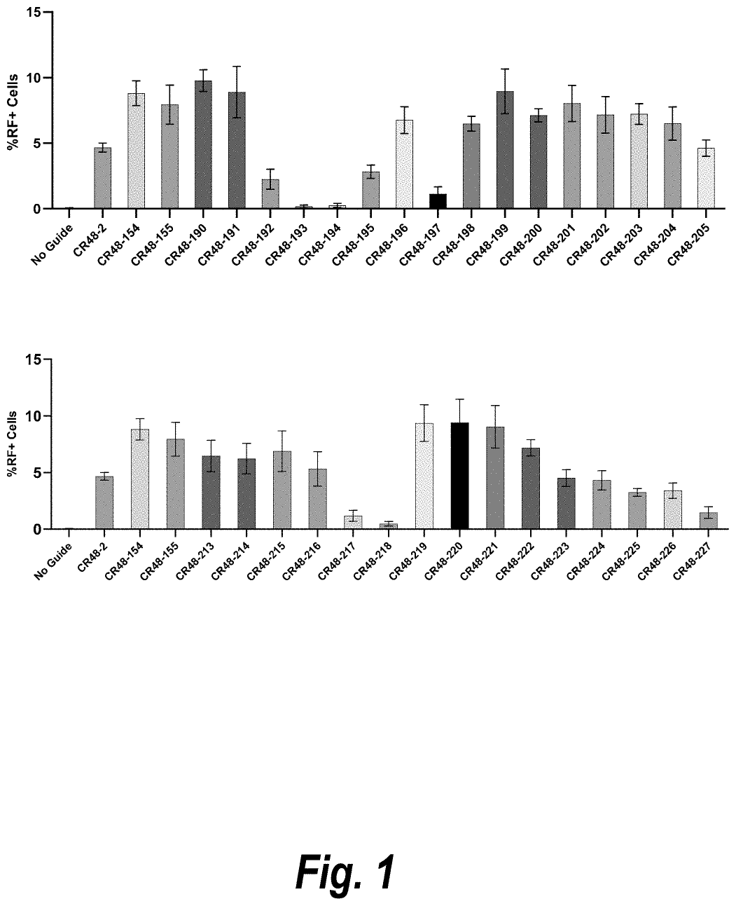

FIG. 1 depicts a screen of 48-nucleotide long crRNA patterns CR48-2, CR48-154, CR48-155, CR48-190 to CR48205, and CR48-213 to CR48-227, each targeting the Nme2Cas9 site. An unmodified Nme tracrRNA was paired with each crRNA. The Traffic Light Reporter Multi-Cas Variant 1 (TLR-MCV1) reporter was used. 50,000 TLR-MCV cells were electroporated with 5 pmol Nme2Cas9, 13.75 pmol annealed gRNA RNPs. The graphs show the percentages of red fluorescent (RF) cells obtained by fluorescence activated cell sorting (FACS) analysis, which was performed 48 hours to 72 hours after electroporation. Data are mean values of three biological replicates and error bars represent s.e.m.

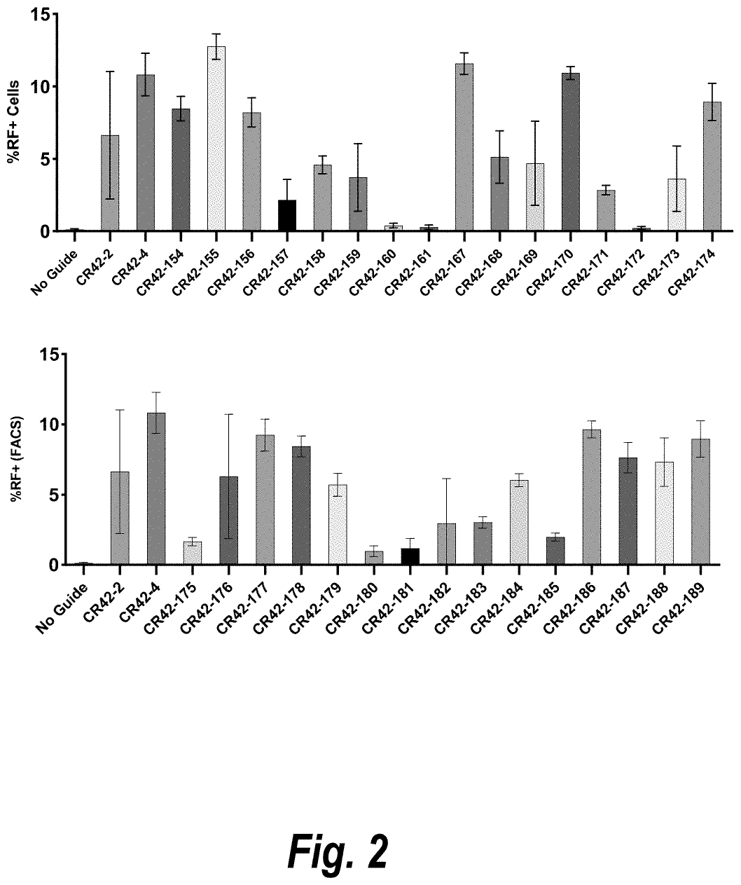

FIG. 2 depicts a screen of 42-nucleotide long crRNA patterns CR42-2, CR42-4, CR42-154 to CR42-161, CR42-167 to CR42-189, each targeting the Nme2Cas9 site. An unmodified Nme tracrRNA was paired with each crRNA. The Traffic Light Reporter Multi-Cas Variant 1 (TLR-MCV1) reporter was used. 50,000 TLR-MCV cells were electroporated with 10 pmol Nme2Cas9, 27.5 pmol annealed gRNA RNPs. The graphs show the percentages of red fluorescent (RF) cells obtained by fluorescence activated cell sorting (FACS) analysis, which was performed 48 hours to 72 hours after electroporation. Data are mean values of three biological replicates and error bars represent s.e.m.

Numerous crRNAs demonstrated efficacy similar to or greater than that of the minimally modified crRNA, CR48-2 or CR-42-2.

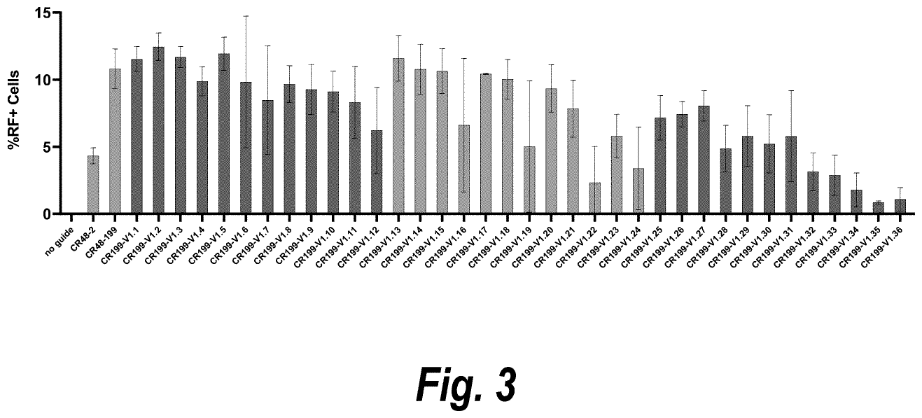

Based on the results of this initial screen, the CR48-199 crRNA chemical modification pattern was used as the basis to generate variants chemical modification patterns, CR48-199 V1.1 to CR48-199 V1.36. The results of the screen are depicted in FIG. 3. An unmodified Nme tracrRNA was paired with each crRNA. The TLR-MCV1 reporter was used. 50,000 TLR-MCV cells were electroporated with 5 pmol Nme2Cas9, 13.75 pmol annealed gRNA RNPs. The graphs show the percentages of RF cells obtained by FACS analysis, which was performed 48 hours to 72 hours after electroporation. Data are mean values of three biological replicates and error bars represent s.e.m.

The CR48-199 variants included 2′-deoxy RNA or 2′-O-methyl modifications at several previously unmodified positions in the crRNA repeat sequence. These positions include position 26, 33, 34, 35, 36, and 37, counting from the 5′ end of a 48-nucleotide long crRNA. Numerous crRNAs demonstrated efficacy similar to that of the CR48-199 pattern, while having a higher percentage of modified nucleotides.

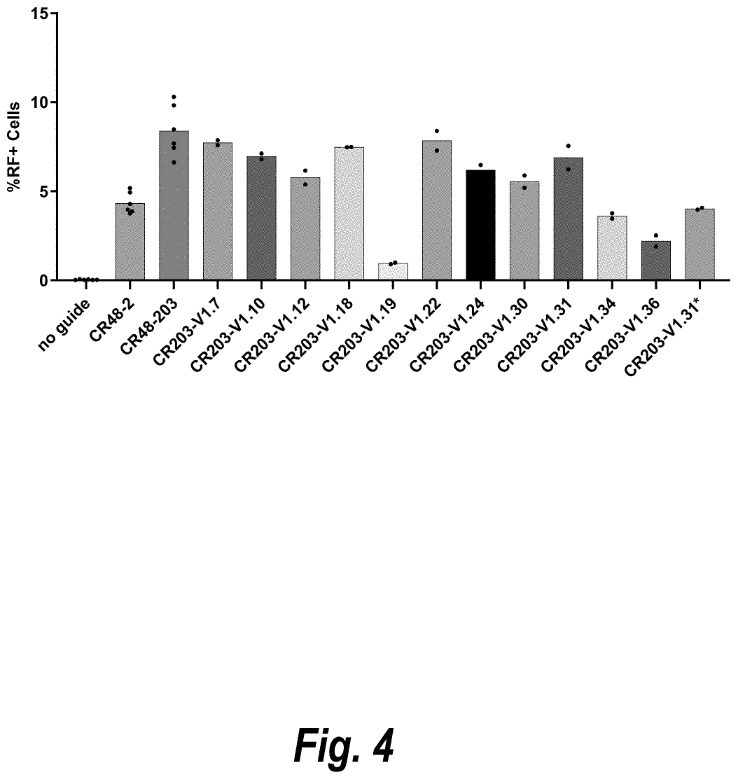

Also based on the results of this initial screen, the CR48-203 crRNA chemical modification pattern was used as the basis to generate variants chemical modification patterns, CR48-203 V1.7, CR48-203 V1.10, CR48-203 V1.12, CR48-203 V1.18, CR48-203 V1.19, CR48-203 V1.22. CR48-203 V1.24, CR48-203 V1.30, CR48-203 V1.31, CR48-203 V1.34, CR48-203 V1.36, CR48-203 V1.31*. The results of the screen are depicted in FIG. 4. An unmodified Nme tracrRNA was paired with each crRNA. The TLR-MCV1 reporter was used. 50,000 TLR-MCV cells were electroporated with 5 pmol Nme2Cas9, 13.75 pmol annealed gRNA RNPs. The graphs show the percentages of RF cells obtained by FACS analysis, which was performed 48 hours to 72 hours after electroporation. Data are mean values of three biological replicates and error bars represent s.e.m.

The CR48-203 variants included 2′-deoxy RNA or 2′-O-methyl modifications at several previously unmodified positions in the crRNA repeat sequence. These positions include position 26, 33, 34, 35, 36, and 37, counting from the 5′ end of a 48-nucleotide long crRNA. Numerous crRNAs demonstrated efficacy similar to that of the CR48-203 pattern, while having a higher percentage of modified nucleotides.

TRACR RNA (tracrRNA) Chemical Modifications

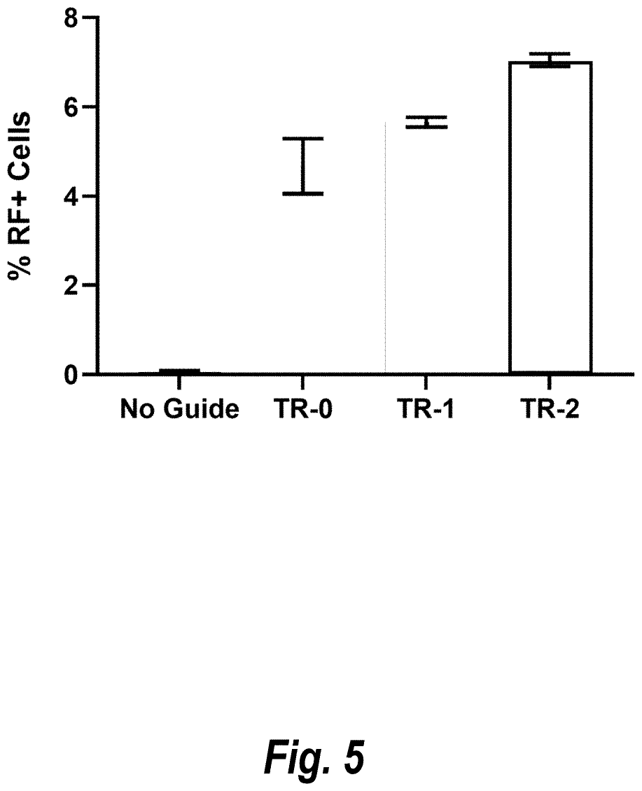

FIG. 5 depicts editing efficiencies of several 93-nucleotide long Nme chemically modified tracrRNAs (TR-0, TR-1, and TR-2). A minimally modified Nme crRNA (CR48-2) was paired with each tracrRNA. The TLR-MCV1 reporter was used. The graphs show the percentages of RF cells obtained by FACS analysis. Data are mean values of three biological replicates and error bars represent s.e.m.

Each of the three chemically modified tracrRNAs demonstrated strong editing efficacy. TR-2 possessed the highest editing efficiency while also possessing the highest percentage of ribose group chemical modifications (about 47% ribose chemical modification).

Single Guide RNA (sgRNA) Chemical Modifications

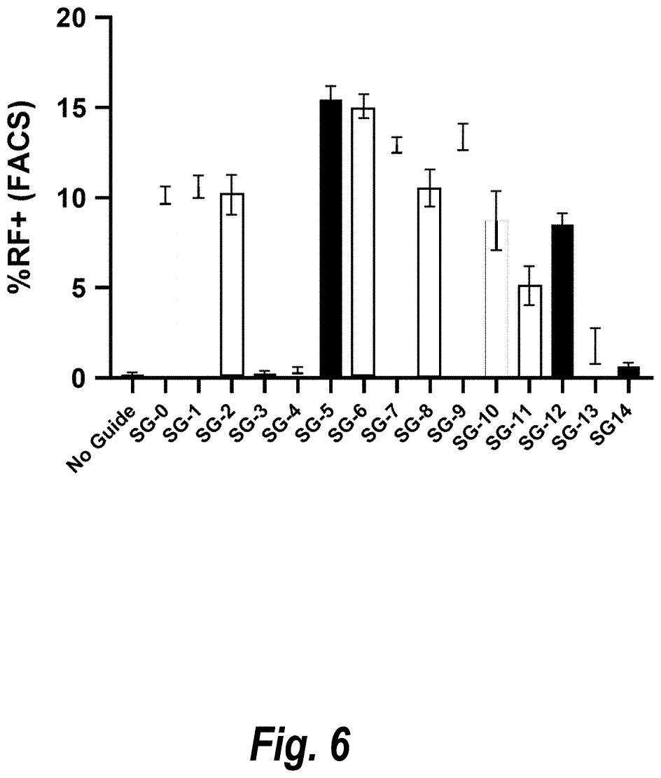

FIG. 6 depicts editing efficiencies of several 103-nucleotide long Nme chemically modified single guide RNAs (sgRNAs) (SG-0 to SG-14). The TLR-MCV1 reporter cell line was used and transfected with an RNP comprising Nme2Cas9 and the gRNAs. 100,000 cells were electroporated with 10 pmol RNP (10 pmol of Nme2Cas9 and 30 pmol of sgRNA). The graphs show the percentages of RF cells obtained by FACS analysis. Data are mean values of three biological replicates and error bars represent s.e.m.

Several Nme chemically modified sgRNAs demonstrated strong editing efficacy. High editing efficiency was observed with ribose group chemical modification of up to about 47%.

### Example 3—Viral Delivery of Nme2Cas9 Paired with Chemically Modified gRNA

Viral delivery methods may be employed to deliver (i.e., express) the Cas9 nuclease in cells and, optionally, express either a crRNA portion or a tracrRNA portion. A crRNA or tracrRNA would be encoded by the viral vector and therefore be un-chemically modified. A chemically modified crRNA or tracrRNA may be co-delivered to complete the dual gRNA in cells transduced with the viral vector. For example, but in no way limiting, cells may be transduced with a viral vector (either in vivo, in vitro, or ex vivo) that expresses a Nme Cas9 nuclease (e.g., Nme2Cas9) and an Nme tracrRNA. A chemically modified Nme crRNA as described herein may be co-delivered with said viral vector, either simultaneously or sequentially. Once in cells, the chemically modified Nme crRNA would pair with the expressed tracrRNA and the Nme Cas9 nuclease, thereby permitting targeting of the Nme Cas9 to a target site in the genome of the cells. This same procedure may be employed with a viral vector encoding the Nme Cas9 co-delivered with a chemically modified sgRNA.

To test the ability to use viral vectors with co-delivered chemically modified gRNAs, 50,000 cells were transfected with a plasmid encoding the AAV genome and expressing Nme2Cas9 and a tracrRNA. 18 hours after seeding wells with the 50,000 cells, the cells were transfected with 200 ng of plasmid DNA encoding Nme2Cas9 (for chemically modified sgRNA co-delivery) or Nme2Cas9 and 93 nt tracrRNA (for chemically modified crRNA co-delivery). 18 hours later cells expressing Nme2Cas9 or Nme2Cas9/tracrRNA were collected and electroporated with 50 pmol of chemically modified sgRNA or chemically modified crRNA. The electroporated cells were incubated for an additional 24 hours before analysis of mCherry signal by flow cytometry.

50 pmole of either sgRNAs SG-1 or SG-5, or crRNAs CR-0, CR-2, CR-4, and CR-128 were co-delivered. CR-4 is based on the chemical modification pattern in SG-5 over the crRNA portion and is 42-nucleotides in length.

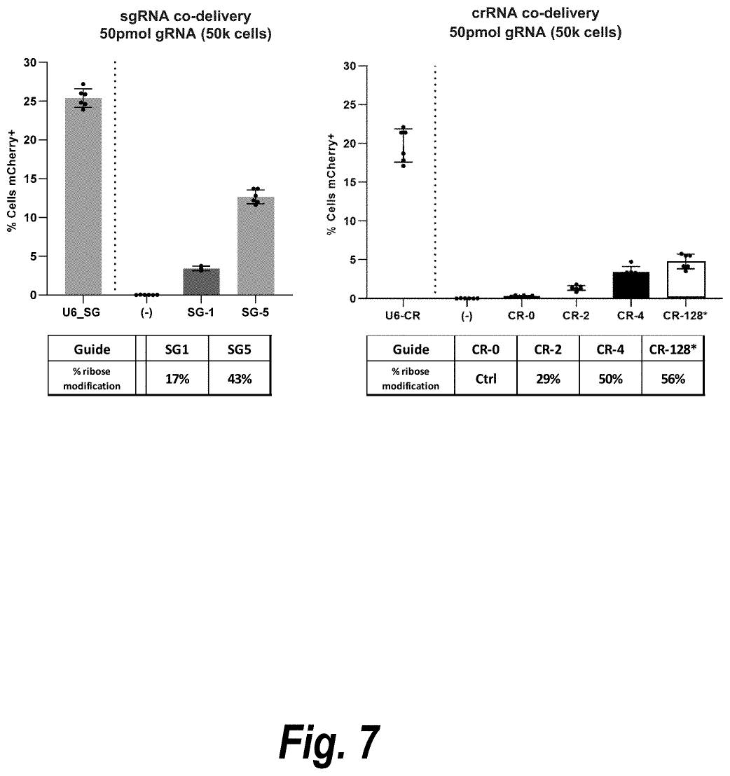

As show in FIG. 7, all chemically modified gRNAs (either sgRNAs or crRNAs) demonstrated editing when combined with the AAV expressing Nme2Cas9. The AAV also expressed a tracrRNA for the crRNA experiments, but did not express a tracrRNA for the sgRNA experiments. Increasing levels of ribose chemical modification lead to higher editing efficiency. As a control, an AAV containing a U6-promoter expressed gRNA was used.

### Example 4—In Vivo Gene Editing

The various chemically modified guide RNAs have displayed substantial gene editing activity in vitro while possessing enhanced stability (e.g., serum stability). The in vivo activity of select chemically modified guide RNAs was next determined in a mouse. A mouse containing the TLR-MCV1 reporter was used in the experiments. Chemically modified sgRNA were buffer exchanged and concentrated using Amicon ultra 0.5 mL 3k MWCO to a concentration of about 230 μM (3× washes 1M NaOAc, 3× washes PBS pH7.4). RNP were formulated in 1 mL of PBS pH7.4, and incubated for 0.5 hrs. Specifically, a 1 to 2.785 Nme2cas9 to Guide molar ratio was used. RNP were further concentrated in a 4 mL Amicon filter 30k MWCO and spun at 4000 g to 35-50 μL. Finally, highly concentrated Shuttle peptide was added to the RNP mixture at a 5× molar ratio to Nme2Cas9 RNP. The resultant mixture (Nme2Cas9 RNP and shuttle peptide) was snap frozen and stored at −80° C. until use.

The shuttle peptide is a small peptide that aids in cell membrane penetration and endosomal escape. The sequence used in this study is named S10 and described in Krishnamurthy et al. (Nat Commun. 10: 4906 (2019)).

3 uL of the RNP solution was injected Bilaterally into the striatum of TLR-MCV1 mice. After 7-days, mice were euthanized according to IACUC protocols. Dissected brains were sectioned and stained for mCherry with IHC 1 week after RNP injections.

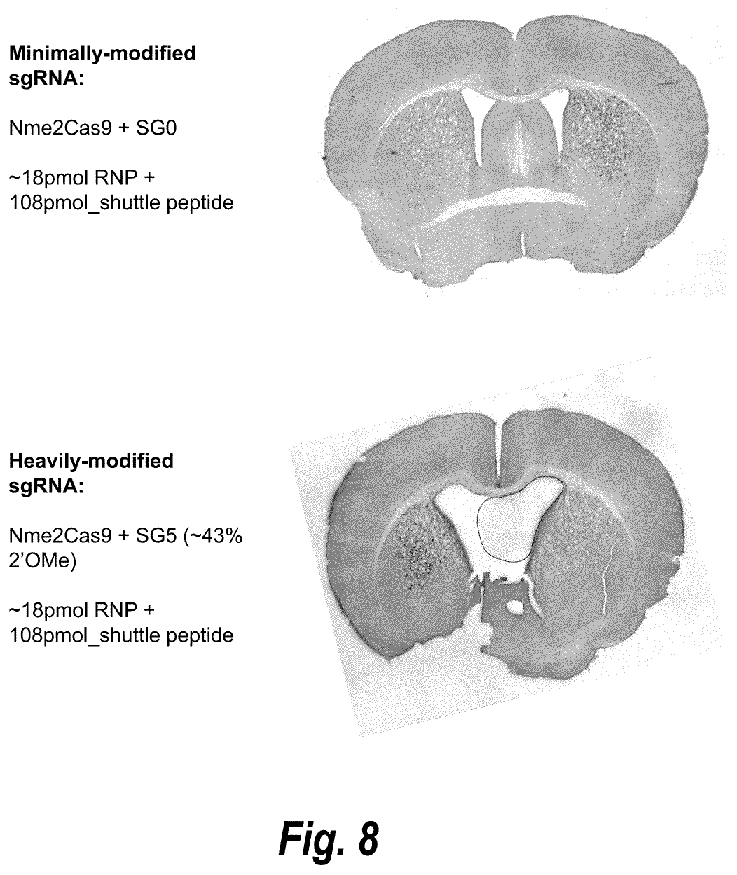

As shown in FIG. 8, the data shows that the chemically modified sgRNAs are capable of gene editing activity in vivo.

### Example 5—Developing and Testing 121-Nucleotide Long Nme sgRNA

103-nucleotide long Nme chemically modified sgRNAs (SG103-0) were compared against 121-nucleotide long Nme chemically modified sgRNAs (SG121-0). The sgRNAs used in the Examples are recited in Table 5.

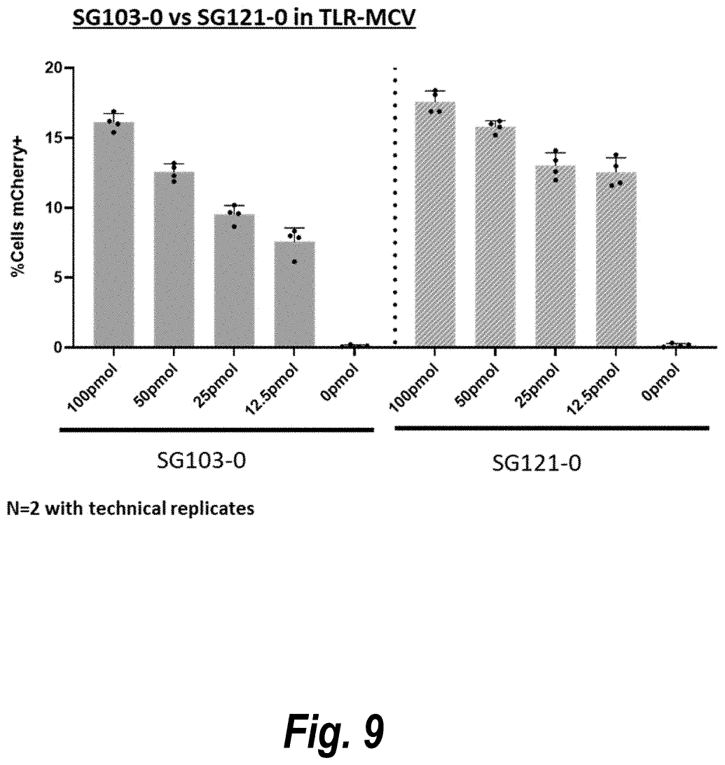

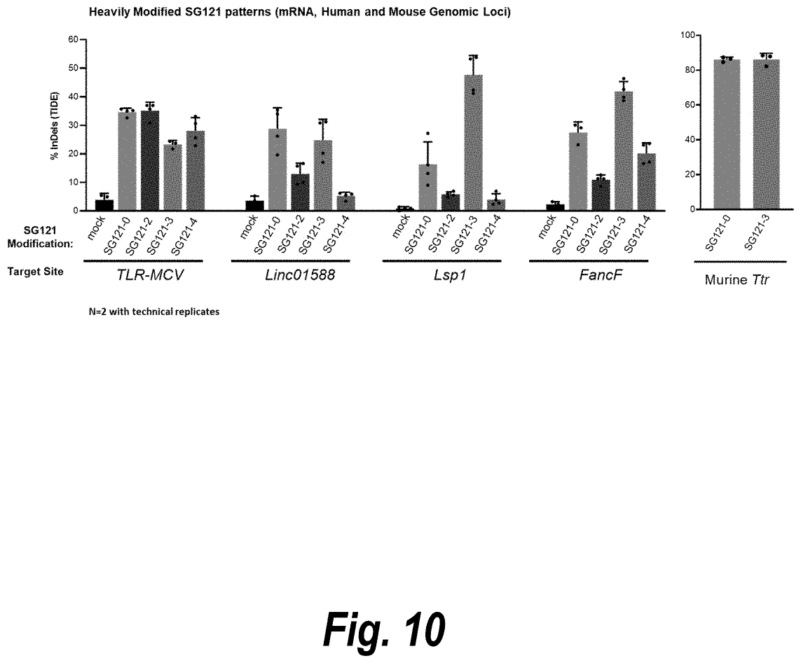

The codelivery of mRNA and sgRNA using 103-nucleotide long Nme chemically modified sgRNAs (SG103-0) vs 121-nucleotide long Nme chemically modified sgRNAs (SG121-0) was measured. Nme2Cas9 mRNA was kept constant and end-modified synthetic sgRNA was titrated. The activity was measured and plotted in FIG. 9. The bars represent activity in the TLR-MCV cell line. Data are mean values of two biological replicates and error bars represent s.e.m.

Further modifications patterns of the 121-nucleotide long Nme chemically modified sgRNAs (SG121) were developed and tested against human HEK293T cells, and mouse Hepa1-6 cells. SG121-3, for example, is ˜45% ribose modified and supports efficient editing at multiple genomic loci. This amount of modification is expected to support in vivo editing when delivered as an LNP. FIG. 10 depicts the comparison of the activity of several of the SG121 modifications (SG121-0, SG121-2, SG121-3, and SG121-4) in targeting human TLR-MCV cell line, HEK293T cells (Linc01588, Lsp1, and FancF), and mouse Hepa1-6 cells (Ttr). SG121 modified guides were electroporated into cells with Nme2Cas9 mRNA. All of the tested sgRNAs displayed higher activity than the mock control background.

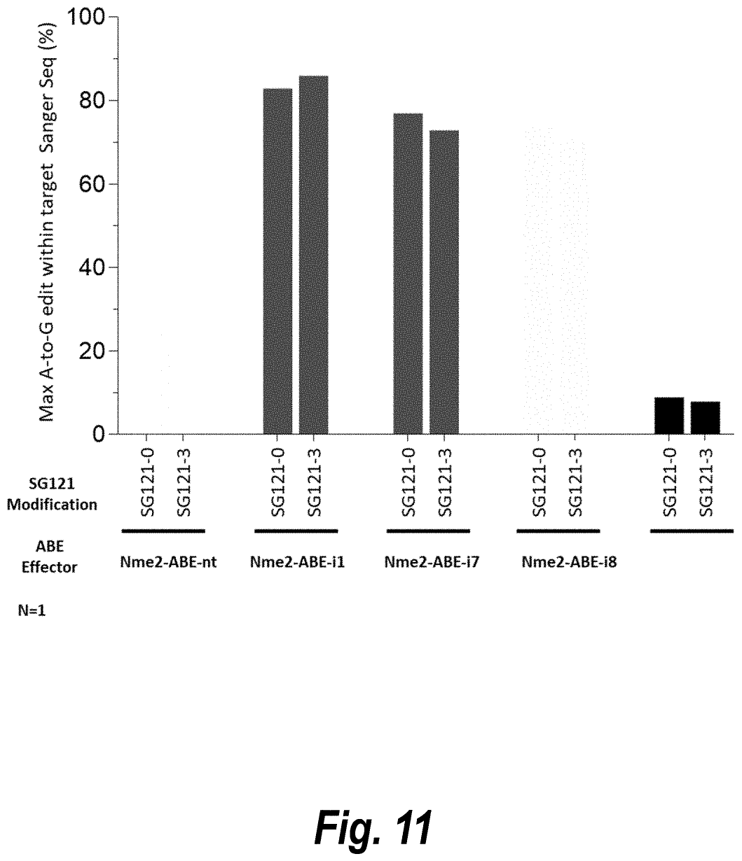

SG121-3 and SG121-0 were also tested using various Nme2-adenosine base editor (ABE) fusion proteins at the Linc01588 genomic site. These modified guide RNAs displayed substantial gene editing activity in vitro. The activities of these sgRNA were plotted as shown in FIG. 11.

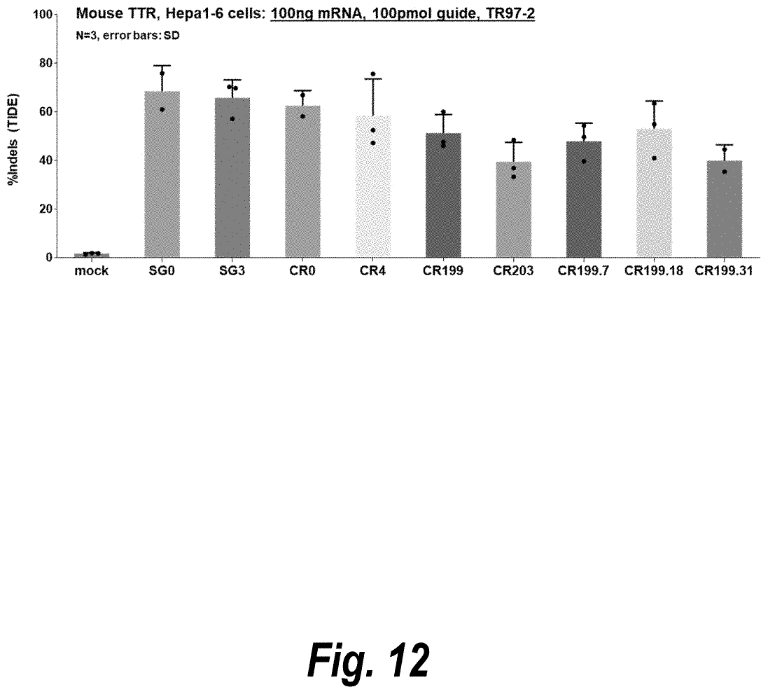

To test which crRNA and tracrRNA modification patterns can extend to desired genomic sites, 100 pmol of the SG121 guides (SG121-0, and SG121-3) and of the heavily modified CR48 variants (CR48-199, CR48-203, CR48-199, 7, CR48-199, 18, and CR48-199.31) were used to target mouse TTR with 100 ng of Nme2Cas9 mRNA and tracrRNA TR97-2. crRNAs were delivered with TR97-2. TR97-2 and SG121-3's tracrRNA sequences have comparable modifications allowing comparison of editing results. The modified crRNAs showed comparable editing activities targeting mouse TTR as those observed via TLR-MCV screening (mCherry reporter cell line) (see FIG. 12). This proves that the crRNA modification patterns observed also extend to alternative genomic loci.

1. A chemically modified Neisseria meningitidis (Nme) guide RNA (gRNA) comprising:
(a) a crRNA portion comprising (i) a guide sequence capable of hybridizing to a target polynucleotide sequence, and (ii) a repeat sequence; and
(b) a tracrRNA portion comprising an anti-repeat nucleotide sequence that is complementary to the repeat sequence,
wherein the crRNA portion comprises at least 40% modified nucleotides, and
wherein the gRNA is capable of binding to an Nme Cas9 nuclease or a variant thereof.

2. The chemically modified Nme gRNA of claim 1, wherein:
the Nme Cas9 nuclease is Nme1Cas9, Nme2Cas9, or Nme3Cas9;
the gRNA is capable of binding to an Nme Cas9 nuclease or variant thereof comprising an amino acid sequence with at least 80% identity to an amino acid sequence set forth in SEQ ID NO: 1, 2, or 3;
the crRNA portion binds to an Nme Cas9 nuclease or variant thereof through one or more nucleobases at positions 2, 11, 12, 14, 17, 20, 34, 35, and 36 from the 5′ end of the crRNA portion;
the repeat sequence of the crRNA portion binds to an Nme Cas9 nuclease or variant thereof through one or more nucleobases at positions 10, 11, and 12 from the 5′ end of the repeat sequence of the crRNA portion;
the guide sequence of the crRNA portion binds to an Nme Cas9 nuclease or variant thereof through one or more nucleobases at positions 5, 8, 11, 13, 14, and 23 from the 3′ end of the guide sequence of the crRNA portion;
the tracrRNA portion comprises at least one modified nucleotide;
the modified nucleotides each independently comprise a modification of a ribose group, a phosphate group, a nucleobase, or a combination thereof;
each modification of the ribose group is independently selected from the group consisting of 2′-O-methyl, 2′-fluoro, 2′-deoxy, 2′-O-(2-methoxyethyl) (MOE), 2′-NH₂ (2′-amino), 4′-thio, a bicyclic nucleotide, a locked nucleic acid (LNA), a 2′-(S)-constrained ethyl (S-cEt), a constrained MOE, and a 2′-O,4′-C-aminomethylene bridged nucleic acid (2′,4′-BNA^{NC});
at least 50%, 80%, 90%, or 100% of the ribose groups are chemically modified;
at least 40%, 80%, 90% or 100% of the ribose groups in the guide sequence of the crRNA portion are chemically modified;
at least 40% of the ribose groups in the repeat sequence of the crRNA portion are chemically modified;
each modification of the phosphate group is independently selected from the group consisting of a phosphorothioate, phosphonoacetate (PACE), thiophosphonoacetate (thioPACE), amide, triazole, phosphonate, and phosphotriester modification; and/or
each modification of the nucleobase group is independently selected from the group consisting of 2-thiouridine, 4-thiouridine, N⁶-methyladenosine, pseudouridine, 2,6-diaminopurine, inosine, thymidine, 5-methylcytosine, 5-substituted pyrimidine, isoguanine, isocytosine, and halogenated aromatic groups.

3-20. (canceled)

21. The chemically modified Nme gRNA of claim 1, wherein:
the gRNA comprises at least 90% or 100% modified nucleotides;
at least one nucleotide of the crRNA portion comprises a 2′-deoxy chemical modification;
one or more of the nucleotides at positions 7, 26, 33, 34, 35, 36, and 37 from the 5′ end of the crRNA portion comprises a 2′-deoxy chemical modification;
one or more of the nucleotides at positions 12, 15, 16, 23, and 42 from the 3′ end of the crRNA portion comprises a 2′-deoxy chemical modification;
the nucleotide at position 7 from the 5′ end of the guide sequence of the crRNA portion comprises a 2′-deoxy chemical modification;
the nucleotide at position 18 from the 3′ end of the guide sequence of the crRNA portion comprises a 2′-deoxy chemical modification;
the nucleotides at positions 2, 9, 10, 11, 12, and 13 from the 5′ end of the repeat sequence of the crRNA portion comprises a 2′-deoxy chemical modification;
at least one nucleotide of the crRNA portion comprises a 2′-fluoro chemical modification;
one or more of the nucleotides at positions 12, 14, 16, 19, 20, and 21 from the 5′ end of the crRNA portion comprises a 2′-fluoro chemical modification;
one or more of the nucleotides at positions 28, 29, 30, 33, 35, and 37 from the 3′ end of the crRNA portion comprises a 2′-fluoro chemical modification;
one or more of the nucleotides at positions 4, 6, 9, 11, and 13 from the 3′ end of the guide sequence of the crRNA portion comprises a 2′-fluoro chemical modification;
one or more of the nucleotides at positions 1, 5, 6, 7, and 8 from the 5′ end of the repeat sequence of the crRNA portion comprise a 2′-fluoro chemical modification;
the nucleotides at positions 33 and 35 from the 3′ end of the crRNA portion comprises a 2′-fluoro chemical modification and position 42 from the 3′ end of the crRNA portion comprises a 2′-deoxy chemical modification;
the nucleotides at positions 9 and 11 from the 3′ end of the guide sequence of the crRNA portion comprises a 2′-fluoro chemical modification and position 18 from the 3′ end of the guide sequence of the crRNA portion comprises a 2′-deoxy chemical modification;
the guide sequence of the crRNA portion is between 18 and 26 nucleotides in length;
the repeat sequence of the crRNA portion is between 12 and 24 nucleotides in length;
the repeat sequence of the crRNA portion is 12, 18, or 24 nucleotides in length;
the crRNA portion is between 32 and 48 nucleotides in length; and/or
the tracrRNA portion is between 60 and 100 nucleotides in length.

22-40. (canceled)

41. The chemically modified Nme gRNA of claim 1, comprising at least one chemical modification in a crRNA portion nucleotide sequence of:
(SEQ ID NO: 8)
(N)ₓ GUUGUAGCUCCCUUUCUC (CR42);
or
(SEQ ID NO: 9)
(N)ₓ GUUGUAGCUCCCUUUCUCAUUUCG (CR48),
wherein N corresponds to any nucleotide and x corresponds to any integer between 18 and 26, optionally wherein:
the crRNA portion modification pattern selected from the group consisting of:

| | |
|---|---|
| CR48-2 | (mN)#(mN)#(mN)#(mN)(mN)(rN)(rN)(rN)(rN)(rN)(rN)(rN)(rN)(rN)(rN)(rN) |
| | (rN)(rN)(rN)(rN)(rN)(rN)(rN)(rN)(rG)(rU)(rU)(rG)(rU)(rA)(rG)(rC)(rU) |
| | (rC)(rC)(rC)(rU)(mU)(mU)(mC)((mU)(mC)(mA)(mU)(mU)#(mU)#(mC)#(mG) |
| | (SEQ ID NO: 13); |
| CR48-154 | (mN)#(mN)#(mN)#(mN)(mN)(mN)dN)#(mN)(mN)(mN)(mN)(fN)(mN)(fN)(mN)(fN) |
| | (mN)mN)(rN)(rN)(rN)(mN)(rN)(rN)(mG)(rU)(rU)(rG)(mU)(mA)(mG)(mC)(rU) |
| | (rC)(rC)(rC)(rU)(mU)(mU)(mC)(mU)(mC)(mA)(mU)(mU)#(mU)#(mC)#(mG) |
| | (SEQ ID NO: 14); |
| CR48-155 | (mN)#(mN)#(mN)#(mN)(mN)(mN)(dN)#(mN)(mN)(mN)(mN)(mN)(mN)(fN)(mN)(fN) |
| | (mN)(mN)(rN)(rN)(rN)(mN)(rN)(rN)(mG)(rU)(rU)(rG)(mU)(mA)(mG)(mC)(rU) |
| | (rC)(rC)(rC)(rU)(mU)(mU)(mC)(mU)(mC)(mA)(mU)(mU)#(mU)#(mC)#(mG) |
| | (SEQ ID NO: 15); |
| CR48-190 | (mN)#(mN)#(mN)#(mN)(mN)(mN)(dN)#(mN)(mN)(mN)(mN)(fN)(mN)(fN)(mN)(fN) |
| | (mN)(mN)(rN)(fN)(mN)(mN)(mN)(mN)(mG)(rU)(rU)(rG)(mU)(mA)(mG)(mC)(rU) |
| | (rC)(rC)(rC)(rU)(mU)(mU)(mC)(mU)(mC)(mA)(mU)(mU)#(mU)#(mC)#(mG) |
| | (SEQ ID NO: 16); |
| CR48-191 | (mN)#(mN)#(mN)#(mN)(mN)(mN)(dN)#(mN)(mN)(mN)(mN)(fN)(mN)(fN)(mN)(fN) |
| | (mN)(mN)(rN)(mN)(fN)(mN)(mN)(mN)(mG)(rU)(rU)(rG)(mU)(mA)(mG)(mC)(rU) |
| | (rC)(rC)(rC)(rU)(mU)(mU)(mC)(mU)(mC)(mA)(mU)(mU)#(mU)#(mC)#(mG) |
| | (SEQ ID NO: 17); |
| CR48-192 | (mN)#(mN)#(mN)#(mN)(mN)(mN)(dN)#(mN)(mN)(mN)(mN)(mN)(mN)(fN)(mN)(fN) |
| | (mN)(mN)(rN)(fN)(mN)(mN)(mN)(mN)(mG)(rU)(rU)(rG)(mU)(mA)(mG)(mC)(rU) |
| | (rC)(rC)(rC)(rU)(mU)(mU)(mC)(mU)(mC)(mA)(mU)(mU)#(mU)#(mC)#(mG) |
| | (SEQ ID NO: 18); |
| CR48-193 | (mN)#(mN)#(mN)#(mN)(mN)(mN)(dN)#(mN)(mN)(mN)(mN)(mN)(mN)(fN)(mN)(fN) |
| | (mN)(mN)(rN)(mN)(fN)(mN)(mN)(mN)(mG)(rU)(rU)(rG)(mU)(mA)(mG)(mC)(rU) |
| | (rC)(rC)(rC)(rU)(mU)(mU)(mC)(mU)(mC)(mA)(mU)(mU)#(mU)#(mC)#(mG) |
| | (SEQ ID NO: 19); |
| CR48-194 | (mN)#(mN)#(mN)#(mN)(mN)(mN)(dN)#(mN)(mN)(mN)(mN)(fN)(mN)(fN)(mN)(fN) |
| | (mN)(mN)(rN)#(fN)(mN)(mN)(mN)(mN)(mG)(rU)(rU)(rG)(mU)(mA)(mG)(mC)(rU) |
| | (rC)(rC)(rC)(rU)(mU)(mU)(mC)(mU)(mC)(mA)(mU)(mU)#(mU)#(mC)#(mG) |
| | (SEQ ID NO: 20); |
| CR48-195 | (mN)#(mN)#(mN)#(mN)(mN)(mN)(dN)#(mN)(mN)(mN)(mN)(fN)(mN)(fN)(mN)(fN) |
| | (mN)(mN)(rN)#(mN)(fN)(mN)(mN)(mN)(mG)(rU)(rU)(rG)(mU)(mA)(mG)(mC)(rU) |
| | (rC)(rC)(rC)(rU)(mU)(mU)(mC)(mU)(mC)(mA)(mU)(mU)#(mU)#(mC)#(mG) |
| | (SEQ ID NO: 21); |
| CR48-196 | (mN)#(mN)#(mN)#(mN)(mN)(mN)(dN)#(mN)(mN)(mN)(mN)(mN)(mN)(fN)(mN)(fN) |
| | (mN)(mN)(rN)#(fN)(mN)(mN)(mN)(mN)(mG)(rU)(rU)(rG)(mU)(mA)(mG)(mC)(rU) |
| | (rC)(rC)(rC)(rU)(mU)(mU)(mC)(mU)(mC)(mA)(mU)(mU)#(mU)#(mC)#(mG) |
| | (SEQ ID NO: 22); |
| CR48-197 | (mN)#(mN)#(mN)#(mN)(mN)(mN)(dN)#(mN)(mN)(mN)(mN)(mN)(mN)(fN)(mN)(fN) |
| | (mN)(mN)(rN)#(mN)(fN)(mN)(mN)(mN)(mG)(rU)(rU)(rG)(mU)(mA)(mG)(mC)(rU) |
| | (rC)(rC)(rC)(rU)(mU)(mU)(mC)(mU)(mC)(mA)(mU)(mU)#(mU)#(mC)#(mG) |
| | (SEQ ID NO: 23); |
| CR48-198 | (mN)#(mN)#(mN)#(mN)(mN)(mN)(dN)#(mN)(mN)(mN)(mN)(fN)(mN)(fN)(mN)(fN) |
| | (mN)(mN)(mN)(fN)(mN)(mN)(mN)(mN)(mG)(rU)(rU)(rG)(mU)(mA)(mG)(mC)(rU) |
| | (rC)(rC)(rC)(rU)(mU)(mU)(mC)(mU)(mC)(mA)(mU)(mU)#(mU)#(mC)#(mG) |
| | (SEQ ID NO: 24); |
| CR48-199 | (mN)#(mN)#(mN)#(mN)(mN)(mN)(dN)#(mN)(mN)(mN)(mN)(fN)(mN)(fN)(mN)(fN) |
| | (mN)(mN)(mN)(mN)(fN)(mN)(mN)(mN)(mG)(rU)(rU)(rG)(mU)(mA)(mG)(mC)(rU) |
| | (rC)(rC)(rC)(rU)(mU)(mU)(mC)(mU)(mC)(mA)(mU)(mU)#(mU)#(mC)#(mG) |
| | (SEQ ID NO: 25); |
| CR48-200 | (mN)#(mN)#(mN)#(mN)(mN)(mN)(dN)#(mN)(mN)(mN)(mN)(mN)(mN)(fN)(mN)(fN) |
| | (mN)(mN)(mN)(fN)(mN)(mN)(mN)(mN)(mG)(rU)(rU)(rG)(mU)(mA)(mG)(mC)(rU) |
| | (rC)(rC)(rC)(rU)(mU)(mU)(mC)(mU)(mC)(mA)(mU)(mU)#(mU)#(mC)#(mG) |
| | (SEQ ID NO: 26); |
| CR48-201 | (mN)#(mN)#(mN)#(mN)(mN)(mN)(dN)#(mN)(mN)(mN)(mN)(mN)(mN)(fN)(mN)(fN) |
| | (mN)(mN)(mN)(mN)(fN)(mN)(mN)(mN)(mG)(rU)(rU)(rG)(mU)(mA)(mG)(mC)(rU) |
| | (rC)(rC)(rC)(rU)(mU)(mU)(mC)(mU)(mC)(mA)(mU)(mU)#(mU)#(mC)#(mG) |
| | (SEQ ID NO: 27); |
| CR48-202 | (mN)#(mN)#(mN)#(mN)(mN)(mN)(dN)#(mN)(mN)(mN)(mN)(fN)(mN)(fN)(mN)(fN) |
| | (mN)(mN)(fN)(fN)(mN)(mN)(mN)(mN)(mG)(rU)(rU)(rG)(mU)(mA)(mG)(mC)(rU) |
| | (rC)(rC)(rC)(rU)(mU)(mU)(mC)(mU)(mC)(mA)(mU)(mU)#(mU)#(mC)#(mG) |
| | (SEQ ID NO: 28); |
| CR48-203 | (mN)#(mN)#(mN)#(mN)(mN)(mN)dN)#(mN)(mN)(mN)(mN)(fN)(mN)(fN)(mN)(fN) |
| | (mN)mN)(fN)(mN)(fN)(mN)(mN)(mN)(mG)(rU)(rU)(rG)(mU)(mA)(mG)(mC)(rU) |
| | (rC)(rC)(rC)(rU)(mU)(mU)(mC)(mU)(mC)(mA)(mU)(mU)#(mU)#(mC)#(mG) |
| | (SEQ ID NO: 29); |
| CR48-204 | (mN)#(mN)#(mN)#(mN)(mN)(mN)(dN)#(mN)(mN)(mN)(mN)(mN)(mN)(fN)(mN)(fN) |
| | (mN)(mN)(fN)(fN)(mN)(mN)(mN)(mN)(mG)(rU)(rU)(rG)(mU)(mA)(mG)(mC)(rU) |
| | (rC)(rC)(rC)(rU)(mU)(mU)(mC)(mU)(mC)(mA)(mU)(mU)#(mU)#(mC)#(mG) |
| | (SEQ ID NO: 30); |
| CR48-205 | (mN)#(mN)#(mN)#(mN)(mN)(mN)(dN)#(mN)(mN)(mN)(mN)(mN)(mN)(fN)(mN)(fN) |
| | (mN)(mN)(fN)(mN)(fN)(mN)(mN)(mN)(mG)(rU)(rU)(rG)(mU)(mA)(mG)(mC)(rU) |
| | (rC)(rC)(rC)(rU)(mU)(mU)(mC)(mU)(mC)(mA)(mU)(mU)#(mU)#(mC)#(mG) |
| | (SEQ ID NO: 31); |
| CR48-213 | (mN)#(mN)#(mN)#(mN)(mN)(mN)(dN)#(mN)(mN)(mN)(mN)(mN)(mN)(fN)(mN)(fN) |
| | (mN)(mN)(rN)(rN)(rN)(mN)(rN)(rN)(mG)(dT)#(rU)(mG)(mU)(mA)(mG)(mC)(rU) |
| | (rC)(rC)(rC)(rU)(mU)(mU)(mC)(mU)(mC)(mA)(mU)(mU)#(mU)#(mC)#(mG) |
| | (SEQ ID NO: 32); |
| CR48-214 | (mN)#(mN)#(mN)#(mN)(mN)(mN)(dN)#(mN)(mN)(mN)(mN)(mN)(mN)(fN)(mN)(fN) |
| | (mN)(mN)(rN)(rN)(rN)(mN)(rN)(rN)(mG)(dT)#(rU)(mG)(mU)(mA)(mG)(mC)(rU) |
| | (rC)(mC)(rC)(rU)(mU)(mU)(mC)(mU)(mC)(mA)(mU)(mU)#(mU)#(mC)#(mG) |
| | (SEQ ID NO: 33); |
| CR48-215 | (mN)#(mN)#(mN)#(mN)(mN)(mN)(dN)#(mN)(mN)(mN)(mN)(mN)(mN)(fN)(mN)(fN) |
| | (mN)(mN)(rN)(rN)(rN)(mN)(rN)(rN)(mG)(dT)#(rU)(mG)(mU)(mA)(mG)(mC)(rU) |
| | (rC)(rC)(mC)(rU)(mU)(mU)(mC)(mU)(mC)(mA)(mU)(mU)#(mU)#(mC)#(mG) |
| | (SEQ ID NO: 34); |
| CR48-216 | (mN)#(mN)#(mN)#(mN)(mN)(mN)(dN)#(mN)(mN)(mN)(mN)(mN)(mN)(fN)(mN)(fN) |
| | (mN)(mN)(rN)(rN)(rN)(mN)(rN)(rN)(mG)(dT)#(rU)(mG)(mU)(mA)(mG)(mC)(rU) |
| | (rC)(mC)(mC)(rU)(mU)(mU)(mC)(mU)(mC)(mA)(mU)(mU)#(mU)#(mC)#(mG) |
| | (SEQ ID NO: 35); |
| CR48-217 | (mN)#(mN)#(mN)#(mN)(mN)(mN)(dN)#(mN)(mN)(mN)(mN)(mN)(mN)(fN)(mN)(fN) |
| | (mN)(mN)(rN)(rN)(rN)(mN)(rN)(rN)(mG)(rU)(rU)(rG)(mU)(mA)(mG)(mC)(dT) |
| | #(rC)(rC)(rC)(rU)(mU)(mU)(mC)(mU)(mC)(mA)(mU)(mU)#(mU)#(mC)#(mG) |
| | (SEQ ID NO: 36); |
| CR48-218 | (mN)#(mN)#(mN)#(mN)(mN)(mN)(dN)#(mN)(mN)(mN)(mN)(mN)(mN)(fN)(mN)(fN) |
| | (mN)(mN)(rN)(rN)(rN)(mN)(rN)(rN)(mG)(rU)(rU)(rG)(mU)(mA)(mG)(mC)(rU) |
| | (dC)#(rC)(rC)(rU)(mU)(mU)(mC)(mU)(mC)(mA)(mU)(mU)#(mU)#(mC)#(mG) |
| | (SEQ ID NO: 37); |
| CR48-219 | (mN)#(mN)#(mN)#(mN)(mN)(mN)(dN)#(mN)(mN)(mN)(mN)(mN)(mN)(fN)(mN)(fN) |
| | (mN)(mN)(rN)(rN)(rN)(mN)(rN)(rN)(mG)(rU)(rU)(rG)(mU)(mA)(mG)(mC)(rU) |
| | (rC)(dC)#(rC)(rU)(mU)(mU)(mC)(mU)(mC)(mA)(mU)(mU)#(mU)#(mC)#(mG) |
| | (SEQ ID NO: 38); |
| CR48-220 | (mN)#(mN)#(mN)#(mN)(mN)(mN)(dN)#(mN)(mN)(mN)(mN)(mN)(mN)(fN)(mN)(fN) |
| | (mN)(mN)(rN)(rN)(rN)(mN)(rN)(rN)(mG)(rU)(rU)(rG)(mU)(mA)(mG)(mC)(rU) |
| | (rC)(rC)(dC)#(rU)(mU)(mU)(mC)(mU)(mC)(mA)(mU)(mU)#(mU)#(mC)#(mG) |
| | (SEQ ID NO: 39); |
| CR48-221 | (mN)#(mN)#(mN)#(mN)(mN)(mN)(dN)#(mN)(mN)(mN)(mN)(mN)(mN)(fN)(mN)(fN) |
| | (mN)(mN)(rN)(rN)(rN)(mN)(rN)(rN)(mG)(rU)(rU)(rG)(mU)(mA)(mG)(mC)(rU) |
| | (rC)(rC)(rC)(dT)#(mU)(mU)(mC)(mU)(mC)(mA)(mU)(mU)#(mU)#(mC)#(mG) |
| | (SEQ ID NO: 40); |
| CR48-222 | (mN)#(mN)#(mN)#(mN)(mN)(mN)(dN)#(mN)(mN)(mN)(mN)(mN)(mN)(fN)(mN)(fN) |
| | (mN)(mN)(rN)(rN)(rN)(mN)(rN)(rN)(mG)(rU)(rU)(rG)(mU)(mA)(mG)(mC)(dT) |
| | #(rC)(dC)#(rC)(dT)#(mU)(mU)(mC)(mU)(mC)(mA)(mU)(mU)#(mU)#(mC)#(mG) |
| | (SEQ ID NO: 41); |
| CR48-223 | (mN)#(mN)#(mN)#(mN)(mN)(mN)(dN)#(mN)(mN)(mN)(mN)(mN)(mN)(fN)(mN)(fN) |
| | (mN)(mN)(rN)(rN)(rN)(mN)(rN)(rN)(mG)(dT)#(rU)(rG)(mU)(mA)(mG)(mC)(dT) |
| | #(dC)#(dC)#(rC)(dT)#(mU)(mU)(mC)(mU)(mC)(mA)(mU)(mU)#(mU)#(mC)#(mG) |
| | (SEQ ID NO: 42); |
| CR48-224 | (mN)#(mN)#(mN)#(mN)(mN)(mN)(dN)#(mN)(mN)(mN)(mN)(mN)(mN)(fN)(mN)(fN) |
| | (mN)(mN)(rN)(rN)(rN)(mN)(rN)(rN)(mG)(dT)#(rU)(mG)(mU)(mA)(mG)(mC)(dT) |
| | #(rC)(mC)(rC)(dT)#(mU)(mU)(mC)(mU)(mC)(mA)(mU)(mU)#(mU)#(mC)#(mG) |
| | (SEQ ID NO: 43); |
| CR48-225 | (mN)#(mN)#(mN)#(mN)(mN)(mN)(dN)#(mN)(mN)(mN)(mN)(mN)(mN)(fN)(mN)(fN) |
| | (mN)(mN)(rN)(rN)(rN)(mN)(rN)(rN)(mG)(dT)#(rU)(mG)(mU)(mA)(mG)(mC)(dT) |
| | #(rC)(dC)#(mC)(dT)#(mU)(mU)(mC)(mU)(mC)(mA)(mU)(mU)#(mU)#(mC)#(mG) |
| | (SEQ ID NO: 44); |
| CR48-226 | (mN)#(mN)#(mN)#(mN)(mN)(mN)(dN)#(mN)(mN)(mN)(mN)(mN)(mN)(fN)(mN)(fN) |
| | (mN)(mN)(rN)(rN)(rN)(mN)(rN)(rN)(mG)(dT)#(rU)(mG)(mU)(mA)(mG)(mC)(dT) |
| | #(rC)(mC)(mC)(dT)#(mU)(mU)(mC)(mU)(mC)(mA)(mU)(mU)#(mU)#(mC)#(mG) |
| | (SEQ ID NO: 45); |
| CR48-227 | (mN)#(mN)#(mN)#(mN)(mN)(mN)(dN)#(mN)(mN)(mN)(mN)(mN)(mN)(fN)(mN)(fN) |
| | (mN)(mN)(rN)(rN)(rN)(mN)(rN)(rN)(mG)(dT)#(rU)(mG)(mU)(mA)(mG)(mC)(dT) |
| | #(dC)#(mC)(mC)dT)#(mU)(mU)(mC)(mU)(mC)(mA)(mU)(mU)#(mU)#(mC)#(mG) |
| | (SEQ ID NO: 46); |
| CR48-199- | (mN)#(mN)#(mN)#(mN)(mN)(mN)(dN)#(mN)(mN)(mN)(mN)(fN)(mN)(fN)(mN)(fN) |
| V1.1 | (mN)(mN)(mN)(mN)(fN)(mN)(mN)(mN)(mG)(rU)(rU)(mG)(mU)(mA)(mG)(mC)(rU) |
| | (rC)(rC)(dC)#(rU)(mU)(mU)(mC)(mU)(mC)(mA)(mU)(mU)#(mU)#(mC)#(mG) |
| | (SEQ ID NO: 47); |
| CR48-199- | (mN)#(mN)#(mN)#(mN)(mN)(mN)(dN)#(mN)(mN)(mN)(mN)(fN)(mN)(fN)(mN)(fN) |
| V1.2 | ((mN)mN)(mN)(mN)(fN)(mN)(mN)(mN)(mG)(rU)(rU)(mG)(mU)(mA)(mG)(mC)(rU) |
| | (rC)(dC)#(rC)(rU)(mU)(mU)(mC)(mU)(mC)(mA)(mU)(mU)#(mU)#(mC)#(mG) |
| | (SEQ ID NO: 48); |
| CR48-199- | (mN)#(mN)#(mN)#(mN)(mN)(mN)(dN)#(mN)(mN)(mN)(mN)(fN)(mN)(fN)(mN)(fN) |
| V1.3 | (mN)(mN)(mN)(mN)(fN)(mN)(mN)(mN)(mG)(rU)(rU)(mG)(mU)(mA)(mG)(mC)(rU) |
| | (rC)(rC)(rC)(dT)#(mU)(mU)(mC)(mU)(mC)(mA)(mU)(mU)#(mU)#(mC)#(mG) |
| | (SEQ ID NO: 49); |
| CR48-199- | (mN)#(mN)#(mN)#(mN)(mN)(mN)(dN)#(mN)(mN)(mN)(mN)(fN)(mN)(fN)(mN)(fN) |
| V1.4 | (mN)(mN)(mN)(mN)(fN)(mN)(mN)(mN)(mG)(rU)(rU)(mG)(mU)(mA)(mG)(mC)(rU) |
| | (rC)(mC)(rC)(rU)(mU)(mU)(mC)(mU)(mC)(mA)(mU)(mU)#(mU)#(mC)#(mG) |
| | (SEQ ID NO: 50); |
| CR48-199- | (mN)#(mN)#(mN)#(mN)(mN)(mN)(dN)#(mN)(mN)(mN)(mN)(fN)(mN)(fN)(mN)(fN) |
| V1.5 | (mN)(mN)(mN)(mN)(fN)(mN)(mN)(mN)(mG)(rU)(rU)(mG)(mU)(mA)(mG)(mC)(rU) |
| | (rC)(dC)#(dC)#(rU)(mU)(mU)(mC)(mU)(mC)(mA)(mU)(mU)#(mU)#(mC)#(mG) |
| | (SEQ ID NO: 51); |
| CR48-199- | (mN)#(mN)#(mN)#(mN)(mN)(mN)(dN)#(mN)(mN)(mN)(mN)(fN)(mN)(fN)(mN)(fN) |
| V1.6 | (mN)(mN)(mN)(mN)(fN)(mN)(mN)(mN)(mG)(rU)(rU)(mG)(mU)(mA)(mG)(mC)(rU) |
| | (rC)(dC)#(rC)(dT)#(mU)(mU)(mC)(mU)(mC)(mA)(mU)(mU)#(mU)#(mC)#(mG) |
| | (SEQ ID NO: 52); |
| CR48-199- | (mN)#(mN)#(mN)#(mN)(mN)(mN)(dN)#(mN)(mN)(mN)(mN)(fN)(mN)(fN)(mN)(fN) |
| V1.7 | (mN)(mN)(mN)(mN)(fN)(mN)(mN)(mN)(mG)(rU)(rU)(mG)(mU)(mA)(mG)(mC)(rU) |
| | (rC)(dC)#(dC)#(dT)#(mU)(mU)(mC)(mU)(mC)(mA)(mU)(mU)#(mU)#(mC)#(mG) |
| | (SEQ ID NO: 53); |
| CR48-199- | (mN)#(mN)#(mN)#(mN)(mN)(mN)(dN)#(mN)(mN)(mN)(mN)(fN)(mN)(fN)(mN)(fN) |
| V1.8 | (mN)(mN)(mN)(mN)(fN)(mN)(mN)(mN)(mG)(rU)(rU)(mG)(mU)(mA)(mG)(mC)(rU) |
| | (rC)(mC)(dC)#(rU)(mU)(mU)(mC)(mU)(mC)(mA)(mU)(mU)#(mU)#(mC)#(mG) |
| | (SEQ ID NO: 54); |
| CR48-199- | (mN)#(mN)#(mN)#(mN)(mN)(mN)(dN)#(mN)(mN)(mN)(mN)(fN)(mN)(fN)(mN)(fN) |
| V1.9 | (mN)(mN)(mN)(mN)(fN)(mN)(mN)(mN)(mG)(rU)(rU)(mG)(mU)(mA)(mG)(mC)(rU) |
| | (rC)(mC)(rC)(dT)#(mU)(mU)(mC)(mU)(mC)(mA)(mU)(mU)#(mU)#(mC)#(mG) |
| | (SEQ ID NO: 55); |
| CR48-199- | (mN)#(mN)#(mN)#(mN)(mN)(mN)(dN)#(mN)(mN)(mN)(mN)(fN)(mN)(fN)(mN)(fN) |
| V1.10 | (mN)(mN)(mN)(mN)(fN)(mN)(mN)(mN)(mG)(rU)(rU)(mG)(mU)(mA)(mG)(mC)(rU) |
| | (rC)(mC)(dC)#(dT)#(mU)(mU)(mC)(mU)(mC)(mA)(mU)(mU)#(mU)#(mC)#(mG) |
| | (SEQ ID NO: 56); |
| CR48-199- | (mN)#(mN)#(mN)#(mN)(mN)(mN)(dN)#(mN)(mN)(mN)(mN)(fN)(mN)(fN)(mN)(fN) |
| V1.11 | (mN)(mN)(mN)(mN)(fN)(mN)(mN)(mN)(mG)(rU)(rU)(mG)(mU)(mA)(mG)(mC)(rU) |
| | (rC)(mC)(mC)(rU)(mU)(mU)(mC)(mU)(mC)(mA)(mU)(mU)#(mU)#(mC)#(mG) |
| | (SEQ ID NO: 57); |
| CR48-199- | (mN)#(mN)#(mN)#(mN)(mN)(mN)(dN)#(mN)(mN)(mN)(mN)(fN)(mN)(fN)(mN)(fN) |
| V1.12 | (mN)(mN)(mN)(mN)(fN)(mN)(mN)(mN)(mG)(rU)(rU)(mG)(mU)(mA)(mG)(mC)(rU) |
| | (rC)(mC)(mC)(dT)#(mU)(mU)(mC)(mU)(mC)(mA)(mU)(mU)#(mU)#(mC)#(mG) |
| | (SEQ ID NO: 58); |
| CR48-199- | (mN)#(mN)#(mN)#(mN)(mN)(mN)(dN)#(mN)(mN)(mN)(mN)(fN)(mN)(fN)(mN)(fN) |
| V1.13 | (mN)(mN)(mN)(mN)(fN)(mN)(mN)(mN)(mG)(rU)(rU)(mG)(mU)(mA)(mG)(mC)(dT) |
| | #(rC)(rC)(dC)#(rU)(mU)(mU)(mC)(mU)(mC)(mA)(mU)(mU)#(mU)#(mC)#(mG) |
| | (SEQ ID NO: 59); |
| CR48-199- | (mN)#(mN)#(mN)#(mN)(mN)(mN)(dN)#(mN)(mN)(mN)(mN)(fN)(mN)(fN)(mN)(fN) |
| V1.14 | (mN)(mN)(mN)(mN)(fN)(mN)(mN)(mN)(mG)(rU)(rU)(mG)(mU)(mA)(mG)(mC)(dT) |
| | #(rC)(dC)#(rC)(rU)(mU)(mU)(mC)(mU)(mC)(mA)(mU)(mU)#(mU)#(mC)#(mG) |
| | (SEQ ID NO: 60); |
| CR48-199- | (mN)#(mN)#(mN)#(mN)(mN)(mN)(dN)#(mN)(mN)(mN)(mN)(fN)(mN)(fN)(mN)(fN) |
| V1.15 | (mN)(mN)(mN)(mN)(fN)(mN)(mN)(mN)(mG)(rU)(rU)(mG)(mU)(mA)(mG)(mC)(dT) |
| | #(rC)(rC)(rC)(dT)#(mU)(mU)(mC)(mU)(mC)(mA)(mU)(mU)#(mU)#(mC)#(mG) |
| | (SEQ ID NO: 61); |
| CR48-199- | (mN)#(mN)#(mN)#(mN)(mN)(mN)(dN)#(mN)(mN)(mN)(mN)(fN)(mN)(fN)(mN)(fN) |
| V1.16 | (mN)(mN)(mN)(mN)(fN)(mN)(mN)(mN)(mG)(rU)(rU)(mG)(mU)(mA)(mG)(mC)(dT) |
| | #(rC)(mC)(rC)(rU)(mU)(mU)(mC)(mU)(mC)(mA)(mU)(mU)#(mU)#(mC)#(mG) |
| | (SEQ ID NO: 62); |
| CR48-199- | (mN)#(mN)#(mN)#(mN)(mN)(mN)(dN)#(mN)(mN)(mN)(mN)(fN)(mN)(fN)(mN)(fN) |
| V1.17 | (mN)(mN)(mN)(mN)(fN)(mN)(mN)(mN)(mG)(rU)(rU)(mG)(mU)(mA)(mG)(mC)(dT) |
| | #(rC)(dC)#(dC)#(rU)(mU)(mU)(mC)(mU)(mC)(mA)(mU)(mU)#(mU)#(mC)#(mG) |
| | (SEQ ID NO: 63); |
| CR48-199- | (mN)#(mN)#(mN)#(mN)(mN)(mN)(dN)#(mN)(mN)(mN)(mN)(fN)(mN)(fN)(mN)(fN) |
| V1.18 | (mN)(mN)(mN)(mN)(fN)(mN)(mN)(mN)(mG)(rU)(rU)(mG)(mU)(mA)(mG)(mC)(dT) |
| | #(rC)(dC)#(rC)(dT)#(mU)(mU)(mC)(mU)(mC)(mA)(mU)(mU)#(mU)#(mC)#(mG) |
| | (SEQ ID NO: 64); |
| CR48-199- | (mN)#(mN)#(mN)#(mN)(mN)(mN)dN)#(mN)(mN)(mN)(mN)(fN)(mN)(fN)(mN)(fN) |
| V1.19 | (mN)mN)(mN)(mN)(fN)(mN)(mN)(mN)(mG)(rU)(rU)(mG)(mU)(mA)(mG)(mC)(dT) |
| | #(rC)(dC)#(dC)#(dT)#(mU)(mU)(mC)(mU)(mC)(mA)(mU)(mU)#(mU)#(mC)#(mG) |
| | (SEQ ID NO: 65); |
| CR48-199- | (mN)#(mN)#(mN)#(mN)(mN)(mN)(dN)#(mN)(mN)(mN)(mN)(fN)(mN)(fN)(mN)(fN) |
| V1.20 | (mN)(mN)(mN)(mN)(fN)(mN)(mN)(mN)(mG)(rU)(rU)(mG)(mU)(mA)(mG)(mC)(dT) |
| | #(rC)(mC)(dC)#(rU)(mU)(mU)(mC)(mU)(mC)(mA)(mU)(mU)#(mU)#(mC)#(mG) |
| | (SEQ ID NO: 66); |
| CR48-199- | (mN)#(mN)#(mN)#(mN)(mN)(mN)(dN)#(mN)(mN)(mN)(mN)(fN)(mN)(fN)(mN)(fN) |
| V1.21 | (mN)(mN)(mN)(mN)(fN)(mN)(mN)(mN)(mG)(rU)(rU)(mG)(mU)(mA)(mG)(mC)(dT) |
| | #(rC)(mC)(rC)(dT)#(mU)(mU)(mC)(mU)(mC)(mA)(mU)(mU)#(mU)#(mC)#(mG) |
| | (SEQ ID NO: 67); |
| CR48-199- | (mN)#(mN)#(mN)#(mN)(mN)(mN)(dN)#(mN)(mN)(mN)(mN)(fN)(mN)(fN)(mN)(fN) |
| V1.22 | (mN)(mN)(mN)(mN)(fN)(mN)(mN)(mN)(mG)(rU)(rU)(mG)(mU)(mA)(mG)(mC)(dT) |
| | #(rC)(mC)(dC)#(dT)#(mU)(mU)(mC)(mU)(mC)(mA)(mU)(mU)#(mU)#(mC)#(mG) |
| | (SEQ ID NO: 68); |
| CR48-199- | (mN)#(mN)#(mN)#(mN)(mN)(mN)(dN)#(mN)(mN)(mN)(mN)(fN)(mN)(fN)(mN)(fN) |
| V1.23 | (mN)(mN)(mN)(mN)(fN)(mN)(mN)(mN)(mG)(rU)(rU)(mG)(mU)(mA)(mG)(mC)(dT) |
| | #(rC)(mC)(mC)(rU)(mU)(mU)(mC)(mU)(mC)(mA)(mU)(mU)#(mU)#(mC)#(mG) |
| | (SEQ ID NO: 69); |
| CR48-199- | (mN)#(mN)#(mN)#(mN)(mN)(mN)(dN)#(mN)(mN)(mN)(mN)(fN)(mN)(fN)(mN)(fN) |
| V1.24 | (mN)(mN)(mN)(mN)(fN)(mN)(mN)(mN)(mG)(rU)(rU)(mG)(mU)(mA)(mG)(mC)(dT) |
| | #(rC)(mC)(mC)(dT)#(mU)(mU)(mC)(mU)(mC)(mA)(mU)(mU)#(mU)#(mC)#(mG) |
| | (SEQ ID NO: 70); |
| CR48-199- | (mN)#(mN)#(mN)#(mN)(mN)(mN)(dN)#(mN)(mN)(mN)(mN)(fN)(mN)(fN)(mN)(fN) |
| V1.25 | (mN)(mN)(mN)(mN)(fN)(mN)(mN)(mN)(mG)(dT)#(rU)(mG)(mU)(mA)(mG)(mC)(dT) |
| | #(rC)(rC)(dC)#(rU)(mU)(mU)(mC)(mU)(mC)(mA)(mU)(mU)#(mU)#(mC)#(mG) |
| | (SEQ ID NO: 71); |
| CR48-199- | (mN)#(mN)#(mN)#(mN)(mN)(mN)(dN)#(mN)(mN)(mN)(mN)(fN)(mN)(fN)(mN)(fN) |
| V1.26 | (mN)(mN)(mN)(mN)(fN)(mN)(mN)(mN)(mG)(dT)#(rU)(mG)(mU)(mA)(mG)(mC)(dT) |
| | #(rC)(dC)#(rC)(rU)(mU)(mU)(mC)(mU)(mC)(mA)(mU)(mU)#(mU)#(mC)#(mG) |
| | (SEQ ID NO: 72); |
| CR48-199- | (mN)#(mN)#(mN)#(mN)(mN)(mN)(dN)#(mN)(mN)(mN)(mN)(fN)(mN)(fN)(mN)(fN) |
| V1.27 | (mN)(mN)(mN)(mN)(fN)(mN)(mN)(mN)(mG)(dT)#(rU)(mG)(mU)(mA)(mG)(mC)(dT) |
| | #(rC)(rC)(rC)(dT)#(mU)(mU)(mC)(mU)(mC)(mA)(mU)(mU)#(mU)#(mC)#(mG) |
| | (SEQ ID NO: 73); |
| CR48-199- | (mN)#(mN)#(mN)#(mN)(mN)(mN)(dN)#(mN)(niN)(mN)(mN)(fN)(mN)(fN)(mN)(fN) |
| V1.28 | (mN)(mN)(mN)(mN)(fN)(mN)(mN)(mN)(mG)(dT)#(rU)(mG)(mU)(mA)(mG)(mC)(dT) |
| | #(rC)(mC)(rC)(rU)(mU)(mU)(mC)(mU)(mC)(mA)(mU)(mU)#(mU)#(mC)#(mG) |
| | (SEQ ID NO: 74); |
| CR48-199- | (mN)#(mN)#(mN)#(mN)(mN)(mN)(dN)#(mN)(niN)(mN)(mN)(fN)(mN)(fN)(mN)(fN) |
| V1.29 | (mN)(mN)(mN)(mN)(fN)(mN)(mN)(mN)(mG)(dT)#(rU)(mG)(mU)(mA)(mG)(mC)(dT) |
| | #(rC)(dC)#(dC)#(rU)(mU)(mU)(mC)(mU)(mC)(mA)(mU)(mU)#(mU)#(mC)#(mG) |
| | (SEQ ID NO: 75); |
| CR48-199- | (mN)#(mN)#(mN)#(mN)(mN)(mN)(dN)#(mN)(niN)(mN)(mN)(fN)(mN)(fN)(mN)(fN) |
| V1.30 | (mN)(mN)(mN)(mN)(fN)(mN)(mN)(mN)(mG)(dT)#(rU)(mG)(mU)(mA)(mG)(mC)(dT) |
| | #(rC)(dC)#(rC)(dT)#(mU)(mU)(mC)(mU)(mC)(mA)(mU)(mU)#(mU)#(mC)#(mG) |
| | (SEQ ID NO: 76); |
| CR48-199- | (mN)#(mN)#(mN)#(mN)(mN)(mN)(dN)#(mN)(mN)(mN)(mN)(fN)(mN)(fN)(mN)(fN) |
| V1.31 | (mN)(mN)(mN)(mN)(fN)(mN)(mN)(mN)(mG)(dT)#(rU)(mG)(mU)(mA)(mG)(mC)(dT) |
| | #(rC)(dC)#(dC)#(dT)#(mU)(mU)(mC)(mU)(mC)(mA)(mU)(mU)#(mU)#(mC)#(mG) |
| | (SEQ ID NO: 77); |
| CR48-199- | (mN)#(mN)#(mN)#(mN)(mN)(mN)(dN)#(mN)(mN)(mN)(mN)(fN)(mN)(fN)(mN)(fN) |
| V1.32 | (mN)(mN)(mN)(mN)(fN)(mN)(mN)(mN)(mG)(dT)#(rU)(mG)(mU)(mA)(mG)(mC)(dT) |
| | #(rC)(mC)(dC)#(rU)(mU)(mU)(mC)(mU)(mC)(mA)(mU)(mU)#(mU)#(mC)#(mG) |
| | (SEQ ID NO: 78); |
| CR48-199- | (mN)#(mN)#(mN)#(mN)(mN)(mN)(dN)#(mN)(mN)(mN)(mN)(fN)(mN)(fN)(mN)(fN) |
| V1.33 | (mN)(mN)(mN)(mN)(fN)(mN)(mN)(mN)(mG)(dT)#(rU)(mG)(mU)(mA)(mG)(mC)(dT) |
| | #(rC)(mC)(rC)(dT)#(mU)(mU)(mC)(mU)(mC)(mA)(mU)(mU)#(mU)#(mC)#(mG) |
| | (SEQ ID NO: 79); |
| CR48-199- | (mN)#(mN)#(mN)#(mN)(mN)(mN)(dN)#(mN)(mN)(mN)(mN)(fN)(mN)(fN)(mN)(fN) |
| V1.34 | (mN)(mN)(mN)(mN)(fN)(mN)(mN)(mN)(mG)(dT)#(rU)(mG)(mU)(mA)(mG)(mC)(dT) |
| | #(rC)(mC)(dC)#(dT)#(mU)(mU)(mC)(mU)(mC)(mA)(mU)(mU)#(mU)#(mC)#(mG) |
| | (SEQ ID NO: 80); |
| CR48-199- | (mN)#(mN)#(mN)#(mN)(mN)(mN)(dN)#(mN)(mN)(mN)(mN)(fN)(mN)(fN)(mN)(fN) |
| V1.35 | (mN)(mN)(mN)(mN)(fN)(mN)(mN)(mN)(mG)(dT)#(rU)(mG)(mU)(mA)(mG)(mC)(dT) |
| | #(rC)(mC)(mC)(rU)(mU)(mU)(mC)(mU)(mC)(mA)(mU)(mU)#(mU)#(mC)#(mG) |
| | (SEQ ID NO: 81); |
| CR48-199- | (mN)#(mN)#(mN)#(mN)(mN)(mN)(dN)#(mN)(mN)(mN)(mN)(fN)(mN)(fN)(mN)(fN) |
| V1.36 | (mN)(mN)(mN)(mN)(fN)(mN)(mN)(mN)(mG)(dT)#(rU)(mG)(mU)(mA)(mG)(mC)(dT) |
| | #(rC)(mC)mC)(dT)#(mU)(mU)(mC)(mU)(mC)(mA)(mU)(mU)#(mU)#(mC)#(mG) |
| | (SEQ ID NO: 82); |
| CR48-203- | (mN)#(mN)#(mN)#(mN)(mN)(mN)(dN)#(mN)(mN)(mN)(mN)(fN)(mN)(fN)(mN)(fN) |
| V1.7 | (mN)(mN)(fN)(mN)(fN)(mN)(mN)(mN)(mG)(rU)(rU)(mG)(mU)(mA)(mG)(mC)(rU) |
| | (rC)(dC)#(dC)#(dT)#(mU)(mU)(mC)(mU)(mC)(mA)(mU)(mU)#(mU)#(mC)#(mG) |
| | (SEQ ID NO: 83); |
| CR48-203- | (mN)#(mN)#(mN)#(mN)(mN)(mN)(dN)#(mN)(mN)(mN)(mN)(fN)(mN)(fN)(mN)(fN) |
| V1.10 | (mN)(mN)(fN)(mN)(fN)(mN)(mN)(mN)(mG)(rU)(rU)(mG)(mU)(mA)(mG)(mC)(rU) |
| | (rC)(mC)(dC)#(dT)#(mU)(mU)(mC)(mU)(mC)(mA)(mU)(mU)#(mU)#(mC)#(mG) |
| | (SEQ ID NO: 84); |
| CR48-203- | (mN)#(mN)#(mN)#(mN)(mN)(mN)(dN)#(mN)(mN)(mN)(mN)(fN)(mN)(fN)(mN)(fN) |
| VI.12 | (mN)(mN)(fN)(mN)(fN)(mN)(mN)(mN)(mG)(rU)(rU)(mG)(mU)(mA)(mG)(mC)(rU) |
| | (rC)(mC)(mC)(dT)#(mU)(mU)(mC)(mU)(mC)(mA)(mU)(mU)#(mU)#(mC)#(mG) |
| | (SEQ ID NO: 85); |
| CR48-203- | (mN)#(mN)#(mN)#(mN)(mN)(mN)(dN)#(mN)(mN)(mN)(mN)(fN)(mN)(fN)(mN)(fN) |
| VI.18 | (mN)(mN)(fN)(mN)(fN)(mN)(mN)(mN)(mG)(rU)(rU)(mG)(mU)(mA)(mG)(mC)(dT) |
| | #(rC)(dC)#(rC)(dT)#(mU)(mU)(mC)(mU)(mC)(mA)(mU)(mU)#(mU)#(mC)#(mG) |
| | (SEQ ID NO: 86); |
| CR48-203- | (mN)#(mN)#(mN)#(mN)(mN)(mN)(dN)#(mN)(mN)(mN)(mN)(fN)(mN)(fN)(mN)(fN) |
| V1.19 | (mN)(mN)(fN)(mN)(fN)(mN)(mN)(mN)(mG)(rU)(rU)(mG)(mU)(mA)(mG)(mC)(dT) |
| | #(rC)(dC)#(dC)#(dT)#(mU)(mU)(mC)(mU)(mC)(mA)(mU)(mU)#(mU)#(mC)#(mG) |
| | (SEQ ID NO: 87); |
| CR48-203- | (mN)#(mN)#(mN)#(mN)(mN)(mN)(dN)#(mN)(mN)(mN)(mN)(fN)(mN)(fN)(mN)(fN) |
| V1.22 | (mN)(mN)(fN)(mN)(fN)(mN)(mN)(mN)(mG)(rU)(rU)(mG)(mU)(mA)(mG)(mC)(dT) |
| | #(rC)(mC)(dC)#(dT)#(mU)(mU)(mC)(mU)(mC)(mA)(mU)(mU)#(mU)#(mC)#(mG) |
| | (SEQ ID NO: 88); |
| CR48-203- | (mN)#(mN)#(mN)#(mN)(mN)(mN)(dN)#(mN)(mN)(mN)(mN)(fN)(mN)(fN)(mN)(fN) |
| V1.24 | (mN)(mN)(fN)(mN)(fN)(mN)(mN)(mN)(mG)(rU)(rU)(mG)(mU)(mA)(mG)(mC)(dT) |
| | #(rC)(mC)(mC)(dT)#(mU)(mU)(mC)(mU)(mC)(mA)(mU)(mU)#(mU)#(mC)#(mG) |
| | (SEQ ID NO: 89); |
| CR48-203- | (mN)#(mN)#(mN)#(mN)(mN)(mN)(dN)#(mN)(mN)(mN)(mN)(fN)(mN)(fN)(mN)(fN) |
| V1.30 | (mN)(mN)(fN)(mN)(fN)(mN)(mN)(mN)(mG)(dT)#(rU)(mG)(mU)(mA)(mG)(mC)(dT) |
| | #(rC)(dC)#(rC)(dT)#(mU)(mU)(mC)(mU)(mC)(mA)(mU)(mU)#(mU)#(mC)#(mG) |
| | (SEQ ID NO: 90); |
| CR48-203- | (mN)#(mN)#(mN)#(mN)(mN)(mN)(dN)#(mN)(mN)(mN)(mN)(fN)(mN)(fN)(mN)(fN) |
| V1.31 | (mN)(mN)(fN)(mN)(fN)(mN)(mN)(mN)(mG)(dT)#(rU)(mG)(mU)(mA)(mG)(mC)(dT) |
| | #(rC)(dC)#(dC)#(dT)#(mU)(mU)(mC)(mU)(mC)(mA)(mU)(mU)#(mU)#(mC)#(mG) |
| | (SEQ ID NO: 91); |
| CR48-203- | (mN)#(mN)#(mN)#(mN)(mN)(mN)(dN)#(mN)(mN)(mN)(mN)(fN)(mN)(fN)(mN)(fN) |
| V1.34 | (mN)(mN)(fN)(mN)(fN)(mN)(mN)(mN)(mG)(dT)#(rU)(mG)(mU)(mA)(mG)(mC)(dT) |
| | #(rC)(mC)(dC)#(dT)#(mU)(mU)(mC)(mU)(mC)(mA)(mU)(mU)#(mU)#(mC)#(mG) |
| | (SEQ ID NO: 92); |
| CR48-203- | (mN)#(mN)#(mN)#(mN)(mN)(mN)(dN)#(mN)(mN)(mN)(mN)(fN)(mN)(fN)(mN)(fN) |
| V1.36 | (mN)(mN)(fN)(mN)(fN)(mN)(mN)(mN)(mG)(dT)#(rU)(mG)(mU)(mA)(mG)(mC)(dT) |
| | #(rC)(mC)(mC)(dT)#(mU)(mU)(mC)(mU)(mC)(mA)(mU)(mU)#(mU)#(mC)#(mG) |
| | (SEQ ID NO: 93) |

wherein rN=RNA, mN=2′-O-methyl RNA, fN=2′-fluoro RNA, dN=2′-deoxy RNA, N #N=phosphorothioate linkage, and N=any nucleotide, optionally wherein:
the chemically modified Nme gRNA comprises a tracrRNA portion nucleotide sequence of:
(SEQ ID NO: 10)
CGAAAUGAGAACCGUUGCUACAAUAAGGCCGUCUGAAAAGAUGUGCCGCA
ACGCUCUGCCCCUUAAAGCUUCUGCUUUAAGGGGCAUCGUUUA
(TR93);
or
(SEQ ID NO: 11)
AGAACCGUUGCUACAAUAAGGCCGUCUGAAAAGAUGUGCCGCAACGCUCU
GCCCCUUAAAGCUCCUGCUUUAAGGGGCAUCGUUUA (TR87),
optionally wherein tracrRNA portion modification
pattern selected from any one of:
(SEQ ID NO: 94)
(mC)#(mG)#(mA)#(rA)(rA)(rU)(rG)(rA)(rG)(rA)(rA)
(rC)(rC)(rG)(rU)(rU)(rG))(rC)(rU)(rA)(rC)(rA)
(rA)(rU)(rA)(rA)(rG)(rG)(rC)(rC)(rG)(rU)(rC)(rU)
(rG)(rA)(rA)(rA)(rA)(rG)(rA)(rU)(rG)(rU)(rG)(rC)
(rC)(rG)(rC)(rA)(rA)(rC)(rG)(rC)(rU)(rC)(rU)(rG)
(rC)(rC)(rC)(rC)(rU)(rU)(rA)(rA)(rA)(rG)(rC)(rU)
(rC)(rC)(rU)(rG)(rC)(rU)(rU)(rU)(rA)(rA)(rG)(rG)
(rG)(rG)(rC)(rA)(rU)(rC)(rG)(rU)#(mU)#(mU)#(mA)
(TR93-0);
(SEQ ID NO: 95)
(mC)#(mG)#(mA)#(rA)(rA)(rU)(rG)(rA)(rG)(rA)(rA)
(rC)(rC)(rG)(rU)(rU)(rG)(rC)(rU)(rA)(rC)(rA)
(rA)(rU)(rA)(rA)(rG)(rG)(rC)(rC)(rG)(rU)(rC)(rU)
(mG)(mA)(mA)(mA)(rA)(rG)(rA)(rU)(rG)(rU)(rG)(rC)
(rC)(rG)(rC)(rA)(rA)(rC)(rG)(rC)(rU)(rC)(rU)(rG)
(rC)(rC)(rC)(rC)(rU)(rU)(rA)(rA)(rA)(rG)(rC)(mU)
(mC)(mC)(mU)(rG)(rC)(rU)(rU)(rU)(rA)(rA)(rG)(rG)
(rG)(rG)(rC)(rA)(rU)(rC)(rG)(rU)#(mU)#(mU)#(mA)
(TR93-1);
(SEQ ID NO: 96)
(mC)#(mG)#(mA)#(mA)(mA)(mU)(mG)(mA)(mG)(mA)(mA)
(mC)(rC)(rG)(rU)(rU)(rG)(rC)(rU)(rA)(rC)(rA)
(rA)(rU)(rA)(rA)(rG)(rG)(rC)(rC)(rG)(mU)(mC)(mU)
(mG)(mA)(mA)(mA)(mA)(mG)(rA)(rU)(rG)(rU)(rG)(rC)
(rC)(rG)(rC)(rA)(rA)(rC)(rG)(rC)(rU)(rC)(rU)(rG)
(rC)(rC)(mC)(mC)(mU)(mU)(mA)(mA)(mA)(mG)(mC)(mU)
(mC)(mC)(mU)(mG)(mC)(mU)(mU)(mU)(mA)(mA)(mG)(mG)
(rG)(rG)(rC)(rA)(rU)(mC)(mG)(mU)#(mU)#(mU)#(mA)
(TR93-2),
wherein rN=RNA, mN=2′-O-methyl RNA, fN=2′-fluoro RNA, N #N=phosphorothioate linkage, and N=any nucleotide.

42-45. (canceled)

46. The chemically modified Nme gRNA of claim 41, wherein:
the tracrRNA portion comprises one or more modified nucleotides each independently selected from a modification of a ribose group, a phosphate group, a nucleobase, or a combination thereof, optionally wherein:
each modification of the ribose group is independently selected from the group consisting of 2′-O-methyl, 2′-fluoro, 2′-deoxy, 2′-O-(2-methoxyethyl) (MOE), 2′-NH₂ (2′-amino), 4′-thio, a bicyclic nucleotide, a locked nucleic acid (LNA), a 2′-(S)-constrained ethyl (5-cEt), a constrained MOE, and a 2′-O,4′-C-aminomethylene bridged nucleic acid (2′,4′-BNA^{Nc}), optionally wherein:
at least 40%, 80%, or 100% of the ribose groups are chemically modified, optionally wherein:
each modification of the phosphate group is independently selected from the group consisting of a phosphorothioate, phosphonoacetate (PACE), thiophosphonoacetate (thioPACE), amide, triazole, phosphonate, and phosphotriester modification, optionally wherein:
each modification of the nucleobase group is independently selected from the group consisting of 2-thiouridine, 4-thiouridine, N⁶-methyladenosine, pseudouridine, 2,6-diaminopurine, inosine, thymidine, 5-methylcytosine, 5-substituted pyrimidine, isoguanine, isocytosine, and halogenated aromatic groups.

47-52. (canceled)

53. The chemically modified Nme gRNA of claim 41, wherein tracrRNA portion comprises at least 40%, 80%, 90%, or 100% modified nucleotides, optionally wherein the tracrRNA portion modification pattern selected from any one of:
(SEQ ID NO: 94)
(mC)#(mG)#(mA)#(rA)(rA)(rU)(rG)(rA)(rG)(rA)(rA)
(rC)(rC)(rG)(rU)(rU)(rG)(rC)(rU)(rA)(rC)(rA)
(rA)(rU)(rA)(rA)(rG)(rG)(rC)(rC)(rG)(rU)(rC)(rU)
(rG)(rA)(rA))(rA)(rA)(rG)(rA)(rU)(rG)(rU)(rG)
(rC)(rC)(rG)(rC)(rA)(rA)(rC)(rG)(rC)(rU)(rC)(rU)
(rG)(rC)(rC)(rC)(rC)(rU)(rU)(rA)(rA)(rA)(rG)(rC)
(rU)(rC)(rC)(rU)(rG)(rC)(rU)(rU)(rU)(rA)(rA)(rG)
(rG)(rG)(rG)(rC)(rA)(rU)(rC)(rG)(rU)#(mU)#(mU)#
(mA) (TR93-0):
(SEP ID NO: 95)
(mC)#(mG)#(mA)#(rA)(rA)(rU)(rG)(rA)(rG)(rA)(rA)
(rC)(rC)(rG)(rU)(rU)(rG)(rC)(rU)(rA)(rC)(rA)
(rA)(rU)(rA)(rA)(rG)(rG)(rC)(rC)(rG)(rU)(rC)(rU)
(mG)(mA)(mA)(mA)(rA)(rG)(rA)(rU)(rG)(rU)(rG)(rC)
(rC)(rG)(rC)(rA)(rA)(rC)(rG)(rC)(rU)(rC)(rU)(rG)
(rC)(rC)(rC)(rC)(rU)(rU)(rA)(rA)(rA)(rG)(rC)(mU)
(mC)(mC)(mU)(rG)(rC)(rU)(rU)(rU)(rA)(rA)(rG)(rG)
(rG)(rG)(rC)(rA)(rU)(rC)(rG)(rU)#(mU)#(mU)#(mA)
(TR93-1);
(SEP ID NO: 96)
(mC)#(mG)#(mA)#(mA)(mA)(mU)(mG)(mA)(mG)(mA)(mA)
(mC)(rC)(rG)(rU)(rU)(rG)(rC)(rU)(rA)(rC)(rA)
(rA)(rU)(rA)(rA)(rG)(rG)(rC)(rC)(rG)(mU)(mC)(mU)
(mG)(mA)(mA)(mA)(mA)(mG)(rA)(rU)(rG)(rU)(rG)(rC)
(rC)(rG)(rC)(rA)(rA)(rC)(rG)(rC)(rU)(rC)(rU)(rG)
(rC)(rC)(mC)(mC)(mU)(mU)(mA)(mA)(mA)(mG)(mC)(mU)
(mC)(mC)(mU)(mG)(mC)(mU)(mU)(mU)(mA)(mA)(mG)(mG)
(rG)(rG)(rC)(rA)(rU)(mC)(mG)(mU)#(mU)#(mU)#(mA)
(TR93-2),
wherein rN=RNA, mN=2′-O-methyl RNA, fN=2′-fluoro RNA, N #N=phosphorothioate linkage, and N=any nucleotide.

54-58. (canceled)

59. The chemically modified Nme gRNA of claim 53, wherein:
the crRNA portion comprises one or more modified nucleotides each independently selected from a modification of a ribose group, a phosphate group, a nucleobase, or a combination thereof, optionally wherein:
each modification of the ribose group is independently selected from the group consisting of 2′-O-methyl, 2′-fluoro, 2′-deoxy, 2′-O-(2-methoxyethyl) (MOE), 2′-NH₂ (2′-amino), 4′-thio, a bicyclic nucleotide, a locked nucleic acid (LNA), a 2′-(S)-constrained ethyl (S-cEt), a constrained MOE, and a 2′-O,4′-C-aminomethylene bridged nucleic acid (2′,4′-BNA^{NC}), optionally wherein at least 50%, 80%, or 100% of the ribose groups are chemically modified;
each modification of the phosphate group is independently selected from the group consisting of a phosphorothioate, phosphonoacetate (PACE), thiophosphonoacetate (thioPACE), amide, triazole, phosphonate, or phosphotriester modification; and/or
each modification of the nucleobase group is independently selected from the group consisting of 2-thiouridine, 4-thiouridine, N⁶-methyladenosine, pseudouridine, 2,6-diaminopurine, inosine, thymidine, 5-methylcytosine, 5-substituted pyrimidine, isoguanine, isocytosine, and halogenated aromatic groups.

60-65. (canceled)

66. The chemically modified Nme gRNA of anyone of claim 59, wherein crRNA portion comprises at least 50%, 80%, 90%, or 90% modified nucleotides, optionally wherein the crRNA portion modification pattern selected from the group consisting of:

| | |
|---|---|
| CR48-2 | (mN)#(mN)#(mN)#(mN)(mN)(rN)(rN)(rN)(rN)(rN)(rN)(rN)(rN)(rN)(rN)(rN) |
| | (rN)(rN)(rN)(rN)(rN)(rN)(rN)(rN)(rG)(rU)(rU)(rG)(rU)(rA)(rG)(rC) |
| | (rU)(rC)(rC)(rC)(rU)(mU)(mU)(mC)(mU)(mC)(mA)(mU)(mU)#(mU)#(mC)#(mG) |
| | (SEQ ID NO: 13); |
| CR48-154 | (mN)#(mN)#(mN)#(mN)(mN)(mN)(dN)#(mN)(mN)(mN)(mN)(fN)(mN)(fN)(mN)(fN) |
| | (mN)(mN)(rN)(rN)(rN)(mN)(rN)(rN)(mG)(rU)(rU)(rG)(mU)(mA)(mG)(mC)(rU) |
| | (rC)(rC)(rC)(rU)(mU)(mU)(mC)(mU)(mC)(mA)(mU)(mU)#(mU)#(mC)#(mG) |
| | (SEQ ID NO: 14); |
| CR48-155 | (mN)#(mN)#(mN)#(mN)(mN)(mN)(dN)#(mN)(mN)(mN)(mN)(mN)(mN)(fN)(mN)(fN) |
| | (mN)(mN)(rN)(rN)(rN)(mN)(rN)(rN)(mG)(rU)(rU)(rG)(mU)(mA)(mG)(mC)(rU) |
| | (rC)(rC)(rC)(rU)(mU)(mU)(mC)(mU)(mC)(mA)(mU)(mU)#(mU)#(mC)#(mG) |
| | (SEQ ID NO: 15); |
| CR48-190 | (mN)#(mN)#(mN)#(mN)(mN)(mN)(dN)#(mN)(mN)(mN)(mN)(fN)(mN)(fN)(mN)(fN) |
| | (mN)(mN)(rN)(fN)(mN)(mN)(mN)(mN)(mG)(rU)(rU)(rG)(mU)(mA)(mG)(mC)(rU) |
| | (rC)(rC)(rC)(rU)(mU)(mU)(mC)(mU)(mC)(mA)(mU)(mU)#(mU)#(mC)#(mG) |
| | (SEQ ID NO: 16); |
| CR48-191 | (mN)#(mN)#(mN)#(mN)(mN)(mN)(dN)#(mN)(mN)(mN)(mN)(fN)(mN)(fN)(mN)(fN) |
| | (mN)(mN)(rN)(mN)(fN)(mN)(mN)(mN)(mG)(rU)(rU)(rG)(mU)(mA)(mG)(mC)(rU) |
| | (rC)(rC)(rC)(rU)(mU)(mU)(mC)(mU)(mC)(mA)(mU)(mU)#(mU)#(mC)#(mG) |
| | (SEQ ID NO: 17); |
| CR48-192 | (mN)#(mN)#(mN)#(mN)(mN)(mN)(dN)#(mN)(mN)(mN)(mN)(mN)(mN)(fN)(mN)(fN) |
| | (mN)(mN)(rN)(fN)(mN)(mN)(mN)(mN)(mG)(rU)(rU)(rG)(mU)(mA)(mG)(mC)(rU) |
| | (rC)(rC)(rC)(rU)(mU)(mU)(mC)(mU)(mC)(mA)(mU)(mU)#(mU)#(mC)#(mG) |
| | (SEQ ID NO: 18); |
| CR48-193 | (mN)#(mN)#(mN)#(mN)(mN)(mN)(dN)#(mN)(mN)(mN)(mN)(mN)(mN)(fN)(mN)(fN) |
| | (mN)(mN)(rN)(mN)(fN)(mN)(mN)(mN)(mG)(rU)(rU)(rG)(mU)(mA)(mG)(mC)(rU) |
| | (rC)(rC)(rC)(rU)(mU)(mU)(mC)(mU)(mC)(mA)(mU)(mU)#(mU)#(mC)#(mG) |
| | (SEQ ID NO: 19); |
| CR48-194 | (mN)#(mN)#(mN)#(mN)(mN)(mN)(dN)#(mN)(mN)(mN)(mN)(fN)(mN)(fN)(mN)(fN) |
| | (mN)(mN)(rN)#(fN)(mN)(mN)(mN)(mN)(mG)(rU)(rU)(rG)(mU)(mA)(mG)(mC)(rU) |
| | (rC)(rC)(rC)(rU)(mU)(mU)(mC)(mU)(mC)(mA)(mU)(mU)#(mU)#(mC)#(mG) |
| | (SEQ ID NO: 20); |
| CR48-195 | (mN)#(mN)#(mN)#(mN)(mN)(mN)(dN)#(mN)(mN)(mN)(mN)(fN)(mN)(fN)(mN)(fN) |
| | (mN)(mN)(rN)#(mN)(fN)(mN)(mN)(mN)(mG)(rU)(rU)(rG)(mU)(mA)(mG)(mC)(rU) |
| | (rC)(rC)(rC)(rU)(mU)(mU)(mC)(mU)(mC)(mA)(mU)(mU)#(mU)#(mC)#(mG) |
| | (SEQ ID NO: 21); |
| CR48-196 | (mN)#(mN)#(mN)#(mN)(mN)(mN)(dN)#(mN)(mN)(mN)(mN)(mN)(mN)(fN)(mN)(fN) |
| | (mN)(mN)(rN)#(fN)(mN)(mN)(mN)(mN)(mG)(rU)(rU)(rG)(mU)(mA)(mG)(mC)(rU) |
| | (rC)(rC)(rC)(rU)(mU)(mU)(mC)(mU)(mC)(mA)(mU)(mU)#(mU)#(mC)#(mG) |
| | (SEQ ID NO: 22); |
| CR48-197 | (mN)#(mN)#(mN)#(mN)(mN)(mN)(dN)#(mN)(mN)(mN)(mN)(mN)(mN)(fN)(mN)(fN) |
| | (mN)(mN)(rN)#(mN)(fN)(mN)(mN)(mN)(mG)(rU)(rU)(rG)(mU)(mA)(mG)(mC)(rU) |
| | (rC)(rC)(rC)(rU)(mU)(mU)(mC)(mU)(mC)(mA)(mU)(mU)#(mU)#(mC)#(mG) |
| | (SEQ ID NO: 23); |
| CR48-198 | (mN)#(mN)#(mN)#(mN)(mN)(mN)(dN)#(mN)(mN)(mN)(mN)(fN)(mN)(fN)(mN)(fN) |
| | (mN)(mN)(mN)(fN)(mN)(mN)(mN)(mN)(mG)(rU)(rU)(rG)(mU)(mA)(mG)(mC)(rU) |
| | (rC)(rC)(rC)(rU)(mU)(mU)(mC)(mU)(mC)(mA)(mU)(mU)#(mU)#(mC)#(mG) |
| | (SEQ ID NO: 24); |
| CR48-199 | (mN)#(mN)#(mN)#(mN)(mN)(mN)(dN)#(mN)(mN)(mN)(mN)(fN)(mN)(fN)(mN)(fN) |
| | (mN)(mN)(mN)(mN)(fN)(mN)(mN)(mN)(mG)(rU)(rU)(rG)(mU)(mA)(mG)(mC)(rU) |
| | (rC)(rC)(rC)(rU)(mU)(mU)(mC)(mU)(mC)(mA)(mU)(mU)#(mU)#(mC)#(mG) |
| | (SEQ ID NO: 25); |
| CR48-200 | (mN)#(mN)#(mN)#(mN)(mN)(mN)(dN)#(mN)(mN)(mN)(mN)(mN)(mN)(fN)(mN)(fN) |
| | (mN)(mN)(mN)(fN)(mN)(mN)(mN)(mN)(mG)(rU)(rU)(rG)(mU)(mA)(mG)(mC)(rU) |
| | (rC)(rC)(rC)(rU)(mU)(mU)(mC)(mU)(mC)(mA)(mU)(mU)#(mU)#(mC)#(mG) |
| | (SEQ ID NO: 26); |
| CR48-201 | (mN)#(mN)#(mN)#(mN)(mN)(mN)(dN)#(mN)(mN)(mN)(mN)(mN)(mN)(fN)(mN)(fN) |
| | (mN)(mN)(mN)(mN)(fN)(mN)(mN)(mN)(mG)(rU)(rU)(rG)(mU)(mA)(mG)(mC)(rU) |
| | (rC)(rC)(rC)(rU)(mU)(mU)(mC)(mU)(mC)(mA)(mU)(mU)#(mU)#(mC)#(mG) |
| | (SEQ ID NO: 27); |
| CR48-202 | (mN)#(mN)#(mN)#(mN)(mN)(mN)(dN)#(mN)(mN)(mN)(mN)(fN)(mN)(fN)(mN)(fN) |
| | (mN)(mN)(fN)(fN)(mN)(mN)(mN)(mN)(mG)(rU)(rU)(rG)(mU)(mA)(mG)(mC)(rU) |
| | (rC)(rC)(rC)(rU)(mU)(mU)(mC)(mU)(mC)(mA)(mU)(mU)#(mU)#(mC)#(mG) |
| | (SEQ ID NO: 28); |
| CR48-203 | (mN)#(mN)#(mN)#(mN)(mN)(mN)(dN)#(mN)(mN)(mN)(mN)(fN)(mN)(fN)(mN)(fN) |
| | (mN)(mN)(fN)(mN)(fN)(mN)(mN)(mN)(mG)(rU)(rU)(rG)(mU)(mA)(mG)(mC)(rU) |
| | (rC)(rC)(rC)(rU)(mU)(mU)(mC)(mU)(mC)(mA)(mU)(mU)#(mU)#(mC)#(mG) |
| | (SEQ ID NO: 29); |
| CR48-204 | (mN)#(mN)#(mN)#(mN)(mN)(mN)(dN)#(mN)(mN)(mN)(mN)(mN)(mN)(fN)(mN)(fN) |
| | (mN)(mN)(fN)(fN)(mN)(mN)(mN)(mN)(mG)(rU)(rU)(rG)(mU)(mA)(mG)(mC)(rU) |
| | (rC)(rC)(rC)(rU)(mU)(mU)(mC)(mU)(mC)(mA)(mU)(mU)#(mU)#(mC)#(mG) |
| | (SEQ ID NO: 30); |
| CR48-205 | (mN)#(mN)#(mN)#(mN)(mN)(mN)(dN)#(mN)(mN)(mN)(mN)(mN)(mN)(fN)(mN)(fN) |
| | (mN)(mN)(fN)(mN)(fN)(mN)(mN)(mN)(mG)(rU)(rU)(rG)(mU)(mA)(mG)(mC)(rU) |
| | (rC)(rC)(rC)(rU)(mU)(mU)(mC)(mU)(mC)(mA)(mU)(mU)#(mU)#(mC)#(mG) |
| | (SEQ ID NO: 31); |
| CR48-213 | (mN)#(mN)#(mN)#(mN)(mN)(mN)(dN)#(mN)(mN)(mN)(mN)(mN)(mN)(fN)(mN)(fN) |
| | (mN)(mN)(rN)(rN)(rN)(mN)(rN)(rN)(mG)(dT)#(rU)(mG)(mU)(mA)(mG)(mC)(rU) |
| | (rC)(rC)(rC)(rU)(mU)(mU)(mC)(mU)(mC)(mA)(mU)(mU)#(mU)#(mC)#(mG) |
| | (SEQ ID NO: 32); |
| CR48-214 | (mN)#(mN)#(mN)#(mN)(mN)(mN)(dN)#(mN)(mN)(mN)(mN)(mN)(mN)(fN)(mN)(fN) |
| | (mN)(mN)(rN)(rN)(rN)(mN)(rN)(rN)(mG)(dT)#(rU)(mG)(mU)(mA)(mG)(mC)(rU) |
| | (rC)(mC)(rC)(rU)(mU)(mU)(mC)(mU)(mC)(mA)(mU)(mU)#(mU)#(mC)#(mG) |
| | (SEQ ID NO: 33); |
| CR48-215 | (mN)#(mN)#(mN)#(mN)(mN)(mN)(dN)#(mN)(mN)(mN)(mN)(mN)(mN)(fN)(mN)(fN) |
| | (mN)(mN)(rN)(rN)(rN)(mN)(rN)(rN)(mG)(dT)#(rU)(mG)(mU)(mA)(mG)(mC)(rU) |
| | (rC)(rC)(mC)(rU)(mU)(mU)(mC)(mU)(mC)(mA)(mU)(mU)#(mU)#(mC)#(mG) |
| | (SEQ ID NO: 34); |
| CR48-216 | (mN)#(mN)#(mN)#(mN)(mN)(mN)(dN)#(mN)(mN)(mN)(mN)(mN)(mN)(fN)(mN)(fN) |
| | (mN)(mN)(rN)(rN)(rN)(mN)(rN)(rN)(mG)(dT)#(rU)(mG)(mU)(mA)(mG)(mC)(rU) |
| | (rC)(mC)(mC)(rU)(mU)(mU)(mC)(mU)(mC)(mA)(mU)(mU)#(mU)#(mC)#(mG) |
| | (SEQ ID NO: 35); |
| CR48-217 | (mN)#(mN)#(mN)#(mN)(mN)(mN)(dN)#(mN)(mN)(mN)(mN)(mN)(mN)(fN)(mN)(fN) |
| | (mN)(mN)(rN)(rN)(rN)(mN)(rN)(rN)(mG)(rU)(rU)(rG)(mU)(mA)(mG)(mC)(dT) |
| | #(rC)(rC)(rC)(rU)(mU)(mU)(mC)(mU)(mC)(mA)(mU)(mU)#(mU)#(mC)#(mG) |
| | (SEQ ID NO: 36); |
| CR48-218 | (mN)#(mN)#(mN)#(mN)(mN)(mN)(dN)#(mN)(mN)(mN)(mN)(mN)(mN)(fN)(mN)(fN) |
| | (mN)(mN)(rN)(rN)(rN)(mN)(rN)(rN)(mG)(rU)(rU)(rG)(mU)(mA)(mG)(mC)(rU) |
| | (dC)#(rC)(rC)(rU)(mU)(mU)(mC)(mU)(mC)(mA)(mU)(mU)#(mU)#(mC)#(mG) |
| | (SEQ ID NO: 37); |
| CR48-219 | (mN)#(mN)#(mN)#(mN)(mN)(mN)(dN)#(mN)(mN)(mN)(mN)(mN)(mN)(fN)(mN)(fN) |
| | (mN)(mN)(rN)(rN)(rN)(mN)(rN)(rN)(mG)(rU)(rU)(rG)(mU)(mA)(mG)(mC)(rU) |
| | (rC)(dC)#(rC)(rU)(mU)(mU)(mC)(mU)(mC)(mA)(mU)(mU)#(mU)#(mC)#(mG) |
| | (SEQ ID NO: 38); |
| CR48-220 | (mN)#(mN)#(mN)#(mN)(mN)(mN)(dN)#(mN)(mN)(mN)(mN)(mN)(mN)(fN)(mN)(fN) |
| | (mN)(mN)(rN)(rN)(rN)(mN)(rN)(rN)(mG)(rU)(rU)(rG)(mU)(mA)(mG)(mC)(rU) |
| | (rC)(rC)(dC)#(rU)(mU)(mU)(mC)(mU)(mC)(mA)(mU)(mU)#(mU)#(mC)#(mG) |
| | (SEQ ID NO: 39); |
| CR48-221 | (mN)#(mN)#(mN)#(mN)(mN)(mN)(dN)#(mN)(mN)(mN)(mN)(mN)(mN)(fN)(mN)(fN) |
| | (mN)(mN)(rN)(rN)(rN)(mN)(rN)(rN)(mG)(rU)(rU)(rG)(mU)(mA)(mG)(mC)(rU) |
| | (rC)(rC)(rC)(dT)#(mU)(mU)(mC)(mU)(mC)(mA)(mU)(mU)#(mU)#(mC)#(mG) |
| | (SEQ ID NO: 40); |
| CR48-222 | (mN)#(mN)#(mN)#(mN)(mN)(mN)(dN)#(mN)(mN)(mN)(mN)(mN)(mN)(fN)(mN)(fN) |
| | (mN)(mN)(rN)(rN)(rN)(mN)(rN)(rN)(mG)(rU)(rU)(rG)(mU)(mA)(mG)(mC)(dT) |
| | #(rC)(dC)#(rC)(dT)#(mU)(mU)(mC)(mU)(mC)(mA)(mU)(mU)#(mU)#(mC)#(mG) |
| | (SEQ ID NO: 41); |
| CR48-223 | (mN)#(mN)#(mN)#(mN)(mN)(mN)(dN)#(mN)(mN)(mN)(mN)(mN)(mN)(fN)(mN)(fN) |
| | (mN)(mN)(rN)(rN)(rN)(mN)(rN)(rN)(mG)(dT)#(rU)(rG)(mU)(mA)(mG)(mC)(dT) |
| | #(dC)#(dC)#(rC)(dT)#(mU)(mU)(mC)(mU)(mC)(mA)(mU)(mU)#(mU)#(mC)#(mG) |
| | (SEQ ID NO: 42); |
| CR48-224 | (mN)#(mN)#(mN)#(mN)(mN)(mN)(dN)#(mN)(mN)(mN)(mN)(mN)(mN)(fN)(mN)(fN) |
| | (mN)(mN)(rN)(rN)(rN)(mN)(rN)(rN)(mG)(dT)#(rU)(mG)(mU)(mA)(mG)(mC)(dT) |
| | #(rC)(mC)(rC)(dT)#(mU)(mU)(mC)(mU)(mC)(mA)(mU)(mU)#(mU)#(mC)#(mG) |
| | (SEQ ID NO: 43); |
| CR48-225 | (mN)#(mN)#(mN)#(mN)(mN)(mN)(dN)#(mN)(mN)(mN)(mN)(mN)(mN)(fN)(mN)(fN) |
| | (mN)(mN)(rN)(rN)(rN)(mN)(rN)(rN)(mG)(dT)#(rU)(mG)(mU)(mA)(mG)(mC)(dT) |
| | #(rC)(dC)#(mC)(dT)#(mU)(mU)(mC)(mU)(mC)(mA)(mU)(mU)#(mU)#(mC)#(mG) |
| | (SEQ ID NO: 44); |
| CR48-226 | (mN)#(mN)#(mN)#(mN)(mN)(mN)(dN)#(mN)(mN)(mN)(mN)(mN)(mN)(fN)(mN)(fN) |
| | (mN)(mN)(rN)(rN)(rN)(mN)(rN)(rN)(mG)(dT)#(rU)(mG)(mU)(mA)(mG)(mC)(dT) |
| | #(rC)(mC)(mC)(dT)#(mU)(mU)(mC)(mU)(mC)(mA)(mU)(mU)#(mU)#(mC)#(mG) |
| | (SEQ ID NO: 45); |
| CR48-227 | (mN)#(mN)#(mN)#(mN)(mN)(mN)(dN)#(mN)(mN)(mN)(mN)(mN)(mN)(fN)(mN)(fN) |
| | (mN)(mN)(rN)(rN)(rN)(mN)(rN)(rN)(mG)(dT)#(rU)(mG)(mU)(mA)(mG)(mC)(dT) |
| | #(dC)#(mC)(mC)(dT)#(mU)(mU)(mC)(mU)(mC)(mA)(mU)(mU)#(mU)#(mC)#(mG) |
| | (SEQ ID NO: 46); |
| CR48-199- | (mN)#(mN)#(mN)#(mN)(mN)(mN)(dN)#(mN)(mN)(mN)(mN)(fN)(mN)(fN)(mN)(fN) |
| V1.1 | (mN)(mN)(mN)(mN)(fN)(mN)(mN)(mN)(mG)(rU)(rU)(mG)(mU)(mA)(mG)(mC)(rU) |
| | (rC)(rC)(dC)#(rU)(mU)(mU)(mC)(mU)(mC)(mA)(mU)(mU)#(mU)#(mC)#(mG) |
| | (SEQ ID NO: 47); |
| CR48-199- | (mN)#(mN)#(mN)#(mN)(mN)(mN)(dN)#(mN)(mN)(mN)(mN)(fN)(mN)(fN)(mN)(fN) |
| V1.2 | (mN)(mN)(mN)(mN)(fN)(mN)(mN)(mN)(mG)(rU)(rU)(mG)(mU)(mA)(mG)(mC)(rU) |
| | (rC)(dC)#(rC)(rU)(mU)(mU)(mC)(mU)(mC)(mA)(mU)(mU)#(mU)#(mC)#(mG) |
| | (SEQ ID NO: 48); |
| CR48-199- | (mN)#(mN)#(mN)#(mN)(mN)(mN)(dN)#(mN)(mN)(mN)(mN)(fN)(mN)(fN)(mN)(fN) |
| V1.3 | (mN)(mN)(mN)(mN)(fN)(mN)(mN)(mN)(mG)(rU)(rU)(mG)(mU)(mA)(mG)(mC)(rU) |
| | (rC)(rC)(rC)(dT)#(mU)(mU)(mC)(mU)(mC)(mA)(mU)(mU)#(mU)#(mC)#(mG) |
| | (SEQ ID NO: 49); |
| CR48-199- | (mN)#(mN)#(mN)#(mN)(mN)(mN)(dN)#(mN)(mN)(mN)(mN)(fN)(mN)(fN)(mN)(fN) |
| V1.4 | (mN)(mN)(mN)(mN)(fN)(mN)(mN)(mN)(mG)(rU)(rU)(mG)(mU)(mA)(mG)(mC)(rU) |
| | (rC)(mC)(rC)(rU)(mU)(mU)(mC)(mU)(mC)(mA)(mU)(mU)#(mU)#(mC)#(mG) |
| | (SEQ ID NO: 50); |
| CR48-199- | (mN)#(mN)#(mN)#(mN)(mN)(mN)(dN)#(mN)(mN)(mN)(mN)(fN)(mN)(fN)(mN)(fN) |
| V1.5 | (mN)(mN)(mN)(mN)(fN)(mN)(mN)(mN)(mG)(rU)(rU)(mG)(mU)(mA)(mG)(mC)(rU) |
| | (rC)(dC)#(dC)#(rU)(mU)(mU)(mC)(mU)(mC)(mA)(mU)(mU)#(mU)#(mC)#(mG) |
| | (SEQ ID NO: 51); |
| CR48-199- | (mN)#(mN)#(mN)#(mN)(mN)(mN)(dN)#(mN)(mN)(mN)(mN)(fN)(mN)(fN)(mN)(fN) |
| V1.6 | (mN)(mN)(mN)(mN)(fN)(mN)(mN)(mN)(mG)(rU)(rU)(mG)(mU)(mA)(mG)(mC)(rU) |
| | (rC)(dC)#(rC)(dT)#(mU)(mU)(mC)(mU)(mC)(mA)(mU)(mU)#(mU)#(mC)#(mG) |
| | (SEQ ID NO: 52); |
| CR48-199- | (mN)#(mN)#(mN)#(mN)(mN)(mN)dN)#(mN)(mN)(mN)(mN)(fN)(mN)(fN)(mN)(fN) |
| V1.7 | (mN)mN)(mN)(mN)(fN)(mN)(mN)(mN)(mG)(rU)(rU)(mG)(mU)(mA)(mG)(mC)(rU) |
| | (rC)(dC)#(dC)#(dT)#(mU)(mU)(mC)(mU)(mC)(mA)(mU)(mU)#(mU)#(mC)#(mG) |
| | (SEQ ID NO: 53); |
| CR48-199- | (mN)#(mN)#(mN)#(mN)(mN)(mN)(dN)#(mN)(mN)(mN)(mN)(fN)(mN)(fN)(mN)(fN) |
| V1.8 | (mN)(mN)(mN)(mN)(fN)(mN)(mN)(mN)(mG)(rU)(rU)(mG)(mU)(mA)(mG)(mC)(rU) |
| | (rC)(mC)(dC)#(rU)(mU)(mU)(mC)(mU)(mC)(mA)(mU)(mU)#(mU)#(mC)#(mG) |
| | (SEQ ID NO: 54); |
| CR48-199- | (mN)#(mN)#(mN)#(mN)(mN)(mN)(dN)#(mN)(mN)(mN)(mN)(fN)(mN)(fN)(mN)(fN) |
| V1.9 | (mN)(mN)(mN)(mN)(fN)(mN)(mN)(mN)(mG)(rU)(rU)(mG)(mU)(mA)(mG)(mC)(rU) |
| | (rC)(mC)(rC)(dT)#(mU)(mU)(mC)(mU)(mC)(mA)(mU)(mU)#(mU)#(mC)#(mG) |
| | (SEQ ID NO: 55); |
| CR48-199- | (mN)#(mN)#(mN)#(mN)(mN)(mN)(dN)#(mN)(mN)(mN)(mN)(fN)(mN)(fN)(mN)(fN) |
| V1.10 | (mN)(mN)(mN)(mN)(fN)(mN)(mN)(mN)(mG)(rU)(rU)(mG)(mU)(mA)(mG)(mC)(rU) |
| | (rC)(mC)(dC)#(dT)#(mU)(mU)(mC)(mU)(mC)(mA)(mU)(mU)#(mU)#(mC)#(mG) |
| | (SEQ ID NO: 56); |
| CR48-199- | (mN)#(mN)#(mN)#(mN)(mN)(mN)(dN)#(mN)(mN)(mN)(mN)(fN)(mN)(fN)(mN)(fN) |
| V1.11 | (mN)(mN)(mN)(mN)(fN)(mN)(mN)(mN)(mG)(rU)(rU)(mG)(mU)(mA)(mG)(mC)(rU) |
| | (rC)(mC)(mC)(rU)(mU)(mU)(mC)(mU)(mC)(mA)(mU)(mU)#(mU)#(mC)#(mG) |
| | (SEQ ID NO: 57); |
| CR48-199- | (mN)#(mN)#(mN)#(mN)(mN)(mN)(dN)#(mN)(mN)(mN)(mN)(fN)(mN)(fN)(mN)(fN) |
| V1.12 | (mN)(mN)(mN)(mN)(fN)(mN)(mN)(mN)(mG)(rU)(rU)(mG)(mU)(mA)(mG)(mC)(rU) |
| | (rC)(mC)(mC)(dT)#(mU)(mU)(mC)(mU)(mC)(mA)(mU)(mU)#(mU)#(mC)#(mG) |
| | (SEQ ID NO: 58); |
| CR48-199- | (mN)#(mN)#(mN)#(mN)(mN)(mN)(dN)#(mN)(mN)(mN)(mN)(fN)(mN)(fN)(mN)(fN) |
| V1.13 | (mN)(mN)(mN)(mN)(fN)(mN)(mN)(mN)(mG)(rU)(rU)(mG)(mU)(mA)(mG)(mC)(dT) |
| | #(rC)(rC)(dC)#(rU)(mU)(mU)(mC)(mU)(mC)(mA)(mU)(mU)#(mU)#(mC)#(mG) |
| | (SEQ ID NO: 59); |
| CR48-199- | (mN)#(mN)#(mN)#(mN)(mN)(mN)(dN)#(mN)(mN)(mN)(mN)(fN)(mN)(fN)(mN)(fN) |
| V1.14 | (mN)(mN)(mN)(mN)(fN)(mN)(mN)(mN)(mG)(rU)(rU)(mG)(mU)(mA)(mG)(mC)(dT) |
| | #(rC)(dC)#(rC)(rU)(mU)(mU)(mC)(mU)(mC)(mA)(mU)(mU)#(mU)#(mC)#(mG) |
| | (SEQ ID NO: 60); |
| CR48-199- | (mN)#(mN)#(mN)#(mN)(mN)(mN)(dN)#(mN)(mN)(mN)(mN)(fN)(mN)(fN)(mN)(fN) |
| V1.15 | (mN)(mN)(mN)(mN)(fN)(mN)(mN)(mN)(mG)(rU)(rU)(mG)(mU)(mA)(mG)(mC)(dT) |
| | #(rC)(rC)(rC)(dT)#(mU)(mU)(mC)(mU)(mC)(mA)(mU)(mU)#(mU)#(mC)#(mG) |
| | (SEQ ID NO: 61); |
| CR48-199- | (mN)#(mN)#(mN)#(mN)(mN)(mN)(dN)#(mN)(mN)(mN)(mN)(fN)(mN)(fN)(mN)(fN) |
| V1.16 | (mN)(mN)(mN)(mN)(fN)(mN)(mN)(mN)(mG)(rU)(rU)(mG)(mU)(mA)(mG)(mC)(dT) |
| | #(rC)(mC)(rC)(rU)(mU)(mU)(mC)(mU)(mC)(mA)(mU)(mU)#(mU)#(mC)#(mG) |
| | (SEQ ID NO: 62); |
| CR48-199- | (mN)#(mN)#(mN)#(mN)(mN)(mN)(dN)#(mN)(mN)(mN)(mN)(fN)(mN)(fN)(mN)(fN) |
| V1.17 | (mN)(mN)(mN)(mN)(fN)(mN)(mN)(mN)(mG)(rU)(rU)(mG)(mU)(mA)(mG)(mC)(dT) |
| | #(rC)(dC)#(dC)#(rU)(mU)(mU)(mC)(mU)(mC)(mA)(mU)(mU)#(mU)#(mC)#(mG) |
| | (SEQ ID NO: 63); |
| CR48-199- | (mN)#(mN)#(mN)#(mN)(mN)(mN)(dN)#(mN)(mN)(mN)(mN)(fN)(mN)(fN)(mN)(fN) |
| V1.18 | (mN)(mN)(mN)(mN)(fN)(mN)(mN)(mN)(mG)(rU)(rU)(mG)(mU)(mA)(mG)(mC)(dT) |
| | #(rC)(dC)#(rC)(dT)#(mU)(mU)(mC)(mU)(mC)(mA)(mU)(mU)#(mU)#(mC)#(mG) |
| | (SEQ ID NO: 64); |
| CR48-199- | (mN)#(mN)#(mN)#(mN)(mN)(mN)(dN)#(mN)(mN)(mN)(mN)(fN)(mN)(fN)(mN)(fN) |
| V1.19 | (mN)(mN)(mN)(mN)(fN)(mN)(mN)(mN)(mG)(rU)(rU)(mG)(mU)(mA)(mG)(mC)(dT) |
| | #(rC)(dC)#(dC)#(dT)#(mU)(mU)(mC)(mU)(mC)(mA)(mU)(mU)#(mU)#(mC)#(mG) |
| | (SEQ ID NO: 65); |
| CR48-199- | (mN)#(mN)#(mN)#(mN)(mN)(mN)(dN)#(mN)(mN)(mN)(mN)(fN)(mN)(fN)(mN)(fN) |
| V1.20 | (mN)(mN)(mN)(mN)(fN)(mN)(mN)(mN)(mG)(rU)(rU)(mG)(mU)(mA)(mG)(mC)(dT) |
| | #(rC)(mC)(dC)#(rU)(mU)(mU)(mC)(mU)(mC)(mA)(mU)(mU)#(mU)#(mC)#(mG) |
| | (SEQ ID NO: 66); |
| CR48-199- | (mN)#(mN)#(mN)#(mN)(mN)(mN)(dN)#(mN)(mN)(mN)(mN)(fN)(mN)(fN)(mN)(fN) |
| V1.21 | (mN)(mN)(mN)(mN)(fN)(mN)(mN)(mN)(mG)(rU)(rU)(mG)(mU)(mA)(mG)(mC)(dT) |
| | #(rC)(mC)(rC)(dT)#(mU)(mU)(mC)(mU)(mC)(mA)(mU)(mU)#(mU)#(mC)#(mG) |
| | (SEQ ID NO: 67); |
| CR48-199- | (mN)#(mN)#(mN)#(mN)(mN)(mN)(dN)#(mN)(mN)(mN)(mN)(fN)(mN)(fN)(mN)(fN) |
| V1.22 | (mN)(mN)(mN)(mN)(fN)(mN)(mN)(mN)(mG)(rU)(rU)(mG)(mU)(mA)(mG)(mC)(dT) |
| | #(rC)(mC)(dC)#(dT)#(mU)(mU)(mC)(mU)(mC)(mA)(mU)(mU)#(mU)#(mC)#(mG) |
| | (SEQ ID NO: 68); |
| CR48-199- | (mN)#(mN)#(mN)#(mN)(mN)(mN)(dN)#(mN)(mN)(mN)(mN)(fN)(mN)(fN)(mN)(fN) |
| V1.23 | (mN)(mN)(mN)(mN)(IN)(mN)(mN)(mN)(mG)(rU)(rU)(mG)(mU)(mA)(mG)(mC)(dT) |
| | #(rC)(mC)(mC)(rU)(mU)(mU)(mC)(mU)(mC)(mA)(mU)(mU)#(mU)#(mC)#(mG) |
| | (SEQ ID NO: 69); |
| CR48-199- | (mN)#(mN)#(mN)#(mN)(mN)(mN)(dN)#(mN)(mN)(mN)(mN)(fN)(mN)(fN)(mN)(fN) |
| V1.24 | (mN)(mN)(mN)(mN)(fN)(mN)(mN)(mN)(mG)(rU)(rU)(mG)(mU)(mA)(mG)(mC)(dT) |
| | #(rC)(mC)(mC)(dT)#(mU)(mU)(mC)(mU)(mC)(mA)(mU)(mU)#(mU)#(mC)#(mG) |
| | (SEQ ID NO: 70); |
| CR48-199- | (mN)#(mN)#(mN)#(mN)(mN)(mN)(dN)#(mN)(mN)(mN)(mN)(fN)(mN)(fN)(mN)(fN) |
| V1.25 | (mN)(mN)(mN)(mN)(fN)(mN)(mN)(mN)(mG)(dT)#(rU)(mG)(mU)(mA)(mG)(mC)(dT) |
| | #(rC)(rC)(dC)#(rU)(mU)(mU)(mC)(mU)(mC)(mA)(mU)(mU)#(mU)#(mC)#(mG) |
| | (SEQ ID NO: 71); |
| CR48-199- | (mN)#(mN)#(mN)#(mN)(mN)(mN)(dN)#(mN)(mN)(mN)(mN)(fN)(mN)(fN)(mN)(fN) |
| V1.26 | (mN)(mN)(mN)(mN)(fN)(mN)(mN)(mN)(mG)(dT)#(rU)(mG)(mU)(mA)(mG)(mC)(dT) |
| | #(rC)(dC)#(rC)(rU)(mU)(mU)(mC)(mU)(mC)(mA)(mU)(mU)#(mU)#(mC)#(mG) |
| | (SEQ ID NO: 72); |
| CR48-199- | (mN)#(mN)#(mN)#(mN)(mN)(mN)(dN)#(mN)(mN)(mN)(mN)(fN)(mN)(fN)(mN)(fN) |
| V1.27 | (mN)(mN)(mN)(mN)(fN)(mN)(mN)(mN)(mG)(dT)#(rU)(mG)(mU)(mA)(mG)(mC)(dT) |
| | #(rC)(rC)(r)(dT)#(mU)(mU)(mC)(mU)(mC)(mA)(mU)(mU)#(mU)#(mC)#(mG) |
| | (SEQ ID NO: 73); |
| CR48-199- | (mN)#(mN)#(mN)#(mN)(mN)(mN)(dN)#(mN)(mN)(mN)(mN)(fN)(mN)(fN)(mN)(fN) |
| V1.28 | (mN)(mN)(mN)(mN)(fN)(mN)(mN)(mN)(mG)(dT)#(rU)(mG)(mU)(mA)(mG)(mC)(dT) |
| | #(rC)(mC)(rC)(rU)(mU)(mU)(mC)(mU)(mC)(mA)(mU)(mU)#(mU)#(mC)#(mG) |
| | (SEQ ID NO: 74); |
| CR48-199- | (mN)#(mN)#(mN)#(mN)(mN)(mN)(dN)#(mN)(mN)(mN)(mN)(fN)(mN)(fN)(mN)(fN) |
| V1.29 | mN(mN)()(mN)(mN)(fN)(mN)(mN)(mN)(mG)(dT)#(rU)(mG)(mU)(mA)(mG)(mC)(dT) |
| | dC#(rC)(dC)#()#(rU)(mU)(mU)(mC)(mU)(mC)(mA)(mU)(mU)#(mU)#(mC)#(mG) |
| | (SEQ ID NO: 75); |
| CR48-199- | (mN)#(mN)#(mN)#(mN)(mN)(mN)(dN)#(mN)(mN)(mN)(mN)(fN)(mN)(fN)(mN)(fN) |
| V1.30 | (mN)(mN)(mN)(mN)(fN)(mN)(mN)(mN)(mG)(dT)#(rU)(mG)(mU)(mA)(mG)(mC)(dT) |
| | #(rC)(dC)#(rC)(dT)#(mU)(mU)(mC)(mU)(mC)(mA)(mU)(mU)#(mU)#(mC)#(mG) |
| | (SEQ ID NO: 76); |
| CR48-199- | (mN)#(mN)#(mN)#(mN)(mN)(mN)(dN)#(mN)(mN)(mN)(mN)(fN)(mN)(fN)(mN)(fN) |
| V1.31 | (mN)(mN)(mN)(mN)(fN)(mN)(mN)(mN)(mG)(dT)#(rU)(mG)(mU)(mA)(mG)(mC)(dT) |
| | #(rC)(dC)#(dC)#(dT)#(mU)(mU)(mC)(mU)(mC)(mA)(mU)(mU)#(mU)#(mC)#(mG) |
| | (SEQ ID NO: 77); |
| CR48-199- | (mN)#(mN)#(mN)#(mN)(mN)(mN)(dN)#(mN)(mN)(mN)(mN)(fN)(mN)(fN)(mN)(fN) |
| V1.32 | (mN)(mN)(mN)(mN)(fN)(mN)(mN)(mN)(mG)(dT)#(rU)(mG)(mU)(mA)(mG)(mC)(dT) |
| | #(rC)(mC)(dC)#(rU)(mU)(mU)(mC)(mU)(mC)(mA)(mU)(mU)#(mU)#(mC)#(mG) |
| | (SEQ ID NO: 78); |
| CR48-199- | (mN)#(mN)#(mN)#(mN)(mN)(mN)(dN)#(mN)(mN)(mN)(mN)(fN)(mN)(fN)(mN)(fN) |
| V1.33 | (mN)(mN)(mN)(mN)(fN)(mN)(mN)(mN)(mG)(dT)#(rU)(mG)(mU)(mA)(mG)(mC)(dT) |
| | #(rC)(mC)(rC)(dT)#(mU)(mU)(mC)(mU)(mC)(mA)(mU)(mU)#(mU)#(mC)#(mG) |
| | (SEQ ID NO: 79); |
| CR48-199- | (mN)#(mN)#(mN)#(mN)(mN)(mN)(dN)#(mN)(mN)(mN)(mN)(fN)(mN)(fN)(mN)(fN) |
| V1.34 | (mN)(mN)(mN)(mN)(fN)(mN)(mN)(mN)(mG)(dT)#(rU)(mG)(mU)(mA)(mG)(mC)(dT) |
| | #(rC)(mC)(dC)#(dT)#(mU)(mU)(mC)(mU)(mC)(mA)(mU)(mU)#(mU)#(mC)#(mG) |
| | (SEQ ID NO: 80); |
| CR48-199- | (mN)#(mN)#(mN)#(mN)(mN)(mN)(dN)#(mN)(mN)(mN)(mN)(fN)(mN)(fN)(mN)(fN) |
| V1.35 | (mN)(mN)(mN)(mN)(fN)(mN)(mN)(mN)(mG)(dT)#(rU)(mG)(mU)(mA)(mG)(mC)(dT) |
| | #(rC)(mC)(mC)(rU)(mU)(mU)(mC)(mU)(mC)(mA)(mU)(mU)#(mU)#(mC)#(mG) |
| | (SEQ ID NO: 81); |
| CR48-199- | (mN)#(mN)#(mN)#(mN)(mN)(mN)(dN)#(mN)(mN)(mN)(mN)(fN)(mN)(fN)(mN)(fN) |
| V1.36 | (mN)(mN)(mN)(mN)(fN)(mN)(mN)(mN)(mG)(dT)#(rU)(mG)(mU)(mA)(mG)(mC)(dT) |
| | #(rC)(mC)(mC)(dT)#(mU)(mU)(mC)(mU)(mC)(mA)(mU)(mU)#(mU)#(mC)#(mG) |
| | (SEQ ID NO: 82); |
| CR48-203- | (mN)#(mN)#(mN)#(mN)(mN)(mN)(dN)#(mN)(mN)(mN)(mN)(fN)(mN)(fN)(mN)(fN) |
| V1.7 | (mN)(mN)(fN)(mN)(fN)(mN)(mN)(mN)(mG)(rU)(rU)(mG)(mU)(mA)(mG)(mC)(rU) |
| | (rC)(dC)#(dC)#(dT)#(mU)(mU)(mC)(mU)(mC)(mA)(mU)(mU)#(mU)#(mC)#(mG) |
| | (SEQ ID NO: 83); |
| CR48-203- | (mN)#(mN)#(mN)#(mN)(mN)(mN)(dN)#(mN)(mN)(mN)(mN)(fN)(mN)(fN)(mN)(fN) |
| V1.10 | (mN)(mN)(fN)(mN)(fN)(mN)(mN)(mN)(mG)(rU)(rU)(mG)(mU)(mA)(mG)(mC)(rU) |
| | (rC)(mC)(dC)#(dT)#(mU)(mU)(mC)(mU)(mC)(mA)(mU)(mU)#(mU)#(mC)#(mG) |
| | (SEQ ID NO: 84); |
| CR48-203- | (mN)#(mN)#(mN)#(mN)(mN)(mN)(dN)#(mN)(mN)(mN)(mN)(fN)(mN)(fN)(mN)(fN) |
| V1.12 | (mN)(mN)(fN)(mN)(fN)(mN)(mN)(mN)(mG)(rU)(rU)(mG)(mU)(mA)(mG)(mC)(rU) |
| | (rC)(mC)(mC)(dT)#(mU)(mU)(mC)(mU)(mC)(mA)(mU)(mU)#(mU)#(mC)#(mG) |
| | (SEQ ID NO: 85); |
| CR48-203- | (mN)#(mN)#(mN)#(mN)(mN)(mN)(dN)#(mN)(mN)(mN)(mN)(fN)(mN)(fN)(mN)(fN) |
| V1.18 | (mN)(mN)(fN)(mN)(fN)(mN)(mN)(mN)(mG)(rU)(rU)(mG)(mU)(mA)(mG)(mC)(dT) |
| | #(rC)(dC)#(rC)(dT)#(mU)(mU)(mC)(mU)(mC)(mA)(mU)(mU)#(mU)#(mC)#(mG) |
| | (SEQ ID NO: 86); |
| CR48-203- | (mN)#(mN)#(mN)#(mN)(mN)(mN)(dN)#(mN)(mN)(mN)(mN)(fN)(mN)(fN)(mN)(fN) |
| V1.19 | m(mN)(N)(fN)(mN)(fN)(mN)(mN)(mN)(mG)(rU)(rU)(mG)(mU)(mA)(mG)(mC)(dT) |
| | #(rC)(dC)#(dC)#(dT)#(mU)(mU)(mC)(mU)(mC)(mA)(mU)(mU)#(mU)#(mC)#(mG) |
| | (SEQ ID NO: 87); |
| CR48-203- | (mN)#(mN)#(mN)#(mN)(mN)(mN)(dN)#(mN)(mN)(mN)(mN)(fN)(mN)(fN)(mN)(fN) |
| V1.22 | (mN)(mN)(fN)(mN)(fN)(mN)(mN)(mN)(mG)(rU)(rU)(mG)(mU)(mA)(mG)(mC)(dT) |
| | #(rC)(mC)(dC)#(dT)#(mU)(mU)(mC)(mU)(mC)(mA)(mU)(mU)#(mU)#(mC)#(mG) |
| | (SEQ ID NO: 88); |
| CR48-203- | (mN)#(mN)#(mN)#(mN)(mN)(mN)(dN)#(mN)(mN)(mN)(mN)(fN)(mN)(fN)(mN)(fN) |
| V1.24 | (mN)(mN)(fN)(mN)(fN)(mN)(mN)(mN)(mG)(rU)(rU)(mG)(mU)(mA)(mG)(mC)(dT) |
| | #(rC)(mC)(mC)(dT)#(mU)(mU)(mC)(mU)(mC)(mA)(mU)(mU)#(mU)#(mC)#(mG) |
| | (SEQ ID NO: 89); |
| CR48-203- | (mN)#(mN)#(mN)#(mN)(mN)(mN)(dN)#(mN)(mN)(mN)(mN)(fN)(mN)(fN)(mN)(fN) |
| V1.30 | (mN)(mN)(fN)(mN)(fN)(mN)(mN)(mN)(mG)(dT)#(rU)(mG)(mU)(mA)(mG)(mC)(dT) |
| | #(rC)(dC)#(rC)(dT)#(mU)(mU)(mC)(mU)(mC)(mA)(mU)(mU)#(mU)#(mC)#(mG) |
| | (SEQ ID NO: 90); |
| CR48-203- | (mN)#(mN)#(mN)#(mN)(mN)(mN)(dN)#(mN)(mN)(mN)(mN)(fN)(mN)(fN)(mN)(fN) |
| V1.31 | (mN)(mN)(fN)(mN)(fN)(mN)(mN)(mN)(mG)(dT)#(rU)(mG)(mU)(mA)(mG)(mC)(dT) |
| | #(rC)(dC)#(dC)#(dT)#(mU)(mU)(mC)(mU)(mC)(mA)(mU)(mU)#(mU)#(mC)#(mG) |
| | (SEQ ID NO: 91); |
| CR48-203- | (mN)#(mN)#(mN)#(mN)(mN)(mN)(dN)#(mN)(mN)(mN)(mN)(fN)(mN)(fN)(mN)(fN) |
| V1.34 | (mN)(mN)(fN)(mN)(fN)(mN)(mN)(mN)(mG)(dT)#(rU)(mG)(mU)(mA)(mG)(mC)(dT) |
| | #(rC)(mC)(dC)#(dT)#(mU)(mU)(mC)(mU)(mC)(mA)(mU)(mU)#(mU)#(mC)#(mG) |
| | (SEQ ID NO: 92); or |
| CR48-203- | (mN)#(mN)#(mN)#(mN)(mN)(mN)(dN)#(mN)(mN)(mN)(mN)(fN)(mN)(fN)(mN)(fN) |
| V1.36 | (mN)(mN)(fN)(mN)(fN)(mN)(mN)(mN)(mG)(dT)#(rU)(mG)(mU)(mA)(mG)(mC)(dT) |
| | #(rC)(mC)(mC)(dT)#(mU)(mU)(mC)(mU)(mC)(mA)(mU)(mU)#(mU)#(mC)#(mG) |
| | (SEQ ID NO: 93) |

wherein rN=RNA, mN=2′-O-methyl RNA, fN=2′-fluoro RNA, N #N=phosphorothioate linkage, and N=any nucleotide.

67-70. (canceled)

71. The chemically modified Nme gRNA of claim 66, further comprising a nucleotide or non-nucleotide loop or linker linking the 3′ end of the crRNA portion to the 5′ end of the tracrRNA portion, optionally wherein:
the nucleotide loop is chemically modified, optionally wherein:
the nucleotide loop comprises the nucleotide sequence of GAAA, optionally wherein:
the nucleotide loop comprises the nucleotide sequence of (mG)(mA)(mA)(mA), wherein mN corresponds to a 2′-O-methyl RNA and N corresponds to any nucleotide, optionally wherein:
the non-nucleotide linker comprises an azide linker, an ethylene glycol oligomer, a tetrazine linker, an alkyl chain, a peptide, an amide, or a carbamate.

72-75. (canceled)

76. A chemically modified Neisseria meningitidis (Nme) single guide RNA (sgRNA) comprising one or more chemical modifications.

77. The chemically modified Nme sgRNA of claim 76, wherein:
the sgRNA is between 99 and 145 nucleotides in length;
the sgRNA is 145 nucleotides in length, 121 nucleotides in length, 111 nucleotides in length, 107 nucleotides in length, 105 nucleotides in length, 103 nucleotides in length, 102 nucleotides in length, 101 nucleotides in length, 100 nucleotides in length, or 99 nucleotides in length;
the one or more chemical modifications are each independently selected from a modification of a ribose group, a phosphate group, a nucleobase, or a combination thereof;
each modification of the ribose group is independently selected from the group consisting of 2′-O-methyl, 2′-fluoro, 2′-deoxy, 2′-O-(2-methoxyethyl) (MOE), 2′-NH₂ (2′-amino), 4′-thio, a bicyclic nucleotide, a locked nucleic acid (LNA), a 2′-(S)-constrained ethyl (5-cEt), a constrained MOE, and a 2′-O,4′-C-aminomethylene bridged nucleic acid (2′,4′-BNA^{NC});
at least 40%, 80%, or 100% of the ribose groups are chemically modified;
each modification of the phosphate group is independently selected from the group consisting of a phosphorothioate, phosphonoacetate (PACE), thiophosphonoacetate (thioPACE), amide, triazole, phosphonate, or phosphotriester modification; and/or
each modification of the nucleobase group is independently selected from the group consisting of 2-thiouridine, 4-thiouridine, N⁶-methyladenosine, pseudouridine, 2,6-diaminopurine, inosine, thymidine, 5-methylcytosine, 5-substituted pyrimidine, isoguanine, isocytosine, and halogenated aromatic groups.

78-85. (canceled)

86. The chemically modified Nme sgRNA of claim 77, comprising:
at least 40%, 80%, 90%, or 100% modified nucleotides;
between 2 and 100 modified nucleotides;
at least one chemical modification in the nucleotide sequence of any one of:
(SEQ ID NO: 97)
(N)ₓGUUGUAGCUCCCUUUCUCAUUUCGGAAACGAAAUGAGAACCGUUGC
UACAAUAAGGCCGUCUGAAAAGAUGUGCCGCAACGCUCUGCCCCUUAAAG
CUUCUGCUUUAAGGGGCAUCGUUUA (SG-145NT);
(SEQ ID NO: 98)
(N)ₓGUUGUAGCUCCCGAAACGUUGCUACAAUAAGGCCGUCUGAAAAGAU
GUGCCGCAACGCUCUGCCCCUUAAAGCUUCUGCUUUAAGGGGCAUCGUU
(SG-121NTv1);
(SEQ ID NO: 99)
(N)ₓGUUGUAGCUCCCGAAACGUUGCUACAAUAAGGCCGUCUGAAAAGAU
GUGCCGCAACGCUCUGCCCCUUAAAGCUUCUGCUUUAAGGGGCAUCGUU
UA (SG-121NTv2);
(SEQ ID NO: 100)
(N)ₓGUUGUAGCUCCCGAAACGUUGCUACAAUAAGGCCGUCUGAAAAGAU
GUGCCGCAACGCUCUGCCCCUUUUCUAAGGGGCAUCGUUUA
(SG-11INT);
(SEQ ID NO: 101)
(N)ₓGUUGUAGCUCCCGAAACGUUGCUACAAUAAGGCCGUCUGAAAAGAU
GUGCCGCAACGCUCUGCCCCUUUCUAGGGGCAUCGUU (SG-107NT);
(SEQ ID NO: 102)
(N)ₓGUUGUAGCUCCCGAAACGUUGCUACAAUAAGGCCGUCUGAAAAGAU
GUGCCGCAACGCUCUGCCCCUUCUGGGGCAUCGUU(SG-105NTv1):
(SEQ ID NO: 103)
(N)ₓGUUGUAGCUCCCGAAACGUUGCUACAAUAAGGCCGUCUGAAAAGAU
GUGCCGCAACGCUCUGCCCCUUUUCUAAGGGGCAU (SG-105NTv2);
(SEQ ID NO: 104)
(N)ₓGUUGUAGCUCCCGAAACGUUGCUACAAUAAGGCCGUCUGAAAAGAU
GUGCCGCAACGCUCUGCCUUCUGGCAUCGUUUA (SG-103NTv1):
(SEQ ID NO: 105)
(N)_{X}GUUGUAGCUCCCGAAACGUUGCUACAAUAAGGCCGUCUGAAAAGAU
GUGCCGCAACGCUCUGCCCUUCUGGGCAUCGUU (SG-103NTV2);
(SEQ ID NO: 106)
(N)ₓGUUGUAGCUCCCGAAACGUUGCUACAAUAAGGCCGUCUGAAAAGAU
GUGCCGCAACGCUCUGCUUCUGCAUCGUUUA (SG-101NTv1);
or
(SEQ ID NO: 107)
(N)ₓGUUGUAGCUCCCGAAACGUUGCUACAAUAAGGCCGUCUGAAAAGAU
GUGCCGCAACGCUCUGCUUCUGCAUCGUU (SG-99NT),
wherein N corresponds to any nucleotide and x corresponds to any integer between 18 and 26.

87-91. (canceled)

92. A chemically modified Neisseria meningitidis (Nme) single guide RNA (sgRNA) comprising a chemical modification pattern selected from anyone of:
(SEQ ID NO: 108)
(mN)#(mN)#(mN)#(rN)(rN)(rN)(rN)(rN)(rN)(rN)(rN)
(rN)(rN)(rN)(rN)(rN)(rN)(rN)(rN)(rN)(rN)(rN)
(rN)(rN)(rG)(rU)(rU)(rG)(rU)(rA)(rG)(rC)(rU)(rC)
(rC)(rC)(mG)(mA)(mA)(mA)(rC)(rG)(rU)(rU)(rG)(rC)
(rU)(rA)(rC)(rA)(rA)(rU)(rA)(rA)(rG)(rG)(rC)(rC)
(rG)(rU)(mC)(mU)(mG)(mA)(mA)(mA)(mA)(mG)(rA)(rU)
(rG)(rU)(rG)(rC)(rC)(rG)(rC)(rA)(rA)(rC)(rG)(rC)
(rU)(rC)(rU)(rG)(rC)(rC)(mU)(mU)(mC)(mU)(rG)(rG)
(rC)(rA)(rU)(rC)(rG)(rU)#(mU)#(mU)#(mA)(SG-1);
(SEQ ID NO: 109)
(mN)#(mN)#(mN)#(rN)(rN)(rN)(rN)(rN)(rN)(rN)(rN)
(rN)(rN)(rN)(rN)(rN)(rN)(rN)(rN)(rN)(rN)(rN)
(rN)(rN)(rG)(rU)(rU)(rG)(rU)(mA)(mG)(mC)(rU)(rC)
(rC)(rC)(mG)(mA)(mA)(mA)(rC)(rG)(rU)(rU)(rG)(rC)
(rU)(rA)(rC)(rA)(rA)(rU)(rA)(rA)(rG)(rG)(mC)(mC)
(rG)(mU)(mC)(mU)(mG)(mA)(mA)(mA)(mA)(mG)(rA)(rU)
(rG)(rU)(rG)(rC)(rC)(rG)(rC)(rA)(rA)(rC)(rG)(rC)
(mU)(mC)(mU)(mG)(rC)(rC)(mU)(mU)(mC)(mU)(rG)(rG)
(rC)(rA)(rU)(mC)(mG)(mU)#(mU)#(mU)#(mA)(SG-2);
(SEQ ID NO: 110)
(mN)#(mN)#(mN)#(mN)(mN)(rN)(rN)(rN)(mN)(mN)(mN)
(rN)(mN)(rN)(rN)(rN)(rN)(mN)(rN)(rN)(rN)(mN)(rN)
(rN)(mG)(rU)(rU)(rG)(mU)(mA)(mG)(mC)(rU)(rC)(rC)
(rC)(mG)(mA)(mA)(mA)(mC)(rG)(mU)(rU)(mG)(mC)(rU)
(mA)(mC)(rA)(rA)(rU)(rA)(mA)(rG)(mG)(mC)(mC)(mG)
(mU)(mC)(mU)(mG)(mA)(mA)(mA)(mA)(mG)(mA)(mU)(rG)
(mU)(mG)(mC)(mC)(mG)(mC)(mA)(mA)(mC)(mG)(mC)(mU)
(mC)(mU)(mG)(mC)(mC)(mU)(mU)(mC)(mU)(rG)(mG)(rC)
(mA)(mU)(mC)(mG)(mU)#(mU)#(mU)#(mA)(SG-3);
(SEQ ID NO: 111)
(mN)#(mN)#(mN)#(mN)(mN)(rN)(rN)(rN)(mN)(mN)(mN)
(rN)(mN)(rN)(rN)(mN)(rN)(mN)(rN)(rN)(rN)(mN)(rN)
(rN)(mG)(rU)(rU)(rG)(mU)(mA)(mG)(mC)(rU)(rC)(rC)
(rC)(mG)(mA)(mA)(mA)(mC)(rG)(mU)(rU)(mG)(mC)(rU)
(mA)(mC)(rA)(rA)(rU)(rA)(mA)(rG)(mG)(fC)(mC)(fG)
(mU)(fC)(mU)(fG)(mA)(fA)(mA)(fA)(mG)(fA)(mU)(rG)
(mU)(fG)(mC)(fC)(mG)(fC)(mA)(fA)(mC)(fG)(mC)(fU)
(mC)(fU)(mG)(fC)(mC)(fU)(mU)(fC)(mU)(rG)(mG)(rC)
(mA)(fU)(mC)(fG)(mU)#(mU)#(mU)#(mA)(SG-4);
(SEQ ID NO: 112)
(mN)#(mN)#(mN)#(mN)(mN)(rN)(rN)(rN)(mN)(mN)(mN)
(rN)(mN)(rN)(rN)(rN)(rN)(mN)(rN)(rN)(rN)(mN)(rN)
(rN)(mG)(rU)(rU)(rG)(mU)(mA)(mG)(mC)(rU)(rC)(rC)
(rC)(mG)(mA)(mA)(mA)(rC)(rG)(rU)(rU)(rG)(rC)(rU)
(rA)(rC)(rA)(rA)(rU)(rA)(rA)(rG)(rG)(mC)(mC)(rG)
(mU)(mC)(mU)(mG)(mA)(mA)(mA)(mA)(mG)(rA)(rU)(rG)
(rU)(rG)(rC)(rC)(rG)(rC)(rA)(rA)(rC)(rG)(rC)(mU)
(mC)(mU)(mG)(rC)(rC)(mU)(mU)(mC)(mU)(rG)(rG)(rC)
(rA)(rU)(mC)(mG)(mU)#(mU)#(mU)#(mA)(SG-5);
(SEQ ID NO: 113)
(mN)#(mN)#(mN)#(rN)(mN)(rN)(rN)(rN)(mN)(mN)(rN)
(rN)(mN)(rN)(rN)(rN)(rN)(mN)(rN)(rN)(rN)(mN)(rN)
(rN)(mG)(rU)(rU)(rG)(rU)(mA)(mG)(mC)(rU)(rC)(rC)
(rC)(mG)(mA)(mA)(mA)(rC)(rG)(rU)(rU)(rG)(rC)(rU)
(rA)(rC)(rA)(rA)(rU)(rA)(rA)(rG)(rG)(mC)(mC)(rG)
(mU)(mC)(mU)(mG)(mA)(mA)(mA)(mA)(mG)(rA)(rU)(rG)
(rU)(rG)(rC)(rC)(rG)(rC)(rA)(rA)(rC)(rG)(rC)(mU)
(mC)(mU)(mG)(rC)(rC)(mU)(mU)(mC)(mU)(rG)(rG)(rC)
(rA)(rU)(mC)(mG)(mU)#(mU)#(mU)#(mA)(SG-6);
(SEQ ID NO: 114)
(mN)#(mN)#(mN)#(rN)(rN)(rN)(rN)(rN)(rN)(rN)(rN)
(rN)(rN)(rN)(rN)(rN)(rN)(mN)(rN)(rN)(rN)(mN)(rN)
(rN)(rG)(rU)(rU)(rG)(rU)(mA)(mG)(mC)(rU)(rC)(rC)
(rC)(mG)(mA)(mA)(mA)(rC)(rG)(rU)(rU)(rG)(rC)(rU)
(rA)(rC)(rA)(rA)(rU)(rA)(rA)(rG)(rG)(mC)(mC)(rG)
(mU)(mC)(mU)(mG)(mA)(mA)(mA)(mA)(mG)(rA)(rU)(rG)
(rU)(rG)(rC)(rC)(rG)(rC)(rA)(rA)(rC)(rG)(rC)(mU)
(mC)(mU)(mG)(rC)(rC)(mU)(mU)(mC)(mU)(rG)(rG)(rC)
(rA)(rU)(mC)(mG)(mU)#(mU)#(mU)#(mA)(SG-7);
(SEQ ID NO: 115)
(mN)#(mN)#(mN)#(mN)(mN)(mN)(mN)(mN)(mN)(mN)(mN)
(mN)(mN)(mN)(rN)(rN)(rN)(rN)(rN)(rN)(rN)(rN)(rN)
(rN)(rG)(rU)(rU)(rG)(rU)(mA)(mG)(mC)(rU)(rC)(rC)
(rC)(mG)(mA)(mA)(mA)(rC)(rG)(rU)(rU)(rG)(rC)(rU)
(rA)(rC)(rA)(rA)(rU)(rA)(rA)(rG)(rG)(mC)(mC)(rG)
(mU)(mC)(mU)(mG)(mA)(mA)(mA)(mA)(mG)(rA)(rU)(rG)
(rU)(rG)(rC)(rC)(rG)(rC)(rA)(rA)(rC)(rG)(rC)(mU)
(mC)(mU)(mG)(rC)(rC)(mU)(mU)(mC)(mU)(rG)(rG)(rC)
(rA)(rU)(mC)(mG)(mU)#(mU)#(mUX#(mA)(SG-8);
(SEQ ID NO: 116)
(mN)#(mN)#(mN)#(mN)(mN)(mN)(mN)(mN)(mN)(mN)(mN)
(mN)(mN)(mN)(rN)(rN)(rN)(mN)(rN)(rN)(rN)(mN)(rN)
(rN)(mG)(rU)(rU)(rG)(mU)(mA)(mG)(mC)(rU)(rC)(rC)
(rC)(mG)(mA)(mA)(mA)(rC)(rG)(rU)(rU)(rG)(rC)(rU)
(rA)(rC)(rA)(rA)(rU)(rA)(rA)(rG)(rG)(mC)(mC)(rG)
(mU)(mC)(mU)(mG)(mA)(mA)(mA)(mA)(mG)(rA)(rU)(rG)
(rU)(rG)(rC)(rC)(rG)(rC)(rA)(rA)(rC)(rG)(rC)(mU)
(mC)(mU)(mG)(rC)(rC)(mU)(mU)(mC)(mU)(rG)(rG)(rC)
(rA)(rU)(mC)(mG)(mU)#(mU)#(mU)#(mA)(SG-9);
(SEQ ID NO: 117)
(mN)#(mN)#(mN)#(mN)(mN)(rN)(rN)(rN)(mN)(mN)(mN)
(rN)(mN)(rN)(rN)(rN)(rN)(mN)(rN)(rN)(rN)(mN)(rN)
(rN)(mG)(rU)(rU)(rG)(mU)(mA)(mG)(mC)(rU)(rC)(rC)
(rC)(mG)(mA)(mA)(mA)(rC)(rG)(rU)(rU)(rG)(rC)(rU)
(rA)(rC)(rA)(rA)(rU)(rA)(mA)(rG)(mG)(mC)(mC)(mG)
(mU)(mC)(mU)(mG)(mA)(mA)(mA)(mA)(mG)(mA)(mU)(rG)
(rU)(mG)(mC)(mC)(rG)(rC)(rA)(rA)(rC)(rG)(rC)(mU)
(mC)(mU)(mG)(rC)(rC)(mU)(mU)(mC)(mU)(rG)(rG)(rC)
(rA)(rU)(mC)(mG)(mU)#(mU)#(mU)#(mA)(SG-10);
(SEQ ID NO: 118)
(mN)#(mN)#(mN)#(mN)(mN)(rN)(rN)(rN)(mN)(mN)(mN)
(rN)(mN)(rN)(rN)(rN)(rN)(mN)(rN)(rN)(rN)(mN)(rN)
(rN)(mG)(rU)(rU)(rG)(mU)(mA)(mG)(mC)(rU)(rC)(rC)
(rC)(mG)(mA)(mA)(mA)(rC)(rG)(rU)(rU)(rG)(rC)(rU)
(rA)(rC)(rA)(rA)(rU)(rA)(rA)(rG)(rG)(mC)(mC)(rG)
(mU)(mC)(mU)(mG)(mA)(mA)(mA)(mA)(mG)(rA)(rU)(rG)
(rU)(rG)(rC)(rC)(mG)(rC)(mA)(mA)(mC)(mG)(mC)(mU)
(mC)(mU)(mG)(mC)(mC)(mU)(mU)(mC)(mU)(rG)(mG)(rC)
(mA)(rU)(mC)(mG)(mU)#(mU)#(mU)#(mA)(SG-11);
(SEQ ID NO: 119)
(mN)#(mN)#(mN)#(mN)(mN)(rN)(rN)(rN)(mN)(mN)(mN)
(rN)(mN)(rN)(rN)(rN)(rN)(mN)(rN)(rN)(rN)(mN)(rN)
(rN)(fG)(rU)(rU)(rG)(fU)(fA)(fG)(fC)(rU)(rC)(rC)
(rC)(mG)(mA)(mA)(mA)(rC)(rG)(rU)(rU)(rG)(rC)(rU)
(rA)(rC)(rA)(rA)(rU)(rA)(rA)(rG)(rG)(fC)(fC)(rG)
(fU)(fC)(fU)(mG)(mA)(mA)(mA)(fA)(fG)(rA)(rU)(rG)
(rU)(rG)(rC)(rC)(rG)(rC)(rA)(rA)(rC)(rG)(rC)(mU)
(mC)(fU)(fG)(rC)(rC)(mU)(mU)(mC)(mU)(rG)(rG)(rC)
(rA)(rU)(fC)(fG)(fU)#(fU)#(mU)#(mA)(SG-12);
(SEQ ID NO: 120)
(mN)#(mN)#(mN)#(mN)(mN)(rN)(rN)(rN)(mN)(mN)(mN)
(rN)(mN)(rN)(rN)(rN)(rN)(mN)(rN)(rN)(rN)(mN)(rN)
(rN)(fG)(rU)(rU)(rG)(fU)(fA)(fG)(fC)(rU)(rC)(rC)
(rC)(mG)(mA)(mA)(mA)(mC)(rG)(mU)(rU)(fG)(fC)(rU)
(fA)(fC)(rA)(rA)(rU)(rA)(rA)(rG)(rG)(mC)(mC)(rG)
(mU)(mC)(mU)(mG)(mA)(mA)(mA)(mA)(mG)(rA)(rU)(rG)
(rU)(rG)(rC)(rC)(rG)(rC)(rA)(rA)(rC)(rG)(rC)(mU)
(mC)(mU)(mG)(rC)(rC)(mU)(mU)(mC)(mU)(rG)(rG)(rC)
(rA)(rU)(mC)(mG)(mU)#(mU)#(mU)#(mA)(SG-13);
(SEQ ID NO: 121)
(mN)#(mN)#(mN)#(mN)(mN)(rN)(rN)(rN)(mN)(mN)(mN)
(rN)(mN)(rN)(rN)(rN)(rN)(mN)(rN)(rN)(rN)(mN)(rN)
(rN)(fG)(rU)(rU)(rG)(fU)(fA)(fG)(fC)(rU)(rC)(rC)
(rC)(mG)(mA)(mA)(mA)(mC)(rG)(mU)(rU)(fG)(fC)(rU)
(fA)(fC)(rA)(rA)(rU)(rA)(rA)(rG)(rG)(fC)(fC)(rG)
(fU)(fC)(fU)(mG)(mA)(mA)(mA)(fA)(fG)(rA)(rU)(rG)
(rU)(rG)(rC)(rC)(rG)(rC)(rA)(rA)(rC)(rG)(rC)(mU)
(mC)(fU)(fG)(rC)(rC)(mU)(mU)(mC)(mU)(rG)(rG)(rC)
(rA)(rU)(fC)(fG)(fU)#(fU)#(mU)#(mA)(SG-14);
(SEQ ID NO: 122)
mN#mN#mN#rNrNrNrNrNrNrNrNrNrNrNrNrNrNrNrNrNrNr
NrNrNrGrUrUrGrUrArGrCrUrCrCrCrGrArArArCrGrUrUr
GrCrUrArCrArArUrArArGrGrCrCrGrUrCrUrGrArArArAr
GrArUrGrUrGrCrCrGrCrArArCrGrCrUrCrUrGrCrCrCrCr
UrUrArArArGrCrUrCrCrUrGrCrUrUrUrArArGrGrGrGrCr
ArUrCrGrUmU#mU#mA(SG121-0);
(SEQ ID NO: 123)
mN#mN#mN#mNmNrNrNrNrNrNrNrNrNrNrNrNrNrNrNrNrNr
NrNrNrGrUrUrGrUrArGrCrUrCrCrCmGmAmAmArCrGrUrUr
GrCrUrArCrArArUrArArGrGrCrCrGmUmCmUmGmAmAmAmAm
GrArUrGrUrGrCrCrGrCrArArCrGrCrUrCrUrGrCrCmCmCm
UmUmAmAmAmGmCmUmCmCmUmGmCmUmUmUmAmAmGmGrGrGrCr
ArUmCmGmU#mU#mU#mA(SG121-2);
(SEQ ID NO: 124)
mN#mN#mN#mNmNrNrNrNmNmNmNrNmNrNrNrNrNmNrNrNrNm
NrNrNmGrUrUrGmUmAmGmCrUrCrCrCmGmAmAmArCrGrUrUr
GrCrUrArCrArArUrArArGrGrCrCrGmUmCmUmGmAmAmAmAm
GrArUrGrUrGrCrCrGrCrArArCrGrCrUrCrUrGrCrCmCmCm
UmUmAmAmAmGmCmUmCmCmUmGmCmUmUmUmAmAmGmGrGrGrCr
ArUmCmGmU#mU#mU#mA(SG121-3);
and
(SEQ ID NO: 125)
mN#mN#mN#mNmNmNmNmNmNmNmNmNmNmNrNrNrNmNrNrNrNm
NrNrNmGrUrUrGmUmAmGmCrUrCrCrCmGmAmAmArCrGrUrUr
GrCrUrArCrArArUrArArGrGrCrCrGmUmCmUmGmAmAmAmAm
GrArUrGrUrGrCrCrGrCrArArCrGrCrUrCrUrGrCrCmCmCm
UmUmAmAmAmGmCmUmCmCmUmGmCmUmUmUmAmAmGmGrGrGrCr
ArUmCmGmU#mU#mU#mA(SG121-4),
wherein rN=RNA, mN=2′-O-methyl RNA, fN=2′-fluoro RNA, N #N=phosphorothioate linkage, and N=any nucleotide.

93. The chemically modified Nme gRNA of claim 1, further comprising at least one moiety conjugated to the gRNA or sgRNA, optionally wherein:
the at least one moiety is conjugated to at least one of the 5′ end of the crRNA portion, the 3′ end of the crRNA portion, the 5′ end of the tracrRNA portion, the 3′ end of the tracrRNA portion, the 5′ end of the sgRNA, and the 3′ end of the sgRNA, optionally wherein:
the at least one moiety increases cellular uptake of the gRNA or sgRNA, optionally wherein:
the at least one moiety promotes specific tissue distribution of the gRNA or sgRNA, optionally wherein:
the at least one moiety is selected from the group consisting of fatty acids, steroids, secosteroids, lipids, gangliosides analogs, nucleoside analogs, endocannabinoids, vitamins, receptor ligands, peptides, aptamers, and alkyl chains, optionally wherein:
the at least one moiety is selected from the group consisting of cholesterol, docosahexaenoic acid (DHA), docosanoic acid (DCA), lithocholic acid (LA), GalNAc, amphiphilic block copolymer (ABC), hydrophilic block copolymer (HBC), poloxamer, Cy5, and Cy3, optionally wherein:
the at least one moiety is conjugated to the gRNA or sgRNA via a linker, optionally wherein:
the linker is selected from the group consisting of an ethylene glycol chain, an alkyl chain, a polypeptide, a polysaccharide, and a block copolymer, optionally wherein:
the at least one moiety is a modified lipid, optionally wherein:
modified lipid is a branched lipid, optionally wherein:
modified lipid is a branched lipid of Formula I,
X-MC(═Y)M-Z-[L-MC(═Y)M-R]n, Formula I:
where X is a moiety that links the lipid to the gRNA or sgRNA, each Y is independently oxygen or sulfur, each M is independently CH₂, NH, O or S, Z is a branching group which allows two or three (“n”) chains to be joined to a chemically modified guide RNA, L is an optional linker moiety, and each R is independently a saturated, monounsaturated or polyunsaturated linear or branched moiety from 2 to 30 atoms in length, a sterol, or other hydrophobic group, optionally wherein:
modified lipid is a headgroup-modified lipid, optionally wherein:
modified lipid is a headgroup-modified lipid of Formula II,
X-MC(═Y)M-Z-[L-MC(═Y)M-R]n-L-K-J, Formula II:
where X is a moiety that links the lipid to the gRNA or sgRNA, each Y is independently oxygen or sulfur, each M is independently CH₂, NH, N-alkyl, 0 or S, Z is a branching group which allows two or three (“n”) chains to be joined to chemically modified guide RNA, each L is independently an optional linker moiety, and R is a saturated, monounsaturated or polyunsaturated linear or branched moiety from 2 to 30 atoms in length, a sterol, or other hydrophobic group, K is a phosphate, sulfate, or amide and J is an aminoalkane or quaternary aminoalkane group.

94-105. (canceled)

106. The chemically modified Nme gRNA of claim 1, wherein the guide RNA binds to a N. meningitidis Cas9 nuclease (NmeCas9) or variant thereof, optionally wherein:
the Nme Cas9 nuclease or variant thereof is Nme1Cas9, Nme2Cas9, or Nme3Cas9, optionally wherein:
the Nme Cas9 nuclease or variant thereof comprises an amino acid sequence with at least 80% identity to an amino acid sequence set forth in SEQ ID NO: 1, 2, or 3, optionally wherein:
the NmeCas9 further comprises one or more nuclear localization signal (NLS) polypeptides, optionally wherein:
the NLS polypeptide comprises one or both of a nucleoplasmin NLS and an SV40 NLS, optionally wherein:
the NmeCas9 is a variant NmeCas9, optionally wherein:
wherein the variant NmeCas9 comprises one or both of a D16A mutation and a H588A mutation, optionally wherein:
wherein the NmeCas9 or variant NmeCas9 is fused to a nucleotide deaminase, optionally wherein:
the nucleotide deaminase is a cytidine deaminase, optionally wherein:
the nucleotide deaminase is an adenosine deaminase, optionally wherein:
NmeCas9 or variant NmeCas9 further comprises a uracil glycosylase inhibitor, optionally wherein:
Cas9 off-target activity is reduced relative to an unmodified gRNA or sgRNA, optionally wherein:
Cas9 on-target activity is increased relative to an unmodified gRNA or sgRNA, optionally wherein:
the chemically modified Nme gRNA or sgRNA comprises at least about 50% activity relative to an unmodified gRNA or sgRNA.

107-119. (canceled)

120. A method of altering expression of a target gene in a cell, comprising contacting the cell with a genome editing system comprising:
the chemically modified Nme gRNA of claim 1; and
an RNA-guided nuclease or a polynucleotide encoding an RNA-guided nuclease.

121. The method of claim 120, wherein:
the target gene is in a cell in an organism;
the contacting occurs in vivo or ex vivo or in vitro;
expression of the target gene is knocked out or knocked down;
the sequence of the target gene is modified, edited, corrected or enhanced;
the guide RNA and the RNA-guided nuclease comprise a ribonucleoprotein (RNP) complex; and/or
the RNA-guided nuclease is an N. meningitidis Cas9 (NmeCas9), optionally wherein:
the Nme Cas9 is Nme1Cas9, Nme2Cas9, or Nme3Cas9, optionally wherein:
the NmeCas9 further comprises one or more nuclear localization signal (NLS) polypeptides, optionally wherein:
the NLS polypeptide comprises one or both of a nucleoplasmin NLS and an SV40 NLS;
the NmeCas9 is a variant NmeCas9, optionally wherein:
the variant NmeCas9 comprises one or both of a D16A mutation and a H588A mutation, optionally wherein:
the NmeCas9 or variant NmeCas9 is fused to a nucleotide deaminase, optionally wherein:
the nucleotide deaminase is a cytidine deaminase or an adenosine deaminase, optionally wherein:
NmeCas9 or variant NmeCas9 further comprises a uracil glycosylase inhibitor, optionally wherein:
the polynucleotide encoding an RNA-guided nuclease comprises a vector, optionally wherein:
the vector further comprises a polynucleotide encoding a crRNA portion or a tracrRNA portion, optionally wherein:
the vector is a viral vector, optionally wherein:
the viral vector is an adeno-associated virus (AAV) vector, an adenovirus vector, or a lentivirus (LV) vector, optionally wherein:
the polynucleotide encoding an RNA-guided nuclease comprises an mRNA, optionally wherein:
the mRNA is formulated in a lipid nanoparticle (LNP), optionally wherein:
the LNP comprises at least one cationic lipid, optionally wherein:
the LNP further comprises a PEGylated lipid, a helper lipid, and cholesterol or derivative thereof, optionally wherein:
expression of the target gene is reduced by at least about 20%.

122-145. (canceled)

146. A CRISPR genome editing system comprising:
a chemically modified gRNA of claim 1; and
an RNA-guided nuclease or a polynucleotide encoding an RNA-guided nuclease.

147. (canceled)

148. A ribonucleoprotein (RNP) complex comprising:
i) a Neisseria meningitidis (Nme) Cas9 nuclease; and
ii) a chemically modified Nme guide RNA (gRNA), wherein the Nme gRNA comprises:
(a) a crRNA portion comprising (i) a guide sequence capable of hybridizing to a target polynucleotide sequence, and (ii) a repeat sequence; and
(b) a tracrRNA portion comprising an anti-repeat nucleotide sequence that is complementary to the repeat sequence,
wherein the crRNA portion comprises at least 40% modified nucleotides.

149. The RNP complex of claim 148, wherein the gRNA is capable of binding to the Nme Cas9 nuclease or variant thereof and/or wherein the Nme Cas9 or variant thereof is Nme1Cas9, Nme2Cas9, or Nme3Cas9.

150. (canceled)
